SEQUENCE LISTING (226 sequences)

SEQ ID NO: 1 - protein, 20 aa, synthetic construct
MRSLSVLALLLLLLLAPASA

SEQ ID NO: 2 - RNA, 774 nt, synthetic construct
  source 1..774: mol_type = other RNA
atgcgttctttatctgttttagctttattattattattattattagctcctgcttctgcttctggtttacgtattaattctgctaaagatgatgctgctggtcaagctgctgctaatcgtgctacttctaatattaaaggtttaactcaagcttctcgtaatgctgctgatggtatttctattgctcaaactactgaaggtgctttaaatgaaattaataataatttacaacgtgttcgtgaattatctgttcaagctactgctggtgctaatgctgatgctgctttaaaagctattcaagctgaaattcaacaacgtttagaagaaattgatcgtgtttctcaacaaactcaagctgctgctgttaaagttttatctcaagataatgctatggctattcaagttggtgctaatgatggtgctgctattactattgatttacaaaaaattgatgttaaatctttaggtttagatggttttaatgttaattctcctggttctactgctaatcctttagcttctattgattctgctttatctaaagttgatgctgttcgttcttctttaggtgctattcaaaatcgttttgattctgctattactaatttaggtaatactgttactaatttaaattctgctcgttctcgtattgaagatgctgattatgctactgaagtttctcaaatgtctaaagctcaaattttagatcaagctggtacttctactttagctcaattagttcctcgtggttctcatcatcatcatcatcatggt

SEQ ID NO: 3 - RNA, 774 nt, synthetic construct
  source 1..774: mol_type = other RNA
atgcgctccttgtccgtcttggccttgttgttgttgttgttgttggcccccgcctccgcctccggcttgcgcatcaactccgccaaggacgacgccgccggccaggccgccgccaaccgcgccacctccaacatcaagggcttgacccaggcctcccgcaacgccgccgacggcatctccatcgcccagaccaccgagggcgccttgaacgagatcaacaacaacttgcagcgcgtccgcgagttgtccgtccaggccaccgccggcgccaacgccgacgccgccttgaaggccatccaggccgagatccagcagcgcttggaggagatcgaccgcgtctcccagcagacccaggccgccgccgtcaaggtcttgtcccaggacaacgccatggccatccaggtcggcgccaacgacggcgccgccatcaccatcgacttgcagaagatcgacgtcaagtccttgggcttggacggcttcaacgtcaactcccccggctccaccgccaaccccttggcctccatcgactccgccttgtccaaggtcgacgccgtccgctcctccttgggcgccatccagaaccgcttcgactccgccatcaccaacttgggcaacaccgtcaccaacttgaactccgcccgctcccgcatcgaggacgccgactacgccaccgaggtctcccagatgtccaaggcccagatcttggaccaggccggcacctccaccttggcccagttggtcccccgcggctcccaccaccaccaccaccacggc

SEQ ID NO: 4 - RNA, 774 nt, synthetic construct
  source 1..774: mol_type = other RNA
atgcgatcactttcagtacttgcacttcttcttcttcttcttcttgcaccagcatcagcatcaggacttcgaataaattcagcaaaagatgatgcagcaggacaagcagcagcaaatcgagcaacatcaaatataaaaggacttacacaagcatcacgaaatgcagcagatggaatatcaatagcacaaacaacagaaggagcacttaatgaaataaataataatcttcaacgagtacgagaactttcagtacaagcaacagcaggagcaaatgcagatgcagcacttaaagcaatacaagcagaaatacaacaacgacttgaagaaatagatcgagtatcacaacaaacacaagcagcagcagtaaaagtactttcacaagataatgcaatggcaatacaagtaggagcaaatgatggagcagcaataacaatagatcttcaaaaaatagatgtaaaatcacttggacttgatggatttaatgtaaattcaccaggatcaacagcaaatccacttgcatcaatagattcagcactttcaaaagtagatgcagtacgatcatcacttggagcaatacaaaatcgatttgattcagcaataacaaatcttggaaatacagtaacaaatcttaattcagcacgatcacgaatagaagatgcagattatgcaacagaagtatcacaaatgtcaaaagcacaaatacttgatcaagcaggaacatcaacacttgcacaacttgtaccacgaggatcacatcatcatcatcatcatgga

SEQ ID NO: 5 - RNA, 774 nt, synthetic construct
  source 1..774: mol_type = other RNA
atgcggtcgctctcggtgctcgcgctcctcctcctcctcctcctcgcgccggcgtcggcgtcggggctccggattaattcggcgaaagatgatgcggcggggcaagcggcggcgaatcgggcgacgtcgaatattaaagggctcacgcaagcgtcgcggaatgcggcggatgggatttcgattgcgcaaacgacggaaggggcgctcaatgaaattaataataatctccaacgggtgcgggaactctcggtgcaagcgacggcgggggcgaatgcggatgcggcgctcaaagcgattcaagcggaaattcaacaacggctcgaagaaattgatcgggtgtcgcaacaaacgcaagcggcggcggtgaaagtgctctcgcaagataatgcgatggcgattcaagtgggggcgaatgatggggcggcgattacgattgatctccaaaaaattgatgtgaaatcgctcgggctcgatgggtttaatgtgaattcgccggggtcgacggcgaatccgctcgcgtcgattgattcggcgctctcgaaagtggatgcggtgcggtcgtcgctcggggcgattcaaaatcggtttgattcggcgattacgaatctcgggaatacggtgacgaatctcaattcggcgcggtcgcggattgaagatgcggattatgcgacggaagtgtcgcaaatgtcgaaagcgcaaattctcgatcaagcggggacgtcgacgctcgcgcaactcgtgccgcgggggtcgcatcatcatcatcatcatggg

SEQ ID NO: 6 - RNA, 774 nt, synthetic construct
  source 1..774: mol_type = other RNA
atgagaagtctaagtgttctagctctactactactactactactagctcctgctagtgctagtggtctaagaattaatagtgctaaagatgatgctgctggtcaagctgctgctaatagagctactagtaatattaaaggtctaactcaagctagtagaaatgctgctgatggtattagtattgctcaaactactgaaggtgctctaaatgaaattaataataatctacaaagagttagagaactaagtgttcaagctactgctggtgctaatgctgatgctgctctaaaagctattcaagctgaaattcaacaaagactagaagaaattgatagagttagtcaacaaactcaagctgctgctgttaaagttctaagtcaagataatgctatggctattcaagttggtgctaatgatggtgctgctattactattgatctacaaaaaattgatgttaaaagtctaggtctagatggttttaatgttaatagtcctggtagtactgctaatcctctagctagtattgatagtgctctaagtaaagttgatgctgttagaagtagtctaggtgctattcaaaatagatttgatagtgctattactaatctaggtaatactgttactaatctaaatagtgctagaagtagaattgaagatgctgattatgctactgaagttagtcaaatgagtaaagctcaaattctagatcaagctggtactagtactctagctcaactagttcctagaggtagtcatcatcatcatcatcatggt

SEQ ID NO: 7 - RNA, 774 nt, synthetic construct
  source 1..774: mol_type = other RNA
atgaggagcctgagcgttctggctctgctgctgctgctgctgctggctcctgctagcgctagcggtctgaggattaatagcgctaaagatgatgctgctggtcaagctgctgctaatagggctactagcaatattaaaggtctgactcaagctagcaggaatgctgctgatggtattagcattgctcaaactactgaaggtgctctgaatgaaattaataataatctgcaaagggttagggaactgagcgttcaagctactgctggtgctaatgctgatgctgctctgaaagctattcaagctgaaattcaacaaaggctggaagaaattgatagggttagccaacaaactcaagctgctgctgttaaagttctgagccaagataatgctatggctattcaagttggtgctaatgatggtgctgctattactattgatctgcaaaaaattgatgttaaaagcctgggtctggatggttttaatgttaatagccctggtagcactgctaatcctctggctagcattgatagcgctctgagcaaagttgatgctgttaggagcagcctgggtgctattcaaaataggtttgatagcgctattactaatctgggtaatactgttactaatctgaatagcgctaggagcaggattgaagatgctgattatgctactgaagttagccaaatgagcaaagctcaaattctggatcaagctggtactagcactctggctcaactggttcctaggggtagccatcatcatcatcatcatggt

SEQ ID NO: 8 - RNA, 717 nt, synthetic construct
  source 1..717: mol_type = other RNA
atgtctggtttacgtattaattctgctaaagatgatgctgctggtcaagctgctgctaatcgtgctacttctaatattaaaggtttaactcaagcttctcgtaatgctgctgatggtatttctattgctcaaactactgaaggtgctttaaatgaaattaataataatttacaacgtgttcgtgaattatctgttcaagctactgctggtgctaatgctgatgctgctttaaaagctattcaagctgaaattcaacaacgtttagaagaaattgatcgtgtttctcaacaaactcaagctgctgctgttaaagttttatctcaagataatgctatggctattcaagttggtgctaatgatggtgctgctattactattgatttacaaaaaattgatgttaaatctttaggtttagatggttttaatgttaattctcctggttctactgctaatcctttagcttctattgattctgctttatctaaagttgatgctgttcgttcttctttaggtgctattcaaaatcgttttgattctgctattactaatttaggtaatactgttactaatttaaattctgctcgttctcgtattgaagatgctgattatgctactgaagtttctcaaatgtctaaagctcaaattttagatcaagctggtacttctactttagctcaattagttcctcgtggttctcatcatcatcatcatcatggt

SEQ ID NO: 9 - RNA, 717 nt, synthetic construct
  source 1..717: mol_type = other RNA
atgtccggcttgcgcatcaactccgccaaggacgacgccgccggccaggccgccgccaaccgcgccacctccaacatcaagggcttgacccaggcctcccgcaacgccgccgacggcatctccatcgcccagaccaccgagggcgccttgaacgagatcaacaacaacttgcagcgcgtccgcgagttgtccgtccaggccaccgccggcgccaacgccgacgccgccttgaaggccatccaggccgagatccagcagcgcttggaggagatcgaccgcgtctcccagcagacccaggccgccgccgtcaaggtcttgtcccaggacaacgccatggccatccaggtcggcgccaacgacggcgccgccatcaccatcgacttgcagaagatcgacgtcaagtccttgggcttggacggcttcaacgtcaactcccccggctccaccgccaaccccttggcctccatcgactccgccttgtccaaggtcgacgccgtccgctcctccttgggcgccatccagaaccgcttcgactccgccatcaccaacttgggcaacaccgtcaccaacttgaactccgcccgctcccgcatcgaggacgccgactacgccaccgaggtctcccagatgtccaaggcccagatcttggaccaggccggcacctccaccttggcccagttggtcccccgcggctcccaccaccaccaccaccacggc

SEQ ID NO: 10 - RNA, 717 nt, synthetic construct
  source 1..717: mol_type = other RNA
atgtcaggacttcgaataaattcagcaaaagatgatgcagcaggacaagcagcagcaaatcgagcaacatcaaatataaaaggacttacacaagcatcacgaaatgcagcagatggaatatcaatagcacaaacaacagaaggagcacttaatgaaataaataataatcttcaacgagtacgagaactttcagtacaagcaacagcaggagcaaatgcagatgcagcacttaaagcaatacaagcagaaatacaacaacgacttgaagaaatagatcgagtatcacaacaaacacaagcagcagcagtaaaagtactttcacaagataatgcaatggcaatacaagtaggagcaaatgatggagcagcaataacaatagatcttcaaaaaatagatgtaaaatcacttggacttgatggatttaatgtaaattcaccaggatcaacagcaaatccacttgcatcaatagattcagcactttcaaaagtagatgcagtacgatcatcacttggagcaatacaaaatcgatttgattcagcaataacaaatcttggaaatacagtaacaaatcttaattcagcacgatcacgaatagaagatgcagattatgcaacagaagtatcacaaatgtcaaaagcacaaatacttgatcaagcaggaacatcaacacttgcacaacttgtaccacgaggatcacatcatcatcatcatcatgga

SEQ ID NO: 11 - RNA, 717 nt, synthetic construct
  source 1..717: mol_type = other RNA
atgtcggggctccggattaattcggcgaaagatgatgcggcggggcaagcggcggcgaatcgggcgacgtcgaatattaaagggctcacgcaagcgtcgcggaatgcggcggatgggatttcgattgcgcaaacgacggaaggggcgctcaatgaaattaataataatctccaacgggtgcgggaactctcggtgcaagcgacggcgggggcgaatgcggatgcggcgctcaaagcgattcaagcggaaattcaacaacggctcgaagaaattgatcgggtgtcgcaacaaacgcaagcggcggcggtgaaagtgctctcgcaagataatgcgatggcgattcaagtgggggcgaatgatggggcggcgattacgattgatctccaaaaaattgatgtgaaatcgctcgggctcgatgggtttaatgtgaattcgccggggtcgacggcgaatccgctcgcgtcgattgattcggcgctctcgaaagtggatgcggtgcggtcgtcgctcggggcgattcaaaatcggtttgattcggcgattacgaatctcgggaatacggtgacgaatctcaattcggcgcggtcgcggattgaagatgcggattatgcgacggaagtgtcgcaaatgtcgaaagcgcaaattctcgatcaagcggggacgtcgacgctcgcgcaactcgtgccgcgggggtcgcatcatcatcatcatcatggg

SEQ ID NO: 12 - RNA, 717 nt, synthetic construct
  source 1..717: mol_type = other RNA
atgagtggtctaagaattaatagtgctaaagatgatgctgctggtcaagctgctgctaatagagctactagtaatattaaaggtctaactcaagctagtagaaatgctgctgatggtattagtattgctcaaactactgaaggtgctctaaatgaaattaataataatctacaaagagttagagaactaagtgttcaagctactgctggtgctaatgctgatgctgctctaaaagctattcaagctgaaattcaacaaagactagaagaaattgatagagttagtcaacaaactcaagctgctgctgttaaagttctaagtcaagataatgctatggctattcaagttggtgctaatgatggtgctgctattactattgatctacaaaaaattgatgttaaaagtctaggtctagatggttttaatgttaatagtcctggtagtactgctaatcctctagctagtattgatagtgctctaagtaaagttgatgctgttagaagtagtctaggtgctattcaaaatagatttgatagtgctattactaatctaggtaatactgttactaatctaaatagtgctagaagtagaattgaagatgctgattatgctactgaagttagtcaaatgagtaaagctcaaattctagatcaagctggtactagtactctagctcaactagttcctagaggtagtcatcatcatcatcatcatggt

SEQ ID NO: 13 - RNA, 717 nt, synthetic construct
  source 1..717: mol_type = other RNA
atgagcggtctgaggattaatagcgctaaagatgatgctgctggtcaagctgctgctaatagggctactagcaatattaaaggtctgactcaagctagcaggaatgctgctgatggtattagcattgctcaaactactgaaggtgctctgaatgaaattaataataatctgcaaagggttagggaactgagcgttcaagctactgctggtgctaatgctgatgctgctctgaaagctattcaagctgaaattcaacaaaggctggaagaaattgatagggttagccaacaaactcaagctgctgctgttaaagttctgagccaagataatgctatggctattcaagttggtgctaatgatggtgctgctattactattgatctgcaaaaaattgatgttaaaagcctgggtctggatggttttaatgttaatagccctggtagcactgctaatcctctggctagcattgatagcgctctgagcaaagttgatgctgttaggagcagcctgggtgctattcaaaataggtttgatagcgctattactaatctgggtaatactgttactaatctgaatagcgctaggagcaggattgaagatgctgattatgctactgaagttagccaaatgagcaaagctcaaattctggatcaagctggtactagcactctggctcaactggttcctaggggtagccatcatcatcatcatcatggt

SEQ ID NO: 14 - RNA, 750 nt, synthetic construct
  source 1..750: mol_type = other RNA
atgtctggtttacgtattaattctgctaaagatgatgctgctggtcaagctattgctaatcgttttacttctaatattaaaggtttaactcaagcttctcgtaatgctgctgatggtatttctattgctcaaactactgaaggtgctttaaatgaaattaataataatttacaacgtgttcgtgaattatctgttcaagctactgctggtgctaatgctgatgctgctttaaaagctattcaagctgaaattcaacaacgtttagaagaaattgatcgtgtttctcaacaaactcaagctgctgctgttaaagttttatctcaagataatgctatggctattcaagttggtgctaatgatggtgctgctattactattgatttacaaaaaattgatgttaaatctttaggtttagatggttttaatgttaattctcctggttctactgctaatcctttagcttctattgattctgctttatctaaagttgatgctgttcgttcttctttaggtgctattcaaaatcgttttgattctgctattactaatttaggtaatactgttactaatttaaattctgctcgttctcgtattgaagatgctgattatgctactgaagtttctcaaatgtctaaagctcaaattttacaacaagctggtacttctgttttagctcaagctaatcaagttcctcaaaatgttttatctttattagttcctcgtggttctcatcatcatcatcatcatggt

SEQ ID NO: 15 - RNA, 750 nt, synthetic construct
  source 1..750: mol_type = other RNA
atgtccggcttgcgcatcaactccgccaaggacgacgccgccggccaggccatcgccaaccgcttcacctccaacatcaagggcttgacccaggcctcccgcaacgccgccgacggcatctccatcgcccagaccaccgagggcgccttgaacgagatcaacaacaacttgcagcgcgtccgcgagttgtccgtccaggccaccgccggcgccaacgccgacgccgccttgaaggccatccaggccgagatccagcagcgcttggaggagatcgaccgcgtctcccagcagacccaggccgccgccgtcaaggtcttgtcccaggacaacgccatggccatccaggtcggcgccaacgacggcgccgccatcaccatcgacttgcagaagatcgacgtcaagtccttgggcttggacggcttcaacgtcaactcccccggctccaccgccaaccccttggcctccatcgactccgccttgtccaaggtcgacgccgtccgctcctccttgggcgccatccagaaccgcttcgactccgccatcaccaacttgggcaacaccgtcaccaacttgaactccgcccgctcccgcatcgaggacgccgactacgccaccgaggtctcccagatgtccaaggcccagatcttgcagcaggccggcacctccgtcttggcccaggccaaccaggtcccccagaacgtcttgtccttgttggtcccccgcggctcccaccaccaccaccaccacggc

SEQ ID NO: 16 - RNA, 750 nt, synthetic construct
  source 1..750: mol_type = other RNA
atgtcaggacttcgaataaattcagcaaaagatgatgcagcaggacaagcaatagcaaatcgatttacatcaaatataaaaggacttacacaagcatcacgaaatgcagcagatggaatatcaatagcacaaacaacagaaggagcacttaatgaaataaataataatcttcaacgagtacgagaactttcagtacaagcaacagcaggagcaaatgcagatgcagcacttaaagcaatacaagcagaaatacaacaacgacttgaagaaatagatcgagtatcacaacaaacacaagcagcagcagtaaaagtactttcacaagataatgcaatggcaatacaagtaggagcaaatgatggagcagcaataacaatagatcttcaaaaaatagatgtaaaatcacttggacttgatggatttaatgtaaattcaccaggatcaacagcaaatccacttgcatcaatagattcagcactttcaaaagtagatgcagtacgatcatcacttggagcaatacaaaatcgatttgattcagcaataacaaatcttggaaatacagtaacaaatcttaattcagcacgatcacgaatagaagatgcagattatgcaacagaagtatcacaaatgtcaaaagcacaaatacttcaacaagcaggaacatcagtacttgcacaagcaaatcaagtaccacaaaatgtactttcacttcttgtaccacgaggatcacatcatcatcatcatcatgga

SEQ ID NO: 17 - RNA, 750 nt, synthetic construct
  source 1..750: mol_type = other RNA
atgtcggggctccggattaattcggcgaaagatgatgcggcggggcaagcgattgcgaatcggtttacgtcgaatattaaagggctcacgcaagcgtcgcggaatgcggcggatgggatttcgattgcgcaaacgacggaaggggcgctcaatgaaattaataataatctccaacgggtgcgggaactctcggtgcaagcgacggcgggggcgaatgcggatgcggcgctcaaagcgattcaagcggaaattcaacaacggctcgaagaaattgatcgggtgtcgcaacaaacgcaagcggcggcggtgaaagtgctctcgcaagataatgcgatggcgattcaagtgggggcgaatgatggggcggcgattacgattgatctccaaaaaattgatgtgaaatcgctcgggctcgatgggtttaatgtgaattcgccggggtcgacggcgaatccgctcgcgtcgattgattcggcgctctcgaaagtggatgcggtgcggtcgtcgctcggggcgattcaaaatcggtttgattcggcgattacgaatctcgggaatacggtgacgaatctcaattcggcgcggtcgcggattgaagatgcggattatgcgacggaagtgtcgcaaatgtcgaaagcgcaaattctccaacaagcggggacgtcggtgctcgcgcaagcgaatcaagtgccgcaaaatgtgctctcgctcctcgtgccgcgggggtcgcatcatcatcatcatcatggg

SEQ ID NO: 18 - RNA, 750 nt, synthetic construct
  source 1..750: mol_type = other RNA
atgagtggtctaagaattaatagtgctaaagatgatgctgctggtcaagctattgctaatagatttactagtaatattaaaggtctaactcaagctagtagaaatgctgctgatggtattagtattgctcaaactactgaaggtgctctaaatgaaattaataataatctacaaagagttagagaactaagtgttcaagctactgctggtgctaatgctgatgctgctctaaaagctattcaagctgaaattcaacaaagactagaagaaattgatagagttagtcaacaaactcaagctgctgctgttaaagttctaagtcaagataatgctatggctattcaagttggtgctaatgatggtgctgctattactattgatctacaaaaaattgatgttaaaagtctaggtctagatggttttaatgttaatagtcctggtagtactgctaatcctctagctagtattgatagtgctctaagtaaagttgatgctgttagaagtagtctaggtgctattcaaaatagatttgatagtgctattactaatctaggtaatactgttactaatctaaatagtgctagaagtagaattgaagatgctgattatgctactgaagttagtcaaatgagtaaagctcaaattctacaacaagctggtactagtgttctagctcaagctaatcaagttcctcaaaatgttctaagtctactagttcctagaggtagtcatcatcatcatcatcatggt

SEQ ID NO: 19 - RNA, 750 nt, synthetic construct
  source 1..750: mol_type = other RNA
atgagcggtctgaggattaatagcgctaaagatgatgctgctggtcaagctattgctaataggtttactagcaatattaaaggtctgactcaagctagcaggaatgctgctgatggtattagcattgctcaaactactgaaggtgctctgaatgaaattaataataatctgcaaagggttagggaactgagcgttcaagctactgctggtgctaatgctgatgctgctctgaaagctattcaagctgaaattcaacaaaggctggaagaaattgatagggttagccaacaaactcaagctgctgctgttaaagttctgagccaagataatgctatggctattcaagttggtgctaatgatggtgctgctattactattgatctgcaaaaaattgatgttaaaagcctgggtctggatggttttaatgttaatagccctggtagcactgctaatcctctggctagcattgatagcgctctgagcaaagttgatgctgttaggagcagcctgggtgctattcaaaataggtttgatagcgctattactaatctgggtaatactgttactaatctgaatagcgctaggagcaggattgaagatgctgattatgctactgaagttagccaaatgagcaaagctcaaattctgcaacaagctggtactagcgttctggctcaagctaatcaagttcctcaaaatgttctgagcctgctggttcctaggggtagccatcatcatcatcatcatggt

SEQ ID NO: 20 - RNA, 750 nt, synthetic construct
  source 1..750: mol_type = other RNA
atgtctggtttacgtattaattctgctaaagatgatgctgctggtcaagctattgctaatcgttttacttctaatattaaaggtttaactcaagcttctcgtaatgctaatgatggtatttctattgctcaaactactgaaggtgctttaaatgaaattaataataatttacaacgtgttcgtgaattatctgttcaagctactaatggtactaattctgattctgatttaaaatctattcaagatgaaattcaacaacgtttagaagaaattgatcgtgtttctaatcaaactcaatttaatggtgttaaagttttatctcaagataatcaaatgaaaattcaagttggtgctaatgatggtgaaactattactattgatttacaaaaaattgatgttaaatctttaggtttagatggttttaatgttaattctcctggttctactgctaatcctttagcttctattgattctgctttatctaaagttgatgctgttcgttcttctttaggtgctattcaaaatcgttttgattctgctattactaatttaggtaatactgttactaatttaaattctgctcgttctcgtattgaagatgctgattatgctactgaagtttctaatatgtctaaagctcaaattttacaacaagctggtacttctgttttagctcaagctaatcaagttcctcaaaatgttttatctttattagttcctcgtggttctcatcatcatcatcatcatggt

SEQ ID NO: 21 - RNA, 750 nt, synthetic construct
  source 1..750: mol_type = other RNA
atgtccggcttgcgcatcaactccgccaaggacgacgccgccggccaggccatcgccaaccgcttcacctccaacatcaagggcttgacccaggcctcccgcaacgccaacgacggcatctccatcgcccagaccaccgagggcgccttgaacgagatcaacaacaacttgcagcgcgtccgcgagttgtccgtccaggccaccaacggcaccaactccgactccgacttgaagtccatccaggacgagatccagcagcgcttggaggagatcgaccgcgtctccaaccagacccagttcaacggcgtcaaggtcttgtcccaggacaaccagatgaagatccaggtcggcgccaacgacggcgagaccatcaccatcgacttgcagaagatcgacgtcaagtccttgggcttggacggcttcaacgtcaactcccccggctccaccgccaaccccttggcctccatcgactccgccttgtccaaggtcgacgccgtccgctcctccttgggcgccatccagaaccgcttcgactccgccatcaccaacttgggcaacaccgtcaccaacttgaactccgcccgctcccgcatcgaggacgccgactacgccaccgaggtctccaacatgtccaaggcccagatcttgcagcaggccggcacctccgtcttggcccaggccaaccaggtcccccagaacgtcttgtccttgttggtcccccgcggctcccaccaccaccaccaccacggc

SEQ ID NO: 22 - RNA, 750 nt, synthetic construct
  source 1..750: mol_type = other RNA
atgtcaggacttcgaataaattcagcaaaagatgatgcagcaggacaagcaatagcaaatcgatttacatcaaatataaaaggacttacacaagcatcacgaaatgcaaatgatggaatatcaatagcacaaacaacagaaggagcacttaatgaaataaataataatcttcaacgagtacgagaactttcagtacaagcaacaaatggaacaaattcagattcagatcttaaatcaatacaagatgaaatacaacaacgacttgaagaaatagatcgagtatcaaatcaaacacaatttaatggagtaaaagtactttcacaagataatcaaatgaaaatacaagtaggagcaaatgatggagaaacaataacaatagatcttcaaaaaatagatgtaaaatcacttggacttgatggatttaatgtaaattcaccaggatcaacagcaaatccacttgcatcaatagattcagcactttcaaaagtagatgcagtacgatcatcacttggagcaatacaaaatcgatttgattcagcaataacaaatcttggaaatacagtaacaaatcttaattcagcacgatcacgaatagaagatgcagattatgcaacagaagtatcaaatatgtcaaaagcacaaatacttcaacaagcaggaacatcagtacttgcacaagcaaatcaagtaccacaaaatgtactttcacttcttgtaccacgaggatcacatcatcatcatcatcatgga

SEQ ID NO: 23 - RNA, 750 nt, synthetic construct
  source 1..750: mol_type = other RNA
atgtcggggctccggattaattcggcgaaagatgatgcggcggggcaagcgattgcgaatcggtttacgtcgaatattaaagggctcacgcaagcgtcgcggaatgcgaatgatgggatttcgattgcgcaaacgacggaaggggcgctcaatgaaattaataataatctccaacgggtgcgggaactctcggtgcaagcgacgaatgggacgaattcggattcggatctcaaatcgattcaagatgaaattcaacaacggctcgaagaaattgatcgggtgtcgaatcaaacgcaatttaatggggtgaaagtgctctcgcaagataatcaaatgaaaattcaagtgggggcgaatgatggggaaacgattacgattgatctccaaaaaattgatgtgaaatcgctcgggctcgatgggtttaatgtgaattcgccggggtcgacggcgaatccgctcgcgtcgattgattcggcgctctcgaaagtggatgcggtgcggtcgtcgctcggggcgattcaaaatcggtttgattcggcgattacgaatctcgggaatacggtgacgaatctcaattcggcgcggtcgcggattgaagatgcggattatgcgacggaagtgtcgaatatgtcgaaagcgcaaattctccaacaagcggggacgtcggtgctcgcgcaagcgaatcaagtgccgcaaaatgtgctctcgctcctcgtgccgcgggggtcgcatcatcatcatcatcatggg

SEQ ID NO: 24 - RNA, 750 nt, synthetic construct
  source 1..750: mol_type = other RNA
atgagtggtctaagaattaatagtgctaaagatgatgctgctggtcaagctattgctaatagatttactagtaatattaaaggtctaactcaagctagtagaaatgctaatgatggtattagtattgctcaaactactgaaggtgctctaaatgaaattaataataatctacaaagagttagagaactaagtgttcaagctactaatggtactaatagtgatagtgatctaaaaagtattcaagatgaaattcaacaaagactagaagaaattgatagagttagtaatcaaactcaatttaatggtgttaaagttctaagtcaagataatcaaatgaaaattcaagttggtgctaatgatggtgaaactattactattgatctacaaaaaattgatgttaaaagtctaggtctagatggttttaatgttaatagtcctggtagtactgctaatcctctagctagtattgatagtgctctaagtaaagttgatgctgttagaagtagtctaggtgctattcaaaatagatttgatagtgctattactaatctaggtaatactgttactaatctaaatagtgctagaagtagaattgaagatgctgattatgctactgaagttagtaatatgagtaaagctcaaattctacaacaagctggtactagtgttctagctcaagctaatcaagttcctcaaaatgttctaagtctactagttcctagaggtagtcatcatcatcatcatcatggt

SEQ ID NO: 25 - RNA, 750 nt, synthetic construct
  source 1..750: mol_type = other RNA
atgagcggtctgaggattaatagcgctaaagatgatgctgctggtcaagctattgctaataggtttactagcaatattaaaggtctgactcaagctagcaggaatgctaatgatggtattagcattgctcaaactactgaaggtgctctgaatgaaattaataataatctgcaaagggttagggaactgagcgttcaagctactaatggtactaatagcgatagcgatctgaaaagcattcaagatgaaattcaacaaaggctggaagaaattgatagggttagcaatcaaactcaatttaatggtgttaaagttctgagccaagataatcaaatgaaaattcaagttggtgctaatgatggtgaaactattactattgatctgcaaaaaattgatgttaaaagcctgggtctggatggttttaatgttaatagccctggtagcactgctaatcctctggctagcattgatagcgctctgagcaaagttgatgctgttaggagcagcctgggtgctattcaaaataggtttgatagcgctattactaatctgggtaatactgttactaatctgaatagcgctaggagcaggattgaagatgctgattatgctactgaagttagcaatatgagcaaagctcaaattctgcaacaagctggtactagcgttctggctcaagctaatcaagttcctcaaaatgttctgagcctgctggttcctaggggtagccatcatcatcatcatcatggt

SEQ ID NO: 26 - RNA, 987 nt, synthetic construct
  source 1..987: mol_type = other RNA
atgcgtggttctcatcatcatcatcatcatggtatggcttctatgactggtggtcaacaaatgggtcgtgatttatatgatgatgatgataaagatcctatggctcaagttattaatactaattctttatctttattaactcaaaataatttaaataaatctcaatcttctttatcttctgctattgaacgtttatcttctggtttacgtattaattctgctaaagatgatgctgctggtcaagctattgctaatcgttttacttctaatattaaaggtttaactcaagcttctcgtaatgctaatgatggtatttctattgctcaaactactgaaggtgctttaaatgaaattaataataatttacaacgtgttcgtgaattatctgttcaagctactaatggtactaattctgattctgatttaaaatctattcaagatgaaattcaacaacgtttagaagaaattgatcgtgtttctaatcaaactcaatttaatggtgttaaagttttatctcaagataatcaaatgaaaattcaagttggtgctaatgatggtgaaactattactattgatttacaaaaaattgatgttaaatctttaggtttagatggttttaatgttaattctcctggtatttctggtggtggtggtggtattttagattctatgggtactttaattaatgaagatgctgctgctgctaaaaaatctactgctaatcctttagcttctattgattctgctttatctaaagttgatgctgttcgttcttctttaggtgctattcaaaatcgttttgattctgctattactaatttaggtaatactgttactaatttaaattctgctcgttctcgtattgaagatgctgattatgctactgaagtttctaatatgtctaaagctcaaattttacaacaagctggtacttctgttttagctcaagctaatcaagttcctcaaaatgttttatctttattacgt

SEQ ID NO: 27 - RNA, 987 nt, synthetic construct
  source 1..987: mol_type = other RNA
atgcgcggctcccaccaccaccaccaccacggcatggcctccatgaccggcggccagcagatgggccgcgacttgtacgacgacgacgacaaggaccccatggcccaggtcatcaacaccaactccttgtccttgttgacccagaacaacttgaacaagtcccagtcctccttgtcctccgccatcgagcgcttgtcctccggcttgcgcatcaactccgccaaggacgacgccgccggccaggccatcgccaaccgcttcacctccaacatcaagggcttgacccaggcctcccgcaacgccaacgacggcatctccatcgcccagaccaccgagggcgccttgaacgagatcaacaacaacttgcagcgcgtccgcgagttgtccgtccaggccaccaacggcaccaactccgactccgacttgaagtccatccaggacgagatccagcagcgcttggaggagatcgaccgcgtctccaaccagacccagttcaacggcgtcaaggtcttgtcccaggacaaccagatgaagatccaggtcggcgccaacgacggcgagaccatcaccatcgacttgcagaagatcgacgtcaagtccttgggcttggacggcttcaacgtcaactcccccggcatctccggcggcggcggcggcatcttggactccatgggcaccttgatcaacgaggacgccgccgccgccaagaagtccaccgccaaccccttggcctccatcgactccgccttgtccaaggtcgacgccgtccgctcctccttgggcgccatccagaaccgcttcgactccgccatcaccaacttgggcaacaccgtcaccaacttgaactccgcccgctcccgcatcgaggacgccgactacgccaccgaggtctccaacatgtccaaggcccagatcttgcagcaggccggcacctccgtcttggcccaggccaaccaggtcccccagaacgtcttgtccttgttgcgc

SEQ ID NO: 28 - RNA, 987 nt, synthetic construct
  source 1..987: mol_type = other RNA
atgcgaggatcacatcatcatcatcatcatggaatggcatcaatgacaggaggacaacaaatgggacgagatctttatgatgatgatgataaagatccaatggcacaagtaataaatacaaattcactttcacttcttacacaaaataatcttaataaatcacaatcatcactttcatcagcaatagaacgactttcatcaggacttcgaataaattcagcaaaagatgatgcagcaggacaagcaatagcaaatcgatttacatcaaatataaaaggacttacacaagcatcacgaaatgcaaatgatggaatatcaatagcacaaacaacagaaggagcacttaatgaaataaataataatcttcaacgagtacgagaactttcagtacaagcaacaaatggaacaaattcagattcagatcttaaatcaatacaagatgaaatacaacaacgacttgaagaaatagatcgagtatcaaatcaaacacaatttaatggagtaaaagtactttcacaagataatcaaatgaaaatacaagtaggagcaaatgatggagaaacaataacaatagatcttcaaaaaatagatgtaaaatcacttggacttgatggatttaatgtaaattcaccaggaatatcaggaggaggaggaggaatacttgattcaatgggaacacttataaatgaagatgcagcagcagcaaaaaaatcaacagcaaatccacttgcatcaatagattcagcactttcaaaagtagatgcagtacgatcatcacttggagcaatacaaaatcgatttgattcagcaataacaaatcttggaaatacagtaacaaatcttaattcagcacgatcacgaatagaagatgcagattatgcaacagaagtatcaaatatgtcaaaagcacaaatacttcaacaagcaggaacatcagtacttgcacaagcaaatcaagtaccacaaaatgtactttcacttcttcga

SEQ ID NO: 29 - RNA, 987 nt, synthetic construct
  source 1..987: mol_type = other RNA
atgcgggggtcgcatcatcatcatcatcatgggatggcgtcgatgacgggggggcaacaaatggggcgggatctctatgatgatgatgataaagatccgatggcgcaagtgattaatacgaattcgctctcgctcctcacgcaaaataatctcaataaatcgcaatcgtcgctctcgtcggcgattgaacggctctcgtcggggctccggattaattcggcgaaagatgatgcggcggggcaagcgattgcgaatcggtttacgtcgaatattaaagggctcacgcaagcgtcgcggaatgcgaatgatgggatttcgattgcgcaaacgacggaaggggcgctcaatgaaattaataataatctccaacgggtgcgggaactctcggtgcaagcgacgaatgggacgaattcggattcggatctcaaatcgattcaagatgaaattcaacaacggctcgaagaaattgatcgggtgtcgaatcaaacgcaatttaatggggtgaaagtgctctcgcaagataatcaaatgaaaattcaagtgggggcgaatgatggggaaacgattacgattgatctccaaaaaattgatgtgaaatcgctcgggctcgatgggtttaatgtgaattcgccggggatttcggggggggggggggggattctcgattcgatggggacgctcattaatgaagatgcggcggcggcgaaaaaatcgacggcgaatccgctcgcgtcgattgattcggcgctctcgaaagtggatgcggtgcggtcgtcgctcggggcgattcaaaatcggtttgattcggcgattacgaatctcgggaatacggtgacgaatctcaattcggcgcggtcgcggattgaagatgcggattatgcgacggaagtgtcgaatatgtcgaaagcgcaaattctccaacaagcggggacgtcggtgctcgcgcaagcgaatcaagtgccgcaaaatgtgctctcgctcctccgg

SEQ ID NO: 30 - RNA, 987 nt, synthetic construct
  source 1..987: mol_type = other RNA
atgagaggtagtcatcatcatcatcatcatggtatggctagtatgactggtggtcaacaaatgggtagagatctatatgatgatgatgataaagatcctatggctcaagttattaatactaatagtctaagtctactaactcaaaataatctaaataaaagtcaaagtagtctaagtagtgctattgaaagactaagtagtggtctaagaattaatagtgctaaagatgatgctgctggtcaagctattgctaatagatttactagtaatattaaaggtctaactcaagctagtagaaatgctaatgatggtattagtattgctcaaactactgaaggtgctctaaatgaaattaataataatctacaaagagttagagaactaagtgttcaagctactaatggtactaatagtgatagtgatctaaaaagtattcaagatgaaattcaacaaagactagaagaaattgatagagttagtaatcaaactcaatttaatggtgttaaagttctaagtcaagataatcaaatgaaaattcaagttggtgctaatgatggtgaaactattactattgatctacaaaaaattgatgttaaaagtctaggtctagatggttttaatgttaatagtcctggtattagtggtggtggtggtggtattctagatagtatgggtactctaattaatgaagatgctgctgctgctaaaaaaagtactgctaatcctctagctagtattgatagtgctctaagtaaagttgatgctgttagaagtagtctaggtgctattcaaaatagatttgatagtgctattactaatctaggtaatactgttactaatctaaatagtgctagaagtagaattgaagatgctgattatgctactgaagttagtaatatgagtaaagctcaaattctacaacaagctggtactagtgttctagctcaagctaatcaagttcctcaaaatgttctaagtctactaaga

SEQ ID NO: 31 - RNA, 987 nt, synthetic construct
  source 1..987: mol_type = other RNA
atgaggggtagccatcatcatcatcatcatggtatggctagcatgactggtggtcaacaaatgggtagggatctgtatgatgatgatgataaagatcctatggctcaagttattaatactaatagcctgagcctgctgactcaaaataatctgaataaaagccaaagcagcctgagcagcgctattgaaaggctgagcagcggtctgaggattaatagcgctaaagatgatgctgctggtcaagctattgctaataggtttactagcaatattaaaggtctgactcaagctagcaggaatgctaatgatggtattagcattgctcaaactactgaaggtgctctgaatgaaattaataataatctgcaaagggttagggaactgagcgttcaagctactaatggtactaatagcgatagcgatctgaaaagcattcaagatgaaattcaacaaaggctggaagaaattgatagggttagcaatcaaactcaatttaatggtgttaaagttctgagccaagataatcaaatgaaaattcaagttggtgctaatgatggtgaaactattactattgatctgcaaaaaattgatgttaaaagcctgggtctggatggttttaatgttaatagccctggtattagcggtggtggtggtggtattctggatagcatgggtactctgattaatgaagatgctgctgctgctaaaaaaagcactgctaatcctctggctagcattgatagcgctctgagcaaagttgatgctgttaggagcagcctgggtgctattcaaaataggtttgatagcgctattactaatctgggtaatactgttactaatctgaatagcgctaggagcaggattgaagatgctgattatgctactgaagttagcaatatgagcaaagctcaaattctgcaacaagctggtactagcgttctggctcaagctaatcaagttcctcaaaatgttctgagcctgctgagg

SEQ ID NO: 32 - RNA, 717 nt, synthetic construct; identical to SEQ ID NO: 8
  source 1..717: mol_type = other RNA

SEQ ID NO: 33 - RNA, 717 nt, synthetic construct; identical to SEQ ID NO: 9
  source 1..717: mol_type = other RNA

SEQ ID NO: 34 - RNA, 717 nt, synthetic construct; identical to SEQ ID NO: 10
  source 1..717: mol_type = other RNA

SEQ ID NO: 35 - RNA, 717 nt, synthetic construct; identical to SEQ ID NO: 11
  source 1..717: mol_type = other RNA

SEQ ID NO: 36 - RNA, 717 nt, synthetic construct; identical to SEQ ID NO: 12
  source 1..717: mol_type = other RNA

SEQ ID NO: 37 - RNA, 717 nt, synthetic construct; identical to SEQ ID NO: 13
  source 1..717: mol_type = other RNA

SEQ ID NO: 38 - RNA, 474 nt, synthetic construct
  source 1..474: mol_type = other RNA
atgaatcaatcttgtcctaataaaacttcttttaaaattgcttttattcaagctcgttggcatgctgatattgttgatgaagctcgtaaatcttttgttgctgaattagctgctaaaactggtggttctgttgaagttgaaatttttgatgttcctggtgcttatgaaattcctttacatgctaaaactttagctcgtactggtcgttatgctgctattgttggtgctgcttttgttattgatggtggtatttatcgtcatgattttgttgctactgctgttattaatggtatgatgcaagttcaattagaaactgaagttcctgttttatctgttgttttaactcctcatcattttcatgaatctaaagaacatcatgatttttttcatgctcattttaaagttaaaggtgttgaagctgctcatgctgctttacaaattgtttctgaacgttctcgtattgctgctttagtt

SEQ ID NO: 39 - RNA, 474 nt, synthetic construct
  source 1..474: mol_type = other RNA
atgaaccagtcctgccccaacaagacctccttcaagatcgccttcatccaggcccgctggcacgccgacatcgtcgacgaggcccgcaagtccttcgtcgccgagttggccgccaagaccggcggctccgtcgaggtcgagatcttcgacgtccccggcgcctacgagatccccttgcacgccaagaccttggcccgcaccggccgctacgccgccatcgtcggcgccgccttcgtcatcgacggcggcatctaccgccacgacttcgtcgccaccgccgtcatcaacggcatgatgcaggtccagttggagaccgaggtccccgtcttgtccgtcgtcttgaccccccaccacttccacgagtccaaggagcaccacgacttcttccacgcccacttcaaggtcaagggcgtcgaggccgcccacgccgccttgcagatcgtctccgagcgctcccgcatcgccgccttggtc

SEQ ID NO: 40 - RNA, 474 nt, synthetic construct
  source 1..474: mol_type = other RNA
atgaatcaatcatgtccaaataaaacatcatttaaaatagcatttatacaagcacgatggcatgcagatatagtagatgaagcacgaaaatcatttgtagcagaacttgcagcaaaaacaggaggatcagtagaagtagaaatatttgatgtaccaggagcatatgaaataccacttcatgcaaaaacacttgcacgaacaggacgatatgcagcaatagtaggagcagcatttgtaatagatggaggaatatatcgacatgattttgtagcaacagcagtaataaatggaatgatgcaagtacaacttgaaacagaagtaccagtactttcagtagtacttacaccacatcattttcatgaatcaaaagaacatcatgatttttttcatgcacattttaaagtaaaaggagtagaagcagcacatgcagcacttcaaatagtatcagaacgatcacgaatagcagcacttgta

SEQ ID NO: 41 - RNA, 474 nt, synthetic construct
  source 1..474: mol_type = other RNA
atgaatcaatcgtgtccgaataaaacgtcgtttaaaattgcgtttattcaagcgcggtggcatgcggatattgtggatgaagcgcggaaatcgtttgtggcggaactcgcggcgaaaacgggggggtcggtggaagtggaaatttttgatgtgccgggggcgtatgaaattccgctccatgcgaaaacgctcgcgcggacggggcggtatgcggcgattgtgggggcggcgtttgtgattgatggggggatttatcggcatgattttgtggcgacggcggtgattaatgggatgatgcaagtgcaactcgaaacggaagtgccggtgctctcggtggtgctcacgccgcatcattttcatgaatcgaaagaacatcatgatttttttcatgcgcattttaaagtgaaaggggtggaagcggcgcatgcggcgctccaaattgtgtcggaacggtcgcggattgcggcgctcgtg

SEQ ID NO: 42 - RNA, 474 nt, synthetic construct
  source 1..474: mol_type = other RNA
atgaatcaaagttgtcctaataaaactagttttaaaattgcttttattcaagctagatggcatgctgatattgttgatgaagctagaaaaagttttgttgctgaactagctgctaaaactggtggtagtgttgaagttgaaatttttgatgttcctggtgcttatgaaattcctctacatgctaaaactctagctagaactggtagatatgctgctattgttggtgctgcttttgttattgatggtggtatttatagacatgattttgttgctactgctgttattaatggtatgatgcaagttcaactagaaactgaagttcctgttctaagtgttgttctaactcctcatcattttcatgaaagtaaagaacatcatgatttttttcatgctcattttaaagttaaaggtgttgaagctgctcatgctgctctacaaattgttagtgaaagaagtagaattgctgctctagtt

SEQ ID NO: 43 - RNA, 474 nt, synthetic construct
  source 1..474: mol_type = other RNA
atgaatcaaagctgtcctaataaaactagctttaaaattgcttttattcaagctaggtggcatgctgatattgttgatgaagctaggaaaagctttgttgctgaactggctgctaaaactggtggtagcgttgaagttgaaatttttgatgttcctggtgcttatgaaattcctctgcatgctaaaactctggctaggactggtaggtatgctgctattgttggtgctgcttttgttattgatggtggtatttataggcatgattttgttgctactgctgttattaatggtatgatgcaagttcaactggaaactgaagttcctgttctgagcgttgttctgactcctcatcattttcatgaaagcaaagaacatcatgatttttttcatgctcattttaaagttaaaggtgttgaagctgctcatgctgctctgcaaattgttagcgaaaggagcaggattgctgctctggtt

SEQ ID NO: 44 - RNA, 504 nt, synthetic construct
  source 1..504: mol_type = other RNA
atgcgtcgtattcaatctattgctcgttctcctattgctattgctttatttatgtctttagctgttgctggttgtgcttctaaaaaaaatttacctaataatgctggtgatttaggtttaggtgctggtgctgctactcctggttcttctcaagattttactgttaatgttggtgatcgtattttttttgatttagattcttctttaattcgtgctgatgctcaacaaactttatctaaacaagctcaatggttacaacgttatcctcaatattctattactattgaaggtcatgctgatgaacgtggtactcgtgaatataatttagctttaggtcaacgtcgtgctgctgctactcgtgattttttagcttctcgtggtgttcctactaatcgtatgcgtactatttcttatggtaatgaacgtcctgttgctgtttgtgatgctgatacttgttggtctcaaaatcgtcgtgctgttactgttttaaatggtgctggtcgt

SEQ ID NO: 45 - RNA, 504 nt, synthetic construct
  source 1..504: mol_type = other RNA
atgcgccgcatccagtccatcgcccgctcccccatcgccatcgccttgttcatgtccttggccgtcgccggctgcgcctccaagaagaacttgcccaacaacgccggcgacttgggcttgggcgccggcgccgccacccccggctcctcccaggacttcaccgtcaacgtcggcgaccgcatcttcttcgacttggactcctccttgatccgcgccgacgcccagcagaccttgtccaagcaggcccagtggttgcagcgctacccccagtactccatcaccatcgagggccacgccgacgagcgcggcacccgcgagtacaacttggccttgggccagcgccgcgccgccgccacccgcgacttcttggcctcccgcggcgtccccaccaaccgcatgcgcaccatctcctacggcaacgagcgccccgtcgccgtctgcgacgccgacacctgctggtcccagaaccgccgcgccgtcaccgtcttgaacggcgccggccgc

SEQ ID NO: 46 - RNA, 504 nt, synthetic construct
  source 1..504: mol_type = other RNA
atgcgacgaatacaatcaatagcacgatcaccaatagcaatagcactttttatgtcacttgcagtagcaggatgtgcatcaaaaaaaaatcttccaaataatgcaggagatcttggacttggagcaggagcagcaacaccaggatcatcacaagattttacagtaaatgtaggagatcgaatattttttgatcttgattcatcacttatacgagcagatgcacaacaaacactttcaaaacaagcacaatggcttcaacgatatccacaatattcaataacaatagaaggacatgcagatgaacgaggaacacgagaatataatcttgcacttggacaacgacgagcagcagcaacacgagattttcttgcatcacgaggagtaccaacaaatcgaatgcgaacaatatcatatggaaatgaacgaccagtagcagtatgtgatgcagatacatgttggtcacaaaatcgacgagcagtaacagtacttaatggagcaggacga

SEQ ID NO: 47 - RNA, 504 nt, synthetic construct
  source 1..504: mol_type = other RNA
atgcggcggattcaatcgattgcgcggtcgccgattgcgattgcgctctttatgtcgctcgcggtggcggggtgtgcgtcgaaaaaaaatctcccgaataatgcgggggatctcgggctcggggcgggggcggcgacgccggggtcgtcgcaagattttacggtgaatgtgggggatcggattttttttgatctcgattcgtcgctcattcgggcggatgcgcaacaaacgctctcgaaacaagcgcaatggctccaacggtatccgcaatattcgattacgattgaagggcatgcggatgaacgggggacgcgggaatataatctcgcgctcgggcaacggcgggcggcggcgacgcgggattttctcgcgtcgcggggggtgccgacgaatcggatgcggacgatttcgtatgggaatgaacggccggtggcggtgtgtgatgcggatacgtgttggtcgcaaaatcggcgggcggtgacggtgctcaatggggcggggcgg

SEQ ID NO: 48 - RNA, 504 nt, synthetic construct
  source 1..504: mol_type = other RNA
atgagaagaattcaaagtattgctagaagtcctattgctattgctctatttatgagtctagctgttgctggttgtgctagtaaaaaaaatctacctaataatgctggtgatctaggtctaggtgctggtgctgctactcctggtagtagtcaagattttactgttaatgttggtgatagaattttttttgatctagatagtagtctaattagagctgatgctcaacaaactctaagtaaacaagctcaatggctacaaagatatcctcaatatagtattactattgaaggtcatgctgatgaaagaggtactagagaatataatctagctctaggtcaaagaagagctgctgctactagagattttctagctagtagaggtgttcctactaatagaatgagaactattagttatggtaatgaaagacctgttgctgtttgtgatgctgatacttgttggagtcaaaatagaagagctgttactgttctaaatggtgctggtaga

SEQ ID NO: 49 - RNA, 504 nt, synthetic construct
  source 1..504: mol_type = other RNA
atgaggaggattcaaagcattgctaggagccctattgctattgctctgtttatgagcctggctgttgctggttgtgctagcaaaaaaaatctgcctaataatgctggtgatctgggtctgggtgctggtgctgctactcctggtagcagccaagattttactgttaatgttggtgataggattttttttgatctggatagcagcctgattagggctgatgctcaacaaactctgagcaaacaagctcaatggctgcaaaggtatcctcaatatagcattactattgaaggtcatgctgatgaaaggggtactagggaatataatctggctctgggtcaaaggagggctgctgctactagggattttctggctagcaggggtgttcctactaataggatgaggactattagctatggtaatgaaaggcctgttgctgtttgtgatgctgatacttgttggagccaaaataggagggctgttactgttctgaatggtgctggtagg

SEQ ID NO: 50 - RNA, 531 nt, synthetic construct
  source 1..531: mol_type = other RNA
atgggtatttctaaagcttctttattatctttagctgctgctggtattgttttagctggttgtcaatcttctcgtttaggtaatttagataatgtttctcctcctcctcctcctgctcctgttaatgctgttcctgctggtactgttcaaaaaggtaatttagattctcctactcaatttcctaatgctccttctactgatatgtctgctcaatctggtactcaagttgcttctttacctcctgcttctgctcctgatttaactcctggtgctgttgctggtgtttggaatgcttctttaggtggtcaatcttgtaaaattgctactcctcaaactaaatatggtcaaggttatcgtgctggtcctttacgttgtcctggtgaattagctaatttagcttcttgggctgttaatggtaaacaattagttttatatgatgctaatggtggtactgttgcttctttatattcttctggtcaaggtcgttttgatggtcaaactactggtggtcaagctgttactttatctcgt

SEQ ID NO: 51 - RNA, 531 nt, synthetic construct
  source 1..531: mol_type = other RNA
atgggcatctccaaggcctccttgttgtccttggccgccgccggcatcgtcttggccggctgccagtcctcccgcttgggcaacttggacaacgtctcccccccccccccccccgcccccgtcaacgccgtccccgccggcaccgtccagaagggcaacttggactcccccacccagttccccaacgccccctccaccgacatgtccgcccagtccggcacccaggtcgcctccttgccccccgcctccgcccccgacttgacccccggcgccgtcgccggcgtctggaacgcctccttgggcggccagtcctgcaagatcgccaccccccagaccaagtacggccagggctaccgcgccggccccttgcgctgccccggcgagttggccaacttggcctcctgggccgtcaacggcaagcagttggtcttgtacgacgccaacggcggcaccgtcgcctccttgtactcctccggccagggccgcttcgacggccagaccaccggcggccaggccgtcaccttgtcccgc

SEQ ID NO: 52 - RNA, 531 nt, synthetic construct
  source 1..531: mol_type = other RNA
atgggaatatcaaaagcatcacttctttcacttgcagcagcaggaatagtacttgcaggatgtcaatcatcacgacttggaaatcttgataatgtatcaccaccaccaccaccagcaccagtaaatgcagtaccagcaggaacagtacaaaaaggaaatcttgattcaccaacacaatttccaaatgcaccatcaacagatatgtcagcacaatcaggaacacaagtagcatcacttccaccagcatcagcaccagatcttacaccaggagcagtagcaggagtatggaatgcatcacttggaggacaatcatgtaaaatagcaacaccacaaacaaaatatggacaaggatatcgagcaggaccacttcgatgtccaggagaacttgcaaatcttgcatcatgggcagtaaatggaaaacaacttgtactttatgatgcaaatggaggaacagtagcatcactttattcatcaggacaaggacgatttgatggacaaacaacaggaggacaagcagtaacactttcacga

SEQ ID NO: 53 - RNA, 531 nt, synthetic construct
  source 1..531: mol_type = other RNA
atggggatttcgaaagcgtcgctcctctcgctcgcggcggcggggattgtgctcgcggggtgtcaatcgtcgcggctcgggaatctcgataatgtgtcgccgccgccgccgccggcgccggtgaatgcggtgccggcggggacggtgcaaaaagggaatctcgattcgccgacgcaatttccgaatgcgccgtcgacggatatgtcggcgcaatcggggacgcaagtggcgtcgctcccgccggcgtcggcgccggatctcacgccgggggcggtggcgggggtgtggaatgcgtcgctcggggggcaatcgtgtaaaattgcgacgccgcaaacgaaatatgggcaagggtatcgggcggggccgctccggtgtccgggggaactcgcgaatctcgcgtcgtgggcggtgaatgggaaacaactcgtgctctatgatgcgaatggggggacggtggcgtcgctctattcgtcggggcaagggcggtttgatgggcaaacgacgggggggcaagcggtgacgctctcgcgg

SEQ ID NO: 54 - RNA, 531 nt, synthetic construct
  source 1..531: mol_type = other RNA
atgggtattagtaaagctagtctactaagtctagctgctgctggtattgttctagctggttgtcaaagtagtagactaggtaatctagataatgttagtcctcctcctcctcctgctcctgttaatgctgttcctgctggtactgttcaaaaaggtaatctagatagtcctactcaatttcctaatgctcctagtactgatatgagtgctcaaagtggtactcaagttgctagtctacctcctgctagtgctcctgatctaactcctggtgctgttgctggtgtttggaatgctagtctaggtggtcaaagttgtaaaattgctactcctcaaactaaatatggtcaaggttatagagctggtcctctaagatgtcctggtgaactagctaatctagctagttgggctgttaatggtaaacaactagttctatatgatgctaatggtggtactgttgctagtctatatagtagtggtcaaggtagatttgatggtcaaactactggtggtcaagctgttactctaagtaga

SEQ ID NO: 55 - RNA, 531 nt, synthetic construct
  source 1..531: mol_type = other RNA
atgggtattagcaaagctagcctgctgagcctggctgctgctggtattgttctggctggttgtcaaagcagcaggctgggtaatctggataatgttagccctcctcctcctcctgctcctgttaatgctgttcctgctggtactgttcaaaaaggtaatctggatagccctactcaatttcctaatgctcctagcactgatatgagcgctcaaagcggtactcaagttgctagcctgcctcctgctagcgctcctgatctgactcctggtgctgttgctggtgtttggaatgctagcctgggtggtcaaagctgtaaaattgctactcctcaaactaaatatggtcaaggttatagggctggtcctctgaggtgtcctggtgaactggctaatctggctagctgggctgttaatggtaaacaactggttctgtatgatgctaatggtggtactgttgctagcctgtatagcagcggtcaaggtaggtttgatggtcaaactactggtggtcaagctgttactctgagcagg

SEQ ID NO: 56 - RNA, 1047 nt, synthetic construct
  source 1..1047: mol_type = other RNA
atggaatttgctactgtttctcctcctgatcctggtactgctgctgctgctcgtgctcgtcaagatactttaactaaacctcgtggtgctttaggtcgtttagaagatttatctgtttggattgctgcttgtcaaggtcaatgtcctcctcgtcaatttgaacgtgctcgtgttgttgtttttgctggtgatcatggtgttgctcgttgtggtgtttctgcttatcctcctgaagttactgctcaaatggttgctaattttgatgctggtggtgctgctattaatgctttagctggtgttgctggtgcttctgttcgtgttgctgatttagctgttgatgctgatcctcctgatgatcgtattggtgctcataaagttcgtcgtggttctggtgatattactgttcaagatgctttaactgctgaagaaactgaacgtgctttatctgctggtgctgctattgctgatgaagaagttgatgctggtgctgatttattaattgctggtgatatgggtattggtaatactactgctgctgctgttttagttgctgctttaactaatgttgaacctgttgttgctgttggttttggtactggtattgatgatgctggttgggctcgtaaaactgctgctgttcgtgatgctttatttcgtgctcgtcgtgttttacctgatcctgttgctttattacgttgtgctggtggtgctgatttagctgctttagctggtttttgtgctcaagctgctgttcgtcgtactcctttattattagatggtatggctgttactgctgctgctttagttgctgaacatttagctcctggtgctcgtttatggtggcaagctggtcatcgttctactgaacctggtcatgctttagctttaactgctttagatttagaacctattttagatttacgtatgcgtttaggtgaaggtactggtgctgctttagctttacctattgttcgtgctgctgttgctgctttatcttctatggctacttttgctcaagctggtgtttctgatccttctgctcatcct

SEQ ID NO: 57 - RNA, 1047 nt, synthetic construct
  source 1..1047: mol_type = other RNA
atggagttcgccaccgtctccccccccgaccccggcaccgccgccgccgcccgcgcccgccaggacaccttgaccaagccccgcggcgccttgggccgcttggaggacttgtccgtctggatcgccgcctgccagggccagtgccccccccgccagttcgagcgcgcccgcgtcgtcgtcttcgccggcgaccacggcgtcgcccgctgcggcgtctccgcctacccccccgaggtcaccgcccagatggtcgccaacttcgacgccggcggcgccgccatcaacgccttggccggcgtcgccggcgcctccgtccgcgtcgccgacttggccgtcgacgccgacccccccgacgaccgcatcggcgcccacaaggtccgccgcggctccggcgacatcaccgtccaggacgccttgaccgccgaggagaccgagcgcgccttgtccgccggcgccgccatcgccgacgaggaggtcgacgccggcgccgacttgttgatcgccggcgacatgggcatcggcaacaccaccgccgccgccgtcttggtcgccgccttgaccaacgtcgagcccgtcgtcgccgtcggcttcggcaccggcatcgacgacgccggctgggcccgcaagaccgccgccgtccgcgacgccttgttccgcgcccgccgcgtcttgcccgaccccgtcgccttgttgcgctgcgccggcggcgccgacttggccgccttggccggcttctgcgcccaggccgccgtccgccgcacccccttgttgttggacggcatggccgtcaccgccgccgccttggtcgccgagcacttggcccccggcgcccgcttgtggtggcaggccggccaccgctccaccgagcccggccacgccttggccttgaccgccttggacttggagcccatcttggacttgcgcatgcgcttgggcgagggcaccggcgccgccttggccttgcccatcgtccgcgccgccgtcgccgccttgtcctccatggccaccttcgcccaggccggcgtctccgacccctccgcccacccc

SEQ ID NO: 58 - RNA, 1047 nt, synthetic construct
  source 1..1047: mol_type = other RNA
atggaatttgcaacagtatcaccaccagatccaggaacagcagcagcagcacgagcacgacaagatacacttacaaaaccacgaggagcacttggacgacttgaagatctttcagtatggatagcagcatgtcaaggacaatgtccaccacgacaatttgaacgagcacgagtagtagtatttgcaggagatcatggagtagcacgatgtggagtatcagcatatccaccagaagtaacagcacaaatggtagcaaattttgatgcaggaggagcagcaataaatgcacttgcaggagtagcaggagcatcagtacgagtagcagatcttgcagtagatgcagatccaccagatgatcgaataggagcacataaagtacgacgaggatcaggagatataacagtacaagatgcacttacagcagaagaaacagaacgagcactttcagcaggagcagcaatagcagatgaagaagtagatgcaggagcagatcttcttatagcaggagatatgggaataggaaatacaacagcagcagcagtacttgtagcagcacttacaaatgtagaaccagtagtagcagtaggatttggaacaggaatagatgatgcaggatgggcacgaaaaacagcagcagtacgagatgcactttttcgagcacgacgagtacttccagatccagtagcacttcttcgatgtgcaggaggagcagatcttgcagcacttgcaggattttgtgcacaagcagcagtacgacgaacaccacttcttcttgatggaatggcagtaacagcagcagcacttgtagcagaacatcttgcaccaggagcacgactttggtggcaagcaggacatcgatcaacagaaccaggacatgcacttgcacttacagcacttgatcttgaaccaatacttgatcttcgaatgcgacttggagaaggaacaggagcagcacttgcacttccaatagtacgagcagcagtagcagcactttcatcaatggcaacatttgcacaagcaggagtatcagatccatcagcacatcca

SEQ ID NO: 59 - RNA, 1047 nt, synthetic construct
  source 1..1047: mol_type = other RNA
atggaatttgcgacggtgtcgccgccggatccggggacggcggcggcggcgcgggcgcggcaagatacgctcacgaaaccgcggggggcgctcgggcggctcgaagatctctcggtgtggattgcggcgtgtcaagggcaatgtccgccgcggcaatttgaacgggcgcgggtggtggtgtttgcgggggatcatggggtggcgcggtgtggggtgtcggcgtatccgccggaagtgacggcgcaaatggtggcgaattttgatgcggggggggcggcgattaatgcgctcgcgggggtggcgggggcgtcggtgcgggtggcggatctcgcggtggatgcggatccgccggatgatcggattggggcgcataaagtgcggcgggggtcgggggatattacggtgcaagatgcgctcacggcggaagaaacggaacgggcgctctcggcgggggcggcgattgcggatgaagaagtggatgcgggggcggatctcctcattgcgggggatatggggattgggaatacgacggcggcggcggtgctcgtggcggcgctcacgaatgtggaaccggtggtggcggtggggtttgggacggggattgatgatgcggggtgggcgcggaaaacggcggcggtgcgggatgcgctctttcgggcgcggcgggtgctcccggatccggtggcgctcctccggtgtgcggggggggcggatctcgcggcgctcgcggggttttgtgcgcaagcggcggtgcggcggacgccgctcctcctcgatgggatggcggtgacggcggcggcgctcgtggcggaacatctcgcgccgggggcgcggctctggtggcaagcggggcatcggtcgacggaaccggggcatgcgctcgcgctcacggcgctcgatctcgaaccgattctcgatctccggatgcggctcggggaagggacgggggcggcgctcgcgctcccgattgtgcgggcggcggtggcggcgctctcgtcgatggcgacgtttgcgcaagcgggggtgtcggatccgtcggcgcatccg

SEQ ID NO: 60 - RNA, 1047 nt, synthetic construct
  source 1..1047: mol_type = other RNA
atggaatttgctactgttagtcctcctgatcctggtactgctgctgctgctagagctagacaagatactctaactaaacctagaggtgctctaggtagactagaagatctaagtgtttggattgctgcttgtcaaggtcaatgtcctcctagacaatttgaaagagctagagttgttgtttttgctggtgatcatggtgttgctagatgtggtgttagtgcttatcctcctgaagttactgctcaaatggttgctaattttgatgctggtggtgctgctattaatgctctagctggtgttgctggtgctagtgttagagttgctgatctagctgttgatgctgatcctcctgatgatagaattggtgctcataaagttagaagaggtagtggtgatattactgttcaagatgctctaactgctgaagaaactgaaagagctctaagtgctggtgctgctattgctgatgaagaagttgatgctggtgctgatctactaattgctggtgatatgggtattggtaatactactgctgctgctgttctagttgctgctctaactaatgttgaacctgttgttgctgttggttttggtactggtattgatgatgctggttgggctagaaaaactgctgctgttagagatgctctatttagagctagaagagttctacctgatcctgttgctctactaagatgtgctggtggtgctgatctagctgctctagctggtttttgtgctcaagctgctgttagaagaactcctctactactagatggtatggctgttactgctgctgctctagttgctgaacatctagctcctggtgctagactatggtggcaagctggtcatagaagtactgaacctggtcatgctctagctctaactgctctagatctagaacctattctagatctaagaatgagactaggtgaaggtactggtgctgctctagctctacctattgttagagctgctgttgctgctctaagtagtatggctacttttgctcaagctggtgttagtgatcctagtgctcatcct

SEQ ID NO: 61 - RNA, 1047 nt, synthetic construct
  source 1..1047: mol_type = other RNA
atggaatttgctactgttagccctcctgatcctggtactgctgctgctgctagggctaggcaagatactctgactaaacctaggggtgctctgggtaggctggaagatctgagcgtttggattgctgcttgtcaaggtcaatgtcctcctaggcaatttgaaagggctagggttgttgtttttgctggtgatcatggtgttgctaggtgtggtgttagcgcttatcctcctgaagttactgctcaaatggttgctaattttgatgctggtggtgctgctattaatgctctggctggtgttgctggtgctagcgttagggttgctgatctggctgttgatgctgatcctcctgatgataggattggtgctcataaagttaggaggggtagcggtgatattactgttcaagatgctctgactgctgaagaaactgaaagggctctgagcgctggtgctgctattgctgatgaagaagttgatgctggtgctgatctgctgattgctggtgatatgggtattggtaatactactgctgctgctgttctggttgctgctctgactaatgttgaacctgttgttgctgttggttttggtactggtattgatgatgctggttgggctaggaaaactgctgctgttagggatgctctgtttagggctaggagggttctgcctgatcctgttgctctgctgaggtgtgctggtggtgctgatctggctgctctggctggtttttgtgctcaagctgctgttaggaggactcctctgctgctggatggtatggctgttactgctgctgctctggttgctgaacatctggctcctggtgctaggctgtggtggcaagctggtcataggagcactgaacctggtcatgctctggctctgactgctctggatctggaacctattctggatctgaggatgaggctgggtgaaggtactggtgctgctctggctctgcctattgttagggctgctgttgctgctctgagcagcatggctacttttgctcaagctggtgttagcgatcctagcgctcatcct

SEQ ID NO: 62 - RNA, 528 nt, synthetic construct
  source 1..528: mol_type = other RNA
atgcgtaatgttcgtaaaactttaattttagctgctgttactggtactttagttactattccttctgctactgctcatgctgatgctgaacaagttggttttgatcctaatgttcctgctgctggtcctgctcctgatgctcctcctcctcctgctcctgatgctcctcctgctcctgctcctgatgctcctcctgctcctgatgctcctcctcctcctgctcctgatgctcctcctcctgctcctgatgctcctcctgctcctggtcctgatgttcctcctcctcctgctcctaaagcttattctgttaattgggatgctattgctcaatgtgaatctggtggtaattggggtattaatactggtaatggttatgctggtggtttacaatttacttcttctacttggcatgctaatggtggttctggttctcctgctggtgcttctcgtgaagaacaaattcgtgttgctgaaaatgttttacattctcaaggtattggtgcttggcctgtttgtggtcgtcgtggt

SEQ ID NO: 63 - RNA, 528 nt, synthetic construct
  source 1..528: mol_type = other RNA
atgcgcaacgtccgcaagaccttgatcttggccgccgtcaccggcaccttggtcaccatcccctccgccaccgcccacgccgacgccgagcaggtcggcttcgaccccaacgtccccgccgccggccccgcccccgacgccccccccccccccgcccccgacgccccccccgcccccgcccccgacgccccccccgcccccgacgccccccccccccccgcccccgacgcccccccccccgcccccgacgccccccccgcccccggccccgacgtcccccccccccccgcccccaaggcctactccgtcaactgggacgccatcgcccagtgcgagtccggcggcaactggggcatcaacaccggcaacggctacgccggcggcttgcagttcacctcctccacctggcacgccaacggcggctccggctcccccgccggcgcctcccgcgaggagcagatccgcgtcgccgagaacgtcttgcactcccagggcatcggcgcctggcccgtctgcggccgccgcggc

SEQ ID NO: 64 - RNA, 528 nt, synthetic construct
  source 1..528: mol_type = other RNA
atgcgaaatgtacgaaaaacacttatacttgcagcagtaacaggaacacttgtaacaataccatcagcaacagcacatgcagatgcagaacaagtaggatttgatccaaatgtaccagcagcaggaccagcaccagatgcaccaccaccaccagcaccagatgcaccaccagcaccagcaccagatgcaccaccagcaccagatgcaccaccaccaccagcaccagatgcaccaccaccagcaccagatgcaccaccagcaccaggaccagatgtaccaccaccaccagcaccaaaagcatattcagtaaattgggatgcaatagcacaatgtgaatcaggaggaaattggggaataaatacaggaaatggatatgcaggaggacttcaatttacatcatcaacatggcatgcaaatggaggatcaggatcaccagcaggagcatcacgagaagaacaaatacgagtagcagaaaatgtacttcattcacaaggaataggagcatggccagtatgtggacgacgagga

SEQ ID NO: 65 - RNA, 528 nt, synthetic construct
  source 1..528: mol_type = other RNA
atgcggaatgtgcggaaaacgctcattctcgcggcggtgacggggacgctcgtgacgattccgtcggcgacggcgcatgcggatgcggaacaagtggggtttgatccgaatgtgccggcggcggggccggcgccggatgcgccgccgccgccggcgccggatgcgccgccggcgccggcgccggatgcgccgccggcgccggatgcgccgccgccgccggcgccggatgcgccgccgccggcgccggatgcgccgccggcgccggggccggatgtgccgccgccgccggcgccgaaagcgtattcggtgaattgggatgcgattgcgcaatgtgaatcgggggggaattgggggattaatacggggaatgggtatgcgggggggctccaatttacgtcgtcgacgtggcatgcgaatggggggtcggggtcgccggcgggggcgtcgcgggaagaacaaattcgggtggcggaaaatgtgctccattcgcaagggattggggcgtggccggtgtgtgggcggcggggg

SEQ ID NO: 66 - RNA, 528 nt, synthetic construct
  source 1..528: mol_type = other RNA
atgagaaatgttagaaaaactctaattctagctgctgttactggtactctagttactattcctagtgctactgctcatgctgatgctgaacaagttggttttgatcctaatgttcctgctgctggtcctgctcctgatgctcctcctcctcctgctcctgatgctcctcctgctcctgctcctgatgctcctcctgctcctgatgctcctcctcctcctgctcctgatgctcctcctcctgctcctgatgctcctcctgctcctggtcctgatgttcctcctcctcctgctcctaaagcttatagtgttaattgggatgctattgctcaatgtgaaagtggtggtaattggggtattaatactggtaatggttatgctggtggtctacaatttactagtagtacttggcatgctaatggtggtagtggtagtcctgctggtgctagtagagaagaacaaattagagttgctgaaaatgttctacatagtcaaggtattggtgcttggcctgtttgtggtagaagaggt

SEQ ID NO: 67 - RNA, 528 nt, synthetic construct
  source 1..528: mol_type = other RNA
atgaggaatgttaggaaaactctgattctggctgctgttactggtactctggttactattcctagcgctactgctcatgctgatgctgaacaagttggttttgatcctaatgttcctgctgctggtcctgctcctgatgctcctcctcctcctgctcctgatgctcctcctgctcctgctcctgatgctcctcctgctcctgatgctcctcctcctcctgctcctgatgctcctcctcctgctcctgatgctcctcctgctcctggtcctgatgttcctcctcctcctgctcctaaagcttatagcgttaattgggatgctattgctcaatgtgaaagcggtggtaattggggtattaatactggtaatggttatgctggtggtctgcaatttactagcagcacttggcatgctaatggtggtagcggtagccctgctggtgctagcagggaagaacaaattagggttgctgaaaatgttctgcatagccaaggtattggtgcttggcctgtttgtggtaggaggggt

SEQ ID NO: 68 - RNA, 390 nt, synthetic construct
  source 1..390: mol_type = other RNA
atggctaaattatctactgatgaattattagatgcttttaaagaaatgactttattagaattatctgattttgttaaaaaatttgaagaaacttttgaagttactgctgctgctcctgttgctgttgctgctgctggtgctgctcctgctggtgctgctgttgaagctgctgaagaacaatctgaatttgatgttattttagaagctgctggtgataaaaaaattggtgttattaaagttgttcgtgaaattgtttctggtttaggtttaaaagaagctaaagatttagttgatggtgctcctaaacctttattagaaaaagttgctaaagaagctgctgatgaagctaaagctaaattagaagctgctggtgctactgttactgttaaa

SEQ ID NO: 69 - RNA, 390 nt, synthetic construct
  source 1..390: mol_type = other RNA
atggccaagttgtccaccgacgagttgttggacgccttcaaggagatgaccttgttggagttgtccgacttcgtcaagaagttcgaggagaccttcgaggtcaccgccgccgcccccgtcgccgtcgccgccgccggcgccgcccccgccggcgccgccgtcgaggccgccgaggagcagtccgagttcgacgtcatcttggaggccgccggcgacaagaagatcggcgtcatcaaggtcgtccgcgagatcgtctccggcttgggcttgaaggaggccaaggacttggtcgacggcgcccccaagcccttgttggagaaggtcgccaaggaggccgccgacgaggccaaggccaagttggaggccgccggcgccaccgtcaccgtcaag

SEQ ID NO: 70 - RNA, 390 nt, synthetic construct
  source 1..390: mol_type = other RNA
atggcaaaactttcaacagatgaacttcttgatgcatttaaagaaatgacacttcttgaactttcagattttgtaaaaaaatttgaagaaacatttgaagtaacagcagcagcaccagtagcagtagcagcagcaggagcagcaccagcaggagcagcagtagaagcagcagaagaacaatcagaatttgatgtaatacttgaagcagcaggagataaaaaaataggagtaataaaagtagtacgagaaatagtatcaggacttggacttaaagaagcaaaagatcttgtagatggagcaccaaaaccacttcttgaaaaagtagcaaaagaagcagcagatgaagcaaaagcaaaacttgaagcagcaggagcaacagtaacagtaaaa

SEQ ID NO: 71 - RNA, 390 nt, synthetic construct
  source 1..390: mol_type = other RNA
atggcgaaactctcgacggatgaactcctcgatgcgtttaaagaaatgacgctcctcgaactctcggattttgtgaaaaaatttgaagaaacgtttgaagtgacggcggcggcgccggtggcggtggcggcggcgggggcggcgccggcgggggcggcggtggaagcggcggaagaacaatcggaatttgatgtgattctcgaagcggcgggggataaaaaaattggggtgattaaagtggtgcgggaaattgtgtcggggctcgggctcaaagaagcgaaagatctcgtggatggggcgccgaaaccgctcctcgaaaaagtggcgaaagaagcggcggatgaagcgaaagcgaaactcgaagcggcgggggcgacggtgacggtgaaa

SEQ ID NO: 72 - RNA, 390 nt, synthetic construct
  source 1..390: mol_type = other RNA
atggctaaactaagtactgatgaactactagatgcttttaaagaaatgactctactagaactaagtgattttgttaaaaaatttgaagaaacttttgaagttactgctgctgctcctgttgctgttgctgctgctggtgctgctcctgctggtgctgctgttgaagctgctgaagaacaaagtgaatttgatgttattctagaagctgctggtgataaaaaaattggtgttattaaagttgttagagaaattgttagtggtctaggtctaaaagaagctaaagatctagttgatggtgctcctaaacctctactagaaaaagttgctaaagaagctgctgatgaagctaaagctaaactagaagctgctggtgctactgttactgttaaa

SEQ ID NO: 73 - RNA, 390 nt, synthetic construct
  source 1..390: mol_type = other RNA
atggctaaactgagcactgatgaactgctggatgcttttaaagaaatgactctgctggaactgagcgattttgttaaaaaatttgaagaaacttttgaagttactgctgctgctcctgttgctgttgctgctgctggtgctgctcctgctggtgctgctgttgaagctgctgaagaacaaagcgaatttgatgttattctggaagctgctggtgataaaaaaattggtgttattaaagttgttagggaaattgttagcggtctgggtctgaaagaagctaaagatctggttgatggtgctcctaaacctctgctggaaaaagttgctaaagaagctgctgatgaagctaaagctaaactggaagctgctggtgctactgttactgttaaa

SEQ ID NO: 74 - RNA, 615 nt, synthetic construct
  source 1..615: mol_type = other RNA
atggctgaaaatcctaatattgatgatttacctgctcctttattagctgctttaggtgctgctgatttagctttagctactgttaatgatttaattgctaatttacgtgaacgtgctgaagaaactcgtgctgaaactcgtactcgtgttgaagaacgtcgtgctcgtttaactaaatttcaagaagatttacctgaacaatttattgaattacgtgataaatttactactgaagaattacgtaaagctgctgaaggttatttagaagctgctactaatcgttataatgaattagttgaacgtggtgaagctgctttacaacgtttacgttctcaaactgcttttgaagatgcttctgctcgtgctgaaggttatgttgatcaagctgttgaattaactcaagaagctttaggtactgttgcttctcaaactcgtgctgttggtgaacgtgctgctaaattagttggtattgaattacctggtaaagctgaagctgctggtaaaaaagctcaaaaagctattgctaaagctcctgctaaaaaagcttctgctaaaaaagctcctgctaaaaaagctcctgctaaaaaagctgctgctaaaaaagttactcaaaaa

SEQ ID NO: 75 - RNA, 615 nt, synthetic construct
  source 1..615: mol_type = other RNA
atggccgagaaccccaacatcgacgacttgcccgcccccttgttggccgccttgggcgccgccgacttggccttggccaccgtcaacgacttgatcgccaacttgcgcgagcgcgccgaggagacccgcgccgagacccgcacccgcgtcgaggagcgccgcgcccgcttgaccaagttccaggaggacttgcccgagcagttcatcgagttgcgcgacaagttcaccaccgaggagttgcgcaaggccgccgagggctacttggaggccgccaccaaccgctacaacgagttggtcgagcgcggcgaggccgccttgcagcgcttgcgctcccagaccgccttcgaggacgcctccgcccgcgccgagggctacgtcgaccaggccgtcgagttgacccaggaggccttgggcaccgtcgcctcccagacccgcgccgtcggcgagcgcgccgccaagttggtcggcatcgagttgcccggcaaggccgaggccgccggcaagaaggcccagaaggccatcgccaaggcccccgccaagaaggcctccgccaagaaggcccccgccaagaaggcccccgccaagaaggccgccgccaagaaggtcacccagaag

SEQ ID NO: 76 - RNA, 615 nt, synthetic construct
  source 1..615: mol_type = other RNA
atggcagaaaatccaaatatagatgatcttccagcaccacttcttgcagcacttggagcagcagatcttgcacttgcaacagtaaatgatcttatagcaaatcttcgagaacgagcagaagaaacacgagcagaaacacgaacacgagtagaagaacgacgagcacgacttacaaaatttcaagaagatcttccagaacaatttatagaacttcgagataaatttacaacagaagaacttcgaaaagcagcagaaggatatcttgaagcagcaacaaatcgatataatgaacttgtagaacgaggagaagcagcacttcaacgacttcgatcacaaacagcatttgaagatgcatcagcacgagcagaaggatatgtagatcaagcagtagaacttacacaagaagcacttggaacagtagcatcacaaacacgagcagtaggagaacgagcagcaaaacttgtaggaatagaacttccaggaaaagcagaagcagcaggaaaaaaagcacaaaaagcaatagcaaaagcaccagcaaaaaaagcatcagcaaaaaaagcaccagcaaaaaaagcaccagcaaaaaaagcagcagcaaaaaaagtaacacaaaaa

SEQ ID NO: 77 - RNA, 615 nt, synthetic construct
  source 1..615: mol_type = other RNA
atggcggaaaatccgaatattgatgatctcccggcgccgctcctcgcggcgctcggggcggcggatctcgcgctcgcgacggtgaatgatctcattgcgaatctccgggaacgggcggaagaaacgcgggcggaaacgcggacgcgggtggaagaacggcgggcgcggctcacgaaatttcaagaagatctcccggaacaatttattgaactccgggataaatttacgacggaagaactccggaaagcggcggaagggtatctcgaagcggcgacgaatcggtataatgaactcgtggaacggggggaagcggcgctccaacggctccggtcgcaaacggcgtttgaagatgcgtcggcgcgggcggaagggtatgtggatcaagcggtggaactcacgcaagaagcgctcgggacggtggcgtcgcaaacgcgggcggtgggggaacgggcggcgaaactcgtggggattgaactcccggggaaagcggaagcggcggggaaaaaagcgcaaaaagcgattgcgaaagcgccggcgaaaaaagcgtcggcgaaaaaagcgccggcgaaaaaagcgccggcgaaaaaagcggcggcgaaaaaagtgacgcaaaaa

SEQ ID NO: 78 - RNA, 615 nt, synthetic construct
  source 1..615: mol_type = other RNA
atggctgaaaatcctaatattgatgatctacctgctcctctactagctgctctaggtgctgctgatctagctctagctactgttaatgatctaattgctaatctaagagaaagagctgaagaaactagagctgaaactagaactagagttgaagaaagaagagctagactaactaaatttcaagaagatctacctgaacaatttattgaactaagagataaatttactactgaagaactaagaaaagctgctgaaggttatctagaagctgctactaatagatataatgaactagttgaaagaggtgaagctgctctacaaagactaagaagtcaaactgcttttgaagatgctagtgctagagctgaaggttatgttgatcaagctgttgaactaactcaagaagctctaggtactgttgctagtcaaactagagctgttggtgaaagagctgctaaactagttggtattgaactacctggtaaagctgaagctgctggtaaaaaagctcaaaaagctattgctaaagctcctgctaaaaaagctagtgctaaaaaagctcctgctaaaaaagctcctgctaaaaaagctgctgctaaaaaagttactcaaaaa

SEQ ID NO: 79 - RNA, 615 nt, synthetic construct
  source 1..615: mol_type = other RNA
atggctgaaaatcctaatattgatgatctgcctgctcctctgctggctgctctgggtgctgctgatctggctctggctactgttaatgatctgattgctaatctgagggaaagggctgaagaaactagggctgaaactaggactagggttgaagaaaggagggctaggctgactaaatttcaagaagatctgcctgaacaatttattgaactgagggataaatttactactgaagaactgaggaaagctgctgaaggttatctggaagctgctactaataggtataatgaactggttgaaaggggtgaagctgctctgcaaaggctgaggagccaaactgcttttgaagatgctagcgctagggctgaaggttatgttgatcaagctgttgaactgactcaagaagctctgggtactgttgctagccaaactagggctgttggtgaaagggctgctaaactggttggtattgaactgcctggtaaagctgaagctgctggtaaaaaagctcaaaaagctattgctaaagctcctgctaaaaaagctagcgctaaaaaagctcctgctaaaaaagctcctgctaaaaaagctgctgctaaaaaagttactcaaaaa

SEQ ID NO: 80 - RNA, 1782 nt, synthetic construct
  source 1..1782: mol_type = other RNA
atgaataaaatttatcgtattatttggaattctgctttaaatgcttgggttgttgtttctgaattaactcgtaatcatactaaacgtgcttctgctactgttgctactgctgttttagctactttattatttgctactgttcaagctaatgctactgatgatgatgatttatatttagaacctgttcaacgtactgctgttgttttatcttttcgttctgataaagaaggtactggtgaaaaagaaggtactgaagattctaattgggctgtttattttgatgaaaaacgtgttttaaaagctggtgctattactttaaaagctggtgataatttaaaaattaaacaaaatactaatgaaaatactaatgattcttcttttacttattctttaaaaaaagatttaactgatttaacttctgttgaaactgaaaaattatcttttggtgctaatggtaataaagttaatattacttctgatactaaaggtttaaattttgctaaagaaactgctggtactaatggtgatcctactgttcatttaaatggtattggttctactttaactgatactttattaaatactggtgctactactaatgttactaatgataatgttactgatgatgaaaaaaaacgtgctgcttctgttaaagatgttttaaatgctggttggaatattaaaggtgttaaacctggtactactgcttctgataatgttgattttgttcgtacttatgatactgttgaatttttatctgctgatactaaaactactactgttaatgttgaatctaaagataatggtaaaaaaactgaagttaaaattggtgctaaaacttctgttattaaagaaaaagatggtaaattagttactggtaaaggtaaagatgaaaatggttcttctactgatgaaggtgaaggtttagttactgctaaagaagttattgatgctgttaataaagctggttggcgtatgaaaactactactgctaatggtcaaactggtcaagctgataaatttgaaactgttacttctggtactaatgttacttttgcttctggtaaaggtactactgctactgtttctaaagatgatcaaggtaatattactgttaaatatgatgttaatgttggtgatgctttaaatgttaatcaattacaaaattctggttggaatttagattctaaagctgttgctggttcttctggtaaagttatttctggtaatgtttctccttctaaaggtaaaatggatgaaactgttaatattaatgctggtaataatattgaaattactcgtaatggtaaaaatattgatattgctacttctatggctcctcaattttcttctgtttctttaggtgctggtgctgatgctcctactttatctgttgatgatgaaggtgctttaaatgttggttctaaagatactaataaacctgttcgtattactaatgttgctcctggtgttaaagaaggtgatgttactaatgttgctcaattaaaaggtgttgctcaaaatttaaataatcgtattgataatgttgatggtaatgctcgtgctggtattgctcaagctattgctactgctggtttagttcaagcttatttacctggtaaatctatgatggctattggtggtgatacttatcgtggtgaagctggttatgctattggttattcttctatttctgatggtggtaattggattattaaaggtactgcttctggtaattctcgtggtcattttggtgcttctgcttctgttggttatcaatgg

SEQ ID NO: 81 - RNA, 1782 nt, synthetic construct
  source 1..1782: mol_type = other RNA
atgaacaagatctaccgcatcatctggaactccgccttgaacgcctgggtcgtcgtctccgagttgacccgcaaccacaccaagcgcgcctccgccaccgtcgccaccgccgtcttggccaccttgttgttcgccaccgtccaggccaacgccaccgacgacgacgacttgtacttggagcccgtccagcgcaccgccgtcgtcttgtccttccgctccgacaaggagggcaccggcgagaaggagggcaccgaggactccaactgggccgtctacttcgacgagaagcgcgtcttgaaggccggcgccatcaccttgaaggccggcgacaacttgaagatcaagcagaacaccaacgagaacaccaacgactcctccttcacctactccttgaagaaggacttgaccgacttgacctccgtcgagaccgagaagttgtccttcggcgccaacggcaacaaggtcaacatcacctccgacaccaagggcttgaacttcgccaaggagaccgccggcaccaacggcgaccccaccgtccacttgaacggcatcggctccaccttgaccgacaccttgttgaacaccggcgccaccaccaacgtcaccaacgacaacgtcaccgacgacgagaagaagcgcgccgcctccgtcaaggacgtcttgaacgccggctggaacatcaagggcgtcaagcccggcaccaccgcctccgacaacgtcgacttcgtccgcacctacgacaccgtcgagttcttgtccgccgacaccaagaccaccaccgtcaacgtcgagtccaaggacaacggcaagaagaccgaggtcaagatcggcgccaagacctccgtcatcaaggagaaggacggcaagttggtcaccggcaagggcaaggacgagaacggctcctccaccgacgagggcgagggcttggtcaccgccaaggaggtcatcgacgccgtcaacaaggccggctggcgcatgaagaccaccaccgccaacggccagaccggccaggccgacaagttcgagaccgtcacctccggcaccaacgtcaccttcgcctccggcaagggcaccaccgccaccgtctccaaggacgaccagggcaacatcaccgtcaagtacgacgtcaacgtcggcgacgccttgaacgtcaaccagttgcagaactccggctggaacttggactccaaggccgtcgccggctcctccggcaaggtcatctccggcaacgtctccccctccaagggcaagatggacgagaccgtcaacatcaacgccggcaacaacatcgagatcacccgcaacggcaagaacatcgacatcgccacctccatggccccccagttctcctccgtctccttgggcgccggcgccgacgcccccaccttgtccgtcgacgacgagggcgccttgaacgtcggctccaaggacaccaacaagcccgtccgcatcaccaacgtcgcccccggcgtcaaggagggcgacgtcaccaacgtcgcccagttgaagggcgtcgcccagaacttgaacaaccgcatcgacaacgtcgacggcaacgcccgcgccggcatcgcccaggccatcgccaccgccggcttggtccaggcctacttgcccggcaagtccatgatggccatcggcggcgacacctaccgcggcgaggccggctacgccatcggctactcctccatctccgacggcggcaactggatcatcaagggcaccgcctccggcaactcccgcggccacttcggcgcctccgcctccgtcggctaccagtgg

SEQ ID NO: 82 - RNA, 1782 nt, synthetic construct
  source 1..1782: mol_type = other RNA
atgaataaaatatatcgaataatatggaattcagcacttaatgcatgggtagtagtatcagaacttacacgaaatcatacaaaacgagcatcagcaacagtagcaacagcagtacttgcaacacttctttttgcaacagtacaagcaaatgcaacagatgatgatgatctttatcttgaaccagtacaacgaacagcagtagtactttcatttcgatcagataaagaaggaacaggagaaaaagaaggaacagaagattcaaattgggcagtatattttgatgaaaaacgagtacttaaagcaggagcaataacacttaaagcaggagataatcttaaaataaaacaaaatacaaatgaaaatacaaatgattcatcatttacatattcacttaaaaaagatcttacagatcttacatcagtagaaacagaaaaactttcatttggagcaaatggaaataaagtaaatataacatcagatacaaaaggacttaattttgcaaaagaaacagcaggaacaaatggagatccaacagtacatcttaatggaataggatcaacacttacagatacacttcttaatacaggagcaacaacaaatgtaacaaatgataatgtaacagatgatgaaaaaaaacgagcagcatcagtaaaagatgtacttaatgcaggatggaatataaaaggagtaaaaccaggaacaacagcatcagataatgtagattttgtacgaacatatgatacagtagaatttctttcagcagatacaaaaacaacaacagtaaatgtagaatcaaaagataatggaaaaaaaacagaagtaaaaataggagcaaaaacatcagtaataaaagaaaaagatggaaaacttgtaacaggaaaaggaaaagatgaaaatggatcatcaacagatgaaggagaaggacttgtaacagcaaaagaagtaatagatgcagtaaataaagcaggatggcgaatgaaaacaacaacagcaaatggacaaacaggacaagcagataaatttgaaacagtaacatcaggaacaaatgtaacatttgcatcaggaaaaggaacaacagcaacagtatcaaaagatgatcaaggaaatataacagtaaaatatgatgtaaatgtaggagatgcacttaatgtaaatcaacttcaaaattcaggatggaatcttgattcaaaagcagtagcaggatcatcaggaaaagtaatatcaggaaatgtatcaccatcaaaaggaaaaatggatgaaacagtaaatataaatgcaggaaataatatagaaataacacgaaatggaaaaaatatagatatagcaacatcaatggcaccacaattttcatcagtatcacttggagcaggagcagatgcaccaacactttcagtagatgatgaaggagcacttaatgtaggatcaaaagatacaaataaaccagtacgaataacaaatgtagcaccaggagtaaaagaaggagatgtaacaaatgtagcacaacttaaaggagtagcacaaaatcttaataatcgaatagataatgtagatggaaatgcacgagcaggaatagcacaagcaatagcaacagcaggacttgtacaagcatatcttccaggaaaatcaatgatggcaataggaggagatacatatcgaggagaagcaggatatgcaataggatattcatcaatatcagatggaggaaattggataataaaaggaacagcatcaggaaattcacgaggacattttggagcatcagcatcagtaggatatcaatgg

SEQ ID NO: 83 - RNA, 1782 nt, synthetic construct
  source 1..1782: mol_type = other RNA
atgaataaaatttatcggattatttggaattcggcgctcaatgcgtgggtggtggtgtcggaactcacgcggaatcatacgaaacgggcgtcggcgacggtggcgacggcggtgctcgcgacgctcctctttgcgacggtgcaagcgaatgcgacggatgatgatgatctctatctcgaaccggtgcaacggacggcggtggtgctctcgtttcggtcggataaagaagggacgggggaaaaagaagggacggaagattcgaattgggcggtgtattttgatgaaaaacgggtgctcaaagcgggggcgattacgctcaaagcgggggataatctcaaaattaaacaaaatacgaatgaaaatacgaatgattcgtcgtttacgtattcgctcaaaaaagatctcacggatctcacgtcggtggaaacggaaaaactctcgtttggggcgaatgggaataaagtgaatattacgtcggatacgaaagggctcaattttgcgaaagaaacggcggggacgaatggggatccgacggtgcatctcaatgggattgggtcgacgctcacggatacgctcctcaatacgggggcgacgacgaatgtgacgaatgataatgtgacggatgatgaaaaaaaacgggcggcgtcggtgaaagatgtgctcaatgcggggtggaatattaaaggggtgaaaccggggacgacggcgtcggataatgtggattttgtgcggacgtatgatacggtggaatttctctcggcggatacgaaaacgacgacggtgaatgtggaatcgaaagataatgggaaaaaaacggaagtgaaaattggggcgaaaacgtcggtgattaaagaaaaagatgggaaactcgtgacggggaaagggaaagatgaaaatgggtcgtcgacggatgaaggggaagggctcgtgacggcgaaagaagtgattgatgcggtgaataaagcggggtggcggatgaaaacgacgacggcgaatgggcaaacggggcaagcggataaatttgaaacggtgacgtcggggacgaatgtgacgtttgcgtcggggaaagggacgacggcgacggtgtcgaaagatgatcaagggaatattacggtgaaatatgatgtgaatgtgggggatgcgctcaatgtgaatcaactccaaaattcggggtggaatctcgattcgaaagcggtggcggggtcgtcggggaaagtgatttcggggaatgtgtcgccgtcgaaagggaaaatggatgaaacggtgaatattaatgcggggaataatattgaaattacgcggaatgggaaaaatattgatattgcgacgtcgatggcgccgcaattttcgtcggtgtcgctcggggcgggggcggatgcgccgacgctctcggtggatgatgaaggggcgctcaatgtggggtcgaaagatacgaataaaccggtgcggattacgaatgtggcgccgggggtgaaagaaggggatgtgacgaatgtggcgcaactcaaaggggtggcgcaaaatctcaataatcggattgataatgtggatgggaatgcgcgggcggggattgcgcaagcgattgcgacggcggggctcgtgcaagcgtatctcccggggaaatcgatgatggcgattgggggggatacgtatcggggggaagcggggtatgcgattgggtattcgtcgatttcggatggggggaattggattattaaagggacggcgtcggggaattcgcgggggcattttggggcgtcggcgtcggtggggtatcaatgg

SEQ ID NO: 84 - RNA, 1782 nt, synthetic construct
  source 1..1782: mol_type = other RNA
atgaataaaatttatagaattatttggaatagtgctctaaatgcttgggttgttgttagtgaactaactagaaatcatactaaaagagctagtgctactgttgctactgctgttctagctactctactatttgctactgttcaagctaatgctactgatgatgatgatctatatctagaacctgttcaaagaactgctgttgttctaagttttagaagtgataaagaaggtactggtgaaaaagaaggtactgaagatagtaattgggctgtttattttgatgaaaaaagagttctaaaagctggtgctattactctaaaagctggtgataatctaaaaattaaacaaaatactaatgaaaatactaatgatagtagttttacttatagtctaaaaaaagatctaactgatctaactagtgttgaaactgaaaaactaagttttggtgctaatggtaataaagttaatattactagtgatactaaaggtctaaattttgctaaagaaactgctggtactaatggtgatcctactgttcatctaaatggtattggtagtactctaactgatactctactaaatactggtgctactactaatgttactaatgataatgttactgatgatgaaaaaaaaagagctgctagtgttaaagatgttctaaatgctggttggaatattaaaggtgttaaacctggtactactgctagtgataatgttgattttgttagaacttatgatactgttgaatttctaagtgctgatactaaaactactactgttaatgttgaaagtaaagataatggtaaaaaaactgaagttaaaattggtgctaaaactagtgttattaaagaaaaagatggtaaactagttactggtaaaggtaaagatgaaaatggtagtagtactgatgaaggtgaaggtctagttactgctaaagaagttattgatgctgttaataaagctggttggagaatgaaaactactactgctaatggtcaaactggtcaagctgataaatttgaaactgttactagtggtactaatgttacttttgctagtggtaaaggtactactgctactgttagtaaagatgatcaaggtaatattactgttaaatatgatgttaatgttggtgatgctctaaatgttaatcaactacaaaatagtggttggaatctagatagtaaagctgttgctggtagtagtggtaaagttattagtggtaatgttagtcctagtaaaggtaaaatggatgaaactgttaatattaatgctggtaataatattgaaattactagaaatggtaaaaatattgatattgctactagtatggctcctcaatttagtagtgttagtctaggtgctggtgctgatgctcctactctaagtgttgatgatgaaggtgctctaaatgttggtagtaaagatactaataaacctgttagaattactaatgttgctcctggtgttaaagaaggtgatgttactaatgttgctcaactaaaaggtgttgctcaaaatctaaataatagaattgataatgttgatggtaatgctagagctggtattgctcaagctattgctactgctggtctagttcaagcttatctacctggtaaaagtatgatggctattggtggtgatacttatagaggtgaagctggttatgctattggttatagtagtattagtgatggtggtaattggattattaaaggtactgctagtggtaatagtagaggtcattttggtgctagtgctagtgttggttatcaatgg

SEQ ID NO: 85 - RNA, 1782 nt, synthetic construct
  source 1..1782: mol_type = other RNA
atgaataaaatttataggattatttggaatagcgctctgaatgcttgggttgttgttagcgaactgactaggaatcatactaaaagggctagcgctactgttgctactgctgttctggctactctgctgtttgctactgttcaagctaatgctactgatgatgatgatctgtatctggaacctgttcaaaggactgctgttgttctgagctttaggagcgataaagaaggtactggtgaaaaagaaggtactgaagatagcaattgggctgtttattttgatgaaaaaagggttctgaaagctggtgctattactctgaaagctggtgataatctgaaaattaaacaaaatactaatgaaaatactaatgatagcagctttacttatagcctgaaaaaagatctgactgatctgactagcgttgaaactgaaaaactgagctttggtgctaatggtaataaagttaatattactagcgatactaaaggtctgaattttgctaaagaaactgctggtactaatggtgatcctactgttcatctgaatggtattggtagcactctgactgatactctgctgaatactggtgctactactaatgttactaatgataatgttactgatgatgaaaaaaaaagggctgctagcgttaaagatgttctgaatgctggttggaatattaaaggtgttaaacctggtactactgctagcgataatgttgattttgttaggacttatgatactgttgaatttctgagcgctgatactaaaactactactgttaatgttgaaagcaaagataatggtaaaaaaactgaagttaaaattggtgctaaaactagcgttattaaagaaaaagatggtaaactggttactggtaaaggtaaagatgaaaatggtagcagcactgatgaaggtgaaggtctggttactgctaaagaagttattgatgctgttaataaagctggttggaggatgaaaactactactgctaatggtcaaactggtcaagctgataaatttgaaactgttactagcggtactaatgttacttttgctagcggtaaaggtactactgctactgttagcaaagatgatcaaggtaatattactgttaaatatgatgttaatgttggtgatgctctgaatgttaatcaactgcaaaatagcggttggaatctggatagcaaagctgttgctggtagcagcggtaaagttattagcggtaatgttagccctagcaaaggtaaaatggatgaaactgttaatattaatgctggtaataatattgaaattactaggaatggtaaaaatattgatattgctactagcatggctcctcaatttagcagcgttagcctgggtgctggtgctgatgctcctactctgagcgttgatgatgaaggtgctctgaatgttggtagcaaagatactaataaacctgttaggattactaatgttgctcctggtgttaaagaaggtgatgttactaatgttgctcaactgaaaggtgttgctcaaaatctgaataataggattgataatgttgatggtaatgctagggctggtattgctcaagctattgctactgctggtctggttcaagcttatctgcctggtaaaagcatgatggctattggtggtgatacttataggggtgaagctggttatgctattggttatagcagcattagcgatggtggtaattggattattaaaggtactgctagcggtaatagcaggggtcattttggtgctagcgctagcgttggttatcaatgg

SEQ ID NO: 86 - RNA, 1134 nt, synthetic construct
  source 1..1134: mol_type = other RNA
atgaataatactgaattttatgatcgtttaggtgtttctaaaaatgcttctgctgatgaaattaaaaaagcttatcgtaaattatctaaaaaatatcatcctgatattaataaagaacctggtgctgaagataaatataaagaagttcaagaagcttatgaaactttatctgatgatcaaaaacgtgctgcttatgatcaatatggtgctgctggtgctaatggtggttttggtggtgctggtggttttggtggttttaatggtgctggtggttttggtggttttgaagatattttttcttctttttttggtggtggtggttcttctcgtaatcctaatgctcctcgtcaaggtgatgatttacaatatcgtgttaatttaacttttgaagaagctatttttggtactgaaaaagaagttaaatatcatcgtgaagctggttgtcgtacttgtaatggttctggtgctaaacctggtacttctcctgttacttgtggtcgttgtcatggtgctggtgttattaatgttgatactcaaactcctttaggtatgatgcgtcgtcaagttacttgtgatgtttgtcatggtcgtggtaaagaaattaaatatccttgtactacttgtcatggtactggtcatgaaaaacaagctcattctgttcatgttaaaattcctgctggtgttgaaactggtcaacaaattcgtttagctggtcaaggtgaagctggttttaatggtggtccttatggtgatttatatgttgttgtttctgttgaagcttctgataaatttgaacgtgaaggtactactattttttataatttaaatttaaattttgttcaagctgctttaggtgatactgttgatattcctactgttcatggtgatgttgaattagttattcctgaaggtactcaaactggtaaaaaatttcgtttacgttctaaaggtgctccttctttacgtggtggtgctgttggtgatcaatatgttactgttaatgttgttactcctactggtttaaatgatcgtcaaaaagttgctttaaaagaatttgctgctgctggtgatttaaaagttaatcctaaaaaaaaaggtttttttgatcatattaaagatgcttttgatggtgaa

SEQ ID NO: 87 - RNA, 1134 nt, synthetic construct
  source 1..1134: mol_type = other RNA
atgaacaacaccgagttctacgaccgcttgggcgtctccaagaacgcctccgccgacgagatcaagaaggcctaccgcaagttgtccaagaagtaccaccccgacatcaacaaggagcccggcgccgaggacaagtacaaggaggtccaggaggcctacgagaccttgtccgacgaccagaagcgcgccgcctacgaccagtacggcgccgccggcgccaacggcggcttcggcggcgccggcggcttcggcggcttcaacggcgccggcggcttcggcggcttcgaggacatcttctcctccttcttcggcggcggcggctcctcccgcaaccccaacgccccccgccagggcgacgacttgcagtaccgcgtcaacttgaccttcgaggaggccatcttcggcaccgagaaggaggtcaagtaccaccgcgaggccggctgccgcacctgcaacggctccggcgccaagcccggcacctcccccgtcacctgcggccgctgccacggcgccggcgtcatcaacgtcgacacccagacccccttgggcatgatgcgccgccaggtcacctgcgacgtctgccacggccgcggcaaggagatcaagtacccctgcaccacctgccacggcaccggccacgagaagcaggcccactccgtccacgtcaagatccccgccggcgtcgagaccggccagcagatccgcttggccggccagggcgaggccggcttcaacggcggcccctacggcgacttgtacgtcgtcgtctccgtcgaggcctccgacaagttcgagcgcgagggcaccaccatcttctacaacttgaacttgaacttcgtccaggccgccttgggcgacaccgtcgacatccccaccgtccacggcgacgtcgagttggtcatccccgagggcacccagaccggcaagaagttccgcttgcgctccaagggcgccccctccttgcgcggcggcgccgtcggcgaccagtacgtcaccgtcaacgtcgtcacccccaccggcttgaacgaccgccagaaggtcgccttgaaggagttcgccgccgccggcgacttgaaggtcaaccccaagaagaagggcttcttcgaccacatcaaggacgccttcgacggcgag

SEQ ID NO: 88 - RNA, 1134 nt, synthetic construct
  source 1..1134: mol_type = other RNA
atgaataatacagaattttatgatcgacttggagtatcaaaaaatgcatcagcagatgaaataaaaaaagcatatcgaaaactttcaaaaaaatatcatccagatataaataaagaaccaggagcagaagataaatataaagaagtacaagaagcatatgaaacactttcagatgatcaaaaacgagcagcatatgatcaatatggagcagcaggagcaaatggaggatttggaggagcaggaggatttggaggatttaatggagcaggaggatttggaggatttgaagatatattttcatcattttttggaggaggaggatcatcacgaaatccaaatgcaccacgacaaggagatgatcttcaatatcgagtaaatcttacatttgaagaagcaatatttggaacagaaaaagaagtaaaatatcatcgagaagcaggatgtcgaacatgtaatggatcaggagcaaaaccaggaacatcaccagtaacatgtggacgatgtcatggagcaggagtaataaatgtagatacacaaacaccacttggaatgatgcgacgacaagtaacatgtgatgtatgtcatggacgaggaaaagaaataaaatatccatgtacaacatgtcatggaacaggacatgaaaaacaagcacattcagtacatgtaaaaataccagcaggagtagaaacaggacaacaaatacgacttgcaggacaaggagaagcaggatttaatggaggaccatatggagatctttatgtagtagtatcagtagaagcatcagataaatttgaacgagaaggaacaacaatattttataatcttaatcttaattttgtacaagcagcacttggagatacagtagatataccaacagtacatggagatgtagaacttgtaataccagaaggaacacaaacaggaaaaaaatttcgacttcgatcaaaaggagcaccatcacttcgaggaggagcagtaggagatcaatatgtaacagtaaatgtagtaacaccaacaggacttaatgatcgacaaaaagtagcacttaaagaatttgcagcagcaggagatcttaaagtaaatccaaaaaaaaaaggattttttgatcatataaaagatgcatttgatggagaa

SEQ ID NO: 89 - RNA, 1134 nt, synthetic construct
  source 1..1134: mol_type = other RNA
atgaataatacggaattttatgatcggctcggggtgtcgaaaaatgcgtcggcggatgaaattaaaaaagcgtatcggaaactctcgaaaaaatatcatccggatattaataaagaaccgggggcggaagataaatataaagaagtgcaagaagcgtatgaaacgctctcggatgatcaaaaacgggcggcgtatgatcaatatggggcggcgggggcgaatggggggtttgggggggcgggggggtttggggggtttaatggggcgggggggtttggggggtttgaagatattttttcgtcgttttttggggggggggggtcgtcgcggaatccgaatgcgccgcggcaaggggatgatctccaatatcgggtgaatctcacgtttgaagaagcgatttttgggacggaaaaagaagtgaaatatcatcgggaagcggggtgtcggacgtgtaatgggtcgggggcgaaaccggggacgtcgccggtgacgtgtgggcggtgtcatggggcgggggtgattaatgtggatacgcaaacgccgctcgggatgatgcggcggcaagtgacgtgtgatgtgtgtcatgggcgggggaaagaaattaaatatccgtgtacgacgtgtcatgggacggggcatgaaaaacaagcgcattcggtgcatgtgaaaattccggcgggggtggaaacggggcaacaaattcggctcgcggggcaaggggaagcggggtttaatggggggccgtatggggatctctatgtggtggtgtcggtggaagcgtcggataaatttgaacgggaagggacgacgattttttataatctcaatctcaattttgtgcaagcggcgctcggggatacggtggatattccgacggtgcatggggatgtggaactcgtgattccggaagggacgcaaacggggaaaaaatttcggctccggtcgaaaggggcgccgtcgctccgggggggggcggtgggggatcaatatgtgacggtgaatgtggtgacgccgacggggctcaatgatcggcaaaaagtggcgctcaaagaatttgcggcggcgggggatctcaaagtgaatccgaaaaaaaaagggttttttgatcatattaaagatgcgtttgatggggaa

SEQ ID NO: 90 - RNA, 1134 nt, synthetic construct
  source 1..1134: mol_type = other RNA
atgaataatactgaattttatgatagactaggtgttagtaaaaatgctagtgctgatgaaattaaaaaagcttatagaaaactaagtaaaaaatatcatcctgatattaataaagaacctggtgctgaagataaatataaagaagttcaagaagcttatgaaactctaagtgatgatcaaaaaagagctgcttatgatcaatatggtgctgctggtgctaatggtggttttggtggtgctggtggttttggtggttttaatggtgctggtggttttggtggttttgaagatatttttagtagtttttttggtggtggtggtagtagtagaaatcctaatgctcctagacaaggtgatgatctacaatatagagttaatctaacttttgaagaagctatttttggtactgaaaaagaagttaaatatcatagagaagctggttgtagaacttgtaatggtagtggtgctaaacctggtactagtcctgttacttgtggtagatgtcatggtgctggtgttattaatgttgatactcaaactcctctaggtatgatgagaagacaagttacttgtgatgtttgtcatggtagaggtaaagaaattaaatatccttgtactacttgtcatggtactggtcatgaaaaacaagctcatagtgttcatgttaaaattcctgctggtgttgaaactggtcaacaaattagactagctggtcaaggtgaagctggttttaatggtggtccttatggtgatctatatgttgttgttagtgttgaagctagtgataaatttgaaagagaaggtactactattttttataatctaaatctaaattttgttcaagctgctctaggtgatactgttgatattcctactgttcatggtgatgttgaactagttattcctgaaggtactcaaactggtaaaaaatttagactaagaagtaaaggtgctcctagtctaagaggtggtgctgttggtgatcaatatgttactgttaatgttgttactcctactggtctaaatgatagacaaaaagttgctctaaaagaatttgctgctgctggtgatctaaaagttaatcctaaaaaaaaaggtttttttgatcatattaaagatgcttttgatggtgaa

SEQ ID NO: 91 - RNA, 1134 nt, synthetic construct
  source 1..1134: mol_type = other RNA
atgaataatactgaattttatgataggctgggtgttagcaaaaatgctagcgctgatgaaattaaaaaagcttataggaaactgagcaaaaaatatcatcctgatattaataaagaacctggtgctgaagataaatataaagaagttcaagaagcttatgaaactctgagcgatgatcaaaaaagggctgcttatgatcaatatggtgctgctggtgctaatggtggttttggtggtgctggtggttttggtggttttaatggtgctggtggttttggtggttttgaagatatttttagcagcttttttggtggtggtggtagcagcaggaatcctaatgctcctaggcaaggtgatgatctgcaatatagggttaatctgacttttgaagaagctatttttggtactgaaaaagaagttaaatatcatagggaagctggttgtaggacttgtaatggtagcggtgctaaacctggtactagccctgttacttgtggtaggtgtcatggtgctggtgttattaatgttgatactcaaactcctctgggtatgatgaggaggcaagttacttgtgatgtttgtcatggtaggggtaaagaaattaaatatccttgtactacttgtcatggtactggtcatgaaaaacaagctcatagcgttcatgttaaaattcctgctggtgttgaaactggtcaacaaattaggctggctggtcaaggtgaagctggttttaatggtggtccttatggtgatctgtatgttgttgttagcgttgaagctagcgataaatttgaaagggaaggtactactattttttataatctgaatctgaattttgttcaagctgctctgggtgatactgttgatattcctactgttcatggtgatgttgaactggttattcctgaaggtactcaaactggtaaaaaatttaggctgaggagcaaaggtgctcctagcctgaggggtggtgctgttggtgatcaatatgttactgttaatgttgttactcctactggtctgaatgataggcaaaaagttgctctgaaagaatttgctgctgctggtgatctgaaagttaatcctaaaaaaaaaggtttttttgatcatattaaagatgcttttgatggtgaa

SEQ ID NO: 92 - RNA, 1413 nt, synthetic construct
  source 1..1413: mol_type = other RNA
atggctaataaagctgttaatgattttattttagctatgaattatgataaaaaaaaattattaactcatcaaggtgaatctattgaaaatcgttttattaaagaaggtaatcaattacctgatgaatttgttgttattgaacgtaaaaaacgttctttatctactaatacttctgatatttctgttactgctactaatgattctcgtttatatcctggtgctttattagttgttgatgaaactttattagaaaataatcctactttattagctgttgatcgtgctcctatgacttattctattgatttacctggtttagcttcttctgattcttttttacaagttgaagatccttctaattcttctgttcgtggtgctgttaatgatttattagctaaatggcatcaagattatggtcaagttaataatgttcctgctcgtatgcaatatgaaaaaattactgctcattctatggaacaattaaaagttaaatttggttctgattttgaaaaaactggtaattctttagatattgattttaattctgttcattctggtgaaaaacaaattcaaattgttaattttaaacaaatttattatactgtttctgttgatgctgttaaaaatcctggtgatgtttttcaagatactgttactgttgaagatttaaaacaacgtggtatttctgctgaacgtcctttagtttatatttcttctgttgcttatggtcgtcaagtttatttaaaattagaaactacttctaaatctgatgaagttgaagctgcttttgaagctttaattaaaggtgttaaagttgctcctcaaactgaatggaaacaaattttagataatactgaagttaaagctgttattttaggtggtgatccttcttctggtgctcgtgttgttactggtaaagttgatatggttgaagatttaattcaagaaggttctcgttttactgctgatcatcctggtttacctatttcttatactacttcttttttacgtgataatgttgttgctacttttcaaaattctactgattatgttgaaactaaagttactgcttatcgtaatggtgatttattattagatcattctggtgcttatgttgctcaatattatattacttgggatgaattatcttatgatcatcaaggtaaagaagttttaactcctaaagcttgggatcgtaatggtcaagatttaactgctcattttactacttctattcctttaaaaggtaatgttcgtaatttatctgttaaaattcgtgaatgtactggtttagcttgggaatggtggcgtactgtttatgaaaaaactgatttacctttagttcgtaaacgtactatttctatttggggtactactttatatcctcaagttgaagataaagttgaaaatgat

SEQ ID NO: 93 - RNA, 1413 nt, synthetic construct
  source 1..1413: mol_type = other RNA
atggccaacaaggccgtcaacgacttcatcttggccatgaactacgacaagaagaagttgttgacccaccagggcgagtccatcgagaaccgcttcatcaaggagggcaaccagttgcccgacgagttcgtcgtcatcgagcgcaagaagcgctccttgtccaccaacacctccgacatctccgtcaccgccaccaacgactcccgcttgtaccccggcgccttgttggtcgtcgacgagaccttgttggagaacaaccccaccttgttggccgtcgaccgcgcccccatgacctactccatcgacttgcccggcttggcctcctccgactccttcttgcaggtcgaggacccctccaactcctccgtccgcggcgccgtcaacgacttgttggccaagtggcaccaggactacggccaggtcaacaacgtccccgcccgcatgcagtacgagaagatcaccgcccactccatggagcagttgaaggtcaagttcggctccgacttcgagaagaccggcaactccttggacatcgacttcaactccgtccactccggcgagaagcagatccagatcgtcaacttcaagcagatctactacaccgtctccgtcgacgccgtcaagaaccccggcgacgtcttccaggacaccgtcaccgtcgaggacttgaagcagcgcggcatctccgccgagcgccccttggtctacatctcctccgtcgcctacggccgccaggtctacttgaagttggagaccacctccaagtccgacgaggtcgaggccgccttcgaggccttgatcaagggcgtcaaggtcgccccccagaccgagtggaagcagatcttggacaacaccgaggtcaaggccgtcatcttgggcggcgacccctcctccggcgcccgcgtcgtcaccggcaaggtcgacatggtcgaggacttgatccaggagggctcccgcttcaccgccgaccaccccggcttgcccatctcctacaccacctccttcttgcgcgacaacgtcgtcgccaccttccagaactccaccgactacgtcgagaccaaggtcaccgcctaccgcaacggcgacttgttgttggaccactccggcgcctacgtcgcccagtactacatcacctgggacgagttgtcctacgaccaccagggcaaggaggtcttgacccccaaggcctgggaccgcaacggccaggacttgaccgcccacttcaccacctccatccccttgaagggcaacgtccgcaacttgtccgtcaagatccgcgagtgcaccggcttggcctgggagtggtggcgcaccgtctacgagaagaccgacttgcccttggtccgcaagcgcaccatctccatctggggcaccaccttgtacccccaggtcgaggacaaggtcgagaacgac

SEQ ID NO: 94 - RNA, 1413 nt, synthetic construct
  source 1..1413: mol_type = other RNA
atggcaaataaagcagtaaatgattttatacttgcaatgaattatgataaaaaaaaacttcttacacatcaaggagaatcaatagaaaatcgatttataaaagaaggaaatcaacttccagatgaatttgtagtaatagaacgaaaaaaacgatcactttcaacaaatacatcagatatatcagtaacagcaacaaatgattcacgactttatccaggagcacttcttgtagtagatgaaacacttcttgaaaataatccaacacttcttgcagtagatcgagcaccaatgacatattcaatagatcttccaggacttgcatcatcagattcatttcttcaagtagaagatccatcaaattcatcagtacgaggagcagtaaatgatcttcttgcaaaatggcatcaagattatggacaagtaaataatgtaccagcacgaatgcaatatgaaaaaataacagcacattcaatggaacaacttaaagtaaaatttggatcagattttgaaaaaacaggaaattcacttgatatagattttaattcagtacattcaggagaaaaacaaatacaaatagtaaattttaaacaaatatattatacagtatcagtagatgcagtaaaaaatccaggagatgtatttcaagatacagtaacagtagaagatcttaaacaacgaggaatatcagcagaacgaccacttgtatatatatcatcagtagcatatggacgacaagtatatcttaaacttgaaacaacatcaaaatcagatgaagtagaagcagcatttgaagcacttataaaaggagtaaaagtagcaccacaaacagaatggaaacaaatacttgataatacagaagtaaaagcagtaatacttggaggagatccatcatcaggagcacgagtagtaacaggaaaagtagatatggtagaagatcttatacaagaaggatcacgatttacagcagatcatccaggacttccaatatcatatacaacatcatttcttcgagataatgtagtagcaacatttcaaaattcaacagattatgtagaaacaaaagtaacagcatatcgaaatggagatcttcttcttgatcattcaggagcatatgtagcacaatattatataacatgggatgaactttcatatgatcatcaaggaaaagaagtacttacaccaaaagcatgggatcgaaatggacaagatcttacagcacattttacaacatcaataccacttaaaggaaatgtacgaaatctttcagtaaaaatacgagaatgtacaggacttgcatgggaatggtggcgaacagtatatgaaaaaacagatcttccacttgtacgaaaacgaacaatatcaatatggggaacaacactttatccacaagtagaagataaagtagaaaatgat

SEQ ID NO: 95 - RNA, 1413 nt, synthetic construct
  source 1..1413: mol_type = other RNA
atggcgaataaagcggtgaatgattttattctcgcgatgaattatgataaaaaaaaactcctcacgcatcaaggggaatcgattgaaaatcggtttattaaagaagggaatcaactcccggatgaatttgtggtgattgaacggaaaaaacggtcgctctcgacgaatacgtcggatatttcggtgacggcgacgaatgattcgcggctctatccgggggcgctcctcgtggtggatgaaacgctcctcgaaaataatccgacgctcctcgcggtggatcgggcgccgatgacgtattcgattgatctcccggggctcgcgtcgtcggattcgtttctccaagtggaagatccgtcgaattcgtcggtgcggggggcggtgaatgatctcctcgcgaaatggcatcaagattatgggcaagtgaataatgtgccggcgcggatgcaatatgaaaaaattacggcgcattcgatggaacaactcaaagtgaaatttgggtcggattttgaaaaaacggggaattcgctcgatattgattttaattcggtgcattcgggggaaaaacaaattcaaattgtgaattttaaacaaatttattatacggtgtcggtggatgcggtgaaaaatccgggggatgtgtttcaagatacggtgacggtggaagatctcaaacaacgggggatttcggcggaacggccgctcgtgtatatttcgtcggtggcgtatgggcggcaagtgtatctcaaactcgaaacgacgtcgaaatcggatgaagtggaagcggcgtttgaagcgctcattaaaggggtgaaagtggcgccgcaaacggaatggaaacaaattctcgataatacggaagtgaaagcggtgattctcgggggggatccgtcgtcgggggcgcgggtggtgacggggaaagtggatatggtggaagatctcattcaagaagggtcgcggtttacggcggatcatccggggctcccgatttcgtatacgacgtcgtttctccgggataatgtggtggcgacgtttcaaaattcgacggattatgtggaaacgaaagtgacggcgtatcggaatggggatctcctcctcgatcattcgggggcgtatgtggcgcaatattatattacgtgggatgaactctcgtatgatcatcaagggaaagaagtgctcacgccgaaagcgtgggatcggaatgggcaagatctcacggcgcattttacgacgtcgattccgctcaaagggaatgtgcggaatctctcggtgaaaattcgggaatgtacggggctcgcgtgggaatggtggcggacggtgtatgaaaaaacggatctcccgctcgtgcggaaacggacgatttcgatttgggggacgacgctctatccgcaagtggaagataaagtggaaaatgat

SEQ ID NO: 96 - RNA, 1413 nt, synthetic construct
  source 1..1413: mol_type = other RNA
atggctaataaagctgttaatgattttattctagctatgaattatgataaaaaaaaactactaactcatcaaggtgaaagtattgaaaatagatttattaaagaaggtaatcaactacctgatgaatttgttgttattgaaagaaaaaaaagaagtctaagtactaatactagtgatattagtgttactgctactaatgatagtagactatatcctggtgctctactagttgttgatgaaactctactagaaaataatcctactctactagctgttgatagagctcctatgacttatagtattgatctacctggtctagctagtagtgatagttttctacaagttgaagatcctagtaatagtagtgttagaggtgctgttaatgatctactagctaaatggcatcaagattatggtcaagttaataatgttcctgctagaatgcaatatgaaaaaattactgctcatagtatggaacaactaaaagttaaatttggtagtgattttgaaaaaactggtaatagtctagatattgattttaatagtgttcatagtggtgaaaaacaaattcaaattgttaattttaaacaaatttattatactgttagtgttgatgctgttaaaaatcctggtgatgtttttcaagatactgttactgttgaagatctaaaacaaagaggtattagtgctgaaagacctctagtttatattagtagtgttgcttatggtagacaagtttatctaaaactagaaactactagtaaaagtgatgaagttgaagctgcttttgaagctctaattaaaggtgttaaagttgctcctcaaactgaatggaaacaaattctagataatactgaagttaaagctgttattctaggtggtgatcctagtagtggtgctagagttgttactggtaaagttgatatggttgaagatctaattcaagaaggtagtagatttactgctgatcatcctggtctacctattagttatactactagttttctaagagataatgttgttgctacttttcaaaatagtactgattatgttgaaactaaagttactgcttatagaaatggtgatctactactagatcatagtggtgcttatgttgctcaatattatattacttgggatgaactaagttatgatcatcaaggtaaagaagttctaactcctaaagcttgggatagaaatggtcaagatctaactgctcattttactactagtattcctctaaaaggtaatgttagaaatctaagtgttaaaattagagaatgtactggtctagcttgggaatggtggagaactgtttatgaaaaaactgatctacctctagttagaaaaagaactattagtatttggggtactactctatatcctcaagttgaagataaagttgaaaatgat

SEQ ID NO: 97 - RNA, 1413 nt, synthetic construct
  source 1..1413: mol_type = other RNA
atggctaataaagctgttaatgattttattctggctatgaattatgataaaaaaaaactgctgactcatcaaggtgaaagcattgaaaataggtttattaaagaaggtaatcaactgcctgatgaatttgttgttattgaaaggaaaaaaaggagcctgagcactaatactagcgatattagcgttactgctactaatgatagcaggctgtatcctggtgctctgctggttgttgatgaaactctgctggaaaataatcctactctgctggctgttgatagggctcctatgacttatagcattgatctgcctggtctggctagcagcgatagctttctgcaagttgaagatcctagcaatagcagcgttaggggtgctgttaatgatctgctggctaaatggcatcaagattatggtcaagttaataatgttcctgctaggatgcaatatgaaaaaattactgctcatagcatggaacaactgaaagttaaatttggtagcgattttgaaaaaactggtaatagcctggatattgattttaatagcgttcatagcggtgaaaaacaaattcaaattgttaattttaaacaaatttattatactgttagcgttgatgctgttaaaaatcctggtgatgtttttcaagatactgttactgttgaagatctgaaacaaaggggtattagcgctgaaaggcctctggtttatattagcagcgttgcttatggtaggcaagtttatctgaaactggaaactactagcaaaagcgatgaagttgaagctgcttttgaagctctgattaaaggtgttaaagttgctcctcaaactgaatggaaacaaattctggataatactgaagttaaagctgttattctgggtggtgatcctagcagcggtgctagggttgttactggtaaagttgatatggttgaagatctgattcaagaaggtagcaggtttactgctgatcatcctggtctgcctattagctatactactagctttctgagggataatgttgttgctacttttcaaaatagcactgattatgttgaaactaaagttactgcttataggaatggtgatctgctgctggatcatagcggtgcttatgttgctcaatattatattacttgggatgaactgagctatgatcatcaaggtaaagaagttctgactcctaaagcttgggataggaatggtcaagatctgactgctcattttactactagcattcctctgaaaggtaatgttaggaatctgagcgttaaaattagggaatgtactggtctggcttgggaatggtggaggactgtttatgaaaaaactgatctgcctctggttaggaaaaggactattagcatttggggtactactctgtatcctcaagttgaagataaagttgaaaatgat

SEQ ID NO: 98 - RNA, 645 nt, synthetic construct
  source 1..645: mol_type = other RNA
atgggtaaaggtgatcctaaaaaacctcgtggtaaaatgtcttcttatgctttttttgttcaaacttgtcgtgaagaacataaaaaaaaacatcctgatgcttctgttaatttttctgaattttctaaaaaatgttctgaacgttggaaaactatgtctgctaaagaaaaaggtaaatttgaagatatggctaaagctgataaagctcgttatgaacgtgaaatgaaaacttatattcctcctaaaggtgaaactaaaaaaaaatttaaagatcctaatgctcctaaacgtcctccttctgctttttttttattttgttctgaatatcgtcctaaaattaaaggtgaacatcctggtttatctattggtgatgttgctaaaaaattaggtgaaatgtggaataatactgctgctgatgataaacaaccttatgaaaaaaaagctgctaaattaaaagaaaaatatgaaaaagatattgctgcttatcgtgctaaaggtaaacctgatgctgctaaaaaaggtgttgttaaagctgaaaaatctaaaaaaaaaaaagaagaagaagaagatgaagaagatgaagaagatgaagaagaagaagaagatgaagaagatgaagatgaagaagaagatgatgatgatgaa

SEQ ID NO: 99 - RNA, 645 nt, synthetic construct
  source 1..645: mol_type = other RNA
atgggcaagggcgaccccaagaagccccgcggcaagatgtcctcctacgccttcttcgtccagacctgccgcgaggagcacaagaagaagcaccccgacgcctccgtcaacttctccgagttctccaagaagtgctccgagcgctggaagaccatgtccgccaaggagaagggcaagttcgaggacatggccaaggccgacaaggcccgctacgagcgcgagatgaagacctacatcccccccaagggcgagaccaagaagaagttcaaggaccccaacgcccccaagcgccccccctccgccttcttcttgttctgctccgagtaccgccccaagatcaagggcgagcaccccggcttgtccatcggcgacgtcgccaagaagttgggcgagatgtggaacaacaccgccgccgacgacaagcagccctacgagaagaaggccgccaagttgaaggagaagtacgagaaggacatcgccgcctaccgcgccaagggcaagcccgacgccgccaagaagggcgtcgtcaaggccgagaagtccaagaagaagaaggaggaggaggaggacgaggaggacgaggaggacgaggaggaggaggaggacgaggaggacgaggacgaggaggaggacgacgacgacgag

SEQ ID NO: 100 - RNA, 645 nt, synthetic construct
  source 1..645: mol_type = other RNA
atgggaaaaggagatccaaaaaaaccacgaggaaaaatgtcatcatatgcattttttgtacaaacatgtcgagaagaacataaaaaaaaacatccagatgcatcagtaaatttttcagaattttcaaaaaaatgttcagaacgatggaaaacaatgtcagcaaaagaaaaaggaaaatttgaagatatggcaaaagcagataaagcacgatatgaacgagaaatgaaaacatatataccaccaaaaggagaaacaaaaaaaaaatttaaagatccaaatgcaccaaaacgaccaccatcagcattttttcttttttgttcagaatatcgaccaaaaataaaaggagaacatccaggactttcaataggagatgtagcaaaaaaacttggagaaatgtggaataatacagcagcagatgataaacaaccatatgaaaaaaaagcagcaaaacttaaagaaaaatatgaaaaagatatagcagcatatcgagcaaaaggaaaaccagatgcagcaaaaaaaggagtagtaaaagcagaaaaatcaaaaaaaaaaaaagaagaagaagaagatgaagaagatgaagaagatgaagaagaagaagaagatgaagaagatgaagatgaagaagaagatgatgatgatgaa

SEQ ID NO: 101 - RNA, 645 nt, synthetic construct
  source 1..645: mol_type = other RNA
atggggaaaggggatccgaaaaaaccgcgggggaaaatgtcgtcgtatgcgttttttgtgcaaacgtgtcgggaagaacataaaaaaaaacatccggatgcgtcggtgaatttttcggaattttcgaaaaaatgttcggaacggtggaaaacgatgtcggcgaaagaaaaagggaaatttgaagatatggcgaaagcggataaagcgcggtatgaacgggaaatgaaaacgtatattccgccgaaaggggaaacgaaaaaaaaatttaaagatccgaatgcgccgaaacggccgccgtcggcgttttttctcttttgttcggaatatcggccgaaaattaaaggggaacatccggggctctcgattggggatgtggcgaaaaaactcggggaaatgtggaataatacggcggcggatgataaacaaccgtatgaaaaaaaagcggcgaaactcaaagaaaaatatgaaaaagatattgcggcgtatcgggcgaaagggaaaccggatgcggcgaaaaaaggggtggtgaaagcggaaaaatcgaaaaaaaaaaaagaagaagaagaagatgaagaagatgaagaagatgaagaagaagaagaagatgaagaagatgaagatgaagaagaagatgatgatgatgaa

SEQ ID NO: 102 - RNA, 645 nt, synthetic construct
  source 1..645: mol_type = other RNA
atgggtaaaggtgatcctaaaaaacctagaggtaaaatgagtagttatgctttttttgttcaaacttgtagagaagaacataaaaaaaaacatcctgatgctagtgttaattttagtgaatttagtaaaaaatgtagtgaaagatggaaaactatgagtgctaaagaaaaaggtaaatttgaagatatggctaaagctgataaagctagatatgaaagagaaatgaaaacttatattcctcctaaaggtgaaactaaaaaaaaatttaaagatcctaatgctcctaaaagacctcctagtgctttttttctattttgtagtgaatatagacctaaaattaaaggtgaacatcctggtctaagtattggtgatgttgctaaaaaactaggtgaaatgtggaataatactgctgctgatgataaacaaccttatgaaaaaaaagctgctaaactaaaagaaaaatatgaaaaagatattgctgcttatagagctaaaggtaaacctgatgctgctaaaaaaggtgttgttaaagctgaaaaaagtaaaaaaaaaaaagaagaagaagaagatgaagaagatgaagaagatgaagaagaagaagaagatgaagaagatgaagatgaagaagaagatgatgatgatgaa

SEQ ID NO: 103 - RNA, 645 nt, synthetic construct
  source 1..645: mol_type = other RNA
atgggtaaaggtgatcctaaaaaacctaggggtaaaatgagcagctatgctttttttgttcaaacttgtagggaagaacataaaaaaaaacatcctgatgctagcgttaattttagcgaatttagcaaaaaatgtagcgaaaggtggaaaactatgagcgctaaagaaaaaggtaaatttgaagatatggctaaagctgataaagctaggtatgaaagggaaatgaaaacttatattcctcctaaaggtgaaactaaaaaaaaatttaaagatcctaatgctcctaaaaggcctcctagcgctttttttctgttttgtagcgaatataggcctaaaattaaaggtgaacatcctggtctgagcattggtgatgttgctaaaaaactgggtgaaatgtggaataatactgctgctgatgataaacaaccttatgaaaaaaaagctgctaaactgaaagaaaaatatgaaaaagatattgctgcttatagggctaaaggtaaacctgatgctgctaaaaaaggtgttgttaaagctgaaaaaagcaaaaaaaaaaaagaagaagaagaagatgaagaagatgaagaagatgaagaagaagaagaagatgaagaagatgaagatgaagaagaagatgatgatgatgaa

SEQ ID NO: 104 - RNA, 1023 nt, synthetic construct
  source 1..1023: mol_type = other RNA
atgaataaaactttaattgctttagctgtttctgctgctgctgttgctactggtgcttatgctgatggtattaatcaatctggtgataaagctggttctactgtttattctgctaaaggtacttctttagaagttggtggtcgtgctgaagctcgtttatctttaaaagatggtaaagctcaagataattctcgtgttcgtttaaattttttaggtaaagctgaaattaatgattctttatatggtgttggtttttatgaaggtgaatttactactaatgatcaaggtaaaaatgcttctaataattctttagataatcgttatacttatgctggtattggtggtacttatggtgaagttacttatggtaaaaatgatggtgctttaggtgttattactgattttactgatattatgtcttatcatggtaatactgctgctgaaaaaattgctgttgctgatcgtgttgataatatgttagcttataaaggtcaatttggtgatttaggtgttaaagcttcttatcgttttgctgatcgtaatgctgttgatgctatgggtaatgttgttactgaaactaatgctgctaaatattctgataatggtgaagatggttattctttatctgctatttatacttttggtgatactggttttaatgttggtgctggttatgctgatcaagatgatcaaaatgaatatatgttagctgcttcttatcgtatggaaaatttatattttgctggtttatttactgatggtgaattagctaaagatgttgattatactggttatgaattagctgctggttataaattaggtcaagctgcttttactgctacttataataatgctgaaactgctaaagaaacttctgctgataattttgctattgatgctacttattattttaaacctaattttcgttcttatatttcttatcaatttaatttattagattctgataaagttggtaaagttgcttctgaagatgaattagctattggtttacgttatgatttt

SEQ ID NO: 105 - RNA, 1008 nt, synthetic construct
  source 1..1008: mol_type = other RNA
atgaacaagaccttgatcgccttggccgtctccgccgccgccgtcgccaccggcgcctacgccgacggcatcaaccagtccggcgacaaggccggctccaccgtctactccgccaagggcacctccttggaggtcggcggccgcgccgaggcccgcttgtccttgaaggacggcaaggcccaggacaactcccgcgtccgcttgaacttcttgggcaaggccgagatcaacgactccttgtacggcgtcggcttctacgagggcgagttcaccaccaacgaccagggcaagaacgcctccaacaactccttggacaaccgctacacctacgccggcatcggcggcacctacggcgaggtcacctacggcaagaacgacggcgccttgggcgtcatcaccgacttcaccgacatctcctaccacggcaacaccgccgccgagaagatcgccgtcgccgaccgcgtcgacaacttggcctacaagggccagttcggcgacttgggcgtcaaggcctcctaccgcttcgccgaccgcaacgccgtcgacgccggcaacgtcgtcaccgagaccaacgccgccaagtactccgacaacggcgaggacggctactccttgtccgccatctacaccttcggcgacaccggcttcaacgtcggcgccggctacgccgaccaggacgaccagaacgagtacttggccgcctcctaccgcgagaacttgtacttcgccggcttgttcaccgacggcgagttggccaaggacgtcgactacaccggctacgagttggccgccggctacaagttgggccaggccgccttcaccgccacctacaacaacgccgagaccgccaaggagacctccgccgacaacttcgccatcgacgccacctactacttcaagcccaacttccgctcctacatctcctaccagttcaacttgttggactccgacaaggtcggcaaggtcgcctccgaggacgagttggccatcggcttgcgctacgacttc

SEQ ID NO: 106 - RNA, 1026 nt, synthetic construct
  source 1..1026: mol_type = other RNA
atgaataaaacacttatagcacttgcagtatcagcagcagcagtagcaacaggagcatatgcagatggaataaatcaatcaggagataaagcaggatcaacagtatattcagcaaaaggaacatcacttgaagtaggaggacgagcagaagcacgactttcacttaaagatggaaaagcacaagataattcacgagtacgacttaattttcttggaaaagcagaaataaatgattcactttatggagtaggattttatgaaggagaatttacaacaaatgatcaaggaaaaaatgcatcaaataattcacttgataatcgatatacatatgcaggaataggaggaacatatggaggagaagtaacatatggaaaaaatgatggagcacttggagtaataacagattttacagatataatgtcatatcatggaaatacagcagcagaaaaaatagcagtagcagatcgagtagataatatgcttgcatataaaggacaatttggagatcttggagtaaaagcatcatatcgatttgcagatcgaaatgcagtagatgcaatgggaaatgtagtaacagaaacaaatgcagcaaaatattcagataatggagaagatggatattcactttcagcaatatatacatttggagatacaggatttaatgtaggagcaggatatgcagatcaagatgatcaaaatgaatatatgcttgcagcatcatatcgaatggaaaatctttattttgcaggactttttacagatggagaacttgcaaaagatgtagattatacaggatatgaacttgcagcaggatataaacttggacaagcagcatttacagcaacatataataatgcagaaacagcaaaagaaacatcagcagataattttgcaatagatgcaacatattattttaaaccaaattttcgatcatatatttcatatcaatttaatcttcttgattcagataaagtaggaaaagtagcatcagaagatgaacttgcaataggacttcgatatgatttt

SEQ ID NO: 107 - RNA, 1014 nt, synthetic construct
  source 1..1014: mol_type = other RNA
atgaataaaacgctcattgcgctcgcggtgtcggcggcggcggtggcgacgggggcgtatgcggatgggattaatcaatcgggggataaagcggggtcgacggtgtattcggcgaaagggacgtcgctcgaagtgggggggcgggcggaagcgcggctctcgctcaaagatgggaaagcgcaagataattcgcgggtgcggctcaattttctcgggaaagcggaaattaatgattcgctctatggggtggggttttatgaaggggaatttacgacgaatgatcaagggaaaaatgcgtcgaataattcgctcgataatcggtatacgtatgcggggggggggacgtatgggggggaagtgacgtatgggaaaaatgatggggcgctcggggtgacggattttacggatattatgtcgtatcatgggaatacggcggcggaaaaaattgcggtggcggatcgggtggataatatgctcgcgtataaagggcaatttggggatctcggggtgaaagcgtcgtatcggtttgcggatcggaatgcggtggatgcgatggggaatgtggtgacggaaacgaatgcggcgaaatattcggataatggggaagatgggtattcgctctcggcgtatacgtttggggatacggggtttaatgtgggggcggggtatgcggatcaagatgatcaaaatgaatatatgctcgcggcgtcgtatcggatggaaaatctctattttgcggggctctttacggatggggaactcgcgaaagatgtggattatacggggtatgaactcgcggcggggtataaactcgggcaagcggcgtttacggcgacgtataataatgcggaaacggcgaaagaaacgtcggcggataattttgcgattgatgcgacgtattattttaaaccgaattttcggtcgtatatttcgtatcaatttaatctcctcgattcggataaagtggggaaagtggcgtcggaagatgaactcgcggggctccggtatgatttt

SEQ ID NO: 108 - RNA, 1023 nt, synthetic construct
  source 1..1023: mol_type = other RNA
atgaataaaactctaattgctctagctgttagtgctgctgctgttgctactggtgcttatgctgatggtattaatcaaagtggtgataaagctggtagtactgtttatagtgctaaaggtactagtctagaagttggtggtagagctgaagctagactaagtctaaaagatggtaaagctcaagataatagtagagttagactaaattttctaggtaaagctgaaattaatgatagtctatatggtgttggtttttatgaaggtgaatttactactaatgatcaaggtaaaaatgctagtaataatagtctagataatagatatacttatgctggtattggtggtacttatggtgaagttacttatggtaaaaatgatggtgctctaggtgttattactgattttactgatattatgagttatcatggtaatactgctgctgaaaaaattgctgttgctgatagagttgataatatgctagcttataaaggtcaatttggtgatctaggtgttaaagctagttatagatttgctgatagaaatgctgttgatgctatgggtaatgttgttactgaaactaatgctgctaaatatagtgataatggtgaagatggttatagtctaagtgctatttatacttttggtgatactggttttaatgttggtgctggttatgctgatcaagatgatcaaaatgaatatatgctagctgctagttatagaatggaaaatctatattttgctggtctatttactgatggtgaactagctaaagatgttgattatactggttatgaactagctgctggttataaactaggtcaagctgcttttactgctacttataataatgctgaaactgctaaagaaactagtgctgataattttgctattgatgctacttattattttaaacctaattttagaagttatattagttatcaatttaatctactagatagtgataaagttggtaaagttgctagtgaagatgaactagctattggtctaagatatgatttt

SEQ ID NO: 109 - RNA, 1023 nt, synthetic construct
  source 1..1023: mol_type = other RNA
atgaataaaactctgattgctctggctgttagcgctgctgctgttgctactggtgcttatgctgatggtattaatcaaagcggtgataaagctggtagcactgtttatagcgctaaaggtactagcctggaagttggtggtagggctgaagctaggctgagcctgaaagatggtaaagctcaagataatagcagggttaggctgaattttctgggtaaagctgaaattaatgatagcctgtatggtgttggtttttatgaaggtgaatttactactaatgatcaaggtaaaaatgctagcaataatagcctggataataggtatacttatgctggtattggtggtacttatggtgaagttacttatggtaaaaatgatggtgctctgggtgttattactgattttactgatattatgagctatcatggtaatactgctgctgaaaaaattgctgttgctgatagggttgataatatgctggcttataaaggtcaatttggtgatctgggtgttaaagctagctataggtttgctgataggaatgctgttgatgctatgggtaatgttgttactgaaactaatgctgctaaatatagcgataatggtgaagatggttatagcctgagcgctatttatacttttggtgatactggttttaatgttggtgctggttatgctgatcaagatgatcaaaatgaatatatgctggctgctagctataggatggaaaatctgtattttgctggtctgtttactgatggtgaactggctaaagatgttgattatactggttatgaactggctgctggttataaactgggtcaagctgcttttactgctacttataataatgctgaaactgctaaagaaactagcgctgataattttgctattgatgctacttattattttaaacctaattttaggagctatattagctatcaatttaatctgctggatagcgataaagttggtaaagttgctagcgaagatgaactggctattggtctgaggtatgatttt

SEQ ID NO: 110 - RNA, 1890 nt, synthetic construct
  source 1..1890: mol_type = other RNA
atgactcgttatcaagatgatttttatgatgctattaatggtgaatggcaacaaactgctgaaattcctgctgataaatctcaaactggtggttttgttgatttagatcaagaaattgaagatttaatgttagctactactgataaatggttagctggtgaagaagttcctgaagatgctattttagaaaattttgttaaatatcatcgtttagttcgtgattttgataaacgtgaagctgatggtattactcctgttttacctttattaaaagaatttcaagaattagaaacttttgctgattttactgctaaattagctgaatttgaattagctggtaaacctaattttttaccttttggtgtttctcctgattttatggatgctcgtattaatgttttatgggcttctgctccttctactattttacctgatactacttattatgctgaagaacatcctcaacgtgaagaattattaactttatggaaagaatcttctgctaatttattaaaagcttatgatttttctgatgaagaaattgaagatttattagaaaaacgtttagaattagatcgtcgtgttgctgctgttgttttatctaatgaagaatcttctgaatatgctaaattatatcatccttattcttatgaagattttaaaaaatttgctcctgctttacctttagatgatttttttaaagctgttattggtcaattacctgataaagttattgttgatgaagaacgtttttggcaagctgctgaacaattttattctgaagaagcttggtctttattaaaagctactttaattttatctgttgttaatttatctacttcttatttaactgaagatattcgtgttttatctggtgcttattctcgtgctttatctggtgttcctgaagctaaagataaagttaaagctgcttatcatttagctcaagaaccttttaaacaagctttaggtttatggtatgctcgtgaaaaattttctcctgaagctaaagctgatgttgaaaaaaaagttgctactatgattgatgtttataaagaacgtttattaaaaaatgattggttaactcctgaaacttgtaaacaagctattgttaaattaaatgttattaaaccttatattggttatcctgaagaattacctgctcgttataaagataaagttgttaatgaaactgcttctttatttgaaaatgctttagcttttgctcgtgttgaaattaaacattcttggtctaaatggaatcaacctgttgattataaagaatggggtatgcctgctcatatggttaatgcttattataatcctcaaaaaaatttaattgtttttcctgctgctattttacaagctcctttttatgatttacatcaatcttcttctgctaattatggtggtattggtgctgttattgctcatgaaatttctcatgcttttgatactaatggtgcttcttttgatgaaaatggttctttaaaagattggtggactgaatctgattatgctgcttttaaagaaaaaactcaaaaagttattgatcaatttgatggtcaagattcttatggtgctactattaatggtaaattaactgtttctgaaaatgttgctgatttaggtggtattgctgctgctttagaagctgctaaacgtgaagctgatttttctgctgaagaatttttttataattttggtcgtatttggcgtatgaaaggtcgtcctgaatttatgaaattattagcttctgttgatgttcatgctcctgctaaattacgtgttaatgttcaagttcctaattttgatgatttttttactacttatgatgttaaagaaggtgatggtatgtggcgttctcctgaagaacgtgttattatttgg

SEQ ID NO: 111 - RNA, 1884 nt, synthetic construct
  source 1..1884: mol_type = other RNA
atgacccgctaccaggacgacttctacgacgccatcaacggcgagtggcagcagaccgccgagatccccgccgacaagtcccagaccggcggcttcgtcgacttggaccaggagatcgaggacttgatgttggccaccaccgacaagtggttggccggcgaggaggtccccgaggacgccatcttggagaacttcgtcaagtaccaccgcttggtccgcgacttcgacaagcgcgaggccgacggcatcacccccgtcttgcccttgttgaaggagttccaggagttggagaccttcgccgacttcaccgccaagttggccgagttcgagttggccggcaagcccaacttcttgcccttcggcgtctcccccgacttcatggacgcccgcatcaacgtcttgtgggcctccgccccctccaccatcttgcccgacaccacctactacgccgaggagcacccccagcgcgaggagttgttgaccttgtggaaggagtcctccgccaacttgttgaaggcctacgacttctccgacgaggagatcgaggacttgttggagaagcgcttggagttggaccgccgcgtcgccgccgtcgtcttgtccaacgaggagtcctccgagtacgccaagttgtaccacccctactcctacgaggacttcaagaagttcgcccccgccttgcccttggacgacttcttcaaggccgtcatcggccagttgcccgacaaggtcatcgtcgacgaggagcgcttctggcaggccgccgagcagttctactccgaggaggcctggtccttgttgaaggccaccttgatcttgtccgtcgtcaacttgtccacctcctacttgaccgaggacatccgcgtcttgtccggcgcctactcccgcgccttgtccggcgtccccgaggccaaggacaaggtcaaggccgcctaccacttggcccaggagcccttcaagcaggccttgggcttgtggtacgcccgcgagaagttctcccccgaggccaaggccgacgtcgagaagaaggtcgccaccatgatcgacgtctacaaggagcgcttgttgaagaacgacttgacccccgagacctgcaagcaggccatcgtcaagttgaacgtcatcaagccctacatcggctaccccgaggagttgcccgcccgctacaaggacaaggtcgtcaacgagaccgcctccttgttcgagaacgccttggccttcgcccgcgtcgagatcaagcactcctccaagtggaaccagcccgtcgactacaaggagtggggcatgcccgcccacatggtcaacgcctactacaacccccagaagaacttgatcgtcttccccgccgccatcttgcaggcccccttctacgacttgcaccagtcctcctccgccaactacggcggcatcggcgccgtcatcgcccacgagatctcccacgccttcgacaccaacggcgcctccttcgacgagaacggctccttgaaggactggtggaccgagtccgactacgccgccttcaaggagaagacccagaaggtcatcgaccagttcgacggccaggactcctacggcgccaccatcaacggcaagttgaccgtctccgagaacgtcgccgacttgggcggcatcgccgccgccttggaggccgccaagcgcgaggccgacttctccgccgaggagttcttctacaacttcggccgcatctggcgcatgaagggccgccccgagttcatgaagttgttggcctccgtcgacgtccacgcccccgccaagttgcgcgtcaacgtccaggtccccaacttcgacgacttcttcaccacctacgacgtcaaggagggcgacggcatgtggcgctcccccgaggagcgcgtcatcatctgg

SEQ ID NO: 112 - RNA, 1890 nt, synthetic construct
  source 1..1890: mol_type = other RNA
atgacacgatatcaagatgatttttatgatgcaataaatggagaatggcaacaaacagcagaaataccagcagataaatcacaaacaggaggatttgtagatcttgatcaagaaatagaagatcttatgcttgcaacaacagataaatggcttgcaggagaagaagtaccagaagatgcaatacttgaaaattttgtaaaatatcatcgacttgtacgagattttgataaacgagaagcagatggaataacaccagtacttccacttcttaaagaatttcaagaacttgaaacatttgcagattttacagcaaaacttgcagaatttgaacttgcaggaaaaccaaattttcttccatttggagtatcaccagattttatggatgcacgaataaatgtactttgggcatcagcaccatcaacaatacttccagatacaacatattatgcagaagaacatccacaacgagaagaacttcttacactttggaaagaatcatcagcaaatcttcttaaagcatatgatttttcagatgaagaaatagaagatcttcttgaaaaacgacttgaacttgatcgacgagtagcagcagtagtactttcaaatgaagaatcatcagaatatgcaaaactttatcatccatattcatatgaagattttaaaaaatttgcaccagcacttccacttgatgatttttttaaagcagtaataggacaacttccagataaagtaatagtagatgaagaacgattttggcaagcagcagaacaattttattcagaagaagcatggtcacttcttaaagcaacacttatactttcagtagtaaatctttcaacatcatatcttacagaagatatacgagtactttcaggagcatattcacgagcactttcaggagtaccagaagcaaaagataaagtaaaagcagcatatcatcttgcacaagaaccatttaaacaagcacttggactttggtatgcacgagaaaaattttcaccagaagcaaaagcagatgtagaaaaaaaagtagcaacaatgatagatgtatataaagaacgacttcttaaaaatgattggcttacaccagaaacatgtaaacaagcaatagtaaaacttaatgtaataaaaccatatataggatatccagaagaacttccagcacgatataaagataaagtagtaaatgaaacagcatcactttttgaaaatgcacttgcatttgcacgagtagaaataaaacattcatggtcaaaatggaatcaaccagtagattataaagaatggggaatgccagcacatatggtaaatgcatattataatccacaaaaaaatcttatagtatttccagcagcaatacttcaagcaccattttatgatcttcatcaatcatcatcagcaaattatggaggaataggagcagtaatagcacatgaaatatcacatgcatttgatacaaatggagcatcatttgatgaaaatggatcacttaaagattggtggacagaatcagattatgcagcatttaaagaaaaaacacaaaaagtaatagatcaatttgatggacaagattcatatggagcaacaataaatggaaaacttacagtatcagaaaatgtagcagatcttggaggaatagcagcagcacttgaagcagcaaaacgagaagcagatttttcagcagaagaatttttttataattttggacgaatatggcgaatgaaaggacgaccagaatttatgaaacttcttgcatcagtagatgtacatgcaccagcaaaacttcgagtaaatgtacaagtaccaaattttgatgatttttttacaacatatgatgtaaaagaaggagatggaatgtggcgatcaccagaagaacgagtaataatatgg

SEQ ID NO: 113 - RNA, 1890 nt, synthetic construct
  source 1..1890: mol_type = other RNA
atgacgcggtatcaagatgatttttatgatgcgattaatggggaatggcaacaaacggcggaaattccggcggataaatcgcaaacgggggggtttgtggatctcgatcaagaaattgaagatctcatgctcgcgacgacggataaatggctcgcgggggaagaagtgccggaagatgcgattctcgaaaattttgtgaaatatcatcggctcgtgcgggattttgataaacgggaagcggatgggattacgccggtgctcccgctcctcaaagaatttcaagaactcgaaacgtttgcggattttacggcgaaactcgcggaatttgaactcgcggggaaaccgaattttctcccgtttggggtgtcgccggattttatggatgcgcggattaatgtgctctgggcgtcggcgccgtcgacgattctcccggatacgacgtattatgcggaagaacatccgcaacgggaagaactcctcacgctctggaaagaatcgtcggcgaatctcctcaaagcgtatgatttttcggatgaagaaattgaagatctcctcgaaaaacggctcgaactcgatcggcgggtggcggcggtggtgctctcgaatgaagaatcgtcggaatatgcgaaactctatcatccgtattcgtatgaagattttaaaaaatttgcgccggcgctcccgctcgatgatttttttaaagcggtgattgggcaactcccggataaagtgattgtggatgaagaacggttttggcaagcggcggaacaattttattcggaagaagcgtggtcgctcctcaaagcgacgctcattctctcggtggtgaatctctcgacgtcgtatctcacggaagatattcgggtgctctcgggggcgtattcgcgggcgctctcgggggtgccggaagcgaaagataaagtgaaagcggcgtatcatctcgcgcaagaaccgtttaaacaagcgctcgggctctggtatgcgcgggaaaaattttcgccggaagcgaaagcggatgtggaaaaaaaagtggcgacgatgattgatgtgtataaagaacggctcctcaaaaatgattggctcacgccggaaacgtgtaaacaagcgattgtgaaactcaatgtgattaaaccgtatattgggtatccggaagaactcccggcgcggtataaagataaagtggtgaatgaaacggcgtcgctctttgaaaatgcgctcgcgtttgcgcgggtggaaattaaacattcgtggtcgaaatggaatcaaccggtggattataaagaatgggggatgccggcgcatatggtgaatgcgtattataatccgcaaaaaaatctcattgtgtttccggcggcgattctccaagcgccgttttatgatctccatcaatcgtcgtcggcgaattatggggggattggggcggtgattgcgcatgaaatttcgcatgcgtttgatacgaatggggcgtcgtttgatgaaaatgggtcgctcaaagattggtggacggaatcggattatgcggcgtttaaagaaaaaacgcaaaaagtgattgatcaatttgatgggcaagattcgtatggggcgacgattaatgggaaactcacggtgtcggaaaatgtggcggatctcggggggattgcggcggcgctcgaagcggcgaaacgggaagcggatttttcggcggaagaatttttttataattttgggcggatttggcggatgaaagggcggccggaatttatgaaactcctcgcgtcggtggatgtgcatgcgccggcgaaactccgggtgaatgtgcaagtgccgaattttgatgatttttttacgacgtatgatgtgaaagaaggggatgggatgtggcggtcgccggaagaacgggtgattatttgg

SEQ ID NO: 114 - RNA, 1890 nt, synthetic construct
  source 1..1890: mol_type = other RNA
atgactagatatcaagatgatttttatgatgctattaatggtgaatggcaacaaactgctgaaattcctgctgataaaagtcaaactggtggttttgttgatctagatcaagaaattgaagatctaatgctagctactactgataaatggctagctggtgaagaagttcctgaagatgctattctagaaaattttgttaaatatcatagactagttagagattttgataaaagagaagctgatggtattactcctgttctacctctactaaaagaatttcaagaactagaaacttttgctgattttactgctaaactagctgaatttgaactagctggtaaacctaattttctaccttttggtgttagtcctgattttatggatgctagaattaatgttctatgggctagtgctcctagtactattctacctgatactacttattatgctgaagaacatcctcaaagagaagaactactaactctatggaaagaaagtagtgctaatctactaaaagcttatgattttagtgatgaagaaattgaagatctactagaaaaaagactagaactagatagaagagttgctgctgttgttctaagtaatgaagaaagtagtgaatatgctaaactatatcatccttatagttatgaagattttaaaaaatttgctcctgctctacctctagatgatttttttaaagctgttattggtcaactacctgataaagttattgttgatgaagaaagattttggcaagctgctgaacaattttatagtgaagaagcttggagtctactaaaagctactctaattctaagtgttgttaatctaagtactagttatctaactgaagatattagagttctaagtggtgcttatagtagagctctaagtggtgttcctgaagctaaagataaagttaaagctgcttatcatctagctcaagaaccttttaaacaagctctaggtctatggtatgctagagaaaaatttagtcctgaagctaaagctgatgttgaaaaaaaagttgctactatgattgatgtttataaagaaagactactaaaaaatgattggctaactcctgaaacttgtaaacaagctattgttaaactaaatgttattaaaccttatattggttatcctgaagaactacctgctagatataaagataaagttgttaatgaaactgctagtctatttgaaaatgctctagcttttgctagagttgaaattaaacatagttggagtaaatggaatcaacctgttgattataaagaatggggtatgcctgctcatatggttaatgcttattataatcctcaaaaaaatctaattgtttttcctgctgctattctacaagctcctttttatgatctacatcaaagtagtagtgctaattatggtggtattggtgctgttattgctcatgaaattagtcatgcttttgatactaatggtgctagttttgatgaaaatggtagtctaaaagattggtggactgaaagtgattatgctgcttttaaagaaaaaactcaaaaagttattgatcaatttgatggtcaagattcttatggtgctactattaatggtaaactaactgttagtgaaaatgttgctgatctaggtggtattgctgctgctctagaagctgctaaaagagaagctgattttagtgctgaagaatttttttataattttggtagaatttggagaatgaaaggtagacctgaatttatgaaactactagctagtgttgatgttcatgctcctgctaaactaagagttaatgttcaagttcctaattttgatgatttttttactacttatgatgttaaagaaggtgatggtatgtggagaagtcctgaagaaagagttattatttgg

SEQ ID NO: 115 - RNA, 1890 nt, synthetic construct
  source 1..1890: mol_type = other RNA
atgactaggtatcaagatgatttttatgatgctattaatggtgaatggcaacaaactgctgaaattcctgctgataaaagccaaactggtggttttgttgatctggatcaagaaattgaagatctgatgctggctactactgataaatggctggctggtgaagaagttcctgaagatgctattctggaaaattttgttaaatatcataggctggttagggattttgataaaagggaagctgatggtattactcctgttctgcctctgctgaaagaatttcaagaactggaaacttttgctgattttactgctaaactggctgaatttgaactggctggtaaacctaattttctgccttttggtgttagccctgattttatggatgctaggattaatgttctgtgggctagcgctcctagcactattctgcctgatactacttattatgctgaagaacatcctcaaagggaagaactgctgactctgtggaaagaaagcagcgctaatctgctgaaagcttatgattttagcgatgaagaaattgaagatctgctggaaaaaaggctggaactggataggagggttgctgctgttgttctgagcaatgaagaaagcagcgaatatgctaaactgtatcatccttatagctatgaagattttaaaaaatttgctcctgctctgcctctggatgatttttttaaagctgttattggtcaactgcctgataaagttattgttgatgaagaaaggttttggcaagctgctgaacaattttatagcgaagaagcttggagcctgctgaaagctactctgattctgagcgttgttaatctgagcactagctatctgactgaagatattagggttctgagcggtgcttatagcagggctctgagcggtgttcctgaagctaaagataaagttaaagctgcttatcatctggctcaagaaccttttaaacaagctctgggtctgtggtatgctagggaaaaatttagccctgaagctaaagctgatgttgaaaaaaaagttgctactatgattgatgtttataaagaaaggctgctgaaaaatgattggctgactcctgaaacttgtaaacaagctattgttaaactgaatgttattaaaccttatattggttatcctgaagaactgcctgctaggtataaagataaagttgttaatgaaactgctagcctgtttgaaaatgctctggcttttgctagggttgaaattaaacatagctggagcaaatggaatcaacctgttgattataaagaatggggtatgcctgctcatatggttaatgcttattataatcctcaaaaaaatctgattgtttttcctgctgctattctgcaagctcctttttatgatctgcatcaaagcagcagcgctaattatggtggtattggtgctgttattgctcatgaaattagccatgcttttgatactaatggtgctagctttgatgaaaatggtagcctgaaagattggtggactgaaagcgattatgctgcttttaaagaaaaaactcaaaaagttattgatcaatttgatggtcaagattcttatggtgctactattaatggtaaactgactgttagcgaaaatgttgctgatctgggtggtattgctgctgctctggaagctgctaaaagggaagctgattttagcgctgaagaatttttttataattttggtaggatttggaggatgaaaggtaggcctgaatttatgaaactgctggctagcgttgatgttcatgctcctgctaaactgagggttaatgttcaagttcctaattttgatgatttttttactacttatgatgttaaagaaggtgatggtatgtggaggagccctgaagaaagggttattatttgg

SEQ ID NO: 116 - RNA, 1044 nt, synthetic construct
  source 1..1044: mol_type = other RNA
atgaaaaaaactgctattgctattgctgttgctttagctggttttgctactgttgctcaagctgctcctaaagataatacttggtatactggtgctaaattaggttggtctcaatatcatgatactggttttattcctaataatggtcctactcatgaaaatcaattaggtgctggtgcttttggtggttatcaagttaatccttatgttggttttgaaatgggttatgattggttaggtcgtatgccttataaaggtgataatattaatggtgcttataaagctcaaggtgttcaattaactgctaaattaggttatcctattactgatgatttagatatttatactcgtttaggtggtatggtttggcgtgctgatactaaagctaatgttcctggtggtgcttcttttaaagatcatgatactggtgtttctcctgtttttgctggtggtgttgaatatgctattactcctgaaattgctactcgtttagaatatcaatggactaataatattggtgatgctaatactattggtactcgtcctgataatggtttattatctttaggtgtttcttatcgttttggtcaaggtgaagctgctcctgttgttgctcctgctcctgctcctgaagttcaaactaaacattttactttaaaatctgatgttttatttaattttaataaagctactttaaaacctgaaggtcaagctgctttagatcaattatattctcaattatctaatttagatcctaaagatggttctgttgttgttttaggttatactgatcgtattggttctgatgcttataatcaaggtttatctgaacgtcgtgctcaatctgttgttgattatttaatttctaaaggtattcctgctgataaaatttctgctcgtggtatgggtgaatctaatcctgttactggtaatacttgtgataatgttaaacaacgtgctgctttaattgattgtttagctcctgatcgtcgtgttgaaattgaagttaaaggtattaaagatgttgttactcaacctcaagct

SEQ ID NO: 117 - RNA, 1044 nt, synthetic construct
  source 1..1044: mol_type = other RNA
atgaagaagaccgccatcgccatcgccgtcgccttggccggcttcgccaccgtcgcccaggccgcccccaaggacaacacctggtacaccggcgccaagttgggctggtcccagtaccacgacaccggcttcatccccaacaacggccccacccacgagaaccagttgggcgccggcgccttcggcggctaccaggtcaacccctacgtcggcttcgagatgggctacgactggttgggccgcatgccctacaagggcgacaacatcaacggcgcctacaaggcccagggcgtccagttgaccgccaagttgggctaccccatcaccgacgacttggacatctacacccgcttgggcggcatggtctggcgcgccgacaccaaggccaacgtccccggcggcgcctccttcaaggaccacgacaccggcgtctcccccgtcttcgccggcggcgtcgagtacgccatcacccccgagatcgccacccgcttggagtaccagtggaccaacaacatcggcgacgccaacaccatcggcacccgccccgacaacggcttgttgtccttgggcgtctcctaccgcttcggccagggcgaggccgcccccgtcgtcgcccccgcccccgcccccgaggtccagaccaagcacttcaccttgaagtccgacgtcttgttcaacttcaacaaggccaccttgaagcccgagggccaggccgccttggaccagttgtactcccagttgtccaacttggaccccaaggacggctccgtcgtcgtcttgggctacaccgaccgcatcggctccgacgcctacaaccagggcttgtccgagcgccgcgcccagtccgtcgtcgactacttgatctccaagggcatccccgccgacaagatctccgcccgcggcatgggcgagtccaaccccgtcaccggcaacacctgcgacaacgtcaagcagcgcgccgccttgatcgactgcttggcccccgaccgccgcgtcgagatcgaggtcaagggcatcaaggacgtcgtcacccagccccaggcc

SEQ ID NO: 118 - RNA, 1044 nt, synthetic construct
  source 1..1044: mol_type = other RNA
atgaaaaaaacagcaatagcaatagcagtagcacttgcaggatttgcaacagtagcacaagcagcaccaaaagataatacatggtatacaggagcaaaacttggatggtcacaatatcatgatacaggatttataccaaataatggaccaacacatgaaaatcaacttggagcaggagcatttggaggatatcaagtaaatccatatgtaggatttgaaatgggatatgattggcttggacgaatgccatataaaggagataatataaatggagcatataaagcacaaggagtacaacttacagcaaaacttggatatccaataacagatgatcttgatatatatacacgacttggaggaatggtatggcgagcagatacaaaagcaaatgtaccaggaggagcatcatttaaagatcatgatacaggagtatcaccagtatttgcaggaggagtagaatatgcaataacaccagaaatagcaacacgacttgaatatcaatggacaaataatataggagatgcaaatacaataggaacacgaccagataatggacttctttcacttggagtatcatatcgatttggacaaggagaagcagcaccagtagtagcaccagcaccagcaccagaagtacaaacaaaacattttacacttaaatcagatgtactttttaattttaataaagcaacacttaaaccagaaggacaagcagcacttgatcaactttattcacaactttcaaatcttgatccaaaagatggatcagtagtagtacttggatatacagatcgaataggatcagatgcatataatcaaggactttcagaacgacgagcacaatcagtagtagattatcttatatcaaaaggaataccagcagataaaatatcagcacgaggaatgggagaatcaaatccagtaacaggaaatacatgtgataatgtaaaacaacgagcagcacttatagattgtcttgcaccagatcgacgagtagaaatagaagtaaaaggaataaaagatgtagtaacacaaccacaagca

SEQ ID NO: 119 - RNA, 1041 nt, synthetic construct
  source 1..1041: mol_type = other RNA
atgaaaaaaacggcgattgcgattgcggtggcgctcgcggggtttgcgacggtggcgcaagcggcgccgaaagataatacgtggtatacgggggcgaaactcgggtggtcgcaatatcatgatacggggtttattccgaataatgggccgacgcatgaaaatcaactcggggcgggggcgtttggggggtatcaagtgaatccgtatgtggggtttgaaatggggtatgattggctcgggcggatgccgtataaaggggataatattaatggggcgtataaagcgcaaggggtgcaactcacggcgaaactcgggtatccgattacggatgatctcgatatttatacgcggctcggggggatggtgtggcgggcggatacgaaagcgaatgtgccggggggggcgtcgtttaaagatcatgatacgggggtgtcgccggtgtttgcggggggggtggaatatgcgacgccggaaattgcgacgcggctcgaatatcaatggacgaataatattggggatgcgaatacgattgggacgcggccggataatgggctcctctcgctcggggtgtcgtatcggtttgggcaaggggaagcggcgccggtggtggcgccggcgccggcgccggaagtgcaaacgaaacattttacgctcaaatcggatgtgctctttaattttaataaagcgacgctcaaaccggaagggcaagcggcgctcgatcaactctattcgcaactctcgaatctcgatccgaaagatgggtcggtggtggtgctcgggtatacggatcggattgggtcggatgcgtataatcaagggctctcggaacggcgggcgcaatcggtggtggattatctcatttcgaaagggattccggcggataaaatttcggcgcgggggatgggggaatcgaatccggtgacggggaatacgtgtgataatgtgaaacaacgggcggcgctcattgattgtctcgcgccggatcggcgggtggaaattgaagtgaaagggattaaagatgtggtgacgcaaccgcaagcg

SEQ ID NO: 120 - RNA, 1044 nt, synthetic construct
  source 1..1044: mol_type = other RNA
atgaaaaaaactgctattgctattgctgttgctctagctggttttgctactgttgctcaagctgctcctaaagataatacttggtatactggtgctaaactaggttggagtcaatatcatgatactggttttattcctaataatggtcctactcatgaaaatcaactaggtgctggtgcttttggtggttatcaagttaatccttatgttggttttgaaatgggttatgattggctaggtagaatgccttataaaggtgataatattaatggtgcttataaagctcaaggtgttcaactaactgctaaactaggttatcctattactgatgatctagatatttatactagactaggtggtatggtttggagagctgatactaaagctaatgttcctggtggtgctagttttaaagatcatgatactggtgttagtcctgtttttgctggtggtgttgaatatgctattactcctgaaattgctactagactagaatatcaatggactaataatattggtgatgctaatactattggtactagacctgataatggtctactaagtctaggtgttagttatagatttggtcaaggtgaagctgctcctgttgttgctcctgctcctgctcctgaagttcaaactaaacattttactctaaaaagtgatgttctatttaattttaataaagctactctaaaacctgaaggtcaagctgctctagatcaactatatagtcaactaagtaatctagatcctaaagatggtagtgttgttgttctaggttatactgatagaattggtagtgatgcttataatcaaggtctaagtgaaagaagagctcaaagtgttgttgattatctaattagtaaaggtattcctgctgataaaattagtgctagaggtatgggtgaaagtaatcctgttactggtaatacttgtgataatgttaaacaaagagctgctctaattgattgtctagctcctgatagaagagttgaaattgaagttaaaggtattaaagatgttgttactcaacctcaagct

SEQ ID NO: 121 - RNA, 1041 nt, synthetic construct
  source 1..1041: mol_type = other RNA
atgaaaaaaactgctattgctattgctgttgctctggctggttttgctactgttgctcaagctgctcctaaagataatacttggtatactggtgctaaactgggttggagccaatatcatgatactggttttattcctaataatggtcctactcatgaaaatcaactgggtgctggtgcttttggtggttatcaagttaatccttatgttggttttgaaatgggttatgattggctgggtaggatgccttataaaggtgataatattaatggtgcttataaagctcaaggtgttcaactgactgctaaactgggttatcctattactgatgatctggatatttatactaggctgggtggtatggtttggagggctgatactaaagctaatgttcctggtggtgctagctttaaagatcatgatactggtgttagccctgtttttgctggtggtgttgaatatgctattactcctgaaattgctactaggctggaatatcaatggactaataatattggtgatgctaatactattggtactaggcctgataatggtctgctgagcctgggtgttagctataggtttggtcaaggtgctgctcctgttgttgctcctgctcctgctcctgaagttcaaactaaacattttactctgaaaagcgatgttctgtttaattttaataaagctactctgaaacctgaaggtcaagctgctctggatcaactgtatagccaactgagcaatctggatcctaaagatggtagcgttgttgttctgggttatactgataggattggtagcgatgcttataatcaaggtttaagcgaaaggagggctcaaagcgttgttgattatctgattagcaaaggtattcctgctgataaaattagcgctaggggtatgggtgaaagcaatcctgttactggtaatacttgtgataatgttaaacaaagggctgctctgattgattgtctggctcctgataggagggttgaaattgaagttaaaggtattaaagatgttgttactcaacctcaagct

SEQ ID NO: 122 - RNA, 1053 nt, synthetic construct
  source 1..1053: mol_type = other RNA
atgaaaaaaactgctattgctattactgttgctttagctggttttgctactgttgctcaagctgctcctaaagataatacttggtatactggtgctaaattaggttggtctcaatatcatgatactggttttattgataataatggtcctactcatgaaaatcaattaggtgctggtgcttttggtggttatcaagttaatccttatgttggttttgaaatgggttatgattggttaggtcgtatgccttataaaggttctgttgaaaatggtgcttataaagctcaaggtgttcaattaactgctaaattaggttatcctattactgatgatttagatgtttatactcgtttaggtggtatggtttggcgtgctgatactaaagctcataataatgttactggtgaatctgaaaaaaatcatgatactggtgtttctcctgtttttgctggtggtgttgaatgggctattactcctgaaattgctactcgtttagaatatcaatggactaataatattggtgatgctcatactattggtactcgtcctgataatggtttattatctttaggtgtttcttatcgttttggtcaaggtgaagctgctcctgttgttgctcctgctcctgctcctgctcctgaagttcaaactaaacattttactttaaaatctgatgttttatttaattttaataaagctactttaaaacctgaaggtcaagctgctttagatcaattatattctcaattatctaatttagatcctaaagatggttctgttgttgttttaggttatactgatcgtattggttctgatgcttataatcaaggtttatctgaacgtcgtgctcaatctgttgttgattatttaatttctaaaggtattcctgctgataaaatttctgctcgtggtatgggtgaatctaatcctgttactggtaatacttgtgataatgttaaacaacgtgctgctttaattgattgtttagctcctgatcgtcgtgttgaaattgaagttaaaggtattaaagatgttgttactcaacctcaagct

SEQ ID NO: 123 - RNA, 1053 nt, synthetic construct
  source 1..1053: mol_type = other RNA
atgaagaagaccgccatcgccatcaccgtcgccttggccggcttcgccaccgtcgcccaggccgcccccaaggacaacacctggtacaccggcgccaagttgggctggtcccagtaccacgacaccggcttcatcgacaacaacggccccacccacgagaaccagttgggcgccggcgccttcggcggctaccaggtcaacccctacgtcggcttcgagatgggctacgactggttgggccgcatgccctacaagggctccgtcgagaacggcgcctacaaggcccagggcgtccagttgaccgccaagttgggctaccccatcaccgacgacttggacgtctacacccgcttgggcggcatggtctggcgcgccgacaccaaggcccacaacaacgtcaccggcgagtccgagaagaaccacgacaccggcgtctcccccgtcttcgccggcggcgtcgagtgggccatcacccccgagatcgccacccgcttggagtaccagtggaccaacaacatcggcgacgcccacaccatcggcacccgccccgacaacggcttgttgtccttgggcgtctcctaccgcttcggccagggcgaggccgcccccgtcgtcgcccccgcccccgcccccgcccccgaggtccagaccaagcacttcaccttgaagtccgacgtcttgttcaacttcaacaaggccaccttgaagcccgagggccaggccgccttggaccagttgtactcccagttgtccaacttggaccccaaggacggctccgtcgtcgtcttgggctacaccgaccgcatcggctccgacgcctacaaccagggcttgtccgagcgccgcgcccagtccgtcgtcgactacttgatctccaagggcatccccgccgacaagatctccgcccgcggcatgggcgagtccaaccccgtcaccggcaacacctgcgacaacgtcaagcagcgcgccgccttgatcgactgcttggcccccgaccgccgcgtcgagatcgaggtcaagggcatcaaggacgtcgtcacccagccccaggcc

SEQ ID NO: 124 - RNA, 1053 nt, synthetic construct
  source 1..1053: mol_type = other RNA
atgaaaaaaacagcaatagcaataacagtagcacttgcaggatttgcaacagtagcacaagcagcaccaaaagataatacatggtatacaggagcaaaacttggatggtcacaatatcatgatacaggatttatagataataatggaccaacacatgaaaatcaacttggagcaggagcatttggaggatatcaagtaaatccatatgtaggatttgaaatgggatatgattggcttggacgaatgccatataaaggatcagtagaaaatggagcatataaagcacaaggagtacaacttacagcaaaacttggatatccaataacagatgatcttgatgtatatacacgacttggaggaatggtatggcgagcagatacaaaagcacataataatgtaacaggagaatcagaaaaaaatcatgatacaggagtatcaccagtatttgcaggaggagtagaatgggcaataacaccagaaatagcaacacgacttgaatatcaatggacaaataatataggagatgcacatacaataggaacacgaccagataatggacttctttcacttggagtatcatatcgatttggacaaggagaagcagcaccagtagtagcaccagcaccagcaccagcaccagaagtacaaacaaaacattttacacttaaatcagatgtactttttaattttaataaagcaacacttaaaccagaaggacaagcagcacttgatcaactttattcacaactttcaaatcttgatccaaaagatggatcagtagtagtacttggatatacagatcgaataggatcagatgcatataatcaaggactttcagaacgacgagcacaatcagtagtagattatcttatatcaaaaggaataccagcagataaaatatcagcacgaggaatgggagaatcaaatccagtaacaggaaatacatgtgataatgtaaaacaacgagcagcacttatagattgtcttgcaccagatcgacgagtagaaatagaagtaaaaggaataaaagatgtagtaacacaaccacaagca

SEQ ID NO: 125 - RNA, 1053 nt, synthetic construct
  source 1..1053: mol_type = other RNA
atgaaaaaaacggcgattgcgattacggtggcgctcgcggggtttgcgacggtggcgcaagcggcgccgaaagataatacgtggtatacgggggcgaaactcgggtggtcgcaatatcatgatacggggtttattgataataatgggccgacgcatgaaaatcaactcggggcgggggcgtttggggggtatcaagtgaatccgtatgtggggtttgaaatggggtatgattggctcgggcggatgccgtataaagggtcggtggaaaatggggcgtataaagcgcaaggggtgcaactcacggcgaaactcgggtatccgattacggatgatctcgatgtgtatacgcggctcggggggatggtgtggcgggcggatacgaaagcgcataataatgtgacgggggaatcggaaaaaaatcatgatacgggggtgtcgccggtgtttgcggggggggtggaatgggcgattacgccggaaattgcgacgcggctcgaatatcaatggacgaataatattggggatgcgcatacgattgggacgcggccggataatgggctcctctcgctcggggtgtcgtatcggtttgggcaaggggaagcggcgccggtggtggcgccggcgccggcgccggcgccggaagtgcaaacgaaacattttacgctcaaatcggatgtgctctttaattttaataaagcgacgctcaaaccggaagggcaagcggcgctcgatcaactctattcgcaactctcgaatctcgatccgaaagatgggtcggtggtggtgctcgggtatacggatcggattgggtcggatgcgtataatcaagggctctcggaacggcgggcgcaatcggtggtggattatctcatttcgaaagggattccggcggataaaatttcggcgcgggggatgggggaatcgaatccggtgacggggaatacgtgtgataatgtgaaacaacgggcggcgctcattgattgtctcgcgccggatcggcgggtggaaattgaagtgaaagggattaaagatgtggtgacgcaaccgcaagcg

SEQ ID NO: 126 - RNA, 1053 nt, synthetic construct
  source 1..1053: mol_type = other RNA
atgaaaaaaactgctattgctattactgttgctctagctggttttgctactgttgctcaagctgctcctaaagataatacttggtatactggtgctaaactaggttggagtcaatatcatgatactggttttattgataataatggtcctactcatgaaaatcaactaggtgctggtgcttttggtggttatcaagttaatccttatgttggttttgaaatgggttatgattggctaggtagaatgccttataaaggtagtgttgaaaatggtgcttataaagctcaaggtgttcaactaactgctaaactaggttatcctattactgatgatctagatgtttatactagactaggtggtatggtttggagagctgatactaaagctcataataatgttactggtgaaagtgaaaaaaatcatgatactggtgttagtcctgtttttgctggtggtgttgaatgggctattactcctgaaattgctactagactagaatatcaatggactaataatattggtgatgctcatactattggtactagacctgataatggtctactaagtctaggtgttagttatagatttggtcaaggtgaagctgctcctgttgttgctcctgctcctgctcctgctcctgaagttcaaactaaacattttactctaaaaagtgatgttctatttaattttaataaagctactctaaaacctgaaggtcaagctgctctagatcaactatatagtcaactaagtaatctagatcctaaagatggtagtgttgttgttctaggttatactgatagaattggtagtgatgcttataatcaaggtctaagtgaaagaagagctcaaagtgttgttgattatctaattagtaaaggtattcctgctgataaaattagtgctagaggtatgggtgaaagtaatcctgttactggtaatacttgtgataatgttaaacaaagagctgctctaattgattgtctagctcctgatagaagagttgaaattgaagttaaaggtattaaagatgttgttactcaacctcaagct

SEQ ID NO: 127 - RNA, 1053 nt, synthetic construct
  source 1..1053: mol_type = other RNA
atgaaaaaaactgctattgctattactgttgctctggctggttttgctactgttgctcaagctgctcctaaagataatacttggtatactggtgctaaactgggttggagccaatatcatgatactggttttattgataataatggtcctactcatgaaaatcaactgggtgctggtgcttttggtggttatcaagttaatccttatgttggttttgaaatgggttatgattggctgggtaggatgccttataaaggtagcgttgaaaatggtgcttataaagctcaaggtgttcaactgactgctaaactgggttatcctattactgatgatctggatgtttatactaggctgggtggtatggtttggagggctgatactaaagctcataataatgttactggtgaaagcgaaaaaaatcatgatactggtgttagccctgtttttgctggtggtgttgaatgggctattactcctgaaattgctactaggctggaatatcaatggactaataatattggtgatgctcatactattggtactaggcctgataatggtctgctgagcctgggtgttagctataggtttggtcaaggtgaagctgctcctgttgttgctcctgctcctgctcctgctcctgaagttcaaactaaacattttactctgaaaagcgatgttctgtttaattttaataaagctactctgaaacctgaaggtcaagctgctctggatcaactgtatagccaactgagcaatctggatcctaaagatggtagcgttgttgttctgggttatactgataggattggtagcgatgcttataatcaaggtctgagcgaaaggagggctcaaagcgttgttgattatctgattagcaaaggtattcctgctgataaaattagcgctaggggtatgggtgaaagcaatcctgttactggtaatacttgtgataatgttaaacaaagggctgctctgattgattgtctggctcctgataggagggttgaaattgaagttaaaggtattaaagatgttgttactcaacctcaagct

SEQ ID NO: 128 - RNA, 813 nt, synthetic construct
  source 1..813: mol_type = other RNA
gttaaacgtactgaagatacttcttctgataaatggggtgttactcaaaatattcaatttgattttgttaaagataaaaaatataataaagatgctttaattttaaaaatgcaaggttttattaattctaaaactacttattataattataaaaatactgatcatattaaagctatgcgttggccttttcaatataatattggtttaaaaactaatgatcctaatgttgatttaattaattatttacctaaaaataaaattgattctgttaatgtttctcaaactttaggttataatattggtggtaattttaattctggtccttctactggtggtaatggttcttttaattattctaaaactatttcttataatcaacaaaattatatttctgaagttgaacatcaaaattctaaatctgttcaatggggtattaaagctaattcttttattacttctttaggtaaaatgtctggtcatgatcctaatttatttgttggttataaaccttattctcaaaatcctcgtgattattttgttcctgataatgaattacctcctttagttcattctggttttaatccttcttttattgctactgtttctcatgaaaaaggttctggtgatacttctgaatttgaaattacttatggtcgtaaaatggatgttactcatgctactcgtcgtactactcattatggtaattcttatttagaaggttctcgtattcataatgcttttgttaatcgtaattatactgttaaatatgaagttaattggaaaactcatgaaattaaagttaaaggtcataat

SEQ ID NO: 129 - RNA, 993 nt, synthetic construct
  source 1..993: mol_type = other RNA
atgaaaaaatctttaattgctttaactttagctgctttacctgttgctgctatggctgatgttactttatatggtactattaaagctggtgttgaaacttctcgttctgttgaacataatggtggtcaagttgtttctgttgaaactggtactggtattgttgatttaggttctaaaattggttttaaaggtcaagaagatttaggtaatggtttaaaagctatttggcaagttgaacaaaaagcttctattgctggtactgattctggttggggtaatcgtcaatcttttattggtttaaaaggtggttttggtaaattacgtgttggtcgtttaaattctgttttaaaagatactggtgatattaatccttgggattctaaatctgattatttaggtgttaataaaattgctgaacctgaagctcgtttaatttctgttcgttatgattctcctgaatttgctggtttatctggttctgttcaatatgctttaaatgataatgctggtaaatataattctgaatcttatcatgctggttttaattataaaaatggtggtttttttgttcaatatggtggtgcttataaacgtcatgttcgtgttgatgaaaatgttaatattgaaaaatatcaaattcatcgtttagtttctggttatgataatgatgctttacatgcttctgttgctgttcaacaacaagatgctaaattagttgaagataattattctcataattctcaaactgaagttgctgctactttagcttatcgttttggtaatgttactcctcgtgtttcttatgctcatggttttaaaggttcttttgatgatgctgatttatctaatgattatgatcaagttgttgttggtgctgaatatgatttttctaaacgtacttctgctttagtttctgctggttggttacaagaaggtaaaggtgaaaataaatttgtttctactgctggtggtgttggtttacgtcataaattt

SEQ ID NO: 130 - RNA, 813 nt, synthetic construct
  source 1..813: mol_type = other RNA
gtaaaacgaacagaagatacatcatcagataaatggggagtaacacaaaatatacaatttgattttgtaaaagataaaaaatataataaagatgcacttatacttaaaatgcaaggatttataaattcaaaaacaacatattataattataaaaatacagatcatataaaagcaatgcgatggccatttcaatataatataggacttaaaacaaatgatccaaatgtagatcttataaattatcttccaaaaaataaaatagattcagtaaatgtatcacaaacacttggatataatataggaggaaattttaattcaggaccatcaacaggaggaaatggatcatttaattattcaaaaacaatatcatataatcaacaaaattatatatcagaagtagaacatcaaaattcaaaatcagtacaatggggaataaaagcaaattcatttataacatcacttggaaaaatgtcaggacatgatccaaatctttttgtaggatataaaccatattcacaaaatccacgagattattttgtaccagataatgaacttccaccacttgtacattcaggatttaatccatcatttatagcaacagtatcacatgaaaaaggatcaggagatacatcagaatttgaaataacatatggacgaaaaatggatgtaacacatgcaacacgacgaacaacacattatggaaattcatatcttgaaggatcacgaatacataatgcatttgtaaatcgaaattatacagtaaaatatgaagtaaattggaaaacacatgaaataaaagtaaaaggacataat

SEQ ID NO: 131 - RNA, 813 nt, synthetic construct
  source 1..813: mol_type = other RNA
gtgaaacggacggaagatacgtcgtcggataaatggggggtgacgcaaaatattcaatttgattttgtgaaagataaaaaatataataaagatgcgctcattctcaaaatgcaagggtttattaattcgaaaacgacgtattataattataaaaatacggatcatattaaagcgatgcggtggccgtttcaatataatattgggctcaaaacgaatgatccgaatgtggatctcattaattatctcccgaaaaataaaattgattcggtgaatgtgtcgcaaacgctcgggtataatattggggggaattttaattcggggccgtcgacgggggggaatgggtcgtttaattattcgaaaacgatttcgtataatcaacaaaattatatttcggaagtggaacatcaaaattcgaaatcggtgcaatgggggattaaagcgaattcgtttattacgtcgctcgggaaaatgtcggggcatgatccgaatctctttgtggggtataaaccgtattcgcaaaatccgcgggattattttgtgccggataatgaactcccgccgctcgtgcattcggggtttaatccgtcgtttattgcgacggtgtcgcatgaaaaagggtcgggggatacgtcggaatttgaaattacgtatgggcggaaaatggatgtgacgcatgcgacgcggcggacgacgcattatgggaattcgtatctcgaagggtcgcggattcataatgcgtttgtgaatcggaattatacggtgaaatatgaagtgaattggaaaacgcatgaaattaaagtgaaagggcataat

SEQ ID NO: 132 - RNA, 813 nt, synthetic construct
  source 1..813: mol_type = other RNA
gttaaaagaactgaagatactagtagtgataaatggggtgttactcaaaatattcaatttgattttgttaaagataaaaaatataataaagatgctctaattctaaaaatgcaaggttttattaatagtaaaactacttattataattataaaaatactgatcatattaaagctatgagatggccttttcaatataatattggtctaaaaactaatgatcctaatgttgatctaattaattatctacctaaaaataaaattgatagtgttaatgttagtcaaactctaggttataatattggtggtaattttaatagtggtcctagtactggtggtaatggtagttttaattatagtaaaactattagttataatcaacaaaattatattagtgaagttgaacatcaaaatagtaaaagtgttcaatggggtattaaagctaatagttttattactagtctaggtaaaatgagtggtcatgatcctaatctatttgttggttataaaccttatagtcaaaatcctagagattattttgttcctgataatgaactacctcctctagttcatagtggttttaatcctagttttattgctactgttagtcatgaaaaaggtagtggtgatactagtgaatttgaaattacttatggtagaaaaatggatgttactcatgctactagaagaactactcattatggtaatagttatctagaaggtagtagaattcataatgcttttgttaatagaaattatactgttaaatatgaagttaattggaaaactcatgaaattaaagttaaaggtcataat

SEQ ID NO: 133 - RNA, 813 nt, synthetic construct
  source 1..813: mol_type = other RNA
gttaaaaggactgaagatactagcagcgataaatggggtgttactcaaaatattcaatttgattttgttaaagataaaaaatataataaagatgctctgattctgaaaatgcaaggttttattaatagcaaaactacttattataattataaaaatactgatcatattaaagctatgaggtggccttttcaatataatattggtctgaaaactaatgatcctaatgttgatctgattaattatctgcctaaaaataaaattgatagcgttaatgttagccaaactctgggttataatattggtggtaattttaatagcggtcctagcactggtggtaatggtagctttaattatagcaaaactattagctataatcaacaaaattatattagcgaagttgaacatcaaaatagcaaaagcgttcaatggggtattaaagctaatagctttattactagcctgggtaaaatgagcggtcatgatcctaatctgtttgttggttataaaccttatagccaaaatcctagggattattttgttcctgataatgaactgcctcctctggttcatagcggttttaatcctagctttattgctactgttagccatgaaaaaggtagcggtgatactagcgaatttgaaattacttatggtaggaaaatggatgttactcatgctactaggaggactactcattatggtaatagctatctggaaggtagcaggattcataatgcttttgttaataggaattatactgttaaatatgaagttaattggaaaactcatgaaattaaagttaaaggtcataat

SEQ ID NO: 134 - RNA, 993 nt, synthetic construct; identical to SEQ ID NO: 129
  source 1..993: mol_type = other RNA

SEQ ID NO: 135 - RNA, 993 nt, synthetic construct
  source 1..993: mol_type = other RNA
atgaagaagtccttgatcgccttgaccttggccgccttgcccgtcgccgccatggccgacgtcaccttgtacggcaccatcaaggccggcgtcgagacctcccgctccgtcgagcacaacggcggccaggtcgtctccgtcgagaccggcaccggcatcgtcgacttgggctccaagatcggcttcaagggccaggaggacttgggcaacggcttgaaggccatctggcaggtcgagcagaaggcctccatcgccggcaccgactccggctggggcaaccgccagtccttcatcggcttgaagggcggcttcggcaagttgcgcgtcggccgcttgaactccgtcttgaaggacaccggcgacatcaacccctgggactccaagtccgactacttgggcgtcaacaagatcgccgagcccgaggcccgcttgatctccgtccgctacgactcccccgagttcgccggcttgtccggctccgtccagtacgccttgaacgacaacgccggcaagtacaactccgagtcctaccacgccggcttcaactacaagaacggcggcttcttcgtccagtacggcggcgcctacaagcgccacgtccgcgtcgacgagaacgtcaacatcgagaagtaccagatccaccgcttggtctccggctacgacaacgacgccttgcacgcctccgtcgccgtccagcagcaggacgccaagttggtcgaggacaactactcccacaactcccagaccgaggtcgccgccaccttggcctaccgcttcggcaacgtcaccccccgcgtctcctacgcccacggcttcaagggctccttcgacgacgccgacttgtccaacgactacgaccaggtcgtcgtcggcgccgagtacgacttctccaagcgcacctccgccttggtctccgccggctggttgcaggagggcaagggcgagaacaagttcgtctccaccgccggcggcgtcggcttgcgccacaagttc

SEQ ID NO: 136 - RNA, 993 nt, synthetic construct
  source 1..993: mol_type = other RNA
atgaaaaaatcacttatagcacttacacttgcagcacttccagtagcagcaatggcagatgtaacactttatggaacaataaaagcaggagtagaaacatcacgatcagtagaacataatggaggacaagtagtatcagtagaaacaggaacaggaatagtagatcttggatcaaaaataggatttaaaggacaagaagatcttggaaatggacttaaagcaatatggcaagtagaacaaaaagcatcaatagcaggaacagattcaggatggggaaatcgacaatcatttataggacttaaaggaggatttggaaaacttcgagtaggacgacttaattcagtacttaaagatacaggagatataaatccatgggattcaaaatcagattatcttggagtaaataaaatagcagaaccagaagcacgacttatatcagtacgatatgattcaccagaatttgcaggactttcaggatcagtacaatatgcacttaatgataatgcaggaaaatataattcagaatcatatcatgcaggatttaattataaaaatggaggattttttgtacaatatggaggagcatataaacgacatgtacgagtagatgaaaatgtaaatatagaaaaatatcaaatacatcgacttgtatcaggatatgataatgatgcacttcatgcatcagtagcagtacaacaacaagatgcaaaacttgtagaagataattattcacataattcacaaacagaagtagcagcaacacttgcatatcgatttggaaatgtaacaccacgagtatcatatgcacatggatttaaaggatcatttgatgatgcagatctttcaaatgattatgatcaagtagtagtaggagcagaatatgatttttcaaaacgaacatcagcacttgtatcagcaggatggcttcaagaaggaaaaggagaaaataaatttgtatcaacagcaggaggagtaggacttcgacataaattt

SEQ ID NO: 137 - RNA, 993 nt, synthetic construct
  source 1..993: mol_type = other RNA
atgaaaaaatcgctcattgcgctcacgctcgcggcgctcccggtggcggcgatggcggatgtgacgctctatgggacgattaaagcgggggtggaaacgtcgcggtcggtggaacataatggggggcaagtggtgtcggtggaaacggggacggggattgtggatctcgggtcgaaaattgggtttaaagggcaagaagatctcgggaatgggctcaaagcgatttggcaagtggaacaaaaagcgtcgattgcggggacggattcggggtgggggaatcggcaatcgtttattgggctcaaaggggggtttgggaaactccgggtggggcggctcaattcggtgctcaaagatacgggggatattaatccgtgggattcgaaatcggattatctcggggtgaataaaattgcggaaccggaagcgcggctcatttcggtgcggtatgattcgccggaatttgcggggctctcggggtcggtgcaatatgcgctcaatgataatgcggggaaatataattcggaatcgtatcatgcggggtttaattataaaaatggggggttttttgtgcaatatgggggggcgtataaacggcatgtgcgggtggatgaaaatgtgaatattgaaaaatatcaaattcatcggctcgtgtcggggtatgataatgatgcgctccatgcgtcggtggcggtgcaacaacaagatgcgaaactcgtggaagataattattcgcataattcgcaaacggaagtggcggcgacgctcgcgtatcggtttgggaatgtgacgccgcgggtgtcgtatgcgcatgggtttaaagggtcgtttgatgatgcggatctctcgaatgattatgatcaagtggtggtgggggcggaatatgatttttcgaaacggacgtcggcgctcgtgtcggcggggtggctccaagaagggaaaggggaaaataaatttgtgtcgacggcggggggggtggggctccggcataaattt

SEQ ID NO: 138 - RNA, 993 nt, synthetic construct
  source 1..993: mol_type = other RNA
atgaaaaaaagtctaattgctctaactctagctgctctacctgttgctgctatggctgatgttactctatatggtactattaaagctggtgttgaaactagtagaagtgttgaacataatggtggtcaagttgttagtgttgaaactggtactggtattgttgatctaggtagtaaaattggttttaaaggtcaagaagatctaggtaatggtctaaaagctatttggcaagttgaacaaaaagctagtattgctggtactgatagtggttggggtaatagacaaagttttattggtctaaaaggtggttttggtaaactaagagttggtagactaaatagtgttctaaaagatactggtgatattaatccttgggatagtaaaagtgattatctaggtgttaataaaattgctgaacctgaagctagactaattagtgttagatatgatagtcctgaatttgctggtctaagtggtagtgttcaatatgctctaaatgataatgctggtaaatataatagtgaaagttatcatgctggttttaattataaaaatggtggtttttttgttcaatatggtggtgcttataaaagacatgttagagttgatgaaaatgttaatattgaaaaatatcaaattcatagactagttagtggttatgataatgatgctctacatgctagtgttgctgttcaacaacaagatgctaaactagttgaagataattatagtcataatagtcaaactgaagttgctgctactctagcttatagatttggtaatgttactcctagagttagttatgctcatggttttaaaggtagttttgatgatgctgatctaagtaatgattatgatcaagttgttgttggtgctgaatatgattttagtaaaagaactagtgctctagttagtgctggttggctacaagaaggtaaaggtgaaaataaatttgttagtactgctggtggtgttggtctaagacataaattt

SEQ ID NO: 139 - RNA, 993 nt, synthetic construct
  source 1..993: mol_type = other RNA
atgaaaaaaagcctgattgctctgactctggctgctctgcctgttgctgctatggctgatgttactctgtatggtactattaaagctggtgttgaaactagcaggagcgttgaacataatggtggtcaagttgttagcgttgaaactggtactggtattgttgatctgggtagcaaaattggttttaaaggtcaagaagatctgggtaatggtctgaaagctatttggcaagttgaacaaaaagctagcattgctggtactgatagcggttggggtaataggcaaagctttattggtctgaaaggtggttttggtaaactgagggttggtaggctgaatagcgttctgaaagatactggtgatattaatccttgggatagcaaaagcgattatctgggtgttaataaaattgctgaacctgaagctaggctgattagcgttaggtatgatagccctgaatttgctggtctgagcggtagcgttcaatatgctctgaatgataatgctggtaaatataatagcgaaagctatcatgctggttttaattataaaaatggtggtttttttgttcaatatggtggtgcttataaaaggcatgttagggttgatgaaaatgttaatattgaaaaatatcaaattcataggctggttagcggttatgataatgatgctctgcatgctagcgttgctgttcaacaacaagatgctaaactggttgaagataattatagccataatagccaaactgaagttgctgctactctggcttataggtttggtaatgttactcctagggttagctatgctcatggttttaaaggtagctttgatgatgctgatctgagcaatgattatgatcaagttgttgttggtgctgaatatgattttagcaaaaggactagcgctctggttagcgctggttggctgcaagaaggtaaaggtgaaaataaatttgttagcactgctggtggtgttggtctgaggcataaattt

SEQ ID NO: 140 - RNA, 285 nt, synthetic construct
  source 1..285: mol_type = other RNA
atgactgaacaacaatggaattttgctggtattgaagctgctgcttctgctattcaaggtaatgttacttctattcattctttattagatgaaggtaaacaatctttaactaaattagctgctgcttggggtggttctggttctgaagcttatcaaggtgttcaacaaaaatgggatgctactgctactgaattaaataatgctttacaaaatttagctcgtactatttctgaagctggtcaagctatggcttctactgaaggtaatgttactggtatgtttgct

SEQ ID NO: 141 - RNA, 285 nt, synthetic construct
  source 1..285: mol_type = other RNA
atgaccgagcagcagtggaacttcgccggcatcgaggccgccgcctccgccatccagggcaacgtcacctccatccactccttgttggacgagggcaagcagtccttgaccaagttggccgccgcctggggcggctccggctccgaggcctaccagggcgtccagcagaagtgggacgccaccgccaccgagttgaacaacgccttgcagaacttggcccgcaccatctccgaggccggccaggccatggcctccaccgagggcaacgtcaccggcatgttcgcc

SEQ ID NO: 142 - RNA, 285 nt, synthetic construct
  source 1..285: mol_type = other RNA
atgacagaacaacaatggaattttgcaggaatagaagcagcagcatcagcaatacaaggaaatgtaacatcaatacattcacttcttgatgaaggaaaacaatcacttacaaaacttgcagcagcatggggaggatcaggatcagaagcatatcaaggagtacaacaaaaatgggatgcaacagcaacagaacttaataatgcacttcaaaatcttgcacgaacaatatcagaagcaggacaagcaatggcatcaacagaaggaaatgtaacaggaatgtttgca

SEQ ID NO: 143 - RNA, 285 nt, synthetic construct
  source 1..285: mol_type = other RNA
atgacggaacaacaatggaattttgcggggattgaagcggcggcgtcggcgattcaagggaatgtgacgtcgattcattcgctcctcgatgaagggaaacaatcgctcacgaaactcgcggcggcgtggggggggtcggggtcggaagcgtatcaaggggtgcaacaaaaatgggatgcgacggcgacggaactcaataatgcgctccaaaatctcgcgcggacgatttcggaagcggggcaagcgatggcgtcgacggaagggaatgtgacggggatgtttgcg

SEQ ID NO: 144 - RNA, 285 nt, synthetic construct
  source 1..285: mol_type = other RNA
atgactgaacaacaatggaattttgctggtattgaagctgctgctagtgctattcaaggtaatgttactagtattcatagtctactagatgaaggtaaacaaagtctaactaaactagctgctgcttggggtggtagtggtagtgaagcttatcaaggtgttcaacaaaaatgggatgctactgctactgaactaaataatgctctacaaaatctagctagaactattagtgaagctggtcaagctatggctagtactgaaggtaatgttactggtatgtttgct

SEQ ID NO: 145 - RNA, 285 nt, synthetic construct
  source 1..285: mol_type = other RNA
atgactgaacaacaatggaattttgctggtattgaagctgctgctagcgctattcaaggtaatgttactagcattcatagcctgctggatgaaggtaaacaaagcctgactaaactggctgctgcttggggtggtagcggtagcgaagcttatcaaggtgttcaacaaaaatgggatgctactgctactgaactgaataatgctctgcaaaatctggctaggactattagcgaagctggtcaagctatggctagcactgaaggtaatgttactggtatgtttgct

SEQ ID NO: 146 - RNA, 1485 nt, synthetic construct
  source 1..1485: mol_type = other RNA
atgaatcaacattttgatgttttaattattggtgctggtttatctggtattggtactgcttgtcatgttactgctgaatttcctgataaaactattgctttattagaacgtcgtgaacgtttaggtggtacttgggatttatttcgttatcctggtgttcgttctgattctgatatgtttacttttggttataaatttcgtccttggcgtgatgttaaagttttagctgatggtgcttctattcgtcaatatattgctgatactgctactgaatttggtgttgatgaaaaaattcattatggtttaaaagttaatactgctgaatggtcttctcgtcaatgtcgttggactgttgctggtgttcatgaagctactggtgaaactcgtacttatacttgtgattatttaatttcttgtactggttattataattatgatgctggttatttacctgattttcctggtgttcatcgttttggtggtcgttgtgttcatcctcaacattggcctgaagatttagattattctggtaaaaaagttgttgttattggttctggtgctactgctgttactttagttcctgctatggctggttctaatcctggttctgctgctcatgttactatgttacaacgttctccttcttatattttttctttacctgctgttgataaaatttctgaagttttaggtcgttttttacctgatcgttgggtttatgaatttggtcgtcgtcgtaatattgctattcaacgtaaattatatcaagcttgtcgtcgttggcctaaattaatgcgtcgtttattattatgggaagttcgtcgtcgtttaggtcgttctgttgatatgtctaattttactcctaattatttaccttgggatgaacgtttatgtgctgttcctaatggtgatttatttaaaactttagcttctggtgctgcttctgttgttactgatcaaattgaaacttttactgaaaaaggtattttatgtaaatctggtcgtgaaattgaagctgatattattgttactgctactggtttaaatattcaaatgttaggtggtatgcgtttaattgttgatggtgctgaatatcaattacctgaaaaaatgacttataaaggtgttttattagaaaatgctcctaatttagcttggattattggttatactaatgcttcttggactttaaaatctgatattgctggtgcttatttatgtcgtttattacgtcatatggctgataatggttatactgttgctactcctcgtgatgctcaagattgtgctttagatgttggtatgtttgatcaattaaattctggttatgttaaacgtggtcaagatattatgcctcgtcaaggttctaaacatccttggcgtgttttaatgcattatgaaaaagatgctaaaattttattagaagatcctattgatgatggtgttttacattttgctgctgctgctcaagatcatgctgctgct

SEQ ID NO: 147 - RNA, 1485 nt, synthetic construct
  source 1..1485: mol_type = other RNA
atgaaccagcacttcgacgtcttgatcatcggcgccggcttgtccggcatcggcaccgcctgccacgtcaccgccgagttccccgacaagaccatcgccttgttggagcgccgcgagcgcttgggcggcacctgggacttgttccgctaccccggcgtccgctccgactccgacatgttcaccttcggctacaagttccgcccctggcgcgacgtcaaggtcttggccgacggcgcctccatccgccagtacatcgccgacaccgccaccgagttcggcgtcgacgagaagatccactacggcttgaaggtcaacaccgccgagtggtcctcccgccagtgccgctggaccgtcgccggcgtccacgaggccaccggcgagacccgcacctacacctgcgactacttgatctcctgcaccggctactacaactacgacgccggctacttgcccgacttccccggcgtccaccgcttcggcggccgctgcgtccacccccagcactggcccgaggacttggactactccggcaagaaggtcgtcgtcatcggctccggcgccaccgccgtcaccttggtccccgccatggccggctccaaccccggctccgccgcccacgtcaccatgttgcagcgctccccctcctacatcttctccttgcccgccgtcgacaagatctccgaggtcttgggccgcttcttgcccgaccgctgggtctacgagttcggccgccgccgcaacatcgccatccagcgcaagttgtaccaggcctgccgccgctggcccaagttgatgcgccgcttgttgttgtgggaggtccgccgccgcttgggccgctccgtcgacatgtccaacttcacccccaactacttgccctgggacgagcgcttgtgcgccgtccccaacggcgacttgttcaagaccttggcctccggcgccgcctccgtcgtcaccgaccagatcgagaccttcaccgagaagggcatcttgtgcaagtccggccgcgagatcgaggccgacatcatcgtcaccgccaccggcttgaacatccagatgttgggcggcatgcgcttgatcgtcgacggcgccgagtaccagttgcccgagaagatgacctacaagggcgtcttgttggagaacgcccccaacttggcctggatcatcggctacaccaacgcctcctggaccttgaagtccgacatcgccggcgcctacttgtgccgcttgttgcgccacatggccgacaacggctacaccgtcgccaccccccgcgacgcccaggactgcgccttggacgtcggcatgttcgaccagttgaactccggctacgtcaagcgcggccaggacatcatgccccgccagggctccaagcacccctggcgcgtcttgatgcactacgagaaggacgccaagatcttgttggaggaccccatcgacgacggcgtcttgcacttcgccgccgccgcccaggaccacgccgccgcc

SEQ ID NO: 148 - RNA, 1485 nt, synthetic construct
  source 1..1485: mol_type = other RNA
atgaatcaacattttgatgtacttataataggagcaggactttcaggaataggaacagcatgtcatgtaacagcagaatttccagataaaacaatagcacttcttgaacgacgagaacgacttggaggaacatgggatctttttcgatatccaggagtacgatcagattcagatatgtttacatttggatataaatttcgaccatggcgagatgtaaaagtacttgcagatggagcatcaatacgacaatatatagcagatacagcaacagaatttggagtagatgaaaaaatacattatggacttaaagtaaatacagcagaatggtcatcacgacaatgtcgatggacagtagcaggagtacatgaagcaacaggagaaacacgaacatatacatgtgattatcttatatcatgtacaggatattataattatgatgcaggatatcttccagattttccaggagtacatcgatttggaggacgatgtgtacatccacaacattggccagaagatcttgattattcaggaaaaaaagtagtagtaataggatcaggagcaacagcagtaacacttgtaccagcaatggcaggatcaaatccaggatcagcagcacatgtaacaatgcttcaacgatcaccatcatatatattttcacttccagcagtagataaaatatcagaagtacttggacgatttcttccagatcgatgggtatatgaatttggacgacgacgaaatatagcaatacaacgaaaactttatcaagcatgtcgacgatggccaaaacttatgcgacgacttcttctttgggaagtacgacgacgacttggacgatcagtagatatgtcaaattttacaccaaattatcttccatgggatgaacgactttgtgcagtaccaaatggagatctttttaaaacacttgcatcaggagcagcatcagtagtaacagatcaaatagaaacatttacagaaaaaggaatactttgtaaatcaggacgagaaatagaagcagatataatagtaacagcaacaggacttaatatacaaatgcttggaggaatgcgacttatagtagatggagcagaatatcaacttccagaaaaaatgacatataaaggagtacttcttgaaaatgcaccaaatcttgcatggataataggatatacaaatgcatcatggacacttaaatcagatatagcaggagcatatctttgtcgacttcttcgacatatggcagataatggatatacagtagcaacaccacgagatgcacaagattgtgcacttgatgtaggaatgtttgatcaacttaattcaggatatgtaaaacgaggacaagatataatgccacgacaaggatcaaaacatccatggcgagtacttatgcattatgaaaaagatgcaaaaatacttcttgaagatccaatagatgatggagtacttcattttgcagcagcagcacaagatcatgcagcagca

SEQ ID NO: 149 - RNA, 1485 nt, synthetic construct
  source 1..1485: mol_type = other RNA
atgaatcaacattttgatgtgctcattattggggcggggctctcggggattgggacggcgtgtcatgtgacggcggaatttccggataaaacgattgcgctcctcgaacggcgggaacggctcggggggacgtgggatctctttcggtatccgggggtgcggtcggattcggatatgtttacgtttgggtataaatttcggccgtggcgggatgtgaaagtgctcgcggatggggcgtcgattcggcaatatattgcggatacggcgacggaatttggggtggatgaaaaaattcattatgggctcaaagtgaatacggcggaatggtcgtcgcggcaatgtcggtggacggtggcgggggtgcatgaagcgacgggggaaacgcggacgtatacgtgtgattatctcatttcgtgtacggggtattataattatgatgcggggtatctcccggattttccgggggtgcatcggtttggggggcggtgtgtgcatccgcaacattggccggaagatctcgattattcggggaaaaaagtggtggtgattgggtcgggggcgacggcggtgacgctcgtgccggcgatggcggggtcgaatccggggtcggcggcgcatgtgacgatgctccaacggtcgccgtcgtatattttttcgctcccggcggtggataaaatttcggaagtgctcgggcggtttctcccggatcggtgggtgtatgaatttgggcggcggcggaatattgcgattcaacggaaactctatcaagcgtgtcggcggtggccgaaactcatgcggcggctcctcctctgggaagtgcggcggcggctcgggcggtcggtggatatgtcgaattttacgccgaattatctcccgtgggatgaacggctctgtgcggtgccgaatggggatctctttaaaacgctcgcgtcgggggcggcgtcggtggtgacggatcaaattgaaacgtttacggaaaaagggattctctgtaaatcggggcgggaaattgaagcggatattattgtgacggcgacggggctcaatattcaaatgctcggggggatgcggctcattgtggatggggcggaatatcaactcccggaaaaaatgacgtataaaggggtgctcctcgaaaatgcgccgaatctcgcgtggattattgggtatacgaatgcgtcgtggacgctcaaatcggatattgcgggggcgtatctctgtcggctcctccggcatatggcggataatgggtatacggtggcgacgccgcgggatgcgcaagattgtgcgctcgatgtggggatgtttgatcaactcaattcggggtatgtgaaacgggggcaagatattatgccgcggcaagggtcgaaacatccgtggcgggtgctcatgcattatgaaaaagatgcgaaaattctcctcgaagatccgattgatgatggggtgctccattttgcggcggcggcgcaagatcatgcggcggcg

SEQ ID NO: 150 - RNA, 1485 nt, synthetic construct
  source 1..1485: mol_type = other RNA
atgaatcaacattttgatgttctaattattggtgctggtctaagtggtattggtactgcttgtcatgttactgctgaatttcctgataaaactattgctctactagaaagaagagaaagactaggtggtacttgggatctatttagatatcctggtgttagaagtgatagtgatatgtttacttttggttataaatttagaccttggagagatgttaaagttctagctgatggtgctagtattagacaatatattgctgatactgctactgaatttggtgttgatgaaaaaattcattatggtctaaaagttaatactgctgaatggagtagtagacaatgtagatggactgttgctggtgttcatgaagctactggtgaaactagaacttatacttgtgattatctaattagttgtactggttattataattatgatgctggttatctacctgattttcctggtgttcatagatttggtggtagatgtgttcatcctcaacattggcctgaagatctagattatagtggtaaaaaagttgttgttattggtagtggtgctactgctgttactctagttcctgctatggctggtagtaatcctggtagtgctgctcatgttactatgctacaaagaagtcctagttatatttttagtctacctgctgttgataaaattagtgaagttctaggtagatttctacctgatagatgggtttatgaatttggtagaagaagaaatattgctattcaaagaaaactatatcaagcttgtagaagatggcctaaactaatgagaagactactactatgggaagttagaagaagactaggtagaagtgttgatatgagtaattttactcctaattatctaccttgggatgaaagactatgtgctgttcctaatggtgatctatttaaaactctagctagtggtgctgctagtgttgttactgatcaaattgaaacttttactgaaaaaggtattctatgtaaaagtggtagagaaattgaagctgatattattgttactgctactggtctaaatattcaaatgctaggtggtatgagactaattgttgatggtgctgaatatcaactacctgaaaaaatgacttataaaggtgttctactagaaaatgctcctaatctagcttggattattggttatactaatgctagttggactctaaaaagtgatattgctggtgcttatctatgtagactactaagacatatggctgataatggttatactgttgctactcctagagatgctcaagattgtgctctagatgttggtatgtttgatcaactaaatagtggttatgttaaaagaggtcaagatattatgcctagacaaggtagtaaacatccttggagagttctaatgcattatgaaaaagatgctaaaattctactagaagatcctattgatgatggtgttctacattttgctgctgctgctcaagatcatgctgctgct

SEQ ID NO: 151 - RNA, 1485 nt, synthetic construct
  source 1..1485: mol_type = other RNA
atgaatcaacattttgatgttctgattattggtgctggtctgagcggtattggtactgcttgtcatgttactgctgaatttcctgataaaactattgctctgctggaaaggagggaaaggctgggtggtacttgggatctgtttaggtatcctggtgttaggagcgatagcgatatgtttacttttggttataaatttaggccttggagggatgttaaagttctggctgatggtgctagcattaggcaatatattgctgatactgctactgaatttggtgttgatgaaaaaattcattatggtctgaaagttaatactgctgaatggagcagcaggcaatgtaggtggactgttgctggtgttcatgaagctactggtgaaactaggacttatacttgtgattatctgattagctgtactggttattataattatgatgctggttatctgcctgattttcctggtgttcataggtttggtggtaggtgtgttcatcctcaacattggcctgaagatctggattatagcggtaaaaaagttgttgttattggtagcggtgctactgctgttactctggttcctgctatggctggtagcaatcctggtagcgctgctcatgttactatgctgcaaaggagccctagctatatttttagcctgcctgctgttgataaaattagcgaagttctgggtaggtttctgcctgataggtgggtttatgaatttggtaggaggaggaatattgctattcaaaggaaactgtatcaagcttgtaggaggtggcctaaactgatgaggaggctgctgctgtgggaagttaggaggaggctgggtaggagcgttgatatgagcaattttactcctaattatctgccttgggatgaaaggctgtgtgctgttcctaatggtgatctgtttaaaactctggctagcggtgctgctagcgttgttactgatcaaattgaaacttttactgaaaaaggtattctgtgtaaaagcggtagggaaattgaagctgatattattgttactgctactggtctgaatattcaaatgctgggtggtatgaggctgattgttgatggtgctgaatatcaactgcctgaaaaaatgacttataaaggtgttctgctggaaaatgctcctaatctggcttggattattggttatactaatgctagctggactctgaaaagcgatattgctggtgcttatctgtgtaggctgctgaggcatatggctgataatggttatactgttgctactcctagggatgctcaagattgtgctctggatgttggtatgtttgatcaactgaatagcggttatgttaaaaggggtcaagatattatgcctaggcaaggtagcaaacatccttggagggttctgatgcattatgaaaaagatgctaaaattctgctggaagatcctattgatgatggtgttctgcattttgctgctgctgctcaagatcatgctgctgct

SEQ ID NO: 152 - RNA, 1110 nt, synthetic construct
  source 1..1110: mol_type = other RNA
atgaaaaaattagctttagttttaggtttattattagttgttggttctgttgcttctgctaaagaagttatgcctgctcctactcctgctcctgaaaaagttgttgaatatgttgaaaaacctgttattgtttatcgtgatcgtgaagttgctcctgcttggcgtcctaatggttctgttgatgttcaatatcgttggtatggtgaagttgaaaaaaaaaatcctaaagatgataaagatgaaaattgggctactggtaaagttaatgctggtcgtttacaaactttaactaaagttaattttactgaaaaacaaactttagaagttcgtactcgtaatcatcatactttaaatgatactgatgctaataataaaaaatctaatggtgctgctgatgaatatcgtttacgtcatttttataattttggtaaattaggttcttctaaagttaatgctacttctcgtgttgaatttaaacaaaaaactaatgatggtgaaaaatctttaggtgcttctgttttatttgattttgctgattatatttattctaataatttttttaaagttgataaattaggtttacgtcctggttataaatatgtttggaaaggtcatggtaatggtgaagaaggtactcctactgttcataatgaatatcatttagcttttgaatctgattttactttaccttttaattttgctttaaatttagaatatgatttatcttataatcgttatcgtgaaaaatttgaaactactgatggtttaaaaaaagctgaatggtatggtgaattaactgctgttttatctaattatactcctttatataaagctggtgcttttgaattaggttttaatgctgaaggtggttatgatacttataatatgcatcaatataaacgtattggtggtgaagatggtacttctgttgatcgtcgtgattatgaattatatttagaacctactttacaagtttcttataaacctactgattttgttaaattatatgctgctgctggtgctgattatcgtaatcgtattactggtgaatctgaagttaaacgttggcgttggcaacctactgcttctgctggtatgaaagttactttt

SEQ ID NO: 153 - RNA, 1110 nt, synthetic construct; identical to SEQ ID NO: 152
  source 1..1110: mol_type = other RNA

SEQ ID NO: 154 - RNA, 1110 nt, synthetic construct; identical to SEQ ID NO: 152
  source 1..1110: mol_type = other RNA

SEQ ID NO: 155 - RNA, 1110 nt, synthetic construct; identical to SEQ ID NO: 152
  source 1..1110: mol_type = other RNA

SEQ ID NO: 156 - RNA, 1110 nt, synthetic construct; identical to SEQ ID NO: 152
  source 1..1110: mol_type = other RNA

SEQ ID NO: 157 - RNA, 1110 nt, synthetic construct; identical to SEQ ID NO: 152
  source 1..1110: mol_type = other RNA

SEQ ID NO: 158 - RNA, 1206 nt, synthetic construct
  source 1..1206: mol_type = other RNA
atgaaaaaattattaaaatctgctttattatttgctgctactggttctgctttatctttacaagctttacctgttggtaatcctgctgaaccttctttattaattgatggtactatgtgggaaggtgcttctggtgatccttgtgatccttgtgctacttggtgtgatgctatttctattcgtgctggttattatggtgattatgtttttgatcgtgttttaaaagttgatgttaataaaactttttctggtatggctgctactcctactcaagctactggtaatgcttctaatactaatcaacctgaagctaatggtcgtcctaatattgcttatggtcgtcatatgcaagatgctgaatggttttctaatgctgcttttttagctttaaatatttgggatcgttttgatattttttgtactttaggtgcttctaatggttattttaaagcttcttctgctgcttttaatttagttggtttaattggtttttctgctgcttcttctatttctactgatttacctatgcaattacctaatgttggtattactcaaggtgttgttgaattttatactgatacttctttttcttggtctgttggtgctcgtggtgctttatgggaatgtggttgtgctactttaggtgctgaatttcaatatgctcaatctaatcctaaaattgaaatgttaaatgttacttcttctcctgctcaatttgttattcataaacctcgtggttataaaggtgcttcttctaattttcctttacctattactgctggtactactgaagctactgatactaaatctgctactattaaatatcatgaatggcaagttggtttagctttatcttatcgtttaaatatgttagttccttatattggtgttaattggtctcgtgctacttttgatgctgatactattcgtattgctcaacctaaattaaaatctgaaattttaaatattactacttggaatccttctttaattggttctactactgctttacctaataattctggtaaagatgttttatctgatgttttacaaattgcttctattcaaattaataaaatgaaatctcgtaaagcttgtggtgttgctgttggtgctactttaattgatgctgataaatggtctattactggtgaagctcgtttaattaatgaacgtgctgctcatatgaatgctcaatttcgtttt

SEQ ID NO: 159 - RNA, 1206 nt, synthetic construct
  source 1..1206: mol_type = other RNA
atgaagaagttgttgaagtccgccttgttgttcgccgccaccggctccgccttgtccttgcaggccttgcccgtcggcaaccccgccgagccctccttgttgatcgacggcaccatgtgggagggcgcctccggcgacccctgcgacccctgcgccacctggtgcgacgccatctccatccgcgccggctactacggcgactacgtcttcgaccgcgtcttgaaggtcgacgtcaacaagaccttctccggcatggccgccacccccacccaggccaccggcaacgcctccaacaccaaccagcccgaggccaacggccgccccaacatcgcctacggccgccacatgcaggacgccgagtggttctccaacgccgccttcttggccttgaacatctgggaccgcttcgacatcttctgcaccttgggcgcctccaacggctacttcaaggcctcctccgccgccttcaacttggtcggcttgatcggcttctccgccgcctcctccatctccaccgacttgcccatgcagttgcccaacgtcggcatcacccagggcgtcgtcgagttctacaccgacacctccttctcctggtccgtcggcgcccgcggcgccttgtgggagtgcggctgcgccaccttgggcgccgagttccagtacgcccagtccaaccccaagatcgagatgttgaacgtcacctcctcccccgcccagttcgtcatccacaagccccgcggctacaagggcgcctcctccaacttccccttgcccatcaccgccggcaccaccgaggccaccgacaccaagtccgccaccatcaagtaccacgagtggcaggtcggcttggccttgtcctaccgcttgaacatgttggtcccctacatcggcgtcaactggtcccgcgccaccttcgacgccgacaccatccgcatcgcccagcccaagttgaagtccgagatcttgaacatcaccacctggaacccctccttgatcggctccaccaccgccttgcccaacaactccggcaaggacgtcttgtccgacgtcttgcagatcgcctccatccagatcaacaagatgaagtcccgcaaggcctgcggcgtcgccgtcggcgccaccttgatcgacgccgacaagtggtccatcaccggcgaggcccgcttgatcaacgagcgcgccgcccacatgaacgcccagttccgcttc

SEQ ID NO: 160 - RNA, 1206 nt, synthetic construct
  source 1..1206: mol_type = other RNA
atgaaaaaacttcttaaatcagcacttctttttgcagcaacaggatcagcactttcacttcaagcacttccagtaggaaatccagcagaaccatcacttcttatagatggaacaatgtgggaaggagcatcaggagatccatgtgatccatgtgcaacatggtgtgatgcaatatcaatacgagcaggatattatggagattatgtatttgatcgagtacttaaagtagatgtaaataaaacattttcaggaatggcagcaacaccaacacaagcaacaggaaatgcatcaaatacaaatcaaccagaagcaaatggacgaccaaatatagcatatggacgacatatgcaagatgcagaatggttttcaaatgcagcatttcttgcacttaatatatgggatcgatttgatatattttgtacacttggagcatcaaatggatattttaaagcatcatcagcagcatttaatcttgtaggacttataggattttcagcagcatcatcaatatcaacagatcttccaatgcaacttccaaatgtaggaataacacaaggagtagtagaattttatacagatacatcattttcatggtcagtaggagcacgaggagcactttgggaatgtggatgtgcaacacttggagcagaatttcaatatgcacaatcaaatccaaaaatagaaatgcttaatgtaacatcatcaccagcacaatttgtaatacataaaccacgaggatataaaggagcatcatcaaattttccacttccaataacagcaggaacaacagaagcaacagatacaaaatcagcaacaataaaatatcatgaatggcaagtaggacttgcactttcatatcgacttaatatgcttgtaccatatataggagtaaattggtcacgagcaacatttgatgcagatacaatacgaatagcacaaccaaaacttaaatcagaaatacttaatataacaacatggaatccatcacttataggatcaacaacagcacttccaaataattcaggaaaagatgtactttcagatgtacttcaaatagcatcaatacaaataaataaaatgaaatcacgaaaagcatgtggagtagcagtaggagcaacacttatagatgcagataaatggtcaataacaggagaagcacgacttataaatgaacgagcagcacatatgaatgcacaatttcgattt

SEQ ID NO: 161 - RNA, 1206 nt, synthetic construct
  source 1..1206: mol_type = other RNA
atgaaaaaactcctcaaatcggcgctcctctttgcggcgacggggtcggcgctctcgctccaagcgctcccggtggggaatccggcggaaccgtcgctcctcattgatgggacgatgtgggaaggggcgtcgggggatccgtgtgatccgtgtgcgacgtggtgtgatgcgatttcgattcgggcggggtattatggggattatgtgtttgatcgggtgctcaaagtggatgtgaataaaacgttttcggggatggcggcgacgccgacgcaagcgacggggaatgcgtcgaatacgaatcaaccggaagcgaatgggcggccgaatattgcgtatgggcggcatatgcaagatgcggaatggttttcgaatgcggcgtttctcgcgctcaatatttgggatcggtttgatattttttgtacgctcggggcgtcgaatgggtattttaaagcgtcgtcggcggcgtttaatctcgtggggctcattgggttttcggcggcgtcgtcgatttcgacggatctcccgatgcaactcccgaatgtggggattacgcaaggggtggtggaattttatacggatacgtcgttttcgtggtcggtgggggcgcggggggcgctctgggaatgtgggtgtgcgacgctcggggcggaatttcaatatgcgcaatcgaatccgaaaattgaaatgctcaatgtgacgtcgtcgccggcgcaatttgtgattcataaaccgcgggggtataaaggggcgtcgtcgaattttccgctcccgattacggcggggacgacggaagcgacggatacgaaatcggcgacgattaaatatcatgaatggcaagtggggctcgcgctctcgtatcggctcaatatgctcgtgccgtatattggggtgaattggtcgcgggcgacgtttgatgcggatacgattcggattgcgcaaccgaaactcaaatcggaaattctcaatattacgacgtggaatccgtcgctcattgggtcgacgacggcgctcccgaataattcggggaaagatgtgctctcggatgtgctccaaattgcgtcgattcaaattaataaaatgaaatcgcggaaagcgtgtggggtggcggtgggggcgacgctcattgatgcggataaatggtcgattacgggggaagcgcggctcattaatgaacgggcggcgcatatgaatgcgcaatttcggttt

SEQ ID NO: 162 - RNA, 1206 nt, synthetic construct
  source 1..1206: mol_type = other RNA
atgaaaaaactactaaaaagtgctctactatttgctgctactggtagtgctctaagtctacaagctctacctgttggtaatcctgctgaacctagtctactaattgatggtactatgtgggaaggtgctagtggtgatccttgtgatccttgtgctacttggtgtgatgctattagtattagagctggttattatggtgattatgtttttgatagagttctaaaagttgatgttaataaaacttttagtggtatggctgctactcctactcaagctactggtaatgctagtaatactaatcaacctgaagctaatggtagacctaatattgcttatggtagacatatgcaagatgctgaatggtttagtaatgctgcttttctagctctaaatatttgggatagatttgatattttttgtactctaggtgctagtaatggttattttaaagctagtagtgctgcttttaatctagttggtctaattggttttagtgctgctagtagtattagtactgatctacctatgcaactacctaatgttggtattactcaaggtgttgttgaattttatactgatactagttttagttggagtgttggtgctagaggtgctctatgggaatgtggttgtgctactctaggtgctgaatttcaatatgctcaaagtaatcctaaaattgaaatgctaaatgttactagtagtcctgctcaatttgttattcataaacctagaggttataaaggtgctagtagtaattttcctctacctattactgctggtactactgaagctactgatactaaaagtgctactattaaatatcatgaatggcaagttggtctagctctaagttatagactaaatatgctagttccttatattggtgttaattggagtagagctacttttgatgctgatactattagaattgctcaacctaaactaaaaagtgaaattctaaatattactacttggaatcctagtctaattggtagtactactgctctacctaataatagtggtaaagatgttctaagtgatgttctacaaattgctagtattcaaattaataaaatgaaaagtagaaaagcttgtggtgttgctgttggtgctactctaattgatgctgataaatggagtattactggtgaagctagactaattaatgaaagagctgctcatatgaatgctcaatttagattt

SEQ ID NO: 163 - RNA, 1206 nt, synthetic construct
  source 1..1206: mol_type = other RNA
atgaaaaaactgctgaaaagcgctctgctgtttgctgctactggtagcgctctgagcctgcaagctctgcctgttggtaatcctgctgaacctagcctgctgattgatggtactatgtgggaaggtgctagcggtgatccttgtgatccttgtgctacttggtgtgatgctattagcattagggctggttattatggtgattatgtttttgatagggttctgaaagttgatgttaataaaacttttagcggtatggctgctactcctactcaagctactggtaatgctagcaatactaatcaacctgaagctaatggtaggcctaatattgcttatggtaggcatatgcaagatgctgaatggtttagcaatgctgcttttctggctctgaatatttgggataggtttgatattttttgtactctgggtgctagcaatggttattttaaagctagcagcgctgcttttaatctggttggtctgattggttttagcgctgctagcagcattagcactgatctgcctatgcaactgcctaatgttggtattactcaaggtgttgttgaattttatactgatactagctttagctggagcgttggtgctaggggtgctctgtgggaatgtggttgtgctactctgggtgctgaatttcaatatgctcaaagcaatcctaaaattgaaatgctgaatgttactagcagccctgctcaatttgttattcataaacctaggggttataaaggtgctagcagcaattttcctctgcctattactgctggtactactgaagctactgatactaaaagcgctactattaaatatcatgaatggcaagttggtctggctctgagctataggctgaatatgctggttccttatattggtgttaattggagcagggctacttttgatgctgatactattaggattgctcaacctaaactgaaaagcgaaattctgaatattactacttggaatcctagcctgattggtagcactactgctctgcctaataatagcggtaaagatgttctgagcgatgttctgcaaattgctagcattcaaattaataaaatgaaaagcaggaaagcttgtggtgttgctgttggtgctactctgattgatgctgataaatggagcattactggtgaagctaggctgattaatgaaagggctgctcatatgaatgctcaatttaggttt

SEQ ID NO: 164 - RNA, 10770 nt, synthetic construct
  source 1..10770: mol_type = other RNA
atgaatactaatttatatcgtttagttttttctcatgttcgtggtatgttagttcctgtttctgaacattgtactgttggtaatactttttgtggtcgtactcgtggtcaagctcgttctggtgctcgtgctacttctttatctgttgctcctaatgctttagcttgggctttaatgttagcttgtactggtttacctttagttactcatgctcaaggtttagttcctcaaggtcaaactcaagttttacaaggtggtaataaagttcctgttgttaatattgctgatcctaattctggtggtgtttctcataataaatttcaacaatttaatgttgctaatcctggtgttgtttttaataatggtttaactgatggtgtttctcgtattggtggtgctttaactaaaaatcctaatttaactcgtcaagcttctgctattttagctgaagttactgatacttctccttctcgtttagctggtactttagaagtttatggtaaaggtgctgatttaattattgctaatcctaatggtatttctgttaatggtttatctactttaaatgcttctaatttaactttaactactggtcgtccttctgttaatggtggtcgtattggtttagatgttcaacaaggtactgttactattgaacgtggtggtgttaatgctactggtttaggttattttgatgttgttgctcgtttagttaaattacaaggtgctgtttcttctaaacaaggtaaacctttagctgatattgctgttgttgctggtgctaatcgttatgatcatgctactcgtcgtgctactcctattgctgctggtgctcgtggtgctgctgctggtgcttatgctattgatggtactgctgctggtgctatgtatggtaaacatattactttagtttcttctgattctggtttaggtgttcgtcaattaggttctttatcttctccttctgctattactgtttcttctcaaggtgaaattgctttaggtgatgctactgttcaacgtggtcctttatctttaaaaggtgctggtgttgtttctgctggtaaattagcttctggtggtggtgctgttaatgttgctggtggtggtgctgttaaaattgcttctgcttcttctgttggtaatttagctgttcaaggtggtggtaaagttcaagctactttattaaatgctggtggtactttattagtttctggtcgtcaagctgttcaattaggtgctgcttcttctcgtcaagctttatctgttaatgctggtggtgctttaaaagctgataaattatctgctactcgtcgtgttgatgttgatggtaaacaagctgttgctttaggttctgcttcttctaatgctttatctgttcgtgctggtggtgctttaaaagctggtaaattatctgctactggtcgtttagatgttgatggtaaacaagctgttactttaggttctgttgcttctgatggtgctttatctgtttctgctggtggtaatttacgtgctaatgaattagtttcttctgctcaattagaagttcgtggtcaacgtgaagttgctttagatgatgcttcttctgctcgtggtatgactgttgttgctgctggtgctttagctgctcgtaatttacaatctaaaggtgctattggtgttcaaggtggtgaagctgtttctgttgctaatgctaattctgatgctgaattacgtgttcgtggtcgtggtcaagttgatttacatgatttatctgctgctcgtggtgctgatatttctggtgaaggtcgtgttaatattggtcgtgctcgttctgattctgatgttaaagtttctgctcatggtgctttatctattgattctatgactgctttaggtgctattggtgttcaagctggtggttctgtttctgctaaagatatgcgttctcgtggtgctgttactgtttctggtggtggtgctgttaatttaggtgatgttcaatctgatggtcaagttcgtgctacttctgctggtgctatgactgttcgtgatgttgctgctgctgctgatttagctttacaagctggtgatgctttacaagctggttttttaaaatctgctggtgctatgactgttaatggtcgtgatgctgttcgtttagatggtgctcatgctggtggtcaattacgtgtttcttctgatggtcaagctgctttaggttctttagctgctaaaggtgaattaactgtttctgctgctcgtgctgctactgttgctgaattaaaatctttagataatatttctgttactggtggtgaacgtgtttctgttcaatctgttaattctgcttctcgtgttgctatttctgctcatggtgctttagatgttggtaaagtttctgctaaatctggtattggtttagaaggttggggtgctgttggtgctgattctttaggttctgatggtgctatttctgtttctggtcgtgatgctgttcgtgttgatcatgctcgttctttagctgatatttctttaggtgctgaaggtggtgctactttaggtgctgttgaagctgctggttctattgatgttcgtggtggttctactgttgctgctaattctttacatgctaatcgtgatgttcgtgtttctggtaaagatgctgttcgtgttactgctgctacttctggtggtggtttacatgtttcttctggtcgtcaattagatttaggtgctgttcaagctcgtggtgctttagctttagatggtggtgctggtgttgctttacaatctgctaaagcttctggtactttacatgttcaaggtggtgaacatttagatttaggtactttagctgctgttggtgctgttgatgttaatggtactggtgatgttcgtgttgctaaattagtttctgatgctggtgctgatttacaagctggtcgttctatgactttaggtattgttgatactactggtgatttacaagctcgtgctcaacaaaaattagaattaggttctgttaaatctgatggtggtttacaagctgctgctggtggtgctttatctttagctgctgctgaagttgctggtgctttagaattatctggtcaaggtgttactgttgatcgtgcttctgcttctcgtgctcgtattgattctactggttctgttggtattggtgctttaaaagctggtgctgttgaagctgcttctcctcgtcgtgctcgtcgtgctttacgtcaagatttttttactcctggttctgttgttgttcgtgctcaaggtaatgttactgttggtcgtggtgatcctcatcaaggtgttttagctcaaggtgatattattatggatgctaaaggtggtactttattattacgtaatgatgctttaactgaaaatggtactgttactatttctgctgattctgctgttttagaacattctactattgaatctaaaatttctcaatctgttttagctgctaaaggtgataaaggtaaacctgctgtttctgttaaagttgctaaaaaattatttttaaatggtactttacgtgctgttaatgataataatgaaactatgtctggtcgtcaaattgatgttgttgatggtcgtcctcaaattactgatgctgttactggtgaagctcgtaaagatgaatctgttgtttctgatgctgctttagttgctgatggtggtcctattgttgttgaagctggtgaattagtttctcatgctggtggtattggtaatggtcgtaataaagaaaatggtgcttctgttactgttcgtactactggtaatttagttaataaaggttatatttctgctggtaaacaaggtgttttagaagttggtggtgctttaactaatgaatttttagttggttctgatggtactcaacgtattgaagctcaacgtattgaaaatcgtggtacttttcaatctcaagctcctgctggtactgctggtgctttagttgttaaagctgctgaagctattgttcatgatggtgttatggctactaaaggtgaaatgcaaattgctggtaaaggtggtggttctcctactgttactgctggtgctaaagctactacttctgctaataaattatctgttgatgttgcttcttgggataatgctggttctttagatattaaaaaaggtggtgctcaagttactgttgctggtcgttatgctgaacatggtgaagtttctattcaaggtgattatactgtttctgctgatgctattgctttagctgctcaagttactcaacgtggtggtgctgctaatttaacttctcgtcatgatactcgtttttctaataaaattcgtttaatgggtcctttacaagttaatgctggtggtcctgtttctaatactggtaatttaaaagttcgtgaaggtgttactgttactgctgcttcttttgataatgaaactggtgctgaagttatggctaaatctgctactttaactacttctggtgctgctcgtaatgctggtaaaatgcaagttaaagaagctgctactattgttgctgcttctgtttctaatcctggtacttttactgctggtaaagatattactgttacttctcgtggtggttttgataatgaaggtaaaatggaatctaataaagatattgttattaaaactgaacaattttctaatggtcgtgttttagatgctaaacatgatttaactgttactgcttctggtcaagctgataatcgtggttctttaaaagctggtcatgattttactgttcaagctcaacgtattgataattctggtactatggctgctggtcatgatgctactttaaaagctcctcatttacgtaatactggtcaagttgttgctggtcatgatattcatattattaattctgctaaattagaaaatactggtcgtgttgatgctcgtaatgatattgctttagatgttgctgattttactaatactggttctttatatgctgaacatgatgctactttaactttagctcaaggtactcaacgtgatttagttgttgatcaagatcatattttacctgttgctgaaggtactttacgtgttaaagctaaatctttaactactgaaattgaaactggtaatcctggttctttaattgctgaagttcaagaaaatattgataataaacaagctattgttgttggtaaagatttaactttatcttctgctcatggtaatgttgctaatgaagctaatgctttattatgggctgctggtgaattaactgttaaagctcaaaatattactaataaacgtgctgctttaattgaagctggtggtaatgctcgtttaactgctgctgttgctttattaaataaattaggtcgtattcgtgctggtgaagatatgcatttagatgctcctcgtattgaaaatactgctaaattatctggtgaagttcaacgtaaaggtgttcaagatgttggtggtggtgaacatggtcgttggtctggtattggttatgttaattattggttacgtgctggtaatggtaaaaaagctggtactattgctgctccttggtatggtggtgatttaactgctgaacaatctttaattgaagttggtaaagatttatatttaaatgctggtgctcgtaaagatgaacatcgtcatttattaaatgaaggtgttattcaagctggtggtcatggtcatattggtggtgatgttgataatcgttctgttgttcgtactgtttctgctatggaatattttaaaactcctttacctgtttctttaactgctttagataatcgtgctggtttatctcctgctacttggaattttcaatctacttatgaattattagattatttattagatcaaaatcgttatgaatatatttggggtttatatcctacttatactgaatggtctgttaatactttaaaaaatttagatttaggttatcaagctaaacctgctcctactgctcctcctatgcctaaagctcctgaattagatttacgtggtcatactttagaatctgctgaaggtcgtaaaatttttggtgaatataaaaaattacaaggtgaatatgaaaaagctaaaatggctgttcaagctgttgaagcttatggtgaagctactcgtcgtgttcatgatcaattaggtcaacgttatggtaaagctttaggtggtatggatgctgaaactaaagaagttgatggtattattcaagaatttgctgctgatttacgtactgtttatgctaaacaagctgatcaagctactattgatgctgaaactgataaagttgctcaacgttataaatctcaaattgatgctgttcgtttacaagctattcaacctggtcgtgttactttagctaaagctttatctgctgctttaggtgctgattggcgtgctttaggtcattctcaattaatgcaacgttggaaagattttaaagctggtaaacgtggtgctgaaattgctttttatcctaaagaacaaactgttttagctgctggtgctggtttaactttatctaatggtgctattcataatggtgaaaatgctgctcaaaatcgtggtcgtcctgaaggtttaaaaattggtgctcattctgctacttctgtttctggttcttttgatgctttacgtgatgttggtttagaaaaacgtttagatattgatgatgctttagctgctgttttagttaatcctcatatttttactcgtattggtgctgctcaaacttctttagctgatggtgctgctggtcctgctttagctcgtcaagctcgtcaagctcctgaaactgatggtatggttgatgctcgtggtttaggttctgctgatgctttagcttctttagcttctttagatgctgctcaaggtttagaagtttctggtcgtcgtaatgctcaagttgctgatgctggtttagctggtccttctgctgttgctgctcctgctgttggtgctgctgatgttggtgttgaacctgttactggtgatcaagttgatcaacctgttgttgctgttggtttagaacaacctgttgctactgttcgtgttgctcctcctgctgttgctttacctcgtcctttatttgaaactcgtattaaatttattgatcaatctaaattttatggttctcgttatttttttgaacaaattggttataaacctgatcgtgctgctcgtgttgctggtgataattattttgatactactttagttcgtgaacaagttcgtcgtgctttaggtggttatgaatctcgtttacctgttcgtggtgttgctttagttgctaaattaatggattctgctggtactgttggtaaagctttaggtttaaaagttggtgttgctcctactgctcaacaattaaaacaagctgatcgtgattttgtttggtatgttgatactgttattgatggtcaaaaagttttagctcctcgtttatatttaactgaagctactcgtcaaggtattactgatcaatatgctggtggtggtgctttaattgcttctggtggtgatgttactgttaatactgatggtcatgatgtttcttctgttaatggtttaattcaaggtcgttctgttaaagttgatgctggtaaaggtaaagttgttgttgctgattctaaaggtgctggtggtggtattgaagctgatgatgaagttgatgtttctggtcgtgatattggtattgaaggtggtaaattacgtggtaaagatgttcgtttaaaagctgatactgttaaagttgctacttctatgcgttatgatgataaaggtcgtttagctgctcgtggtgatggtgctttagatgctcaaggtggtcaattacatattgaagctaaacgtttagaaactgctggtgctactttaaaaggtggtaaagttaaattagatgttgatgatgttaaattaggtggtgtttatgaagctggttcttcttatgaaaataaatcttctactcctttaggttctttatttgctattttatcttctactactgaaactaatcaatctgctcatgctaatcattatggtactcgtattgaagctggtactttagaaggtaaaatgcaaaatttagaaattgaaggtggttctgttgatgctgctcatactgatttatctgttgctcgtgatgctcgttttaaagctgctgctgattttgctcatgctgaacatgaaaaagatgttcgtcaattatctttaggtgctaaagttggtgctggtggttatgaagctggtttttctttaggttctgaatctggtttagaagctcatgctggtcgtggtatgactgctggtgctgaagttaaagttggttatcgtgcttctcatgaacaatcttctgaaactgaaaaatcttatcgtaatgctaatttaaattttggtggtggttctgttgaagctggtaatgttttagatattggtggtgctgatattaatcgtaatcgttatggtggtgctgctaaaggtaatgctggtactgaagaagctttacgtatgcgtgctaaaaaagttgaatctactaaatatgtttctgaacaaacttctcaatcttctggttggtctgttgaagttgcttctactgcttctgctcgttcttctttattaactgctgctactcgtttaggtgattctgttgctcaaaatgttgaagatggtcgtgaaattcgtggtgaattaatggctgctcaagttgctgctgaagctactcaattagttactgctgatactgctgctgttgctttatctgctggtatttctgctgattttgattcttctcattctcgttctacttctcaaaatactcaatatttaggtggtaatttatctattgaagctactgaaggtgatgctactttagttggtgctaaatttggtggtggtgatcaagtttctttaaaagctgctaaatctgttaatttaatggctgctgaatctacttttgaatcttattctgaatctcataattttcatgcttctgctgatgctaatttaggtgctaatgctgttcaaggtgctgttggtttaggtttaactgctggtatgggtacttctcatcaaattactaatgaaactggtaaaacttatgctggtacttctgttgatgctgctaatgtttctattgatgctggtaaagatttaaatttatctggttctcgtgttcgtggtaaacatgttgttttagatgttgaaggtgatattaatgctacttctaaacaagatgaacgtaattataattcttctggtggtggttgggatgcttctgctggtgttgctattcaaaatcgtactttagttgctcctgttggttctgctggttttaattttaatactgaacatgataattctcgtttaactaatgatggtgctgctggtgttgttgcttctgatggtttaactggtcatgttaaaggtgatgctaatttaactggtgctactattgctgatttatctggtaaaggtaatttaaaagttgatggtgctgttaatgctcaaaatttaaaagattatcgtgataaagatggtggttctggtggtttaaatgttggtatttcttctactactttagctcctactgttggtgttgcttttggtcgtgttgctggtgaagattatcaagctgaacaacgtgctactattgatgttggtcaaactaaagatcctgctcgtttacaagttggtggtggtgttaaaggtactttaaatcaagatgctgctcaagctactgttgttcaacgtaataaacattgggctggtggtggttctgaattttctgttgctggtaaatctttaaaaaaaaaaaatcaagttcgtcctgttgaaactcctactcctgatgttgttgatggtcctccttctcgtcctactactcctcctgcttctcctcaacctattcgtgctactgttgaagtttcttctcctcctcctgtttctgttgctactgttgaagttgttcctcgtcctaaagttgaaactggttctgctgcttctgcttctgctggtggtgctcaagttgttcctgttactcctcctaaagttgaagttgctaaagttgaagttgttcctcgtcctaaagttgaaactgctcaacctttacctcctcgtcctgttgttgctgaaaaagttactactcctgctgttcaacctcaattagctaaagttgaaactgttcaacctgttaaacctgaaactactaaacctttacctaaacctttacctgttgctaaagttactaaagctcctcctcctgttgttgaaactgctcaacctttacctcctgttaaacctcaaaaagctactcctggtcctgttgctgaagttggtaaagctactgttactactgttcaagttcaatctgctcctcctaaacctgctcctgttgctaaacaacctgctcctgctcctaaacctaaacctaaacctaaacctaaagctgaacgtcctaaacctggtaaaactactcctttatctggtcgtcatgttgttcaacaacaagttcaagttttacaacgtcaagcttctgatattaataatactaaatctttacctggtggtaaattacctaaacctgttactgttaaattaactgatgaaaatggtaaacctcaaacttatactattaatcgtcgtgaagatttaatgaaattaaatggtaaagttttatctactaaaactactttaggtttagaacaaacttttcgtttacgtgttgaagatattggtggtaaaaattatcgtgttttttatgaaactaataaa

SEQ ID NO: 165 - RNA, 10770 nt, synthetic construct
  source 1..10770: mol_type = other RNA
atgaacaccaacttgtaccgcttggtcttctcccacgtccgcggcatgttggtccccgtctccgagcactgcaccgtcggcaacaccttctgcggccgcacccgcggccaggcccgctccggcgcccgcgccacctccttgtccgtcgcccccaacgccttggcctgggccttgatgttggcctgcaccggcttgcccttggtcacccacgcccagggcttggtcccccagggccagacccaggtcttgcagggcggcaacaaggtccccgtcgtcaacatcgccgaccccaactccggcggcgtctcccacaacaagttccagcagttcaacgtcgccaaccccggcgtcgtcttcaacaacggcttgaccgacggcgtctcccgcatcggcggcgccttgaccaagaaccccaacttgacccgccaggcctccgccatcttggccgaggtcaccgacacctccccctcccgcttggccggcaccttggaggtctacggcaagggcgccgacttgatcatcgccaaccccaacggcatctccgtcaacggcttgtccaccttgaacgcctccaacttgaccttgaccaccggccgcccctccgtcaacggcggccgcatcggcttggacgtccagcagggcaccgtcaccatcgagcgcggcggcgtcaacgccaccggcttgggctacttcgacgtcgtcgcccgcttggtcaagttgcagggcgccgtctcctccaagcagggcaagcccttggccgacatcgccgtcgtcgccggcgccaaccgctacgaccacgccacccgccgcgccacccccatcgccgccggcgcccgcggcgccgccgccggcgcctacgccatcgacggcaccgccgccggcgccatgtacggcaagcacatcaccttggtctcctccgactccggcttgggcgtccgccagttgggctccttgtcctccccctccgccatcaccgtctcctcccagggcgagatcgccttgggcgacgccaccgtccagcgcggccccttgtccttgaagggcgccggcgtcgtctccgccggcaagttggcctccggcggcggcgccgtcaacgtcgccggcggcggcgccgtcaagatcgcctccgcctcctccgtcggcaacttggccgtccagggcggcggcaaggtccaggccaccttgttgaacgccggcggcaccttgttggtctccggccgccaggccgtccagttgggcgccgcctcctcccgccaggccttgtccgtcaacgccggcggcgccttgaaggccgacaagttgtccgccacccgccgcgtcgacgtcgacggcaagcaggccgtcgccttgggctccgcctcctccaacgccttgtccgtccgcgccggcggcgccttgaaggccggcaagttgtccgccaccggccgcttggacgtcgacggcaagcaggccgtcaccttgggctccgtcgcctccgacggcgccttgtccgtctccgccggcggcaacttgcgcgccaacgagttggtctcctccgcccagttggaggtccgcggccagcgcgaggtcgccttggacgacgcctcctccgcccgcggcatgaccgtcgtcgccgccggcgccttggccgcccgcaacttgcagtccaagggcgccatcggcgtccagggcggcgaggccgtctccgtcgccaacgccaactccgacgccgagttgcgcgtccgcggccgcggccaggtcgacttgcacgacttgtccgccgcccgcggcgccgacatctccggcgagggccgcgtcaacatcggccgcgcccgctccgactccgacgtcaaggtctccgcccacggcgccttgtccatcgactccatgaccgccttgggcgccatcggcgtccaggccggcggctccgtctccgccaaggacatgcgctcccgcggcgccgtcaccgtctccggcggcggcgccgtcaacttgggcgacgtccagtccgacggccaggtccgcgccacctccgccggcgccatgaccgtccgcgacgtcgccgccgccgccgacttggccttgcaggccggcgacgccttgcaggccggcttcttgaagtccgccggcgccatgaccgtcaacggccgcgacgccgtccgcttggacggcgcccacgccggcggccagttgcgcgtctcctccgacggccaggccgccttgggctccttggccgccaagggcgagttgaccgtctccgccgcccgcgccgccaccgtcgccgagttgaagtccttggacaacatctccgtcaccggcggcgagcgcgtctccgtccagtccgtcaactccgcctcccgcgtcgccatctccgcccacggcgccttggacgtcggcaaggtctccgccaagtccggcatcggcttggagggctggggcgccgtcggcgccgactccttgggctccgacggcgccatctccgtctccggccgcgacgccgtccgcgtcgaccacgcccgctccttggccgacatctccttgggcgccgagggcggcgccaccttgggcgccgtcgaggccgccggctccatcgacgtccgcggcggctccaccgtcgccgccaactccttgcacgccaaccgcgacgtccgcgtctccggcaaggacgccgtccgcgtcaccgccgccacctccggcggcggcttgcacgtctcctccggccgccagttggacttgggcgccgtccaggcccgcggcgccttggccttggacggcggcgccggcgtcgccttgcagtccgccaaggcctccggcaccttgcacgtccagggcggcgagcacttggacttgggcaccttggccgccgtcggcgccgtcgacgtcaacggcaccggcgacgtccgcgtcgccaagttggtctccgacgccggcgccgacttgcaggccggccgctccatgaccttgggcatcgtcgacaccaccggcgacttgcaggcccgcgcccagcagaagttggagttgggctccgtcaagtccgacggcggcttgcaggccgccgccggcggcgccttgtccttggccgccgccgaggtcgccggcgccttggagttgtccggccagggcgtcaccgtcgaccgcgcctccgcctcccgcgcccgcatcgactccaccggctccgtcggcatcggcgccttgaaggccggcgccgtcgaggccgcctccccccgccgcgcccgccgcgccttgcgccaggacttcttcacccccggctccgtcgtcgtccgcgcccagggcaacgtcaccgtcggccgcggcgacccccaccagggcgtcttggcccagggcgacatcatcatggacgccaagggcggcaccttgttgttgcgcaacgacgccttgaccgagaacggcaccgtcaccatctccgccgactccgccgtcttggagcactccaccatcgagtccaagatctcccagtccgtcttggccgccaagggcgacaagggcaagcccgccgtctccgtcaaggtcgccaagaagttgttcttgaacggcaccttgcgcgccgtcaacgacaacaacgagaccatgtccggccgccagatcgacgtcgtcgacggccgcccccagatcaccgacgccgtcaccggcgaggcccgcaaggacgagtccgtcgtctccgacgccgccttggtcgccgacggcggccccatcgtcgtcgaggccggcgagttggtctcccacgccggcggcatcggcaacggccgcaacaaggagaacggcgcctccgtcaccgtccgcaccaccggcaacttggtcaacaagggctacatctccgccggcaagcagggcgtcttggaggtcggcggcgccttgaccaacgagttcttggtcggctccgacggcacccagcgcatcgaggcccagcgcatcgagaaccgcggcaccttccagtcccaggcccccgccggcaccgccggcgccttggtcgtcaaggccgccgaggccatcgtccacgacggcgtcatggccaccaagggcgagatgcagatcgccggcaagggcggcggctcccccaccgtcaccgccggcgccaaggccaccacctccgccaacaagttgtccgtcgacgtcgcctcctgggacaacgccggctccttggacatcaagaagggcggcgcccaggtcaccgtcgccggccgctacgccgagcacggcgaggtctccatccagggcgactacaccgtctccgccgacgccatcgccttggccgcccaggtcacccagcgcggcggcgccgccaacttgacctcccgccacgacacccgcttctccaacaagatccgcttgatgggccccttgcaggtcaacgccggcggccccgtctccaacaccggcaacttgaaggtccgcgagggcgtcaccgtcaccgccgcctccttcgacaacgagaccggcgccgaggtcatggccaagtccgccaccttgaccacctccggcgccgcccgcaacgccggcaagatgcaggtcaaggaggccgccaccatcgtcgccgcctccgtctccaaccccggcaccttcaccgccggcaaggacatcaccgtcacctcccgcggcggcttcgacaacgagggcaagatggagtccaacaaggacatcgtcatcaagaccgagcagttctccaacggccgcgtcttggacgccaagcacgacttgaccgtcaccgcctccggccaggccgacaaccgcggctccttgaaggccggccacgacttcaccgtccaggcccagcgcatcgacaactccggcaccatggccgccggccacgacgccaccttgaaggccccccacttgcgcaacaccggccaggtcgtcgccggccacgacatccacatcatcaactccgccaagttggagaacaccggccgcgtcgacgcccgcaacgacatcgccttggacgtcgccgacttcaccaacaccggctccttgtacgccgagcacgacgccaccttgaccttggcccagggcacccagcgcgacttggtcgtcgaccaggaccacatcttgcccgtcgccgagggcaccttgcgcgtcaaggccaagtccttgaccaccgagatcgagaccggcaaccccggctccttgatcgccgaggtccaggagaacatcgacaacaagcaggccatcgtcgtcggcaaggacttgaccttgtcctccgcccacggcaacgtcgccaacgaggccaacgccttgttgtgggccgccggcgagttgaccgtcaaggcccagaacatcaccaacaagcgcgccgccttgatcgaggccggcggcaacgcccgcttgaccgccgccgtcgccttgttgaacaagttgggccgcatccgcgccggcgaggacatgcacttggacgccccccgcatcgagaacaccgccaagttgtccggcgaggtccagcgcaagggcgtccaggacgtcggcggcggcgagcacggccgctggtccggcatcggctacgtcaactactggttgcgcgccggcaacggcaagaaggccggcaccatcgccgccccctggtacggcggcgacttgaccgccgagcagtccttgatcgaggtcggcaaggacttgtacttgaacgccggcgcccgcaaggacgagcaccgccacttgttgaacgagggcgtcatccaggccggcggccacggccacatcggcggcgacgtcgacaaccgctccgtcgtccgcaccgtctccgccatggagtacttcaagacccccttgcccgtctccttgaccgccttggacaaccgcgccggcttgtcccccgccacctggaacttccagtccacctacgagttgttggactacttgttggaccagaaccgctacgagtacatctggggcttgtaccccacctacaccgagtggtccgtcaacaccttgaagaacttggacttgggctaccaggccaagcccgcccccaccgccccccccatgcccaaggcccccgagttggacttgcgcggccacaccttggagtccgccgagggccgcaagatcttcggcgagtacaagaagttgcagggcgagtacgagaaggccaagatggccgtccaggccgtcgaggcctacggcgaggccacccgccgcgtccacgaccagttgggccagcgctacggcaaggccttgggcggcatggacgccgagaccaaggaggtcgacggcatcatccaggagttcgccgccgacttgcgcaccgtctacgccaagcaggccgaccaggccaccatcgacgccgagaccgacaaggtcgcccagcgctacaagtcccagatcgacgccgtccgcttgcaggccatccagcccggccgcgtcaccttggccaaggccttgtccgccgccttgggcgccgactggcgcgccttgggccactcccagttgatgcagcgctggaaggacttcaaggccggcaagcgcggcgccgagatcgccttctaccccaaggagcagaccgtcttggccgccggcgccggcttgaccttgtccaacggcgccatccacaacggcgagaacgccgcccagaaccgcggccgccccgagggcttgaagatcggcgcccactccgccacctccgtctccggctccttcgacgccttgcgcgacgtcggcttggagaagcgcttggacatcgacgacgccttggccgccgtcttggtcaacccccacatcttcacccgcatcggcgccgcccagacctccttggccgacggcgccgccggccccgccttggcccgccaggcccgccaggcccccgagaccgacggcatggtcgacgcccgcggcttgggctccgccgacgccttggcctccttggcctccttggacgccgcccagggcttggaggtctccggccgccgcaacgcccaggtcgccgacgccggcttggccggcccctccgccgtcgccgcccccgccgtcggcgccgccgacgtcggcgtcgagcccgtcaccggcgaccaggtcgaccagcccgtcgtcgccgtcggcttggagcagcccgtcgccaccgtccgcgtcgccccccccgccgtcgccttgccccgccccttgttcgagacccgcatcaagttcatcgaccagtccaagttctacggctcccgctacttcttcgagcagatcggctacaagcccgaccgcgccgcccgcgtcgccggcgacaactacttcgacaccaccttggtccgcgagcaggtccgccgcgccttgggcggctacgagtcccgcttgcccgtccgcggcgtcgccttggtcgccaagttgatggactccgccggcaccgtcggcaaggccttgggcttgaaggtcggcgtcgcccccaccgcccagcagttgaagcaggccgaccgcgacttcgtctggtacgtcgacaccgtcatcgacggccagaaggtcttggccccccgcttgtacttgaccgaggccacccgccagggcatcaccgaccagtacgccggcggcggcgccttgatcgcctccggcggcgacgtcaccgtcaacaccgacggccacgacgtctcctccgtcaacggcttgatccagggccgctccgtcaaggtcgacgccggcaagggcaaggtcgtcgtcgccgactccaagggcgccggcggcggcatcgaggccgacgacgaggtcgacgtctccggccgcgacatcggcatcgagggcggcaagttgcgcggcaaggacgtccgcttgaaggccgacaccgtcaaggtcgccacctccatgcgctacgacgacaagggccgcttggccgcccgcggcgacggcgccttggacgcccagggcggccagttgcacatcgaggccaagcgcttggagaccgccggcgccaccttgaagggcggcaaggtcaagttggacgtcgacgacgtcaagttgggcggcgtctacgaggccggctcctcctacgagaacaagtcctccacccccttgggctccttgttcgccatcttgtcctccaccaccgagaccaaccagtccgcccacgccaaccactacggcacccgcatcgaggccggcaccttggagggcaagatgcagaacttggagatcgagggcggctccgtcgacgccgcccacaccgacttgtccgtcgcccgcgacgcccgcttcaaggccgccgccgacttcgcccacgccgagcacgagaaggacgtccgccagttgtccttgggcgccaaggtcggcgccggcggctacgaggccggcttctccttgggctccgagtccggcttggaggcccacgccggccgcggcatgaccgccggcgccgaggtcaaggtcggctaccgcgcctcccacgagcagtcctccgagaccgagaagtcctaccgcaacgccaacttgaacttcggcggcggctccgtcgaggccggcaacgtcttggacatcggcggcgccgacatcaaccgcaaccgctacggcggcgccgccaagggcaacgccggcaccgaggaggccttgcgcatgcgcgccaagaaggtcgagtccaccaagtacgtctccgagcagacctcccagtcctccggctggtccgtcgaggtcgcctccaccgcctccgcccgctcctccttgttgaccgccgccacccgcttgggcgactccgtcgcccagaacgtcgaggacggccgcgagatccgcggcgagttgatggccgcccaggtcgccgccgaggccacccagttggtcaccgccgacaccgccgccgtcgccttgtccgccggcatctccgccgacttcgactcctcccactcccgctccacctcccagaacacccagtacttgggcggcaacttgtccatcgaggccaccgagggcgacgccaccttggtcggcgccaagttcggcggcggcgaccaggtctccttgaaggccgccaagtccgtcaacttgatggccgccgagtccaccttcgagtcctactccgagtcccacaacttccacgcctccgccgacgccaacttgggcgccaacgccgtccagggcgccgtcggcttgggcttgaccgccggcatgggcacctcccaccagatcaccaacgagaccggcaagacctacgccggcacctccgtcgacgccgccaacgtctccatcgacgccggcaaggacttgaacttgtccggctcccgcgtccgcggcaagcacgtcgtcttggacgtcgagggcgacatcaacgccacctccaagcaggacgagcgcaactacaactcctccggcggcggctgggacgcctccgccggcgtcgccatccagaaccgcaccttggtcgcccccgtcggctccgccggcttcaacttcaacaccgagcacgacaactcccgcttgaccaacgacggcgccgccggcgtcgtcgcctccgacggcttgaccggccacgtcaagggcgacgccaacttgaccggcgccaccatcgccgacttgtccggcaagggcaacttgaaggtcgacggcgccgtcaacgcccagaacttgaaggactaccgcgacaaggacggcggctccggcggcttgaacgtcggcatctcctccaccaccttggcccccaccgtcggcgtcgccttcggccgcgtcgccggcgaggactaccaggccgagcagcgcgccaccatcgacgtcggccagaccaaggaccccgcccgcttgcaggtcggcggcggcgtcaagggcaccttgaaccaggacgccgcccaggccaccgtcgtccagcgcaacaagcactgggccggcggcggctccgagttctccgtcgccggcaagtccttgaagaagaagaaccaggtccgccccgtcgagacccccacccccgacgtcgtcgacggccccccctcccgccccaccaccccccccgcctccccccagcccatccgcgccaccgtcgaggtctcctcccccccccccgtctccgtcgccaccgtcgaggtcgtcccccgccccaaggtcgagaccggctccgccgcctccgcctccgccggcggcgcccaggtcgtccccgtcaccccccccaaggtcgaggtcgccaaggtcgaggtcgtcccccgccccaaggtcgagaccgcccagcccttgcccccccgccccgtcgtcgccgagaaggtcaccacccccgccgtccagccccagttggccaaggtcgagaccgtccagcccgtcaagcccgagaccaccaagcccttgcccaagcccttgcccgtcgccaaggtcaccaaggcccccccccccgtcgtcgagaccgcccagcccttgccccccgtcaagccccagaaggccacccccggccccgtcgccgaggtcggcaaggccaccgtcaccaccgtccaggtccagtccgccccccccaagcccgcccccgtcgccaagcagcccgcccccgcccccaagcccaagcccaagcccaagcccaaggccgagcgccccaagcccggcaagaccacccccttgtccggccgccacgtcgtccagcagcaggtccaggtcttgcagcgccaggcctccgacatcaacaacaccaagtccttgcccggcggcaagttgcccaagcccgtcaccgtcaagttgaccgacgagaacggcaagccccagacctacaccatcaaccgccgcgaggacttgatgaagttgaacggcaaggtcttgtccaccaagaccaccttgggcttggagcagaccttccgcttgcgcgtcgaggacatcggcggcaagaactaccgcgtcttctacgagaccaacaag

SEQ ID NO: 166 - RNA, 10770 nt, synthetic construct
  source 1..10770: mol_type = other RNA
atgaatacaaatctttatcgacttgtattttcacatgtacgaggaatgcttgtaccagtatcagaacattgtacagtaggaaatacattttgtggacgaacacgaggacaagcacgatcaggagcacgagcaacatcactttcagtagcaccaaatgcacttgcatgggcacttatgcttgcatgtacaggacttccacttgtaacacatgcacaaggacttgtaccacaaggacaaacacaagtacttcaaggaggaaataaagtaccagtagtaaatatagcagatccaaattcaggaggagtatcacataataaatttcaacaatttaatgtagcaaatccaggagtagtatttaataatggacttacagatggagtatcacgaataggaggagcacttacaaaaaatccaaatcttacacgacaagcatcagcaatacttgcagaagtaacagatacatcaccatcacgacttgcaggaacacttgaagtatatggaaaaggagcagatcttataatagcaaatccaaatggaatatcagtaaatggactttcaacacttaatgcatcaaatcttacacttacaacaggacgaccatcagtaaatggaggacgaataggacttgatgtacaacaaggaacagtaacaatagaacgaggaggagtaaatgcaacaggacttggatattttgatgtagtagcacgacttgtaaaacttcaaggagcagtatcatcaaaacaaggaaaaccacttgcagatatagcagtagtagcaggagcaaatcgatatgatcatgcaacacgacgagcaacaccaatagcagcaggagcacgaggagcagcagcaggagcatatgcaatagatggaacagcagcaggagcaatgtatggaaaacatataacacttgtatcatcagattcaggacttggagtacgacaacttggatcactttcatcaccatcagcaataacagtatcatcacaaggagaaatagcacttggagatgcaacagtacaacgaggaccactttcacttaaaggagcaggagtagtatcagcaggaaaacttgcatcaggaggaggagcagtaaatgtagcaggaggaggagcagtaaaaatagcatcagcatcatcagtaggaaatcttgcagtacaaggaggaggaaaagtacaagcaacacttcttaatgcaggaggaacacttcttgtatcaggacgacaagcagtacaacttggagcagcatcatcacgacaagcactttcagtaaatgcaggaggagcacttaaagcagataaactttcagcaacacgacgagtagatgtagatggaaaacaagcagtagcacttggatcagcatcatcaaatgcactttcagtacgagcaggaggagcacttaaagcaggaaaactttcagcaacaggacgacttgatgtagatggaaaacaagcagtaacacttggatcagtagcatcagatggagcactttcagtatcagcaggaggaaatcttcgagcaaatgaacttgtatcatcagcacaacttgaagtacgaggacaacgagaagtagcacttgatgatgcatcatcagcacgaggaatgacagtagtagcagcaggagcacttgcagcacgaaatcttcaatcaaaaggagcaataggagtacaaggaggagaagcagtatcagtagcaaatgcaaattcagatgcagaacttcgagtacgaggacgaggacaagtagatcttcatgatctttcagcagcacgaggagcagatatatcaggagaaggacgagtaaatataggacgagcacgatcagattcagatgtaaaagtatcagcacatggagcactttcaatagattcaatgacagcacttggagcaataggagtacaagcaggaggatcagtatcagcaaaagatatgcgatcacgaggagcagtaacagtatcaggaggaggagcagtaaatcttggagatgtacaatcagatggacaagtacgagcaacatcagcaggagcaatgacagtacgagatgtagcagcagcagcagatcttgcacttcaagcaggagatgcacttcaagcaggatttcttaaatcagcaggagcaatgacagtaaatggacgagatgcagtacgacttgatggagcacatgcaggaggacaacttcgagtatcatcagatggacaagcagcacttggatcacttgcagcaaaaggagaacttacagtatcagcagcacgagcagcaacagtagcagaacttaaatcacttgataatatatcagtaacaggaggagaacgagtatcagtacaatcagtaaattcagcatcacgagtagcaatatcagcacatggagcacttgatgtaggaaaagtatcagcaaaatcaggaataggacttgaaggatggggagcagtaggagcagattcacttggatcagatggagcaatatcagtatcaggacgagatgcagtacgagtagatcatgcacgatcacttgcagatatatcacttggagcagaaggaggagcaacacttggagcagtagaagcagcaggatcaatagatgtacgaggaggatcaacagtagcagcaaattcacttcatgcaaatcgagatgtacgagtatcaggaaaagatgcagtacgagtaacagcagcaacatcaggaggaggacttcatgtatcatcaggacgacaacttgatcttggagcagtacaagcacgaggagcacttgcacttgatggaggagcaggagtagcacttcaatcagcaaaagcatcaggaacacttcatgtacaaggaggagaacatcttgatcttggaacacttgcagcagtaggagcagtagatgtaaatggaacaggagatgtacgagtagcaaaacttgtatcagatgcaggagcagatcttcaagcaggacgatcaatgacacttggaatagtagatacaacaggagatcttcaagcacgagcacaacaaaaacttgaacttggatcagtaaaatcagatggaggacttcaagcagcagcaggaggagcactttcacttgcagcagcagaagtagcaggagcacttgaactttcaggacaaggagtaacagtagatcgagcatcagcatcacgagcacgaatagattcaacaggatcagtaggaataggagcacttaaagcaggagcagtagaagcagcatcaccacgacgagcacgacgagcacttcgacaagatttttttacaccaggatcagtagtagtacgagcacaaggaaatgtaacagtaggacgaggagatccacatcaaggagtacttgcacaaggagatataataatggatgcaaaaggaggaacacttcttcttcgaaatgatgcacttacagaaaatggaacagtaacaatatcagcagattcagcagtacttgaacattcaacaatagaatcaaaaatatcacaatcagtacttgcagcaaaaggagataaaggaaaaccagcagtatcagtaaaagtagcaaaaaaactttttcttaatggaacacttcgagcagtaaatgataataatgaaacaatgtcaggacgacaaatagatgtagtagatggacgaccacaaataacagatgcagtaacaggagaagcacgaaaagatgaatcagtagtatcagatgcagcacttgtagcagatggaggaccaatagtagtagaagcaggagaacttgtatcacatgcaggaggaataggaaatggacgaaataaagaaaatggagcatcagtaacagtacgaacaacaggaaatcttgtaaataaaggatatatatcagcaggaaaacaaggagtacttgaagtaggaggagcacttacaaatgaatttcttgtaggatcagatggaacacaacgaatagaagcacaacgaatagaaaatcgaggaacatttcaatcacaagcaccagcaggaacagcaggagcacttgtagtaaaagcagcagaagcaatagtacatgatggagtaatggcaacaaaaggagaaatgcaaatagcaggaaaaggaggaggatcaccaacagtaacagcaggagcaaaagcaacaacatcagcaaataaactttcagtagatgtagcatcatgggataatgcaggatcacttgatataaaaaaaggaggagcacaagtaacagtagcaggacgatatgcagaacatggagaagtatcaatacaaggagattatacagtatcagcagatgcaatagcacttgcagcacaagtaacacaacgaggaggagcagcaaatcttacatcacgacatgatacacgattttcaaataaaatacgacttatgggaccacttcaagtaaatgcaggaggaccagtatcaaatacaggaaatcttaaagtacgagaaggagtaacagtaacagcagcatcatttgataatgaaacaggagcagaagtaatggcaaaatcagcaacacttacaacatcaggagcagcacgaaatgcaggaaaaatgcaagtaaaagaagcagcaacaatagtagcagcatcagtatcaaatccaggaacatttacagcaggaaaagatataacagtaacatcacgaggaggatttgataatgaaggaaaaatggaatcaaataaagatatagtaataaaaacagaacaattttcaaatggacgagtacttgatgcaaaacatgatcttacagtaacagcatcaggacaagcagataatcgaggatcacttaaagcaggacatgattttacagtacaagcacaacgaatagataattcaggaacaatggcagcaggacatgatgcaacacttaaagcaccacatcttcgaaatacaggacaagtagtagcaggacatgatatacatataataaattcagcaaaacttgaaaatacaggacgagtagatgcacgaaatgatatagcacttgatgtagcagattttacaaatacaggatcactttatgcagaacatgatgcaacacttacacttgcacaaggaacacaacgagatcttgtagtagatcaagatcatatacttccagtagcagaaggaacacttcgagtaaaagcaaaatcacttacaacagaaatagaaacaggaaatccaggatcacttatagcagaagtacaagaaaatatagataataaacaagcaatagtagtaggaaaagatcttacactttcatcagcacatggaaatgtagcaaatgaagcaaatgcacttctttgggcagcaggagaacttacagtaaaagcacaaaatataacaaataaacgagcagcacttatagaagcaggaggaaatgcacgacttacagcagcagtagcacttcttaataaacttggacgaatacgagcaggagaagatatgcatcttgatgcaccacgaatagaaaatacagcaaaactttcaggagaagtacaacgaaaaggagtacaagatgtaggaggaggagaacatggacgatggtcaggaataggatatgtaaattattggcttcgagcaggaaatggaaaaaaagcaggaacaatagcagcaccatggtatggaggagatcttacagcagaacaatcacttatagaagtaggaaaagatctttatcttaatgcaggagcacgaaaagatgaacatcgacatcttcttaatgaaggagtaatacaagcaggaggacatggacatataggaggagatgtagataatcgatcagtagtacgaacagtatcagcaatggaatattttaaaacaccacttccagtatcacttacagcacttgataatcgagcaggactttcaccagcaacatggaattttcaatcaacatatgaacttcttgattatcttcttgatcaaaatcgatatgaatatatatggggactttatccaacatatacagaatggtcagtaaatacacttaaaaatcttgatcttggatatcaagcaaaaccagcaccaacagcaccaccaatgccaaaagcaccagaacttgatcttcgaggacatacacttgaatcagcagaaggacgaaaaatatttggagaatataaaaaacttcaaggagaatatgaaaaagcaaaaatggcagtacaagcagtagaagcatatggagaagcaacacgacgagtacatgatcaacttggacaacgatatggaaaagcacttggaggaatggatgcagaaacaaaagaagtagatggaataatacaagaatttgcagcagatcttcgaacagtatatgcaaaacaagcagatcaagcaacaatagatgcagaaacagataaagtagcacaacgatataaatcacaaatagatgcagtacgacttcaagcaatacaaccaggacgagtaacacttgcaaaagcactttcagcagcacttggagcagattggcgagcacttggacattcacaacttatgcaacgatggaaagattttaaagcaggaaaacgaggagcagaaatagcattttatccaaaagaacaaacagtacttgcagcaggagcaggacttacactttcaaatggagcaatacataatggagaaaatgcagcacaaaatcgaggacgaccagaaggacttaaaataggagcacattcagcaacatcagtatcaggatcatttgatgcacttcgagatgtaggacttgaaaaacgacttgatatagatgatgcacttgcagcagtacttgtaaatccacatatatttacacgaataggagcagcacaaacatcacttgcagatggagcagcaggaccagcacttgcacgacaagcacgacaagcaccagaaacagatggaatggtagatgcacgaggacttggatcagcagatgcacttgcatcacttgcatcacttgatgcagcacaaggacttgaagtatcaggacgacgaaatgcacaagtagcagatgcaggacttgcaggaccatcagcagtagcagcaccagcagtaggagcagcagatgtaggagtagaaccagtaacaggagatcaagtagatcaaccagtagtagcagtaggacttgaacaaccagtagcaacagtacgagtagcaccaccagcagtagcacttccacgaccactttttgaaacacgaataaaatttatagatcaatcaaaattttatggatcacgatatttttttgaacaaataggatataaaccagatcgagcagcacgagtagcaggagataattattttgatacaacacttgtacgagaacaagtacgacgagcacttggaggatatgaatcacgacttccagtacgaggagtagcacttgtagcaaaacttatggattcagcaggaacagtaggaaaagcacttggacttaaagtaggagtagcaccaacagcacaacaacttaaacaagcagatcgagattttgtatggtatgtagatacagtaatagatggacaaaaagtacttgcaccacgactttatcttacagaagcaacacgacaaggaataacagatcaatatgcaggaggaggagcacttatagcatcaggaggagatgtaacagtaaatacagatggacatgatgtatcatcagtaaatggacttatacaaggacgatcagtaaaagtagatgcaggaaaaggaaaagtagtagtagcagattcaaaaggagcaggaggaggaatagaagcagatgatgaagtagatgtatcaggacgagatataggaatagaaggaggaaaacttcgaggaaaagatgtacgacttaaagcagatacagtaaaagtagcaacatcaatgcgatatgatgataaaggacgacttgcagcacgaggagatggagcacttgatgcacaaggaggacaacttcatatagaagcaaaacgacttgaaacagcaggagcaacacttaaaggaggaaaagtaaaacttgatgtagatgatgtaaaacttggaggagtatatgaagcaggatcatcatatgaaaataaatcatcaacaccacttggatcactttttgcaatactttcatcaacaacagaaacaaatcaatcagcacatgcaaatcattatggaacacgaatagaagcaggaacacttgaaggaaaaatgcaaaatcttgaaatagaaggaggatcagtagatgcagcacatacagatctttcagtagcacgagatgcacgatttaaagcagcagcagattttgcacatgcagaacatgaaaaagatgtacgacaactttcacttggagcaaaagtaggagcaggaggatatgaagcaggattttcacttggatcagaatcaggacttgaagcacatgcaggacgaggaatgacagcaggagcagaagtaaaagtaggatatcgagcatcacatgaacaatcatcagaaacagaaaaatcatatcgaaatgcaaatcttaattttggaggaggatcagtagaagcaggaaatgtacttgatataggaggagcagatataaatcgaaatcgatatggaggagcagcaaaaggaaatgcaggaacagaagaagcacttcgaatgcgagcaaaaaaagtagaatcaacaaaatatgtatcagaacaaacatcacaatcatcaggatggtcagtagaagtagcatcaacagcatcagcacgatcatcacttcttacagcagcaacacgacttggagattcagtagcacaaaatgtagaagatggacgagaaatacgaggagaacttatggcagcacaagtagcagcagaagcaacacaacttgtaacagcagatacagcagcagtagcactttcagcaggaatatcagcagattttgattcatcacattcacgatcaacatcacaaaatacacaatatcttggaggaaatctttcaatagaagcaacagaaggagatgcaacacttgtaggagcaaaatttggaggaggagatcaagtatcacttaaagcagcaaaatcagtaaatcttatggcagcagaatcaacatttgaatcatattcagaatcacataattttcatgcatcagcagatgcaaatcttggagcaaatgcagtacaaggagcagtaggacttggacttacagcaggaatgggaacatcacatcaaataacaaatgaaacaggaaaaacatatgcaggaacatcagtagatgcagcaaatgtatcaatagatgcaggaaaagatcttaatctttcaggatcacgagtacgaggaaaacatgtagtacttgatgtagaaggagatataaatgcaacatcaaaacaagatgaacgaaattataattcatcaggaggaggatgggatgcatcagcaggagtagcaatacaaaatcgaacacttgtagcaccagtaggatcagcaggatttaattttaatacagaacatgataattcacgacttacaaatgatggagcagcaggagtagtagcatcagatggacttacaggacatgtaaaaggagatgcaaatcttacaggagcaacaatagcagatctttcaggaaaaggaaatcttaaagtagatggagcagtaaatgcacaaaatcttaaagattatcgagataaagatggaggatcaggaggacttaatgtaggaatatcatcaacaacacttgcaccaacagtaggagtagcatttggacgagtagcaggagaagattatcaagcagaacaacgagcaacaatagatgtaggacaaacaaaagatccagcacgacttcaagtaggaggaggagtaaaaggaacacttaatcaagatgcagcacaagcaacagtagtacaacgaaataaacattgggcaggaggaggatcagaattttcagtagcaggaaaatcacttaaaaaaaaaaatcaagtacgaccagtagaaacaccaacaccagatgtagtagatggaccaccatcacgaccaacaacaccaccagcatcaccacaaccaatacgagcaacagtagaagtatcatcaccaccaccagtatcagtagcaacagtagaagtagtaccacgaccaaaagtagaaacaggatcagcagcatcagcatcagcaggaggagcacaagtagtaccagtaacaccaccaaaagtagaagtagcaaaagtagaagtagtaccacgaccaaaagtagaaacagcacaaccacttccaccacgaccagtagtagcagaaaaagtaacaacaccagcagtacaaccacaacttgcaaaagtagaaacagtacaaccagtaaaaccagaaacaacaaaaccacttccaaaaccacttccagtagcaaaagtaacaaaagcaccaccaccagtagtagaaacagcacaaccacttccaccagtaaaaccacaaaaagcaacaccaggaccagtagcagaagtaggaaaagcaacagtaacaacagtacaagtacaatcagcaccaccaaaaccagcaccagtagcaaaacaaccagcaccagcaccaaaaccaaaaccaaaaccaaaaccaaaagcagaacgaccaaaaccaggaaaaacaacaccactttcaggacgacatgtagtacaacaacaagtacaagtacttcaacgacaagcatcagatataaataatacaaaatcacttccaggaggaaaacttccaaaaccagtaacagtaaaacttacagatgaaaatggaaaaccacaaacatatacaataaatcgacgagaagatcttatgaaacttaatggaaaagtactttcaacaaaaacaacacttggacttgaacaaacatttcgacttcgagtagaagatataggaggaaaaaattatcgagtattttatgaaacaaataaa

SEQ ID NO: 167 - RNA, 10770 nt, synthetic construct
  source 1..10770: mol_type = other RNA
atgaatacgaatctctatcggctcgtgttttcgcatgtgcgggggatgctcgtgccggtgtcggaacattgtacggtggggaatacgttttgtgggcggacgcgggggcaagcgcggtcgggggcgcgggcgacgtcgctctcggtggcgccgaatgcgctcgcgtgggcgctcatgctcgcgtgtacggggctcccgctcgtgacgcatgcgcaagggctcgtgccgcaagggcaaacgcaagtgctccaaggggggaataaagtgccggtggtgaatattgcggatccgaattcggggggggtgtcgcataataaatttcaacaatttaatgtggcgaatccgggggtggtgtttaataatgggctcacggatggggtgtcgcggattgggggggcgctcacgaaaaatccgaatctcacgcggcaagcgtcggcgattctcgcggaagtgacggatacgtcgccgtcgcggctcgcggggacgctcgaagtgtatgggaaaggggcggatctcattattgcgaatccgaatgggatttcggtgaatgggctctcgacgctcaatgcgtcgaatctcacgctcacgacggggcggccgtcggtgaatggggggcggattgggctcgatgtgcaacaagggacggtgacgattgaacgggggggggtgaatgcgacggggctcgggtattttgatgtggtggcgcggctcgtgaaactccaaggggcggtgtcgtcgaaacaagggaaaccgctcgcggatattgcggtggtggcgggggcgaatcggtatgatcatgcgacgcggcgggcgacgccgattgcggcgggggcgcggggggcggcggcgggggcgtatgcgattgatgggacggcggcgggggcgatgtatgggaaacatattacgctcgtgtcgtcggattcggggctcggggtgcggcaactcgggtcgctctcgtcgccgtcggcgattacggtgtcgtcgcaaggggaaattgcgctcggggatgcgacggtgcaacgggggccgctctcgctcaaaggggcgggggtggtgtcggcggggaaactcgcgtcgggggggggggcggtgaatgtggcgggggggggggcggtgaaaattgcgtcggcgtcgtcggtggggaatctcgcggtgcaagggggggggaaagtgcaagcgacgctcctcaatgcgggggggacgctcctcgtgtcggggcggcaagcggtgcaactcggggcggcgtcgtcgcggcaagcgctctcggtgaatgcggggggggcgctcaaagcggataaactctcggcgacgcggcgggtggatgtggatgggaaacaagcggtggcgctcgggtcggcgtcgtcgaatgcgctctcggtgcgggcggggggggcgctcaaagcggggaaactctcggcgacggggcggctcgatgtggatgggaaacaagcggtgacgctcgggtcggtggcgtcggatggggcgctctcggtgtcggcgggggggaatctccgggcgaatgaactcgtgtcgtcggcgcaactcgaagtgcgggggcaacgggaagtggcgctcgatgatgcgtcgtcggcgcgggggatgacggtggtggcggcgggggcgctcgcggcgcggaatctccaatcgaaaggggcgattggggtgcaagggggggaagcggtgtcggtggcgaatgcgaattcggatgcggaactccgggtgcgggggcgggggcaagtggatctccatgatctctcggcggcgcggggggcggatatttcgggggaagggcgggtgaatattgggcgggcgcggtcggattcggatgtgaaagtgtcggcgcatggggcgctctcgattgattcgatgacggcgctcggggcgattggggtgcaagcgggggggtcggtgtcggcgaaagatatgcggtcgcggggggcggtgacggtgtcgggggggggggcggtgaatctcggggatgtgcaatcggatgggcaagtgcgggcgacgtcggcgggggcgatgacggtgcgggatgtggcggcggcggcggatctcgcgctccaagcgggggatgcgctccaagcggggtttctcaaatcggcgggggcgatgacggtgaatgggcgggatgcggtgcggctcgatggggcgcatgcgggggggcaactccgggtgtcgtcggatgggcaagcggcgctcgggtcgctcgcggcgaaaggggaactcacggtgtcggcggcgcgggcggcgacggtggcggaactcaaatcgctcgataatatttcggtgacggggggggaacgggtgtcggtgcaatcggtgaattcggcgtcgcgggtggcgatttcggcgcatggggcgctcgatgtggggaaagtgtcggcgaaatcggggattgggctcgaagggtggggggcggtgggggcggattcgctcgggtcggatggggcgatttcggtgtcggggcgggatgcggtgcgggtggatcatgcgcggtcgctcgcggatatttcgctcggggcggaagggggggcgacgctcggggcggtggaagcggcggggtcgattgatgtgcggggggggtcgacggtggcggcgaattcgctccatgcgaatcgggatgtgcgggtgtcggggaaagatgcggtgcgggtgacggcggcgacgtcggggggggggctccatgtgtcgtcggggcggcaactcgatctcggggcggtgcaagcgcggggggcgctcgcgctcgatgggggggcgggggtggcgctccaatcggcgaaagcgtcggggacgctccatgtgcaagggggggaacatctcgatctcgggacgctcgcggcggtgggggcggtggatgtgaatgggacgggggatgtgcgggtggcgaaactcgtgtcggatgcgggggcggatctccaagcggggcggtcgatgacgctcgggattgtggatacgacgggggatctccaagcgcgggcgcaacaaaaactcgaactcgggtcggtgaaatcggatggggggctccaagcggcggcggggggggcgctctcgctcgcggcggcggaagtggcgggggcgctcgaactctcggggcaaggggtgacggtggatcgggcgtcggcgtcgcgggcgcggattgattcgacggggtcggtggggattggggcgctcaaagcgggggcggtggaagcggcgtcgccgcggcgggcgcggcgggcgctccggcaagatttttttacgccggggtcggtggtggtgcgggcgcaagggaatgtgacggtggggcggggggatccgcatcaaggggtgctcgcgcaaggggatattattatggatgcgaaaggggggacgctcctcctccggaatgatgcgctcacggaaaatgggacggtgacgatttcggcggattcggcggtgctcgaacattcgacgattgaatcgaaaatttcgcaatcggtgctcgcggcgaaaggggataaagggaaaccggcggtgtcggtgaaagtggcgaaaaaactctttctcaatgggacgctccgggcggtgaatgataataatgaaacgatgtcggggcggcaaattgatgtggtggatgggcggccgcaaattacggatgcggtgacgggggaagcgcggaaagatgaatcggtggtgtcggatgcggcgctcgtggcggatggggggccgattgtggtggaagcgggggaactcgtgtcgcatgcgggggggattgggaatgggcggaataaagaaaatggggcgtcggtgacggtgcggacgacggggaatctcgtgaataaagggtatatttcggcggggaaacaaggggtgctcgaagtggggggggcgctcacgaatgaatttctcgtggggtcggatgggacgcaacggattgaagcgcaacggattgaaaatcgggggacgtttcaatcgcaagcgccggcggggacggcgggggcgctcgtggtgaaagcggcggaagcgattgtgcatgatggggtgatggcgacgaaaggggaaatgcaaattgcggggaaagggggggggtcgccgacggtgacggcgggggcgaaagcgacgacgtcggcgaataaactctcggtggatgtggcgtcgtgggataatgcggggtcgctcgatattaaaaaagggggggcgcaagtgacggtggcggggcggtatgcggaacatggggaagtgtcgattcaaggggattatacggtgtcggcggatgcgattgcgctcgcggcgcaagtgacgcaacgggggggggcggcgaatctcacgtcgcggcatgatacgcggttttcgaataaaattcggctcatggggccgctccaagtgaatgcgggggggccggtgtcgaatacggggaatctcaaagtgcgggaaggggtgacggtgacggcggcgtcgtttgataatgaaacgggggcggaagtgatggcgaaatcggcgacgctcacgacgtcgggggcggcgcggaatgcggggaaaatgcaagtgaaagaagcggcgacgattgtggcggcgtcggtgtcgaatccggggacgtttacggcggggaaagatattacggtgacgtcgcggggggggtttgataatgaagggaaaatggaatcgaataaagatattgtgattaaaacggaacaattttcgaatgggcgggtgctcgatgcgaaacatgatctcacggtgacggcgtcggggcaagcggataatcgggggtcgctcaaagcggggcatgattttacggtgcaagcgcaacggattgataattcggggacgatggcggcggggcatgatgcgacgctcaaagcgccgcatctccggaatacggggcaagtggtggcggggcatgatattcatattattaattcggcgaaactcgaaaatacggggcgggtggatgcgcggaatgatattgcgctcgatgtggcggattttacgaatacggggtcgctctatgcggaacatgatgcgacgctcacgctcgcgcaagggacgcaacgggatctcgtggtggatcaagatcatattctcccggtggcggaagggacgctccgggtgaaagcgaaatcgctcacgacggaaattgaaacggggaatccggggtcgctcattgcggaagtgcaagaaaatattgataataaacaagcgattgtggtggggaaagatctcacgctctcgtcggcgcatgggaatgtggcgaatgaagcgaatgcgctcctctgggcggcgggggaactcacggtgaaagcgcaaaatattacgaataaacgggcggcgctcattgaagcgggggggaatgcgcggctcacggcggcggtggcgctcctcaataaactcgggcggattcgggcgggggaagatatgcatctcgatgcgccgcggattgaaaatacggcgaaactctcgggggaagtgcaacggaaaggggtgcaagatgtgggggggggggaacatgggcggtggtcggggattgggtatgtgaattattggctccgggcggggaatgggaaaaaagcggggacgattgcggcgccgtggtatgggggggatctcacggcggaacaatcgctcattgaagtggggaaagatctctatctcaatgcgggggcgcggaaagatgaacatcggcatctcctcaatgaaggggtgattcaagcgggggggcatgggcatattgggggggatgtggataatcggtcggtggtgcggacggtgtcggcgatggaatattttaaaacgccgctcccggtgtcgctcacggcgctcgataatcgggcggggctctcgccggcgacgtggaattttcaatcgacgtatgaactcctcgattatctcctcgatcaaaatcggtatgaatatatttgggggctctatccgacgtatacggaatggtcggtgaatacgctcaaaaatctcgatctcgggtatcaagcgaaaccggcgccgacggcgccgccgatgccgaaagcgccggaactcgatctccgggggcatacgctcgaatcggcggaagggcggaaaatttttggggaatataaaaaactccaaggggaatatgaaaaagcgaaaatggcggtgcaagcggtggaagcgtatggggaagcgacgcggcgggtgcatgatcaactcgggcaacggtatgggaaagcgctcggggggatggatgcggaaacgaaagaagtggatgggattattcaagaatttgcggcggatctccggacggtgtatgcgaaacaagcggatcaagcgacgattgatgcggaaacggataaagtggcgcaacggtataaatcgcaaattgatgcggtgcggctccaagcgattcaaccggggcgggtgacgctcgcgaaagcgctctcggcggcgctcggggcggattggcgggcgctcgggcattcgcaactcatgcaacggtggaaagattttaaagcggggaaacggggggcggaaattgcgttttatccgaaagaacaaacggtgctcgcggcgggggcggggctcacgctctcgaatggggcgattcataatggggaaaatgcggcgcaaaatcgggggcggccggaagggctcaaaattggggcgcattcggcgacgtcggtgtcggggtcgtttgatgcgctccgggatgtggggctcgaaaaacggctcgatattgatgatgcgctcgcggcggtgctcgtgaatccgcatatttttacgcggattggggcggcgcaaacgtcgctcgcggatggggcggcggggccggcgctcgcgcggcaagcgcggcaagcgccggaaacggatgggatggtggatgcgcgggggctcgggtcggcggatgcgctcgcgtcgctcgcgtcgctcgatgcggcgcaagggctcgaagtgtcggggcggcggaatgcgcaagtggcggatgcggggctcgcggggccgtcggcggtggcggcgccggcggtgggggcggcggatgtgggggtggaaccggtgacgggggatcaagtggatcaaccggtggtggcggtggggctcgaacaaccggtggcgacggtgcgggtggcgccgccggcggtggcgctcccgcggccgctctttgaaacgcggattaaatttattgatcaatcgaaattttatgggtcgcggtatttttttgaacaaattgggtataaaccggatcgggcggcgcgggtggcgggggataattattttgatacgacgctcgtgcgggaacaagtgcggcgggcgctcggggggtatgaatcgcggctcccggtgcggggggtggcgctcgtggcgaaactcatggattcggcggggacggtggggaaagcgctcgggctcaaagtgggggtggcgccgacggcgcaacaactcaaacaagcggatcgggattttgtgtggtatgtggatacggtgattgatgggcaaaaagtgctcgcgccgcggctctatctcacggaagcgacgcggcaagggattacggatcaatatgcgggggggggggcgctcattgcgtcggggggggatgtgacggtgaatacggatgggcatgatgtgtcgtcggtgaatgggctcattcaagggcggtcggtgaaagtggatgcggggaaagggaaagtggtggtggcggattcgaaaggggcggggggggggattgaagcggatgatgaagtggatgtgtcggggcgggatattgggattgaaggggggaaactccgggggaaagatgtgcggctcaaagcggatacggtgaaagtggcgacgtcgatgcggtatgatgataaagggcggctcgcggcgcggggggatggggcgctcgatgcgcaaggggggcaactccatattgaagcgaaacggctcgaaacggcgggggcgacgctcaaaggggggaaagtgaaactcgatgtggatgatgtgaaactcgggggggtgtatgaagcggggtcgtcgtatgaaaataaatcgtcgacgccgctcgggtcgctctttgcgattctctcgtcgacgacggaaacgaatcaatcggcgcatgcgaatcattatgggacgcggattgaagcggggacgctcgaagggaaaatgcaaaatctcgaaattgaaggggggtcggtggatgcggcgcatacggatctctcggtggcgcgggatgcgcggtttaaagcggcggcggattttgcgcatgcggaacatgaaaaagatgtgcggcaactctcgctcggggcgaaagtgggggcgggggggtatgaagcggggttttcgctcgggtcggaatcggggctcgaagcgcatgcggggcgggggatgacggcgggggcggaagtgaaagtggggtatcgggcgtcgcatgaacaatcgtcggaaacggaaaaatcgtatcggaatgcgaatctcaattttgggggggggtcggtggaagcggggaatgtgctcgatattgggggggcggatattaatcggaatcggtatgggggggcggcgaaagggaatgcggggacggaagaagcgctccggatgcgggcgaaaaaagtggaatcgacgaaatatgtgtcggaacaaacgtcgcaatcgtcggggtggtcggtggaagtggcgtcgacggcgtcggcgcggtcgtcgctcctcacggcggcgacgcggctcggggattcggtggcgcaaaatgtggaagatgggcgggaaattcggggggaactcatggcggcgcaagtggcggcggaagcgacgcaactcgtgacggcggatacggcggcggtggcgctctcggcggggatttcggcggattttgattcgtcgcattcgcggtcgacgtcgcaaaatacgcaatatctcggggggaatctctcgattgaagcgacggaaggggatgcgacgctcgtgggggcgaaatttggggggggggatcaagtgtcgctcaaagcggcgaaatcggtgaatctcatggcggcggaatcgacgtttgaatcgtattcggaatcgcataattttcatgcgtcggcggatgcgaatctcggggcgaatgcggtgcaaggggcggtggggctcgggctcacggcggggatggggacgtcgcatcaaattacgaatgaaacggggaaaacgtatgcggggacgtcggtggatgcggcgaatgtgtcgattgatgcggggaaagatctcaatctctcggggtcgcgggtgcgggggaaacatgtggtgctcgatgtggaaggggatattaatgcgacgtcgaaacaagatgaacggaattataattcgtcggggggggggtgggatgcgtcggcgggggtggcgattcaaaatcggacgctcgtggcgccggtggggtcggcggggtttaattttaatacggaacatgataattcgcggctcacgaatgatggggcggcgggggtggtggcgtcggatgggctcacggggcatgtgaaaggggatgcgaatctcacgggggcgacgattgcggatctctcggggaaagggaatctcaaagtggatggggcggtgaatgcgcaaaatctcaaagattatcgggataaagatggggggtcgggggggctcaatgtggggatttcgtcgacgacgctcgcgccgacggtgggggtggcgtttgggcgggtggcgggggaagattatcaagcggaacaacgggcgacgattgatgtggggcaaacgaaagatccggcgcggctccaagtgggggggggggtgaaagggacgctcaatcaagatgcggcgcaagcgacggtggtgcaacggaataaacattgggcggggggggggtcggaattttcggtggcggggaaatcgctcaaaaaaaaaaatcaagtgcggccggtggaaacgccgacgccggatgtggtggatgggccgccgtcgcggccgacgacgccgccggcgtcgccgcaaccgattcgggcgacggtggaagtgtcgtcgccgccgccggtgtcggtggcgacggtggaagtggtgccgcggccgaaagtggaaacggggtcggcggcgtcggcgtcggcggggggggcgcaagtggtgccggtgacgccgccgaaagtggaagtggcgaaagtggaagtggtgccgcggccgaaagtggaaacggcgcaaccgctcccgccgcggccggtggtggcggaaaaagtgacgacgccggcggtgcaaccgcaactcgcgaaagtggaaacggtgcaaccggtgaaaccggaaacgacgaaaccgctcccgaaaccgctcccggtggcgaaagtgacgaaagcgccgccgccggtggtggaaacggcgcaaccgctcccgccggtgaaaccgcaaaaagcgacgccggggccggtggcggaagtggggaaagcgacggtgacgacggtgcaagtgcaatcggcgccgccgaaaccggcgccggtggcgaaacaaccggcgccggcgccgaaaccgaaaccgaaaccgaaaccgaaagcggaacggccgaaaccggggaaaacgacgccgctctcggggcggcatgtggtgcaacaacaagtgcaagtgctccaacggcaagcgtcggatattaataatacgaaatcgctcccgggggggaaactcccgaaaccggtgacggtgaaactcacggatgaaaatgggaaaccgcaaacgtatacgattaatcggcgggaagatctcatgaaactcaatgggaaagtgctctcgacgaaaacgacgctcgggctcgaacaaacgtttcggctccgggtggaagatattggggggaaaaattatcgggtgttttatgaaacgaataaa

SEQ ID NO: 168 - RNA, 10770 nt, synthetic construct
  source 1..10770: mol_type = other RNA
atgaatactaatctatatagactagtttttagtcatgttagaggtatgctagttcctgttagtgaacattgtactgttggtaatactttttgtggtagaactagaggtcaagctagaagtggtgctagagctactagtctaagtgttgctcctaatgctctagcttgggctctaatgctagcttgtactggtctacctctagttactcatgctcaaggtctagttcctcaaggtcaaactcaagttctacaaggtggtaataaagttcctgttgttaatattgctgatcctaatagtggtggtgttagtcataataaatttcaacaatttaatgttgctaatcctggtgttgtttttaataatggtctaactgatggtgttagtagaattggtggtgctctaactaaaaatcctaatctaactagacaagctagtgctattctagctgaagttactgatactagtcctagtagactagctggtactctagaagtttatggtaaaggtgctgatctaattattgctaatcctaatggtattagtgttaatggtctaagtactctaaatgctagtaatctaactctaactactggtagacctagtgttaatggtggtagaattggtctagatgttcaacaaggtactgttactattgaaagaggtggtgttaatgctactggtctaggttattttgatgttgttgctagactagttaaactacaaggtgctgttagtagtaaacaaggtaaacctctagctgatattgctgttgttgctggtgctaatagatatgatcatgctactagaagagctactcctattgctgctggtgctagaggtgctgctgctggtgcttatgctattgatggtactgctgctggtgctatgtatggtaaacatattactctagttagtagtgatagtggtctaggtgttagacaactaggtagtctaagtagtcctagtgctattactgttagtagtcaaggtgaaattgctctaggtgatgctactgttcaaagaggtcctctaagtctaaaaggtgctggtgttgttagtgctggtaaactagctagtggtggtggtgctgttaatgttgctggtggtggtgctgttaaaattgctagtgctagtagtgttggtaatctagctgttcaaggtggtggtaaagttcaagctactctactaaatgctggtggtactctactagttagtggtagacaagctgttcaactaggtgctgctagtagtagacaagctctaagtgttaatgctggtggtgctctaaaagctgataaactaagtgctactagaagagttgatgttgatggtaaacaagctgttgctctaggtagtgctagtagtaatgctctaagtgttagagctggtggtgctctaaaagctggtaaactaagtgctactggtagactagatgttgatggtaaacaagctgttactctaggtagtgttgctagtgatggtgctctaagtgttagtgctggtggtaatctaagagctaatgaactagttagtagtgctcaactagaagttagaggtcaaagagaagttgctctagatgatgctagtagtgctagaggtatgactgttgttgctgctggtgctctagctgctagaaatctacaaagtaaaggtgctattggtgttcaaggtggtgaagctgttagtgttgctaatgctaatagtgatgctgaactaagagttagaggtagaggtcaagttgatctacatgatctaagtgctgctagaggtgctgatattagtggtgaaggtagagttaatattggtagagctagaagtgatagtgatgttaaagttagtgctcatggtgctctaagtattgatagtatgactgctctaggtgctattggtgttcaagctggtggtagtgttagtgctaaagatatgagaagtagaggtgctgttactgttagtggtggtggtgctgttaatctaggtgatgttcaaagtgatggtcaagttagagctactagtgctggtgctatgactgttagagatgttgctgctgctgctgatctagctctacaagctggtgatgctctacaagctggttttctaaaaagtgctggtgctatgactgttaatggtagagatgctgttagactagatggtgctcatgctggtggtcaactaagagttagtagtgatggtcaagctgctctaggtagtctagctgctaaaggtgaactaactgttagtgctgctagagctgctactgttgctgaactaaaaagtctagataatattagtgttactggtggtgaaagagttagtgttcaaagtgttaatagtgctagtagagttgctattagtgctcatggtgctctagatgttggtaaagttagtgctaaaagtggtattggtctagaaggttggggtgctgttggtgctgatagtctaggtagtgatggtgctattagtgttagtggtagagatgctgttagagttgatcatgctagaagtctagctgatattagtctaggtgctgaaggtggtgctactctaggtgctgttgaagctgctggtagtattgatgttagaggtggtagtactgttgctgctaatagtctacatgctaatagagatgttagagttagtggtaaagatgctgttagagttactgctgctactagtggtggtggtctacatgttagtagtggtagacaactagatctaggtgctgttcaagctagaggtgctctagctctagatggtggtgctggtgttgctctacaaagtgctaaagctagtggtactctacatgttcaaggtggtgaacatctagatctaggtactctagctgctgttggtgctgttgatgttaatggtactggtgatgttagagttgctaaactagttagtgatgctggtgctgatctacaagctggtagaagtatgactctaggtattgttgatactactggtgatctacaagctagagctcaacaaaaactagaactaggtagtgttaaaagtgatggtggtctacaagctgctgctggtggtgctctaagtctagctgctgctgaagttgctggtgctctagaactaagtggtcaaggtgttactgttgatagagctagtgctagtagagctagaattgatagtactggtagtgttggtattggtgctctaaaagctggtgctgttgaagctgctagtcctagaagagctagaagagctctaagacaagatttttttactcctggtagtgttgttgttagagctcaaggtaatgttactgttggtagaggtgatcctcatcaaggtgttctagctcaaggtgatattattatggatgctaaaggtggtactctactactaagaaatgatgctctaactgaaaatggtactgttactattagtgctgatagtgctgttctagaacatagtactattgaaagtaaaattagtcaaagtgttctagctgctaaaggtgataaaggtaaacctgctgttagtgttaaagttgctaaaaaactatttctaaatggtactctaagagctgttaatgataataatgaaactatgagtggtagacaaattgatgttgttgatggtagacctcaaattactgatgctgttactggtgaagctagaaaagatgaaagtgttgttagtgatgctgctctagttgctgatggtggtcctattgttgttgaagctggtgaactagttagtcatgctggtggtattggtaatggtagaaataaagaaaatggtgctagtgttactgttagaactactggtaatctagttaataaaggttatattagtgctggtaaacaaggtgttctagaagttggtggtgctctaactaatgaatttctagttggtagtgatggtactcaaagaattgaagctcaaagaattgaaaatagaggtacttttcaaagtcaagctcctgctggtactgctggtgctctagttgttaaagctgctgaagctattgttcatgatggtgttatggctactaaaggtgaaatgcaaattgctggtaaaggtggtggtagtcctactgttactgctggtgctaaagctactactagtgctaataaactaagtgttgatgttgctagttgggataatgctggtagtctagatattaaaaaaggtggtgctcaagttactgttgctggtagatatgctgaacatggtgaagttagtattcaaggtgattatactgttagtgctgatgctattgctctagctgctcaagttactcaaagaggtggtgctgctaatctaactagtagacatgatactagatttagtaataaaattagactaatgggtcctctacaagttaatgctggtggtcctgttagtaatactggtaatctaaaagttagagaaggtgttactgttactgctgctagttttgataatgaaactggtgctgaagttatggctaaaagtgctactctaactactagtggtgctgctagaaatgctggtaaaatgcaagttaaagaagctgctactattgttgctgctagtgttagtaatcctggtacttttactgctggtaaagatattactgttactagtagaggtggttttgataatgaaggtaaaatggaaagtaataaagatattgttattaaaactgaacaatttagtaatggtagagttctagatgctaaacatgatctaactgttactgctagtggtcaagctgataatagaggtagtctaaaagctggtcatgattttactgttcaagctcaaagaattgataatagtggtactatggctgctggtcatgatgctactctaaaagctcctcatctaagaaatactggtcaagttgttgctggtcatgatattcatattattaatagtgctaaactagaaaatactggtagagttgatgctagaaatgatattgctctagatgttgctgattttactaatactggtagtctatatgctgaacatgatgctactctaactctagctcaaggtactcaaagagatctagttgttgatcaagatcatattctacctgttgctgaaggtactctaagagttaaagctaaaagtctaactactgaaattgaaactggtaatcctggtagtctaattgctgaagttcaagaaaatattgataataaacaagctattgttgttggtaaagatctaactctaagtagtgctcatggtaatgttgctaatgaagctaatgctctactatgggctgctggtgaactaactgttaaagctcaaaatattactaataaaagagctgctctaattgaagctggtggtaatgctagactaactgctgctgttgctctactaaataaactaggtagaattagagctggtgaagatatgcatctagatgctcctagaattgaaaatactgctaaactaagtggtgaagttcaaagaaaaggtgttcaagatgttggtggtggtgaacatggtagatggagtggtattggttatgttaattattggctaagagctggtaatggtaaaaaagctggtactattgctgctccttggtatggtggtgatctaactgctgaacaaagtctaattgaagttggtaaagatctatatctaaatgctggtgctagaaaagatgaacatagacatctactaaatgaaggtgttattcaagctggtggtcatggtcatattggtggtgatgttgataatagaagtgttgttagaactgttagtgctatggaatattttaaaactcctctacctgttagtctaactgctctagataatagagctggtctaagtcctgctacttggaattttcaaagtacttatgaactactagattatctactagatcaaaatagatatgaatatatttggggtctatatcctacttatactgaatggagtgttaatactctaaaaaatctagatctaggttatcaagctaaacctgctcctactgctcctcctatgcctaaagctcctgaactagatctaagaggtcatactctagaaagtgctgaaggtagaaaaatttttggtgaatataaaaaactacaaggtgaatatgaaaaagctaaaatggctgttcaagctgttgaagcttatggtgaagctactagaagagttcatgatcaactaggtcaaagatatggtaaagctctaggtggtatggatgctgaaactaaagaagttgatggtattattcaagaatttgctgctgatctaagaactgtttatgctaaacaagctgatcaagctactattgatgctgaaactgataaagttgctcaaagatataaaagtcaaattgatgctgttagactacaagctattcaacctggtagagttactctagctaaagctctaagtgctgctctaggtgctgattggagagctctaggtcatagtcaactaatgcaaagatggaaagattttaaagctggtaaaagaggtgctgaaattgctttttatcctaaagaacaaactgttctagctgctggtgctggtctaactctaagtaatggtgctattcataatggtgaaaatgctgctcaaaatagaggtagacctgaaggtctaaaaattggtgctcatagtgctactagtgttagtggtagttttgatgctctaagagatgttggtctagaaaaaagactagatattgatgatgctctagctgctgttctagttaatcctcatatttttactagaattggtgctgctcaaactagtctagctgatggtgctgctggtcctgctctagctagacaagctagacaagctcctgaaactgatggtatggttgatgctagaggtctaggtagtgctgatgctctagctagtctagctagtctagatgctgctcaaggtctagaagttagtggtagaagaaatgctcaagttgctgatgctggtctagctggtcctagtgctgttgctgctcctgctgttggtgctgctgatgttggtgttgaacctgttactggtgatcaagttgatcaacctgttgttgctgttggtctagaacaacctgttgctactgttagagttgctcctcctgctgttgctctacctagacctctatttgaaactagaattaaatttattgatcaaagtaaattttatggtagtagatatttttttgaacaaattggttataaacctgatagagctgctagagttgctggtgataattattttgatactactctagttagagaacaagttagaagagctctaggtggttatgaaagtagactacctgttagaggtgttgctctagttgctaaactaatggatagtgctggtactgttggtaaagctctaggtctaaaagttggtgttgctcctactgctcaacaactaaaacaagctgatagagattttgtttggtatgttgatactgttattgatggtcaaaaagttctagctcctagactatatctaactgaagctactagacaaggtattactgatcaatatgctggtggtggtgctctaattgctagtggtggtgatgttactgttaatactgatggtcatgatgttagtagtgttaatggtctaattcaaggtagaagtgttaaagttgatgctggtaaaggtaaagttgttgttgctgatagtaaaggtgctggtggtggtattgaagctgatgatgaagttgatgttagtggtagagatattggtattgaaggtggtaaactaagaggtaaagatgttagactaaaagctgatactgttaaagttgctactagtatgagatatgatgataaaggtagactagctgctagaggtgatggtgctctagatgctcaaggtggtcaactacatattgaagctaaaagactagaaactgctggtgctactctaaaaggtggtaaagttaaactagatgttgatgatgttaaactaggtggtgtttatgaagctggtagtagttatgaaaataaaagtagtactcctctaggtagtctatttgctattctaagtagtactactgaaactaatcaaagtgctcatgctaatcattatggtactagaattgaagctggtactctagaaggtaaaatgcaaaatctagaaattgaaggtggtagtgttgatgctgctcatactgatctaagtgttgctagagatgctagatttaaagctgctgctgattttgctcatgctgaacatgaaaaagatgttagacaactaagtctaggtgctaaagttggtgctggtggttatgaagctggttttagtctaggtagtgaaagtggtctagaagctcatgctggtagaggtatgactgctggtgctgaagttaaagttggttatagagctagtcatgaacaaagtagtgaaactgaaaaaagttatagaaatgctaatctaaattttggtggtggtagtgttgaagctggtaatgttctagatattggtggtgctgatattaatagaaatagatatggtggtgctgctaaaggtaatgctggtactgaagaagctctaagaatgagagctaaaaaagttgaaagtactaaatatgttagtgaacaaactagtcaaagtagtggttggagtgttgaagttgctagtactgctagtgctagaagtagtctactaactgctgctactagactaggtgatagtgttgctcaaaatgttgaagatggtagagaaattagaggtgaactaatggctgctcaagttgctgctgaagctactcaactagttactgctgatactgctgctgttgctctaagtgctggtattagtgctgattttgatagtagtcatagtagaagtactagtcaaaatactcaatatctaggtggtaatctaagtattgaagctactgaaggtgatgctactctagttggtgctaaatttggtggtggtgatcaagttagtctaaaagctgctaaaagtgttaatctaatggctgctgaaagtacttttgaaagttatagtgaaagtcataattttcatgctagtgctgatgctaatctaggtgctaatgctgttcaaggtgctgttggtctaggtctaactgctggtatgggtactagtcatcaaattactaatgaaactggtaaaacttatgctggtactagtgttgatgctgctaatgttagtattgatgctggtaaagatctaaatctaagtggtagtagagttagaggtaaacatgttgttctagatgttgaaggtgatattaatgctactagtaaacaagatgaaagaaattataatagtagtggtggtggttgggatgctagtgctggtgttgctattcaaaatagaactctagttgctcctgttggtagtgctggttttaattttaatactgaacatgataatagtagactaactaatgatggtgctgctggtgttgttgctagtgatggtctaactggtcatgttaaaggtgatgctaatctaactggtgctactattgctgatctaagtggtaaaggtaatctaaaagttgatggtgctgttaatgctcaaaatctaaaagattatagagataaagatggtggtagtggtggtctaaatgttggtattagtagtactactctagctcctactgttggtgttgcttttggtagagttgctggtgaagattatcaagctgaacaaagagctactattgatgttggtcaaactaaagatcctgctagactacaagttggtggtggtgttaaaggtactctaaatcaagatgctgctcaagctactgttgttcaaagaaataaacattgggctggtggtggtagtgaatttagtgttgctggtaaaagtctaaaaaaaaaaaatcaagttagacctgttgaaactcctactcctgatgttgttgatggtcctcctagtagacctactactcctcctgctagtcctcaacctattagagctactgttgaagttagtagtcctcctcctgttagtgttgctactgttgaagttgttcctagacctaaagttgaaactggtagtgctgctagtgctagtgctggtggtgctcaagttgttcctgttactcctcctaaagttgaagttgctaaagttgaagttgttcctagacctaaagttgaaactgctcaacctctacctcctagacctgttgttgctgaaaaagttactactcctgctgttcaacctcaactagctaaagttgaaactgttcaacctgttaaacctgaaactactaaacctctacctaaacctctacctgttgctaaagttactaaagctcctcctcctgttgttgaaactgctcaacctctacctcctgttaaacctcaaaaagctactcctggtcctgttgctgaagttggtaaagctactgttactactgttcaagttcaaagtgctcctcctaaacctgctcctgttgctaaacaacctgctcctgctcctaaacctaaacctaaacctaaacctaaagctgaaagacctaaacctggtaaaactactcctctaagtggtagacatgttgttcaacaacaagttcaagttctacaaagacaagctagtgatattaataatactaaaagtctacctggtggtaaactacctaaacctgttactgttaaactaactgatgaaaatggtaaacctcaaacttatactattaatagaagagaagatctaatgaaactaaatggtaaagttctaagtactaaaactactctaggtctagaacaaacttttagactaagagttgaagatattggtggtaaaaattatagagttttttatgaaactaataaa

SEQ ID NO: 169 - RNA, 10770 nt, synthetic construct
  source 1..10770: mol_type = other RNA
atgaatactaatctgtataggctggtttttagccatgttaggggtatgctggttcctgttagcgaacattgtactgttggtaatactttttgtggtaggactaggggtcaagctaggagcggtgctagggctactagcctgagcgttgctcctaatgctctggcttgggctctgatgctggcttgtactggtctgcctctggttactcatgctcaaggtctggttcctcaaggtcaaactcaagttctgcaaggtggtaataaagttcctgttgttaatattgctgatcctaatagcggtggtgttagccataataaatttcaacaatttaatgttgctaatcctggtgttgtttttaataatggtctgactgatggtgttagcaggattggtggtgctctgactaaaaatcctaatctgactaggcaagctagcgctattctggctgaagttactgatactagccctagcaggctggctggtactctggaagtttatggtaaaggtgctgatctgattattgctaatcctaatggtattagcgttaatggtctgagcactctgaatgctagcaatctgactctgactactggtaggcctagcgttaatggtggtaggattggtctggatgttcaacaaggtactgttactattgaaaggggtggtgttaatgctactggtctgggttattttgatgttgttgctaggctggttaaactgcaaggtgctgttagcagcaaacaaggtaaacctctggctgatattgctgttgttgctggtgctaataggtatgatcatgctactaggagggctactcctattgctgctggtgctaggggtgctgctgctggtgcttatgctattgatggtactgctgctggtgctatgtatggtaaacatattactctggttagcagcgatagcggtctgggtgttaggcaactgggtagcctgagcagccctagcgctattactgttagcagccaaggtgaaattgctctgggtgatgctactgttcaaaggggtcctctgagcctgaaaggtgctggtgttgttagcgctggtaaactggctagcggtggtggtgctgttaatgttgctggtggtggtgctgttaaaattgctagcgctagcagcgttggtaatctggctgttcaaggtggtggtaaagttcaagctactctgctgaatgctggtggtactctgctggttagcggtaggcaagctgttcaactgggtgctgctagcagcaggcaagctctgagcgttaatgctggtggtgctctgaaagctgataaactgagcgctactaggagggttgatgttgatggtaaacaagctgttgctctgggtagcgctagcagcaatgctctgagcgttagggctggtggtgctctgaaagctggtaaactgagcgctactggtaggctggatgttgatggtaaacaagctgttactctgggtagcgttgctagcgatggtgctctgagcgttagcgctggtggtaatctgagggctaatgaactggttagcagcgctcaactggaagttaggggtcaaagggaagttgctctggatgatgctagcagcgctaggggtatgactgttgttgctgctggtgctctggctgctaggaatctgcaaagcaaaggtgctattggtgttcaaggtggtgaagctgttagcgttgctaatgctaatagcgatgctgaactgagggttaggggtaggggtcaagttgatctgcatgatctgagcgctgctaggggtgctgatattagcggtgaaggtagggttaatattggtagggctaggagcgatagcgatgttaaagttagcgctcatggtgctctgagcattgatagcatgactgctctgggtgctattggtgttcaagctggtggtagcgttagcgctaaagatatgaggagcaggggtgctgttactgttagcggtggtggtgctgttaatctgggtgatgttcaaagcgatggtcaagttagggctactagcgctggtgctatgactgttagggatgttgctgctgctgctgatctggctctgcaagctggtgatgctctgcaagctggttttctgaaaagcgctggtgctatgactgttaatggtagggatgctgttaggctggatggtgctcatgctggtggtcaactgagggttagcagcgatggtcaagctgctctgggtagcctggctgctaaaggtgaactgactgttagcgctgctagggctgctactgttgctgaactgaaaagcctggataatattagcgttactggtggtgaaagggttagcgttcaaagcgttaatagcgctagcagggttgctattagcgctcatggtgctctggatgttggtaaagttagcgctaaaagcggtattggtctggaaggttggggtgctgttggtgctgatagcctgggtagcgatggtgctattagcgttagcggtagggatgctgttagggttgatcatgctaggagcctggctgatattagcctgggtgctgaaggtggtgctactctgggtgctgttgaagctgctggtagcattgatgttaggggtggtagcactgttgctgctaatagcctgcatgctaatagggatgttagggttagcggtaaagatgctgttagggttactgctgctactagcggtggtggtctgcatgttagcagcggtaggcaactggatctgggtgctgttcaagctaggggtgctctggctctggatggtggtgctggtgttgctctgcaaagcgctaaagctagcggtactctgcatgttcaaggtggtgaacatctggatctgggtactctggctgctgttggtgctgttgatgttaatggtactggtgatgttagggttgctaaactggttagcgatgctggtgctgatctgcaagctggtaggagcatgactctgggtattgttgatactactggtgatctgcaagctagggctcaacaaaaactggaactgggtagcgttaaaagcgatggtggtctgcaagctgctgctggtggtgctctgagcctggctgctgctgaagttgctggtgctctggaactgagcggtcaaggtgttactgttgatagggctagcgctagcagggctaggattgatagcactggtagcgttggtattggtgctctgaaagctggtgctgttgaagctgctagccctaggagggctaggagggctctgaggcaagatttttttactcctggtagcgttgttgttagggctcaaggtaatgttactgttggtaggggtgatcctcatcaaggtgttctggctcaaggtgatattattatggatgctaaaggtggtactctgctgctgaggaatgatgctctgactgaaaatggtactgttactattagcgctgatagcgctgttctggaacatagcactattgaaagcaaaattagccaaagcgttctggctgctaaaggtgataaaggtaaacctgctgttagcgttaaagttgctaaaaaactgtttctgaatggtactctgagggctgttaatgataataatgaaactatgagcggtaggcaaattgatgttgttgatggtaggcctcaaattactgatgctgttactggtgaagctaggaaagatgaaagcgttgttagcgatgctgctctggttgctgatggtggtcctattgttgttgaagctggtgaactggttagccatgctggtggtattggtaatggtaggaataaagaaaatggtgctagcgttactgttaggactactggtaatctggttaataaaggttatattagcgctggtaaacaaggtgttctggaagttggtggtgctctgactaatgaatttctggttggtagcgatggtactcaaaggattgaagctcaaaggattgaaaataggggtacttttcaaagccaagctcctgctggtactgctggtgctctggttgttaaagctgctgaagctattgttcatgatggtgttatggctactaaaggtgaaatgcaaattgctggtaaaggtggtggtagccctactgttactgctggtgctaaagctactactagcgctaataaactgagcgttgatgttgctagctgggataatgctggtagcctggatattaaaaaaggtggtgctcaagttactgttgctggtaggtatgctgaacatggtgaagttagcattcaaggtgattatactgttagcgctgatgctattgctctggctgctcaagttactcaaaggggtggtgctgctaatctgactagcaggcatgatactaggtttagcaataaaattaggctgatgggtcctctgcaagttaatgctggtggtcctgttagcaatactggtaatctgaaagttagggaaggtgttactgttactgctgctagctttgataatgaaactggtgctgaagttatggctaaaagcgctactctgactactagcggtgctgctaggaatgctggtaaaatgcaagttaaagaagctgctactattgttgctgctagcgttagcaatcctggtacttttactgctggtaaagatattactgttactagcaggggtggttttgataatgaaggtaaaatggaaagcaataaagatattgttattaaaactgaacaatttagcaatggtagggttctggatgctaaacatgatctgactgttactgctagcggtcaagctgataataggggtagcctgaaagctggtcatgattttactgttcaagctcaaaggattgataatagcggtactatggctgctggtcatgatgctactctgaaagctcctcatctgaggaatactggtcaagttgttgctggtcatgatattcatattattaatagcgctaaactggaaaatactggtagggttgatgctaggaatgatattgctctggatgttgctgattttactaatactggtagcctgtatgctgaacatgatgctactctgactctggctcaaggtactcaaagggatctggttgttgatcaagatcatattctgcctgttgctgaaggtactctgagggttaaagctaaaagcctgactactgaaattgaaactggtaatcctggtagcctgattgctgaagttcaagaaaatattgataataaacaagctattgttgttggtaaagatctgactctgagcagcgctcatggtaatgttgctaatgaagctaatgctctgctgtgggctgctggtgaactgactgttaaagctcaaaatattactaataaaagggctgctctgattgaagctggtggtaatgctaggctgactgctgctgttgctctgctgaataaactgggtaggattagggctggtgaagatatgcatctggatgctcctaggattgaaaatactgctaaactgagcggtgaagttcaaaggaaaggtgttcaagatgttggtggtggtgaacatggtaggtggagcggtattggttatgttaattattggctgagggctggtaatggtaaaaaagctggtactattgctgctccttggtatggtggtgatctgactgctgaacaaagcctgattgaagttggtaaagatctgtatctgaatgctggtgctaggaaagatgaacataggcatctgctgaatgaaggtgttattcaagctggtggtcatggtcatattggtggtgatgttgataataggagcgttgttaggactgttagcgctatggaatattttaaaactcctctgcctgttagcctgactgctctggataatagggctggtctgagccctgctacttggaattttcaaagcacttatgaactgctggattatctgctggatcaaaataggtatgaatatatttggggtctgtatcctacttatactgaatggagcgttaatactctgaaaaatctggatctgggttatcaagctaaacctgctcctactgctcctcctatgcctaaagctcctgaactggatctgaggggtcatactctggaaagcgctgaaggtaggaaaatttttggtgaatataaaaaactgcaaggtgaatatgaaaaagctaaaatggctgttcaagctgttgaagcttatggtgaagctactaggagggttcatgatcaactgggtcaaaggtatggtaaagctctgggtggtatggatgctgaaactaaagaagttgatggtattattcaagaatttgctgctgatctgaggactgtttatgctaaacaagctgatcaagctactattgatgctgaaactgataaagttgctcaaaggtataaaagccaaattgatgctgttaggctgcaagctattcaacctggtagggttactctggctaaagctctgagcgctgctctgggtgctgattggagggctctgggtcatagccaactgatgcaaaggtggaaagattttaaagctggtaaaaggggtgctgaaattgctttttatcctaaagaacaaactgttctggctgctggtgctggtctgactctgagcaatggtgctattcataatggtgaaaatgctgctcaaaataggggtaggcctgaaggtctgaaaattggtgctcatagcgctactagcgttagcggtagctttgatgctctgagggatgttggtctggaaaaaaggctggatattgatgatgctctggctgctgttctggttaatcctcatatttttactaggattggtgctgctcaaactagcctggctgatggtgctgctggtcctgctctggctaggcaagctaggcaagctcctgaaactgatggtatggttgatgctaggggtctgggtagcgctgatgctctggctagcctggctagcctggatgctgctcaaggtctggaagttagcggtaggaggaatgctcaagttgctgatgctggtctggctggtcctagcgctgttgctgctcctgctgttggtgctgctgatgttggtgttgaacctgttactggtgatcaagttgatcaacctgttgttgctgttggtctggaacaacctgttgctactgttagggttgctcctcctgctgttgctctgcctaggcctctgtttgaaactaggattaaatttattgatcaaagcaaattttatggtagcaggtatttttttgaacaaattggttataaacctgatagggctgctagggttgctggtgataattattttgatactactctggttagggaacaagttaggagggctctgggtggttatgaaagcaggctgcctgttaggggtgttgctctggttgctaaactgatggatagcgctggtactgttggtaaagctctgggtctgaaagttggtgttgctcctactgctcaacaactgaaacaagctgatagggattttgtttggtatgttgatactgttattgatggtcaaaaagttctggctcctaggctgtatctgactgaagctactaggcaaggtattactgatcaatatgctggtggtggtgctctgattgctagcggtggtgatgttactgttaatactgatggtcatgatgttagcagcgttaatggtctgattcaaggtaggagcgttaaagttgatgctggtaaaggtaaagttgttgttgctgatagcaaaggtgctggtggtggtattgaagctgatgatgaagttgatgttagcggtagggatattggtattgaaggtggtaaactgaggggtaaagatgttaggctgaaagctgatactgttaaagttgctactagcatgaggtatgatgataaaggtaggctggctgctaggggtgatggtgctctggatgctcaaggtggtcaactgcatattgaagctaaaaggctggaaactgctggtgctactctgaaaggtggtaaagttaaactggatgttgatgatgttaaactgggtggtgtttatgaagctggtagcagctatgaaaataaaagcagcactcctctgggtagcctgtttgctattctgagcagcactactgaaactaatcaaagcgctcatgctaatcattatggtactaggattgaagctggtactctggaaggtaaaatgcaaaatctggaaattgaaggtggtagcgttgatgctgctcatactgatctgagcgttgctagggatgctaggtttaaagctgctgctgattttgctcatgctgaacatgaaaaagatgttaggcaactgagcctgggtgctaaagttggtgctggtggttatgaagctggttttagcctgggtagcgaaagcggtctggaagctcatgctggtaggggtatgactgctggtgctgaagttaaagttggttatagggctagccatgaacaaagcagcgaaactgaaaaaagctataggaatgctaatctgaattttggtggtggtagcgttgaagctggtaatgttctggatattggtggtgctgatattaataggaataggtatggtggtgctgctaaaggtaatgctggtactgaagaagctctgaggatgagggctaaaaaagttgaaagcactaaatatgttagcgaacaaactagccaaagcagcggttggagcgttgaagttgctagcactgctagcgctaggagcagcctgctgactgctgctactaggctgggtgatagcgttgctcaaaatgttgaagatggtagggaaattaggggtgaactgatggctgctcaagttgctgctgaagctactcaactggttactgctgatactgctgctgttgctctgagcgctggtattagcgctgattttgatagcagccatagcaggagcactagccaaaatactcaatatctgggtggtaatctgagcattgaagctactgaaggtgatgctactctggttggtgctaaatttggtggtggtgatcaagttagcctgaaagctgctaaaagcgttaatctgatggctgctgaaagcacttttgaaagctatagcgaaagccataattttcatgctagcgctgatgctaatctgggtgctaatgctgttcaaggtgctgttggtctgggtctgactgctggtatgggtactagccatcaaattactaatgaaactggtaaaacttatgctggtactagcgttgatgctgctaatgttagcattgatgctggtaaagatctgaatctgagcggtagcagggttaggggtaaacatgttgttctggatgttgaaggtgatattaatgctactagcaaacaagatgaaaggaattataatagcagcggtggtggttgggatgctagcgctggtgttgctattcaaaataggactctggttgctcctgttggtagcgctggttttaattttaatactgaacatgataatagcaggctgactaatgatggtgctgctggtgttgttgctagcgatggtctgactggtcatgttaaaggtgatgctaatctgactggtgctactattgctgatctgagcggtaaaggtaatctgaaagttgatggtgctgttaatgctcaaaatctgaaagattatagggataaagatggtggtagcggtggtctgaatgttggtattagcagcactactctggctcctactgttggtgttgcttttggtagggttgctggtgaagattatcaagctgaacaaagggctactattgatgttggtcaaactaaagatcctgctaggctgcaagttggtggtggtgttaaaggtactctgaatcaagatgctgctcaagctactgttgttcaaaggaataaacattgggctggtggtggtagcgaatttagcgttgctggtaaaagcctgaaaaaaaaaaatcaagttaggcctgttgaaactcctactcctgatgttgttgatggtcctcctagcaggcctactactcctcctgctagccctcaacctattagggctactgttgaagttagcagccctcctcctgttagcgttgctactgttgaagttgttcctaggcctaaagttgaaactggtagcgctgctagcgctagcgctggtggtgctcaagttgttcctgttactcctcctaaagttgaagttgctaaagttgaagttgttcctaggcctaaagttgaaactgctcaacctctgcctcctaggcctgttgttgctgaaaaagttactactcctgctgttcaacctcaactggctaaagttgaaactgttcaacctgttaaacctgaaactactaaacctctgcctaaacctctgcctgttgctaaagttactaaagctcctcctcctgttgttgaaactgctcaacctctgcctcctgttaaacctcaaaaagctactcctggtcctgttgctgaagttggtaaagctactgttactactgttcaagttcaaagcgctcctcctaaacctgctcctgttgctaaacaacctgctcctgctcctaaacctaaacctaaacctaaacctaaagctgaaaggcctaaacctggtaaaactactcctctgagcggtaggcatgttgttcaacaacaagttcaagttctgcaaaggcaagctagcgatattaataatactaaaagcctgcctggtggtaaactgcctaaacctgttactgttaaactgactgatgaaaatggtaaacctcaaacttatactattaataggagggaagatctgatgaaactgaatggtaaagttctgagcactaaaactactctgggtctggaacaaacttttaggctgagggttgaagatattggtggtaaaaattatagggttttttatgaaactaataaa

SEQ ID NO: 170 - RNA, 987 nt, synthetic construct
  source 1..987: mol_type = other RNA
atgaaatttggttctaaaattcgtcgtttagctgttgctgctgttgctggtgctattgctttaggtgcttcttttgctgttgctcaagctcctactttttttcgtattggtactggtggtactgctggtacttattatcctattggtggtttaattgctaatgctatttctggtgctggtgaaaaaggtgttcctggtttagttgctactgctgtttcttctaatggttctgttgctaatattaatgctattaaatctggtgctttagaatctggttttactcaatctgatgttgcttattgggcttataatggtactggtttatatgatggtaaaggtaaagttgaagatttacgtttattagctactttatatcctgaaactattcatattgttgctcgtaaagatgctaatattaaatctgttgctgatttaaaaggtaaacgtgtttctttagatgaacctggttctggtactattgttgatgctcgtattgttttagaagcttatggtttaactgaagatgatattaaagctgaacatttaaaacctggtcctgctggtgaacgtttaaaagatggtgctttagatgcttatttttttgttggtggttatcctactggtgctatttctgaattagctatttctaatggtatttctttagttcctatttctggtcctgaagctgataaaattttagaaaaatattcttttttttctaaagatgttgttcctgctggtgcttataaagatgttgctgaaactcctactttagctgttgctgctcaatgggttacttctgctaaacaacctgatgatttaatttataatattactaaagttttatggaatgaagatactcgtaaagctttagatgctggtcatgctaaaggtaaattaattaaattagattctgctacttcttctttaggtattcctttacatcctggtgctgaacgtttttataaagaagctggtgttttaaaa

SEQ ID NO: 171 - RNA, 987 nt, synthetic construct; identical to SEQ ID NO: 170
  source 1..987: mol_type = other RNA

SEQ ID NO: 172 - RNA, 987 nt, synthetic construct
  source 1..987: mol_type = other RNA
atgaaatttggatcaaaaatacgacgacttgcagtagcagcagtagcaggagcaatagcacttggagcatcatttgcagtagcacaagcaccaacattttttcgaataggaacaggaggaacagcaggaacatattatccaataggaggacttatagcaaatgcaatatcaggagcaggagaaaaaggagtaccaggacttgtagcaacagcagtatcatcaaatggatcagtagcaaatataaatgcaataaaatcaggagcacttgaatcaggatttacacaatcagatgtagcatattgggcatataatggaacaggactttatgatggaaaaggaaaagtagaagatcttcgacttcttgcaacactttatccagaaacaatacatatagtagcacgaaaagatgcaaatataaaatcagtagcagatcttaaaggaaaacgagtatcacttgatgaaccaggatcaggaacaatagtagatgcacgaatagtacttgaagcatatggacttacagaagatgatataaaagcagaacatcttaaaccaggaccagcaggagaacgacttaaagatggagcacttgatgcatatttttttgtaggaggatatccaacaggagcaatatcagaacttgcaatatcaaatggaatatcacttgtaccaatatcaggaccagaagcagataaaatacttgaaaaatattcatttttttcaaaagatgtagtaccagcaggagcatataaagatgtagcagaaacaccaacacttgcagtagcagcacaatgggtaacatcagcaaaacaaccagatgatcttatatataatataacaaaagtactttggaatgaagatacacgaaaagcacttgatgcaggacatgcaaaaggaaaacttataaaacttgattcagcaacatcatcacttggaataccacttcatccaggagcagaacgattttataaagaagcaggagtacttaaa

SEQ ID NO: 173 - RNA, 987 nt, synthetic construct
  source 1..987: mol_type = other RNA
atgaaatttgggtcgaaaattcggcggctcgcggtggcggcggtggcgggggcgattgcgctcggggcgtcgtttgcggtggcgcaagcgccgacgttttttcggattgggacgggggggacggcggggacgtattatccgattggggggctcattgcgaatgcgatttcgggggcgggggaaaaaggggtgccggggctcgtggcgacggcggtgtcgtcgaatgggtcggtggcgaatattaatgcgattaaatcgggggcgctcgaatcggggtttacgcaatcggatgtggcgtattgggcgtataatgggacggggctctatgatgggaaagggaaagtggaagatctccggctcctcgcgacgctctatccggaaacgattcatattgtggcgcggaaagatgcgaatattaaatcggtggcggatctcaaagggaaacgggtgtcgctcgatgaaccggggtcggggacgattgtggatgcgcggattgtgctcgaagcgtatgggctcacggaagatgatattaaagcggaacatctcaaaccggggccggcgggggaacggctcaaagatggggcgctcgatgcgtatttttttgtgggggggtatccgacgggggcgatttcggaactcgcgatttcgaatgggatttcgctcgtgccgatttcggggccggaagcggataaaattctcgaaaaatattcgtttttttcgaaagatgtggtgccggcgggggcgtataaagatgtggcggaaacgccgacgctcgcggtggcggcgcaatgggtgacgtcggcgaaacaaccggatgatctcatttataatattacgaaagtgctctggaatgaagatacgcggaaagcgctcgatgcggggcatgcgaaagggaaactcattaaactcgattcggcgacgtcgtcgctcgggattccgctccatccgggggcggaacggttttataaagaagcgggggtgctcaaa

SEQ ID NO: 174 - RNA, 987 nt, synthetic construct
  source 1..987: mol_type = other RNA
atgaaatttggtagtaaaattagaagactagctgttgctgctgttgctggtgctattgctctaggtgctagttttgctgttgctcaagctcctactttttttagaattggtactggtggtactgctggtacttattatcctattggtggtctaattgctaatgctattagtggtgctggtgaaaaaggtgttcctggtctagttgctactgctgttagtagtaatggtagtgttgctaatattaatgctattaaaagtggtgctctagaaagtggttttactcaaagtgatgttgcttattgggcttataatggtactggtctatatgatggtaaaggtaaagttgaagatctaagactactagctactctatatcctgaaactattcatattgttgctagaaaagatgctaatattaaaagtgttgctgatctaaaaggtaaaagagttagtctagatgaacctggtagtggtactattgttgatgctagaattgttctagaagcttatggtctaactgaagatgatattaaagctgaacatctaaaacctggtcctgctggtgaaagactaaaagatggtgctctagatgcttatttttttgttggtggttatcctactggtgctattagtgaactagctattagtaatggtattagtctagttcctattagtggtcctgaagctgataaaattctagaaaaatatagtttttttagtaaagatgttgttcctgctggtgcttataaagatgttgctgaaactcctactctagctgttgctgctcaatgggttactagtgctaaacaacctgatgatctaatttataatattactaaagttctatggaatgaagatactagaaaagctctagatgctggtcatgctaaaggtaaactaattaaactagatagtgctactagtagtctaggtattcctctacatcctggtgctgaaagattttataaagaagctggtgttctaaaa

SEQ ID NO: 175 - RNA, 987 nt, synthetic construct
  source 1..987: mol_type = other RNA
atgaaatttggtagcaaaattaggaggctggctgttgctgctgttgctggtgctattgctctgggtgctagctttgctgttgctcaagctcctactttttttaggattggtactggtggtactgctggtacttattatcctattggtggtctgattgctaatgctattagcggtgctggtgaaaaaggtgttcctggtctggttgctactgctgttagcagcaatggtagcgttgctaatattaatgctattaaaagcggtgctctggaaagcggttttactcaaagcgatgttgcttattgggcttataatggtactggtctgtatgatggtaaaggtaaagttgaagatctgaggctgctggctactctgtatcctgaaactattcatattgttgctaggaaagatgctaatattaaaagcgttgctgatctgaaaggtaaaagggttagcctggatgaacctggtagcggtactattgttgatgctaggattgttctggaagcttatggtctgactgaagatgatattaaagctgaacatctgaaacctggtcctgctggtgaaaggctgaaagatggtgctctggatgcttatttttttgttggtggttatcctactggtgctattagcgaactggctattagcaatggtattagcctggttcctattagcggtcctgaagctgataaaattctggaaaaatatagcttttttagcaaagatgttgttcctgctggtgcttataaagatgttgctgaaactcctactctggctgttgctgctcaatgggttactagcgctaaacaacctgatgatctgatttataatattactaaagttctgtggaatgaagatactaggaaagctctggatgctggtcatgctaaaggtaaactgattaaactggatagcgctactagcagcctgggtattcctctgcatcctggtgctgaaaggttttataaagaagctggtgttctgaaa

SEQ ID NO: 176 - protein, 258 aa, synthetic construct
MRSLSVLALLLLLLLAPASASGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQLVPRGSHHHHHHG

SEQ ID NO: 177 - protein, 239 aa, synthetic construct
MSGLRINSAKDDAAGQAAANRATSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGSTANPLASIDSALSKVDAVRSSLGAIQNRFDSAITNLGNTVTNLNSARSRIEDADYATEVSQMSKAQILDQAGTSTLAQLVPRGSHHHHHHG

SEQ ID NO: 178 - protein, 246 aa, synthetic construct
MSGLRINSAKDDAAGQAIANRFTSNIKGLTQASRNAADGISIAQTTEGALNEINNNLQRVRELSVQATAGANADAALKAIQAEIQQRLEEIDRVSQQTQAAAVKVLSQDNAMAIQVGANDGAAITIDLQKIDVKSLGLDGFNVNSPGNPLASIDSALSKVDAVRSSLGAIQNRFDSAITNLGNTVTNLNSARSIEDADYATEVSQMSKAQILQQAGTSVLAQANQVPQNVLSLLVPRGSHHHHHHG

SEQ ID NO: 179 - protein, 209 aa, synthetic construct
MRGSHHHHHHGMASMTGGQQMGRDLYDDDDKDPMAQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIDLQKIDVKSLGLDGFNVN

SEQ ID NO: 180 - protein, 239 aa, synthetic construct; identical to SEQ ID NO: 177

SEQ ID NO: 181 - protein, 258 aa, synthetic construct; identical to SEQ ID NO: 176

SEQ ID NO: 182 - protein, 158 aa, synthetic construct
MNQSCPNKTSFKIAFIQARWHADIVDEARKSFVAELAAKTGGSVEVEIFDVPGAYEIPLHAKTLARTGRYAAIVGAAFVIDGGIYRHDFVATAVINGMMQVQLETEVPVLSVVLTPHHFHESKEHHDFFHAHFKVKGVEAAHAALQIVSERSRIAALV

SEQ ID NO: 183 - protein, 168 aa, synthetic construct
MRRIQSIARSPIAIALFMSLAVAGCASKKNLPNNAGDLGLGAGAATPGSSQDFTVNVGDRIFFDLDSSLIRADAQQTLSKQAQWLQRYPQYSITIEGHADERGTREYNLALGQRRAAATRDFLASRGVPTNRMRTISYGNERPVAVCDADTCWSQNRRAVTVLNGAGR

SEQ ID NO: 184 - protein, 177 aa, synthetic construct
MGISKASLLSLAAAGIVLAGCQSSRLGNLDNVSPPPPPAPVNAVPAGTVQKGNLDSPTQFPNAPSTDMSAQSGTQVASLPPASAPDLTPGAVAGVWNASLGGQSCKIATPQTKYGQGYRAGPLRCPGELANLASWAVNGKQLVLYDANGGTVASLYSSGQGRFDGQTTGGQAVTLSR

SEQ ID NO: 185 - protein, 349 aa, synthetic construct
MEFATVSPPDPGTAAAARARQDTLTKPRGALGRLEDLSVWIAACQGQCPPRQFERARVVVFAGDHGVARCGVSAYPPEVTAQMVANFDAGGAAINALAGVAGASVRVADLAVDADPPDDRIGAHKVRRGSGDITVQDALTAEETERALSAGAAIADEEVDAGADLLIAGDMGIGNTTAAAVLVAALTNVEPVVAVGFGTGIDDAGWARKTAAVRDALFRARRVLPDPVALLRCAGGADLAALAGFCAQAAVRRTPLLLDGMAVTAAALVAEHLAPGARLWWQAGHRSTEPGHALALTALDLEPILDLRMRLGEGTGAALALPIVRAAVAALSSMATFAQAGVSDPSAHP

SEQ ID NO: 186 - protein, 176 aa, synthetic construct
MRNVRKTLILAAVTGTLVTIPSATAHADAEQVGFDPNVPAAGPAPDAPPPPAPDAPPAPAPDAPPAPDAPPPPAPDAPPPAPDAPPAPGPDVPPPPAPKAYSVNWDAIAQCESGGNWGINTGNGYAGGLQFTSSTWHANGGSGSPAGASREEQIRVAENVLHSQGIGAWPVCGRRG

SEQ ID NO: 187 - protein, 130 aa, synthetic construct
MAKLSTDELLDAFKEMTLLELSDFVKKFEETFEVTAAAPVAVAAAGAAPAGAAVEAAEEQSEFDVILEAAGDKKIGVIKVVREIVSGLGLKEAKDLVDGAPKPLLEKVAKEAADEAKAKLEAAGATVTVK

SEQ ID NO: 188 - protein, 205 aa, synthetic construct
MAENPNIDDLPAPLLAALGAADLALATVNDLIANLRERAEETRAETRTRVEERRARLTKFQEDLPEQFIELRDKFTTEELRKAAEGYLEAATNRYNELVERGEAALQRLRSQTAFEDASARAEGYVDQAVELTQEALGTVASQTRAVGERAAKLVGIELPGKAEAAGKKAQKAIAKAPAKKASAKKAPAKKAPAKKAAAKKVTQK

SEQ ID NO: 189 - protein, 594 aa, synthetic construct
MNKIYRIIWNSALNAWVVVSELTRNHTKRASATVATAVLATLLFATVQANATDDDDLYLEPVQRTAVVLSFRSDKEGTGEKEGTEDSNWAVYFDEKRVLKAGAITLKAGDNLKIKQNTNENTNDSSFTYSLKKDLTDLTSVETEKLSFGANGNKVNITSDTKGLNFAKETAGTNGDPTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKGKDENGSSTDEGEGLVTAKEVIDAVNKAGWRMKTTTANGQTGQADKFETVTSGTNVTFASGKGTTATVSKDDQGNITVKYDVNVGDALNVNQLQNSGWNLDSKAVAGSSGKVISGNVSPSKGKMDETVNINAGNNIEITRNGKNIDIATSMAPQFSSVSLGAGADAPTLSVDDEGALNVGSKDTNKPVRITNVAPGVKEGDVTNVAQLKGVAQNLNNRIDNVDGNARAGIAQAIATAGLVQAYLPGKSMMAIGGDTYRGEAGYAIGYSSISDGGNWIIKGTASGNSRGHFGASASVGYQW

SEQ ID NO: 190 - protein, 378 aa, Streptococcus pneumoniae
MNNTEFYDRLGVSKNASADEIKKAYRKLSKKYHPDINKEPGAEDKYKEVQEAYETLSDDQKRAAYDQYGAAGANGGFGGAGGFGGFNGAGGFGGFEDIFSSFFGGGGSSRNPNAPRQGDDLQYRVNLTFEEAIFGTEKEVKYHREAGCRTCNGSGAKPGTSPVTCGRCHGAGVINVDTQTPLGMMRRQVTCDVCHGRGKEIKYPCTTCHGTGHEKQAHSVHVKIPAGVETGQQIRLAGQGEAGFNGGPYGDLYVVVSVEASDKFEREGTTIFYNLNLNFVQAALGDTVDIPTVHGDVELVIPEGTQTGKKFRLRSKGAPSLRGGAVGDQYVTVNVVTPTGLNDRQKVALKEFAAAGDLKVNPKKKGFFDHIKDAFDGE

SEQ ID NO: 191 - protein, 471 aa, synthetic construct
MANKAVNDFILAMNYDKKKLLTHQGESIENRFIKEGNQLPDEFVVIERKKRSLSTNTSDISVTATNDSRLYPGALLVVDETLLENNPTLLAVDRAPMTYSIDLPGLASSDSFLQVEDPSNSSVRGAVNDLLAKWHQDYGQVNNVPARMQYEKITAHSMEQLKVKFGSDFEKTGNSLDIDFNSVHSGEKQIQIVNFKQIYYTVSVDAVKNPGDVFQDTVTVEDLKQRGISAERPLVYISSVAYGRQVYLKLETTSKSDEVEAAFEALIKGVKVAPQTEWKQILDNTEVKAVILGGDPSSGARVVTGKVDMVEDLIQEGSRFTADHPGLPISYTTSFLRDNVVATFQNSTDYVETKVTAYRNGDLLLDHSGAYVAQYYITWDELSYDHQGKEVLTPKAWDRNGQDLTAHFTTSIPLKGNVRNLSVKIRECTGLAWEWWRTVYEKTDLPLVRKRTISIWGTTLYPQVEDKVEND

SEQ ID NO: 192 - protein, 215 aa, synthetic construct
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 193 - protein, 341 aa, Vibrio cholerae
MNKTLIALAVSAAAVATGAYADGINQSGDKAGSTVYSAKGTSLEVGGRAEARLSLKDGKAQDNSRVRLNFLGKAEINDSLYGVGFYEGEFTTNDQGKNASNNSLDNRYTYAGIGGTYGEVTYGKNDGALGVITDFTDIMSYHGNTAAEKIAVADRVDNMLAYKGQFGDLGVKASYRFADRNAVDAMGNVVTETNAAKYSDNGEDGYSLSAIYTFGDTGFNVGAGYADQDDQNEYMLAASYRMENLYFAGLFTDGELAKDVDYTGYELAAGYKLGQAAFTATYNNAETAKETSADNFAIDATYYFKPNFRSYISYQFNLLDSDKVGKVASEDELAIGLRYDF

SEQ ID NO: 194 - protein, 630 aa, Streptococcus pneumoniae
MTRYQDDFYDAINGEWQQTAEIPADKSQTGGFVDLDQEIEDLMLATTDKWLAGEEVPEDAILENFVKYHRLVRDFDKREADGITPVLPLLKEFQELETFADFTAKLAEFELAGKPNFLPFGVSPDFMDARINVLWASAPSTILPDTTYYAEEHPQREELLTLWKESSANLLKAYDFSDEEIEDLLEKRLELDRRVAAVVLSNEESSEYAKLYHPYSYEDFKKFAPALPLDDFFKAVIGQLPDKVIVDEERFWQAAEQFYSEEAWSLLKATLILSVVNLSTSYLTEDIRVLSGAYSRALSGVPEAKDKVKAAYHLAQEPFKQALGLWYAREKFSPEAKADVEKKVATMIDVYKERLLKNDWLTPETCKQAIVKLNVIKPYIGYPEELPARYKDKVVNETASLFENALAFARVEIKHSWSKWNQPVDYKEWGMPAHMVNAYYNPQKNLIVFPAAILQAPFYDLHQSSSANYGGIGAVIAHEISHAFDTNGASFDENGSLKDWWTESDYAAFKEKTQKVIDQFDGQDSYGATINGKLTVSENVADLGGIAAALEAAKREADFSAEEFFYNFGRIWRMKGRPEFMKLLASVDVHAPAKLRVNVQVPNFDDFFTTYDVKEGDGMWRSPEERVIIW

SEQ ID NO: 195 - protein, 348 aa, Shigella flexneri
MKKTAIAIAVALAGFATVAQAAPKDNTWYTGAKLGWSQYHDTGFIPNNGPTHENQLGAGAFGGYQVNPYVGFEMGYDWLGRMPYKGDNINGAYKAQGVQLTAKLGYPITDDLDIYTRLGGMVWRADTKANVPGGASFKDHDTGVSPVFAGGVEYAITPEIATRLEYQWTNNIGDANTIGTRPDNGLLSLGVSYRFGQGEAAPVVAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 196 - protein, 351 aa, Shigella dysenteriae
MKKTAIAITVALAGFATVAQAAPKDNTWYTGAKLGWSQYHDTGFIDNNGPTHENQLGAGAFGGYQVNPYVGFEMGYDWLGRMPYKGSVENGAYKAQGVQLTAKLGYPITDDLDVYTRLGGMVWRADTKAHNNVTGESEKNHDTGVSPVFAGGVEWAITPEIATRLEYQWTNNIGDAHTIGTRPDNGLLSLGVSYRFGQGEAAPVVAPAPAPAPEVQTKHFTLKSDVLFNFNKATLKPEGQAALDQLYSQLSNLDPKDGSVVVLGYTDRIGSDAYNQGLSERRAQSVVDYLISKGIPADKISARGMGESNPVTGNTCDNVKQRAALIDCLAPDRRVEIEVKGIKDVVTQPQA

SEQ ID NO: 197 - protein, 271 aa, Staphylococcus aureus
VKRTEDTSSDKWGVTQNIQFDFVKDKKYNKDALILKMQGFINSKTTYYNYKNTDHIKAMRWPFQYNIGLKTNDPNVDLINYLPKNKIDSVNVSQTLGYNIGGNFNSGPSTGGNGSFNYSKTISYNQQNYISEVEHQNSKSVQWGIKANSFITSLGKMSGHDPNLFVGYKPYSQNPRDYFVPDNELPPLVHSGFNPSFIATVSHEKGSGDTSEFEITYGRKMDVTHATRRTTHYGNSYLEGSRIHNAFVNRNYTVKYEVNWKTHEIKVKGHN

SEQ ID NO: 198 - protein, 331 aa, Neisseria meningitidis
MKKSLIALTLAALPVAAMADVTLYGTIKAGVETSRSVEHNGGQVVSVETGTGIVDLGSKIGFKGQEDLGNGLKAIWQVEQKASIAGTDSGWGNRQSFIGLKGGFGKLRVGRLNSVLKDTGDINPWDSKSDYLGVNKIAEPEARLISVRYDSPEFAGLSGSVQYALNDNAGKYNSESYHAGFNYKNGGFFVQYGGAYKRHVRVDENVNIEKYQIHRLVSGYDNDALHASVAVQQQDAKLVEDNYSHNSQTEVAATLAYRFGNVTPRVSYAHGFKGSFDDADLSNDYDQVVVGAEYDFSKRTSALVSAGWLQEGKGENKFVSTAGGVGLRHKF

SEQ ID NO: 199 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 200 - protein, 495 aa, Mycobacterium tuberculosis
MNQHFDVLIIGAGLSGIGTACHVTAEFPDKTIALLERRERLGGTWDLFRYPGVRSDSDMFTFGYKFRPWRDVKVLADGASIRQYIADTATEFGVDEKIHYGLKVNTAEWSSRQCRWTVAGVHEATGETRTYTCDYLISCTGYYNYDAGYLPDFPGVHRFGGRCVHPQHWPEDLDYSGKKVVVIGSGATAVTLVPAMAGSNPGSAAHVTMLQRSPSYIFSLPAVDKISEVLGRFLPDRWVYEFGRRRNIAIQRKLYQACRRWPKLMRRLLLWEVRRRLGRSVDMSNFTPNYLPWDERLCAVPNGDLFKTLASGAASVVTDQIETFTEKGILCKSGREIEADIIVTATGLNIQMLGGMRLIVDGAEYQLPEKMTYKGVLLENAPNLAWIIGYTNASWTLKSDIAGAYLCRLLRHMADNGYTVATPRDAQDCALDVGMFDQLNSGYVKRGQDIMPRQGSKHPWRVLMHYEKDAKILLEDPIDDGVLHFAAAAQDHAAA

SEQ ID NO: 201 - protein, 370 aa, Fusobacterium nucleatum
MKKLALVLGLLLVVGSVASAKEVMPAPTPAPEKVVEYVEKPVIVYRDREVAPAWRPNGSVDVQYRWYGEVEKKNPKDDKDENWATGKVNAGRLQTLTKVNFTEKQTLEVRTRNHHTLNDTDANNKKSNGAADEYRLRHFYNFGKLGSSKVNATSRVEFKQKTNDGEKSLGASVLFDFADYIYSNNFFKVDKLGLRPGYKYVWKGHGNGEEGTPTVHNEYHLAFESDFTLPFNFALNLEYDLSYNRYREKFETTDGLKKAEWYGELTAVLSNYTPLYKAGAFELGFNAEGGYDTYNMHQYKRIGGEDGTSVDRRDYELYLEPTLQVSYKPTDFVKLYAAAGADYRNRITGESEVKRWRWQPTASAGMKVTF

SEQ ID NO: 202 - protein, 402 aa, Chlamydia trachomatis
MKKLLKSALLFAATGSALSLQALPVGNPAEPSLLIDGTMWEGASGDPCDPCATWCDAISIRAGYYGDYVFDRVLKVDVNKTFSGMAATPTQATGNASNTNQPEANGRPNIAYGRHMQDAEWFSNAAFLALNIWDRFDIFCTLGASNGYFKASSAAFNLVGLIGFSAASSISTDLPMQLPNVGITQGVVEFYTDTSFSWSVGARGALWECGCATLGAEFQYAQSNPKIEMLNVTSSPAQFVIHKPRGYKGASSNFPLPITAGTTEATDTKSATIKYHEWQVGLALSYRLNMLVPYIGVNWSRATFDADTIRIAQPKLKSEILNITTWNPSLIGSTTALPNNSGKDVLSDVLQIASIQINKMKSRKACGVAVGATLIDADKWSITGEARLINERAAHMNAQFRF

SEQ ID NO: 203 - protein, 3590 aa, Bordetella pertussis
MNTNLYRLVFSHVRGMLVPVSEHCTVGNTFCGRTRGQARSGARATSLSVAPNALAWALMLACTGLPLVTHAQGLVPQGQTQVLQGGNKVPVVNIADPNSGGVSHNKFQQFNVANPGVVFNNGLTDGVSRIGGALTKNPNLTRQASAILAEVTDTSPSRLAGTLEVYGKGADLIIANPNGISVNGLSTLNASNLTLTTGRPSVNGGRIGLDVQQGTVTIERGGVNATGLGYFDVVARLVKLQGAVSSKQGKPLADIAVVAGANRYDHATRRATPIAAGARGAAAGAYAIDGTAAGAMYGKHITLVSSDSGLGVRQLGSLSSPSAITVSSQGEIALGDATVQRGPLSLKGAGVVSAGKLASGGGAVNVAGGGAVKIASASSVGNLAVQGGGKVQATLLNAGGTLLVSGRQAVQLGAASSRQALSVNAGGALKADKLSATRRVDVDGKQAVALGSASSNALSVRAGGALKAGKLSATGRLDVDGKQAVTLGSVASDGALSVSAGGNLRANELVSSAQLEVRGQREVALDDASSARGMTVVAAGALAARNLQSKGAIGVQGGEAVSVANANSDAELRVRGRGQVDLHDLSAARGADISGEGRVNIGRARSDSDVKVSAHGALSIDSMTALGAIGVQAGGSVSAKDMRSRGAVTVSGGGAVNLGDVQSDGQVRATSAGAMTVRDVAAAADLALQAGDALQAGFLKSAGAMTVNGRDAVRLDGAHAGGQLRVSSDGQAALGSLAAKGELTVSAARAATVAELKSLDNISVTGGERVSVQSVNSASRVAISAHGALDVGKVSAKSGIGLEGWGAVGADSLGSDGAISVSGRDAVRVDHARSLADISLGAEGGATLGAVEAAGSIDVRGGSTVAANSLHANRDVRVSGKDAVRVTAATSGGGLHVSSGRQLDLGAVQARGALALDGGAGVALQSAKASGTLHVQGGEHLDLGTLAAVGAVDVNGTGDVRVAKLVSDAGADLQAGRSMTLGIVDTTGDLQARAQQKLELGSVKSDGGLQAAAGGALSLAAAEVAGALELSGQGVTVDRASASRARIDSTGSVGIGALKAGAVEAASPRRARRALRQDFFTPGSVVVRAQGNVTVGRGDPHQGVLAQGDIIMDAKGGTLLLRNDALTENGTVTISADSAVLEHSTIESKISQSVLAAKGDKGKPAVSVKVAKKLFLNGTLRAVNDNNETMSGRQIDVVDGRPQITDAVTGEARKDESVVSDAALVADGGPIVVEAGELVSHAGGIGNGRNKENGASVTVRTTGNLVNKGYISAGKQGVLEVGGALTNEFLVGSDGTQRIEAQRIENRGTFQSQAPAGTAGALVVKAAEAIVHDGVMATKGEMQIAGKGGGSPTVTAGAKATTSANKLSVDVASWDNAGSLDIKKGGAQVTVAGRYAEHGEVSIQGDYTVSADAIALAAQVTQRGGAANLTSRHDTRFSNKIRLMGPLQVNAGGPVSNTGNLKVREGVTVTAASFDNETGAEVMAKSATLTTSGAARNAGKMQVKEAATIVAASVSNPGTFTAGKDITVTSRGGFDNEGKMESNKDIVIKTEQFSNGRVLDAKHDLTVTASGQADNRGSLKAGHDFTVQAQRIDNSGTMAAGHDATLKAPHLRNTGQVVAGHDIHIINSAKLENTGRVDARNDIALDVADFTNTGSLYAEHDATLTLAQGTQRDLVVDQDHILPVAEGTLRVKAKSLTTEIETGNPGSLIAEVQENIDNKQAIVVGKDLTLSSAHGNVANEANALLWAAGELTVKAQNITNKRAALIEAGGNARLTAAVALLNKLGRIRAGEDMHLDAPRIENTAKLSGEVQRKGVQDVGGGEHGRWSGIGYVNYWLRAGNGKKAGTIAAPWYGGDLTAEQSLIEVGKDLYLNAGARKDEHRHLLNEGVIQAGGHGHIGGDVDNRSVVRTVSAMEYFKTPLPVSLTALDNRAGLSPATWNFQSTYELLDYLLDQNRYEYIWGLYPTYTEWSVNTLKNLDLGYQAKPAPTAPPMPKAPELDLRGHTLESAEGRKIFGEYKKLQGEYEKAKMAVQAVEAYGEATRRVHDQLGQRYGKALGGMDAETKEVDGIIQEFAADLRTVYAKQADQATIDAETDKVAQRYKSQIDAVRLQAIQPGRVTLAKALSAALGADWRALGHSQLMQRWKDFKAGKRGAEIAFYPKEQTVLAAGAGLTLSNGAIHNGENAAQNRGRPEGLKIGAHSATSVSGSFDALRDVGLEKRLDIDDALAAVLVNPHIFTRIGAAQTSLADGAAGPALARQARQAPETDGMVDARGLGSADALASLASLDAAQGLEVSGRRNAQVADAGLAGPSAVAAPAVGAADVGVEPVTGDQVDQPVVAVGLEQPVATVRVAPPAVALPRPLFETRIKFIDQSKFYGSRYFFEQIGYKPDRAARVAGDNYFDTTLVREQVRRALGGYESRLPVRGVALVAKLMDSAGTVGKALGLKVGVAPTAQQLKQADRDFVWYVDTVIDGQKVLAPRLYLTEATRQGITDQYAGGGALIASGGDVTVNTDGHDVSSVNGLIQGRSVKVDAGKGKVVVADSKGAGGGIEADDEVDVSGRDIGIEGGKLRGKDVRLKADTVKVATSMRYDDKGRLAARGDGALDAQGGQLHIEAKRLETAGATLKGGKVKLDVDDVKLGGVYEAGSSYENKSSTPLGSLFAILSSTTETNQSAHANHYGTRIEAGTLEGKMQNLEIEGGSVDAAHTDLSVARDARFKAAADFAHAEHEKDVRQLSLGAKVGAGGYEAGFSLGSESGLEAHAGRGMTAGAEVKVGYRASHEQSSETEKSYRNANLNFGGGSVEAGNVLDIGGADINRNRYGGAAKGNAGTEEALRMRAKKVESTKYVSEQTSQSSGWSVEVASTASARSSLLTAATRLGDSVAQNVEDGREIRGELMAAQVAAEATQLVTADTAAVALSAGISADFDSSHSRSTSQNTQYLGGNLSIEATEGDATLVGAKFGGGDQVSLKAAKSVNLMAAESTFESYSESHNFHASADANLGANAVQGAVGLGLTAGMGTSHQITNETGKTYAGTSVDAANVSIDAGKDLNLSGSRVRGKHVVLDVEGDINATSKQDERNYNSSGGGWDASAGVAIQNRTLVAPVGSAGFNFNTEHDNSRLTNDGAAGVVASDGLTGHVKGDANLTGATIADLSGKGNLKVDGAVNAQNLKDYRDKDGGSGGLNVGISSTTLAPTVGVAFGRVAGEDYQAEQRATIDVGQTKDPARLQVGGGVKGTLNQDAAQATVVQRNKHWAGGGSEFSVAGKSLKKKNQVRPVETPTPDVVDGPPSRPTTPPASPQPIRATVEVSSPPPVSVATVEVVPRPKVETGSAASASAGGAQVVPVTPPKVEVAKVEVVPRPKVETAQPLPPRPVVAEKVTTPAVQPQLAKVETVQPVKPETTKPLPKPLPVAKVTKAPPPVVETAQPLPPVKPQKATPGPVAEVGKATVTTVQVQSAPPKPAPVAKQPAPAPKPKPKPKPKAERPKPGKTTPLSGRHVVQQQVQVLQRQASDINNTKSLPGGKLPKPVTVKLTDENGKPQTYTINRREDLMKLNGKVLSTKTTLGLEQTFRLRVEDIGGKNYRVFYETNK

SEQ ID NO: 204 - protein, 329 aa, Brucella melitensis
MKFGSKIRRLAVAAVAGAIALGASFAVAQAPTFFRIGTGGTAGTYYPIGGLIANAISGAGEKGVPGLVATAVSSNGSVANINAIKSGALESGFTQSDVAYWAYNGTGLYDGKGKVEDLRLLATLYPETIHIVARKDANIKSVADLKGKRVSLDEPGSGTIVDARIVLEAYGLTEDDIKAEHLKPGPAGERLKDGALDAYFFVGGYPTGAISELAISNGISLVPISGPEADKILEKYSFFSKDVVPAGAYKDVAETPTLAVAAQWVTSAKQPDDLIYNITKVLWNEDTRKALDAGHAKGKLIKLDSATSSLGIPLHPGAERFYKEAGVLK

SEQ ID NO: 205 - protein, 84 aa, synthetic construct
MRSLSVLALLLLLLLAPASAAPGRMKQIEDKIEEILSKIYHIENEIARIKKLIGERGGSGSGGARVGRSLPTEDCENEKEQAVH

SEQ ID NO: 206 - protein, 84 aa, synthetic construct
MRSLSVLALLLLLLLAPASAAPGRMKQIEDKIEEILSKIYHIENEIARIKKLIGERGGSGSGGADYMGWMDFGRRSAEEYEYPS

SEQ ID NO: 207 - protein, 84 aa, synthetic construct
MRSLSVLALLLLLLLAPASAAPGRMKQIEDKIEEILSKIYHIENEIARIKKLIGERGGSGSGGAHQDSRDNCPTVPNSAQQDSD

SEQ ID NO: 208 - protein, 114 aa, synthetic construct
MRSLSVLALLLLLLLAPASAAPGRMKQIEDKIEEILSKIYHIENEIARIKKLIGERGGSGSGGALLAREQQSTGRVGRSLPTEDCENEKEQAVHNVVQLLPGVGTFYNLGTALY

SEQ ID NO: 209 - protein, 114 aa, synthetic construct
MRSLSVLALLLLLLLAPASAAPGRMKQIEDKIEEILSKIYHIENEIARIKKLIGERGGSGSGGASLGAHVAGEAGSKTPGLSRITGLDPVEASFESTPEEVRLDPSDADFVDVI

SEQ ID NO: 210 - protein, 114 aa, synthetic construct
MRSLSVLALLLLLLLAPASAAPGRMKQIEDKIEEILSKIYHIENEIARIKKLIGERGGSGSGGADSDQDKDGDGHQDSRDNCPTVPNSAQQDSDSDGQGDACDEDDDNDGVPDS

SEQ ID NO: 211 - protein, 114 aa, synthetic construct
MRSLSVLALLLLLLLAPASAAPGRMKQIEDKIEEILSKIYHIENEIARIKKLIGERGGSGSGGALAEDGKRCVAVDYCASENHGCEHECVNADGSYLCQCHEGFALNPDKKTCT

SEQ ID NO: 212 - protein, 85 aa, synthetic construct
MRSLSVLALLLLLLLAPASAAPGRMKQIEDKIEEILSKIYHIENEIARIKKLIGERGGSGSGGALLLGPAGARAQEDEDGDYEEL

SEQ ID NO: 213 - protein, 112 aa, synthetic construct
MRSLSVLALLLLLLLAPASAAPGRMKQLEDKIEELLSKIYHLENEIARLKKLIGERGGSGSGGASKTPGLSRITGLDPVEASFESTPEEVRLDPSDADFVDVIHTDAAPLIP

SEQ ID NO: 214 - protein, 64 aa, synthetic construct
MRSLSVLALLLLLLLAPASAAPGRMKQLEDKIEELLSKIYHLENEIARLKKLIGERGGSGSGGA

SEQ ID NO: 215 - protein, 100 aa, synthetic construct
MRSLSVLALLLLLLLAPASAAPGRMKQLEDKIEELLSKIYHLENEIARLKKLIGERGGSGSGGANLPEEEEEEEEEEEEERKGLLPGALGSALQGPAQVS

SEQ ID NO: 216 - protein, 114 aa, synthetic construct
MRSLSVLALLLLLLLAPASAAPGRMKQLEDKIEELLSKIYHLENEIARLKKLIGERGGSGSGGAQVLIQHLRGLQKGRSTERNVSVEALASALQLLAREQQSTGRVGRSLPTED

SEQ ID NO: 217 - protein, 95 aa, synthetic construct
MRSLSVLALLLLLLLAPASAAPGRMKQLEDKIEELLSKIYHLENEIARLKKLIGERGGSGSGGAQVLIQHLRGLQKGRSTERNVSRVGRSLPTED

SEQ ID NO: 218 - protein, 12 aa, synthetic construct
SGLRINSAKDDA

SEQ ID NO: 219 - skipped

SEQ ID NO: 220 - protein, 6 aa, synthetic construct
EDADYA

SEQ ID NO: 221 - protein, 6 aa, synthetic construct
LVPRGS

SEQ ID NO: 222 - protein, 6 aa, synthetic construct
HHHHHH

SEQ ID NO: 223 - protein, 20 aa, synthetic construct
MRSLSVLALLLLLLLAPASA

SEQ ID NO: 224 - protein, 33 aa, synthetic construct
RMKQIEDKIEEILSKIYHIENEIARIKKLIGER

SEQ ID NO: 225 - protein, 33 aa, synthetic construct
RMKQLEDKIEELLSKIYHLENEIARLKKLIGER

SEQ ID NO: 226 - DNA, 777 bp, synthetic construct
  source 1..777: mol_type = other DNA
atgcgcagcctgagcgtgctggccctgctgctgctcctgctcctggcccctgcttctgccagtggcttgaggattaatagcgctaaggacgacgctgcgggtcaggctgccgctaaccgcgctacatccaatatcaaagggcttacccaagcctcaaggaacgctgccgacggcatttcaatagcccagactactgaaggcgcccttaacgaaatcaataacaatctgcagagggtgagagaactgagtgtacaggcgacggctggcgccaatgcagatgctgctctgaaggccattcaggcggagatccagcagcgcctggaagagattgatagagtgtctcaacagacccaggctgctgccgttaaggtgctttcacaggataatgctatggccatccaggttggagctaacgatggagccgctatcactattgacctccagaaaatcgacgttaaatctctgggactcgatgggttcaatgttaactcccctggatcaacagccaaccctctggcatctatcgactcagccttgagcaaggtggatgcggtgagaagcagcttgggtgctatccagaataggtttgatagtgccattacgaacttgggtaacacagtcaccaacttgaatagcgcacggtcccgcattgaggatgctgattacgccacggaagtaagccagatgtccaaggctcagattctggaccaggctggaacaagcacactggcgcagctggttccgcgaggaagtcaccaccatcaccaccatggataa

(19) United States
(12) Patent Application Publication
(10) Pub. No.: US 2025/0161438 A1
(43) Pub. Date: May 22, 2025

(54) RNA ADJUVANTS, METHODS AND USES THEREOF

(71) Applicant: Flag Bio, Inc., East Aurora, NY (US)

(72) Inventors: Ekaterina L. Andrianova, East Aurora, NY (US); Ilya Gitlin, East Aurora, NY (US)

(21) Appl. No.: 18/963,077

(22) Filed: Nov. 27, 2024

Related U.S. Application Data
(63) Continuation of application No. PCT/US2023/067691, filed on May 31, 2023.
(60) Provisional application No. 63/347,635, filed on Jun. 1, 2022.

(51) Int. Cl. A61K 39/39; A61K 39/00; A61K 39/215; C07K 14/195

(52) U.S. Cl. CPC A61K 39/39; A61K 39/215; C07K 14/195; A61K 2039/53; A61K 2039/55516; A61K 2039/6018

(57) ABSTRACT

Provided herein are adjuvants, compositions, and methods for the prevention and treatment of infectious diseases and cancer. Various toll-like receptor (TLR) agonists and RNA encoding TLR agonists are provided herein. The adjuvants provided herein can be complexed with a carrier or formulated with a delivery vehicle for administration to a subject. Further provided are adjuvants that can be delivered with vaccine compositions or as part of a vaccine composition to enhance the innate immune response in a subject.

### CROSS REFERENCE TO RELATED APPLICATIONS

This application is a continuation of International Application No. PCT/US23/67691, filed May 31, 2023, which claims priority to U.S. Provisional Patent Application No. 63/347,635, filed Jun. 1, 2022, the contents of both are entirely incorporated herein by reference for all purposes.

### SEQUENCE LISTING

This application incorporates by reference a Sequence Listing XML submitted via the USPTO patent electronic filing system. The Sequence Listing XML, entitled 207481-701601_SL.xml, was created on May 31, 2023, and is 427,686 bytes in size.

### BACKGROUND

RNA vaccines have emerged as a promising therapeutic for preventing the spread of infectious diseases, such as COVID-19. However, RNA vaccines are costly, require a high dose of RNA per vaccine, and lack efficacy long term for infectious disease prevention. Furthermore, RNA is translated using host cell machinery that is modulated by the interferon system which can reduce the amount of RNA translated in vivo. Furthermore, current vaccine compositions can cause unwanted side effects. Therefore, there is a need for adjuvants that can reduce RNA vaccine side effects, optimize RNA dosage in RNA vaccines, and promote innate and adaptive immune responses that enhance RNA vaccine efficacy.

### BRIEF SUMMARY

Provided herein are compositions for inducing an immune response following administration to a subject, the compositions comprising: (a) a nucleic acid (e.g., an RNA a DNA, or an RNA adjuvant) encoding a synthetic protein, wherein the synthetic protein comprises at least a functional fragment of a toll-like receptor 5 (TLR5) agonist that is capable of activating a TLR5 pathway in a cell upon contact with the cell; and (b) one or more delivery vehicle formulated for intranasal administration, oral administration, intradermal administration, intranodal administration, intrasplenic administration, intratumoral administration, intravenous administration, or intramuscular administration, wherein following administration of an effective amount of the composition to a subject in the presence of an antigen, an initial innate immune response is induced in the subject.

Provided herein are compositions, wherein the compositions comprise: (a) a carrier (e.g., a lipid carrier); and (b) an RNA sequence encoding at least a functional fragment of a bacterial motility protein, wherein the functional fragment of the bacterial motility protein is a TLR5 agonist. In some embodiments, the functional fragment activates a TLR5 pathway in a cell upon contact with a cell.

Provided herein are compositions for inducing an immune response following administration to a subject, the compositions comprising: (a) a nucleic acid (e.g., an RNA a DNA, or an RNA adjuvant) encoding a synthetic protein, wherein the synthetic protein comprises at least a functional fragment of a toll-like receptor 4 (TLR4) agonist that is capable of activating a TLR4 pathway in a cell upon contact with the cell; and (b) one or more delivery vehicle formulated for intranasal administration, oral administration, intradermal administration, intranodal administration, intrasplenic administration, intratumoral administration, intravenous administration, or intramuscular administration, wherein following administration of an effective amount of the composition to a subject in the presence of an antigen, an initial innate immune response is induced in the subject.

Provided herein are compositions, wherein the compositions comprise: (a) a carrier (e.g., a lipid carrier); and (b) one or more RNA sequences, wherein the one or more RNA sequences encode for at least a functional fragment of a TLR4 agonist, wherein the functional fragment of the TLR4 agonist is capable of activating a TLR4 pathway in a cell upon contact with the cell.

Provided herein are compositions for inducing an immune response following administration to a subject, the compositions comprising: (a) a nucleic acid (e.g., an RNA a DNA, or an RNA adjuvant) encoding a synthetic protein, wherein the synthetic protein comprises at least a functional fragment of a toll-like receptor 2 (TLR2) agonist that is capable of activating a TLR2 pathway in a cell upon contact with the cell; and (b) one or more delivery vehicle formulated for intranasal administration, oral administration, intradermal administration, intranodal administration, intrasplenic administration, intratumoral administration, intravenous administration, or intramuscular administration, wherein following administration of an effective amount of the composition to a subject in the presence of an antigen, an initial innate immune response is induced in the subject.

Provided herein are compositions, wherein the compositions comprise: (a) a carrier (e.g., a lipid carrier); and (b) one or more RNA sequences, wherein the one or more RNA sequences encode for at least a functional fragment of a TLR2 agonist, wherein the functional fragment activates a TLR2 pathway in a cell upon contact with a cell.

Provided herein are compositions for inducing an immune response upon administration to a subject, the compositions comprising: (a) one or more carriers (e.g., lipid carriers); (b) one or more nucleic acids (e.g., RNA or DNA), wherein the one or more nucleic acids encode for: (i) at least a functional fragment of a TLR4 agonist, a TLR2 agonist, or a TLR5 agonist, or any combination thereof; (ii) at least one RNA encoding a viral antigen; or (iii) a combination of (i) and (ii); and (c) a delivery vehicle, wherein following the administration of an effective amount of the composition, an innate immune response is induced in the subject in the presence of the viral antigen, thereby inducing an immune response to the viral antigen. In some embodiments, the delivery vehicle is formulated for intranasal administration, oral administration, intradermal administration, intranodal administration, intrasplenic administration, intratumoral administration, intravenous administration, or intramuscular administration.

Provided herein are compositions, wherein the compositions comprise: (a) an RNA adjuvant comprising a nucleic acid sequence selected from any one of SEQ ID NOS: 2 to 175; and (b) a delivery vehicle, wherein the delivery vehicle is selected from a diluent, an aqueous solution, a particle, an emulsion, a suspension, a lipid carrier, a vesicle, a yeast cell, or any combination thereof.

Provided herein are compositions, wherein the compositions comprise: (a) a DNA sequence that encodes for an RNA adjuvant comprising a nucleic acid sequence selected from any one of SEQ ID NOS: 2 to 175; and (b) a delivery vehicle, wherein the delivery vehicle is selected from a diluent, an aqueous solution, a particle, an emulsion, a suspension, a lipid carrier, a vesicle, a yeast cell, or any combination thereof.

Provided herein are compositions, wherein the compositions comprise: (a) a nucleic acid encoding for a synthetic protein comprising an amino acid sequence selected from any one of SEQ ID NOS: 176 to 217; and (b) a delivery vehicle, wherein the delivery vehicle is selected from a diluent, an aqueous solution, a particle, an emulsion, a suspension, a lipid carrier, a vesicle, a yeast cell, or any combination thereof. Further provided herein are nucleic acids encoding an amino acid sequence that is at least 80% identical to SEQ ID NO: 206. Further provided herein are nucleic acids encoding an amino acid sequence that is at least 80% identical to SEQ ID NO: 210. Further provided herein are nucleic acids encoding an amino acid sequence that is at least 75% identical to SEQ ID NO: 212. Further provided herein are nucleic acids encoding an amino acid sequence that is at least 85% identical to SEQ ID NO: 213. Further provided herein are nucleic acids encoding an amino acid sequence that is at least 75% identical to SEQ ID NO: 215. Further provided herein are nucleic acids encoding an amino acid sequence that is at least 75% identical to SEQ ID NO: 217.

Provided herein are compositions, wherein the compositions comprise: (a) a synthetic protein comprising an amino acid sequence selected from any one of SEQ ID NOS: 176 to 217; and (b) a delivery vehicle, wherein the delivery vehicle is selected from a diluent, an aqueous solution, a particle, an emulsion, a suspension, a lipid carrier, a vesicle, a yeast cell, or any combination thereof.

Provided herein are compositions, wherein the compositions comprise: a synthetic protein comprising at least one protein construct, wherein the at least one protein construct comprises an amino acid sequence selected from SEQ ID NOS: 176 to 217. Further provided herein are compositions comprising two or more, three or more, four or more, or five or more protein constructs.

Provided herein are compositions, wherein the compositions comprise: a nucleic acid encoding for a synthetic protein comprising at least one protein construct, wherein the at least one protein construct comprises an amino acid sequence selected from SEQ ID NOS: 176 to 217. Further provided herein are compositions comprising two or more, three or more, four or more, or five or more protein constructs.

Provided herein are compositions, wherein the compositions comprise: (a) a yeast cell, wherein the yeast cell comprises a permeable cell wall; and (b) one or more nucleic acids (e.g., DNA, RNA, or a combination thereof), wherein the one or more nucleic acid encode for at least a functional fragment of a TLR4 agonist, a TLR2 agonist, a TLR5 agonist, or a combination thereof, wherein the functional fragment activates a TLR4, a TLR5, or a TLR2 pathway in a cell upon contact with the cell.

Provided herein are compositions for inducing an immune response upon administration to a subject, wherein the compositions comprise: (a) one or more carriers (e.g., lipid carriers); (b) one or more nucleic acids, wherein the one or more nucleic acids encode for: (i) at least a functional fragment of a TLR4 agonist, a TLR2 agonist, a TLR5 agonist, or any combination thereof; (ii) a tumor antigen; (iii) an immune regulatory protein; or (iv) a combination thereof; and (c) a delivery vehicle, wherein following the administration of an effective amount of the composition, an innate immune response is induced in the subject in the presence of the tumor antigen, thereby inducing an immune response to the tumor antigen. In some embodiments, the functional fragment activates a TLR4, a TLR5, or a TLR2 pathway in a cell upon contact with the cell.

Provided herein are vectors, wherein the vectors comprise: one or more nucleic acids (e.g., DNA, RNA or a combination thereof) encoding: a TLR5 agonist, a TLR4 agonist, a TLR2 agonist, or a combination thereof.

Provided herein are compositions, wherein the compositions comprise a vector provided herein; and a carrier (e.g., a lipid carrier).

Provided herein are vaccine compositions, wherein the vaccine compositions comprise a composition provided herein; and a pharmaceutically acceptable excipient.

Provided herein are vaccine compositions, wherein the vaccine compositions comprise: (a) a lipid carrier; (b) one or more RNA adjuvants, wherein the one or more RNA adjuvants comprise a sequence that is at least 85% identical to the sequence of any one of SEQ ID NOS: 2 to 175; and (c) one or more RNA sequence encoding for an antigen. Further provided herein are vaccine compositions, wherein the one or more RNA sequence encoding for an antigen each encode a viral antigen, a bacterial antigen, a fungal antigen, a parasitic antigen, a tumor antigen, or any combination thereof.

Provided herein are vaccine compositions, wherein the vaccine compositions comprise: (a) a lipid carrier; (b) one or more nucleic acids encoding an RNA adjuvant provided herein, wherein the one or more RNA adjuvant comprises a sequence that is at least 85% identical to the sequence of any one of SEQ ID NOS: 2 to 175; and (c) one or more nucleic acids encoding an antigen. Further provided herein are vaccine compositions, wherein the one or more nucleic acids encoding an antigen each encode a viral antigen, a bacterial antigen, a fungal antigen, a parasitic antigen, a tumor antigen, or any combination thereof. In some embodiments, the one or more nucleic acids comprise DNA. In some embodiments, the one or more nucleic acids comprise RNA. In some embodiments, the one or more nucleic acids encoding for the RNA adjuvant are DNA, RNA, or a combination thereof.

Provided herein are methods stimulating an immune response in a subject, the methods comprising: administering to the subject an effective amount of a composition provided herein, a vector provided herein, or a vaccine composition provided herein, thereby stimulating the immune response. Provided herein are methods of enhancing an immune response to an antigen encoded by a nucleic acid (e.g., an RNA) in an vaccine composition (e.g., an RNA vaccine), the method comprising: (a) administering to the subject an effective amount of a composition provided herein or the vaccine composition provided herein; and (b) administering to the subject an RNA vaccine composition, wherein the RNA vaccine composition comprises an RNA encoding for an antigen, thereby enhancing the immune response to the antigen encoded by the RNA. Provided herein are methods for the treatment of an infection in a subject, the methods comprising: administering to a subject an effective amount of a composition provided herein, a vector provided herein, or a vaccine composition provided herein, thereby treating an infection in the subject.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic providing the mechanism of action of an RNA adjuvant provided herein.

FIGS. 2A-2D are schematics demonstrating several configurations of the RNA adjuvant and the antigen-encoding RNA sequences. FIG. 2A shows a schematic of configuration 1. FIG. 2B shows a schematic of configuration 2. FIG. 2C shows a schematic of configuration 3. FIG. 2D shows a schematic of configuration 4.

FIGS. 3A-3C show schematics and graphs that demonstrate the effects of an RNA encoded TLR5 agonist (SEQ ID NO: 2, SEQ ID NO: 176) on HEK293 cells with a nuclear factor-kappa B (NF-κB)-luciferase reporter and HEK293 cells without the reporter. FIG. 3A shows a schematic of the experimental scheme of cell transfection and cellular conditions. FIG. 3B shows a graph of luciferase activity (RLU) driven by NF-κB activation over time (0, 18, and 40 hours post-transfection). Y-axis: Luciferase activity; X-axis: Hours post-transfection for each condition as indicated. FIG. 3C shows a graph of TLR5 agonist concentration over time (0, 18, and 40 hours post-transfection). Y-axis: TLR5 agonist concentration (nanograms per milliliter); X-axis: Hours post-transfection for each condition as indicated.

FIGS. 4A-4B show graphs of the effect of lipid nanoparticle (LNP)-encapsulated toll-like receptor agonist RNA on packaging and NF-κB signaling. FIG. 4A shows a schematic of the experimental scheme and a graph of luciferase activity over time for HEK293 cells treated with TLR5 Agonist 1 RNA encapsulated in a lipid nanoparticle (LNP), TLR5 Agonist Protein alone, or a control LNP(GFP). Luciferase activity driven by NF-κB activation was measured at various time points up to 48 hours post-transfection. Y-axis: Luciferase activity (RLU); X-axis: Hours after treatment with LNP(TLR5 Agonist 1 RNA), TLR5 Agonist 1 protein, or LNP(GFP). FIG. 4B shows a schematic of the experimental scheme and a graph of luciferase activity for secreted TLR5 agonist protein from cell media and cell culture relative to LNPs without RNA encoding a TLR5 agonist. Y-axis (left): Luciferase activity (RLU); Y-axis (right): Fluorescent signals (RFU); X-axis: Hours after treatment with TLR5 Agonist 1 Protein or LNP(TLR5 Agonist 1 RNA).

FIGS. 5A-5B show schematics and graphs of TLR5 Agonist 1 effects on HEK293-hTLR5-Luc and HEK293-null-Luc cells. FIG. 5A shows a schematic of the experimental scheme and a graph of TLR5 agonist protein release after transduction of LNP+TLR5 agonist into HEK293 cells with a human TLR5-luciferase reporter relative to HEK293 cells that do not express a TLR5. Cells were sampled up to 23 hours post-delivery of the LNP+TLR5 agonist. Y-axis: Conditions as indicated; X-axis: Luciferase activity (RLU). FIG. 5B shows a graph of TLR5 Agonist 1 protein release after transduction of LNP(TLR5 Agonist 1) into HEK293-hTLR5-Luc and HEK293-null-Luc cells. Y-axis: TLR5 agonist protein (nanograms per milliliter); X-axis: Hours after treatment.

FIG. 6 show luminescence images of the effects of LNP(mRNA TLR5 agonist) in vivo in BALB/c-Tg(IκBα-luc)Xen NF-κB luciferase reporter mice. Mice were administered LNPs encapsulating mRNA encoding TLR5 agonist 1, PBS, or LNPs with a GFP reported alone. Mice treated with LNP(TLR5 Agonist 1 RNA) had the highest levels of luminescence relative to the other conditions 5 hours after treatment.

FIG. 7 shows a graph of the dynamic of response to LNP(mRNA encoding TLR5 agonist 1) in vivo (NF-κB reporter mice) at 0, 3, 5, 6, 8, 10, 12, 15, 18, and 36 hours post-injection. From left to right, bars show LNP(TLR5 Agonist 1); LNP(GFP); and PBS controls. Y-axis: Total luminescence; X-axis: Hours post-injection.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION OF THE INVENTION

Provided herein are RNA adjuvant compositions and vaccine compositions, methods, and uses thereof for inducing an immune response to an antigen for the prevention of an infectious disease and cancer treatment. Briefly, further described herein are (1) RNA adjuvant compositions; (2) nucleic acids encoding for antigens; (3) lipid carriers; (4) yeast cell compositions; (5) pharmaceutical vaccine compositions, dosing, and administration; and (6) therapeutic applications.

### Definitions

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document. All references disclosed herein, including patent references and non-patent references, are hereby incorporated by reference in their entirety as if each was incorporated individually. However, where a patent, patent application, or publication containing express definitions is incorporated by reference, those express definitions should be understood to apply to the incorporated patent, patent application, or publication in which they are found, and not necessarily to the text of this application, in particular the claims of this application, in which instance, the definitions provided herein are meant to supersede.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as anon-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, “optional” or “optionally” means that the subsequently described circumstance may or may not occur, so that the description includes instances where the circumstance occurs and instances where it does not.

As used herein, the term “about” or “approximately” means a range of up to ±20%, of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “effective amount” or “therapeutically effective amount” refers to an amount that is sufficient to achieve or at least partially achieve the desired effect.

### (1) RNA Adjuvant Compositions

Provided herein are compositions comprising nucleic acids. Further provided herein are compositions comprising an RNA adjuvant. Further provided herein are compositions (e.g., pharmaceutical compositions and vaccine compositions), wherein the vaccine composition comprises a nucleic acid (e.g., an RNA adjuvant provided herein) and nucleic acid (e.g., an RNA) encoding for an antigen. In some embodiments, the nucleic acids provided herein encode for a protein that activates a toll-like receptor (TLR). In some embodiments, the nucleic acids activate the TLR. TLRs are largely classified into two subfamilies based on their localization, cell surface TLRs and intracellular TLRs. Cell surface TLRs include TLR1, TLR2, TLR4, TLR5, TLR6, and TLR10, whereas intracellular TLRs are localized in the endosome and include TLR3, TLR7, TLR8, TLR9, TLR11, TLR12, and TLR13. Cell surface TLRs generally recognize microbial membrane components such as lipids, lipoproteins, and proteins. For example, TLR4 recognizes bacterial lipopolysaccharide (LPS). TLR2 along with TLR1 or TLR6 recognizes a wide variety of PAMPs including lipoproteins, peptidoglycans, lipoteichoic acids, zymosan, mannan, and tGPI-mucin. TLR5 recognizes bacterial flagellin proteins. The human TLR10 collaborates with TLR2 to recognize ligands from Listeria. TLR10 can also sense influenza A virus infections.

TLRs recognize pathogen-associated molecular patterns (PAMPs) derived from microbial organisms and signal through the recruitment of specific adaptor molecules, leading to activation of the transcription factors (e.g., NF-κB), which modulates the innate immune response to the PAMP or a microbial organism expressing a PAMP. The innate immune response is the first line of defense to an intruding pathogen. The innate immune response is antigen-independent or a non-specific defense mechanism that is used by the host immediately or within hours of encountering an antigen. The innate immune response has no immunologic memory and, therefore, it is unable to recognize or “memorize” the same pathogen should the body be exposed to it in the future. Adaptive immunity, on the other hand, is antigen-dependent and antigen-specific and, therefore, involves a lag time between exposure to the antigen and maximal response. The hallmark of adaptive immunity is the capacity for memory which enables the host to mount a more rapid and efficient immune response upon subsequent exposure to the antigen. Innate and adaptive immunity are not mutually exclusive mechanisms of host defense, but rather are complementary, with defects in either system resulting in host vulnerability or inappropriate responses.

The advantage of the compositions provided herein is that the RNA adjuvants enhance innate immune signaling pathways via TLR signaling while promoting the adaptive immune response. This effect can lower the effective amount of RNA needed to encode an antigen in vaccine compositions, reducing the dose needed for mRNA encoding antigens as compared with vaccine compositions that do not comprise an mRNA adjuvant. mRNA adjuvants provided herein activate nuclear factor-kappa B (NF-κB) signaling downstream TLRs which is necessary to stimulate T-cell activation. TLR5-mediated activation of NF-κB, unlike other TLRs, considerably less involve the production of tumor necrosis factor alpha (TNF-α) and interleukin 1 beta (IL-1β) due to a unique tissue specificity of TLR5 expression that is largely limited to epithelial and endothelial cells and does not involve T-cells and macrophages. This contributes to safety of mRNA vaccines combined with TLR5 agonist-based adjuvants as TNF-α and IL-1β can result in negative side effects, e.g., septic shock, fever, and irritation at the administration site. Furthermore, the mRNA adjuvants provided herein attract and promote adaptive immune responses targeted to an antigen and long-term memory of pathogens by the immune system.

The nucleic acids provided herein (e.g., RNA adjuvants) encode for a TLR agonist or a functional fragment thereof. The functional fragment is capable of activating a TLR pathway or a TLR in a cell upon contact with a cell. In some embodiments, the TLR agonist binds directly to the TLR. In some embodiments, the TLR agonist or the functional fragment thereof is a naturally-occurring TLR agonist, protein, or nucleic acid. In some embodiments, the TLR agonist or the functional fragment thereof is a non-naturally-occurring TLR agonist, protein, or nucleic acid. In some embodiments, the non-naturally occurring TLR agonist is an RNA sequence encoding a TLR agonist protein. In some embodiments, the TLR agonist or the functional fragment thereof is a synthetic protein (also called an engineered protein). A synthetic protein provided herein is non-naturally occurring and can be engineered using known methods, e.g., molecular cloning techniques. In some embodiments, a composition provided herein comprises a plurality of mRNA adjuvants provided herein. In some embodiments, the mRNA adjuvants provided herein activate a TLR2; a TLR4; and/or a TLR5.

In some embodiments, the activation of a TLR provided herein increases the level or activity of NF-κB. NF-κB is a transcription factor that modulates cellular responses to infectious agents and acts as a mediator of innate and adaptive immune reactions. NF-κB also mediates the production of proinflammatory cytokines.

In some embodiments, a composition provided herein comprises at least a functional fragment of a TLR5 agonist, wherein the TLR agonist comprises a functional fragment of a bacterial motility protein. In some embodiments, the bacterial motility protein is a bacterial flagellin protein, a derivative, or a functional fragment thereof. In some embodiments, the bacterial motility protein provided herein is a truncated form of a bacterial flagellin protein, a secreted form of a bacterial flagellin protein, or a combination thereof. Flagellin contains two to four structural domains. The common D0 and D1 domains are buried in the core of the flagellar filament by mediating inter-flagellin interactions and are conserved among bacterial species due to their functional importance in filament formation. The flagellin D1 domain possesses a common molecular pattern for the TLR5 interaction, despite the differences in the sequences and domains between flagellins. The D0 and D1 domains stimulate TLR5 activation. In three- and four-domain flagellins, the D1 domain is extended to ancillary domains (D2 and D3) located on the surface of the flagellar filament. In contrast to D0 and D1, the D2 or D3 domains exhibit substantial variation in sequence and structure and are considered to activate adaptive immunity. Many Gram-positive bacteria, such as, e.g., Bacillus subtilis and Clostridium difficile, express flagellin that lacks the hypervariable domains and thus contains the minimal regions (D0 and D1 domains) necessary for TLR5 activation and flagellin polymerization into the flagellar filament. Surprisingly, not all bacterial flagellins activate TLR5. For example, flagellins from Campylobacter jejuni and Helicobacter pylori that belong to c-proteobacteria do not induce TLR5 signaling and can escape from TLR5-mediated immune surveillance. The main functional difference between TLR5-activating flagellins and non-activators can be ascribed to sequence variations.

A protein provided herein (e.g., a synthetic protein or a protein encoded by a nucleic acid provided herein) can comprise two (D0 and D1), three (D0, D1, and D2), or four (D0, D1, D2, and D3) domains from a bacterial flagellin. Provided herein are compositions, wherein the functional fragment of a toll-like receptor 5 (TLR5) agonist comprises a D0 region and a D1 region of the bacterial flagellin protein. In some embodiments, the functional fragment of a toll-like receptor 5 (TLR5) agonist comprises a D0 region, a D1 region, and a D2 region of the bacterial flagellin protein. In some embodiments, the functional fragment of a toll-like receptor 5 (TLR5) agonist comprises a D0 region, a D1 region, a D2 region, and a D3 region of the bacterial flagellin protein.

In some embodiments, a composition provided herein comprises at least a functional fragment of a TLR4 agonist. In some embodiments, the TLR4 agonist comprises at least a functional fragment of a high mobility group box 1 (HMGB1) protein, a nicotinate-nucleotide—dimethylbenzimidazole phosphoribosyltransferase, a transglycosylase, a 50S ribosomal protein, a heparin-binding hemagglutinin protein, a YadA family autotransporter adhesin, a dnaJ protein, a pneumolysin protein, an Omp19 protein, a 6,7-dimethyl-8-ribityllumazine synthase, or any combination thereof. In some embodiments, the functional fragment activates a TLR4 pathway in a cell upon contact with a cell.

In some embodiments, a composition provided herein comprises at least a functional fragment of a TLR2 agonist. In some embodiments, the TLR2 agonist comprises at least a functional fragment of an outer membrane protein (Omp), a Panton-Valentine bi-component leukocidin (PVL) protein, a porin protein, a TRAP transporter protein, a hemagglutinin protein, an oxidoreductase protein, or a type VII secretion system. In some embodiments, the functional fragment activates a TLR2 pathway in a cell upon contact with a cell.

Proteins encoded by nucleic acids provided herein (e.g., DNA sequences, RNA sequences, or RNA adjuvants) can be designed from the amino acid sequence of any species of bacteria or mammal that encodes immunostimulatory pathways. Synthetic proteins provided herein can be a chimeric protein comprising fragments of a protein derived from two or more different organisms to form a functional TLR agonist that activates a TLR receptor, e.g., the human TLR5, human TLR4, or human TLR2.

The proteins encoded by the RNA adjuvants or nucleic acids provided herein can comprise modifications that improve the stability, targeting, or function of the protein. In some embodiments, the proteins comprise a signal peptide for targeted cellular localization. In some embodiments, the proteins provided herein further comprise a signal sequence of: MRSLSVLALLLLLLLAPASA (SEQ ID NO: 1). In some embodiments, the proteins provided herein do not comprise a signal sequence. In some embodiments, the proteins provided herein further comprise an N-terminal spoke region. In some embodiments, the N-terminal spoke region comprises a sequence of: SGLRINSAKDDA (SEQ ID NO: 218). In some embodiments, the proteins provided herein further comprise a linker. A linker is a molecular entity that can directly or indirectly connect at two parts of a composition, e.g., a first protein construct and a second protein construct. Linkers can be configured according to a specific need, e.g., stability or length between the first and second protein constructs. As another example, linkers can be configured to have a sufficient length and flexibility. In some embodiments, linkers can be configured to facilitate expression and purification of the protein provided herein. In some embodiments, the linker comprises a sequence of: SPG. In some embodiments, the protein comprises a C-terminal spoke region. In some embodiments, the C-terminal spoke region comprises the sequence of: EDADYA (SEQ ID NO: 220). In some embodiments, the proteins provided herein comprise a thrombin cleavage site. In some embodiments, the thrombin cleavage site comprises the sequence of: LVPRGS (SEQ ID NO: 221). In some embodiments, the proteins provided herein comprise a peptide tag. In some embodiments, the peptide tag is a Histidine (His) tag (e.g., comprising the sequence: HHHHHH (SEQ ID NO: 222).

A composition provided herein can comprise any one of the RNA adjuvants or a sequence listed in Table 1, derivatives, variants, or functional fragments thereof. A composition provided herein can also comprise a DNA sequence that encodes for an RNA adjuvant or an amino acid sequence listed in Table 1, derivatives, variants, or functional fragments thereof. In some embodiments, the RNA adjuvant provided herein encodes for a synthetic protein and/or a TLR agonist, derivative, or a functional fragment thereof comprising at least 75%, 80%, 85%, 90%, 95%, 96%, 97%, 98%, or 99% sequence similarity to a sequence listed Table 1. In some embodiments, the nucleic acid sequences or the amino acid sequences provided herein comprise a sequence that is at least 75%, 80%, 85%, 90%, 95%, 96%, 97%, 98%, 99% or 100% sequence similarity to a sequence listed Table 1. In some embodiments, the RNA sequences provided herein comprises a sequence identical to a sequence listed in Table 1 or encodes for an amino acid sequence that is identical to a sequence listed in Table 1. Percent (%) sequence identity (or similarity) for a given sequence relative to a reference sequence is defined as the percentage of identical residues identified after aligning the two sequences and introducing gaps if necessary, to achieve the maximum percent sequence identity. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

In some embodiments, a composition provided herein comprises a sequence listed in Table 1; and further comprises one or more sequence modifications. In some embodiments, a sequence provided herein is a codon-optimized sequence. Sequence modification(s) can include, e.g., a substitution, a deletion, an insertion, a chemical modification of one or more nucleobases; or chemical modifications to the phosphate backbone, a nucleotide, a nucleobase, or a nucleoside. Such modifications can be made to the RNA adjuvant sequence, a DNA sequence that encodes an RNA adjuvant sequence provided herein, or any sequence disclosed herein (e.g., mRNA encoding an antigen). Methods of modifying a nucleic acid or amino acid sequence are known. One of ordinary skill in the art will appreciate that the modification(s) may be located at any position(s) of a nucleic acid such that the function of the nucleic acid or synthetic protein is not substantially decreased. For example, software can be used to match identical or similar sequences by assigning degrees of homology to various substitutions, deletions, and/or other modifications. Nucleic acids provided herein can be prepared according to any available technique including, but not limited to chemical synthesis, enzymatic synthesis, which is generally termed in vitro-transcription, cloning, enzymatic, or chemical cleavage, etc. In some cases, the nucleic acids provided herein are not uniformly modified along the entire length of the molecule. Different nucleotide modifications and/or backbone structures can exist at various positions within the nucleic acid.

TABLE 1
RNA Adjuvants.

| | RNA | | Target |
|---|---|---|---|
| RNA Adjuvant | SEQ ID NOS: | Amino Acid Sequence (SEQ ID NO:) | TLR |
| TLR5 Agonist 1 | SEQ ID NOS: | MRSLSVLALLLLLLLAPASASGLRINSAKDDAAGQAAAN | TLR5 |
| | 2-7 | RATSNIKGLTQASRNAADGISIAQTTEGALNEINNNLQRV | |
| | | RELSVQATAGANADAALKAIQAEIQQRLEEIDRVSQQTQ | |
| | | AAAVKVLSQDNAMAIQVGANDGAAITIDLQKIDVKSLGL | |
| | | DGFNVNSPGSTANPLASIDSALSKVDAVRSSLGAIQNRFD | |
| | | SAITNLGNTVTNLNSARSRIEDADYATEVSQMSKAQILD | |
| | | QAGTSTLAQLVPRGSHHHHHHG (SEQ ID NO: 176) | |
| TLR5 Agonist 2 | SEQ ID NOS: | MSGLRINSAKDDAAGQAAANRATSNIKGLTQASRNAAD | TLR5 |
| | 8-13 | GISIAQTTEGALNEINNNLQRVRELSVQATAGANADAAL | |
| | | KAIQAEIQQRLEEIDRVSQQTQAAAVKVLSQDNAMAIQV | |
| | | GANDGAAITIDLQKIDVKSLGLDGFNVNSPGSTANPLASI | |
| | | DSALSKVDAVRSSLGAIQNRFDSAITNLGNTVTNLNSARS | |
| | | RIEDADYATEVSQMSKAQILDQAGTSTLAQLVPRGSHHH | |
| | | HHHG (SEQ ID NO: 177) | |
| TLR5 Agonist 3 | SEQ ID NOS: | MSGLRINSAKDDAAGQAIANRFTSNIKGLTQASRNAADG | TLR5 |
| | 14-19 | ISIAQTTEGALNEINNNLQRVRELSVQATAGANADAALK | |
| | | AIQAEIQQRLEEIDRVSQQTQAAAVKVLSQDNAMAIQVG | |
| | | ANDGAAITIDLQKIDVKSLGLDGFNVNSPGNPLASIDSAL | |
| | | SKVDAVRSSLGAIQNRFDSAITNLGNTVTNLNSARSIEDA | |
| | | DYATEVSQMSKAQILQQAGTSVLAQANQVPQNVLSLLV | |
| | | PRGSHHHHHHG (SEQ ID NO: 178) | |
| TLR5 Agonist 4 | SEQ ID NOS: | MRGSHHHHHHGMASMTGGQQMGRDLYDDDDKDPMA | TLR5 |
| | 20-25 | QVINTNSLSLLTQNNLNKSQSSLSSAIERLSSGLRINSAKD | |
| | | DAAGQAIANRFTSNIKGLTQASRNANDGISIAQTTEGALN | |
| | | EINNNLQRVRELSVQATNGTNSDSDLKSIQDEIQQRLEEI | |
| | | DRVSNQTQFNGVKVLSQDNQMKIQVGANDGETITIDLQ | |
| | | KIDVKSLGLDGFNVN (SEQ ID NO: 179) | |
| TLR5 Agonist 5 | SEQ ID NOS: | MSGLRINSAKDDAAGQAAANRATSNIKGLTQASRNAAD | TLR5 |
| | 26-31 | GISIAQTTEGALNEINNNLQRVRELSVQATAGANADAAL | |
| | | KAIQAEIQQRLEEIDRVSQQTQAAAVKVLSQDNAMAIQV | |
| | | GANDGAAITIDLQKIDVKSLGLDGFNVNSPGSTANPLASI | |
| | | DSALSKVDAVRSSLGAIQNRFDSAITNLGNTVTNLNSARS | |
| | | RIEDADYATEVSQMSKAQILDQAGTSTLAQLVPRGSHHH | |
| | | HHHG (SEQ ID NO: 180) | |
| TLR5 Agonist 6 | SEQ ID NOS: | MRSLSVLALLLLLLLAPASASGLRINSAKDDAAGQAAAN | TLR5 |
| | 32-37 | RATSNIKGLTQASRNAADGISIAQTTEGALNEINNNLQRV | |
| | | RELSVQATAGANADAALKAIQAEIQQRLEEIDRVSQQTQ | |
| | | AAAVKVLSQDNAMAIQVGANDGAAITIDLQKIDVKSLGL | |
| | | DGFNVNSPGSTANPLASIDSALSKVDAVRSSLGAIQNRFD | |
| | | SAITNLGNTVTNLNSARSRIEDADYATEVSQMSKAQILD | |
| | | QAGTSTLAQLVPRGSHHHHHHG (SEQ ID NO: 181) | |
| TLR4 Agonist 1 | SEQ ID NOS: | MNQSCPNKTSFKIAFIQARWHADIVDEARKSFVAELAAK | TLR4 |
| 6,7-dimethy1-8- | 38-43 | TGGSVEVEIFDVPGAYEIPLHAKTLARTGRYAAIVGAAFV | |
| ribityllumazine | | IDGGIYRHDFVATAVINGMMQVQLETEVPVLSVVLTPHH | |
| synthase 2 | | FHESKEHHDFFHAHFKVKGVEAAHAALQIVSERSRIAAL | |
| | | V (SEQ ID NO: 182) | |
| TLR4 Agonist 2 | SEQ ID NOS: | MRRIQSIARSPIAIALFMSLAVAGCASKKNLPNNAGDLGL | TLR4 |
| Peptidoglycan- | 44-49 | GAGAATPGSSQDFTVNVGDRIFFDLDSSLIRADAQQTLSK | |
| associated protein | | QAQWLQRYPQYSITIEGHADERGTREYNLALGQRRAAA | |
| | | TRDFLASRGVPTNRMRTISYGNERPVAVCDADTCWSQN | |
| | | RRAVTVLNGAGR (SEQ ID NO: 183) | |
| TLR4 Agonist 3 | SEQ ID NOS: | MGISKASLLSLAAAGIVLAGCQSSRLGNLDNVSPPPPPAP | TLR4 |
| Outer membrane | 50-55 | VNAVPAGTVQKGNLDSPTQFPNAPSTDMSAQSGTQVAS | |
| lipoprotein omp19 | | LPPASAPDLTPGAVAGVWNASLGGQSCKIATPQTKYGQ | |
| | | GYRAGPLRCPGELANLASWAVNGKQLVLYDANGGTVA | |
| | | SLYSSGQGRFDGQTTGGQAVTLSR (SEQ ID NO: 184) | |
| TLR4 Agonist 4 | SEQ ID NOS: | MEFATVSPPDPGTAAAARARQDTLTKPRGALGRLEDLSV | TLR4 |
| Nicotinate- | 56-61 | WIAACQGQCPPRQFERARVVVFAGDHGVARCGVSAYPP | |
| nucleotide-- | | EVTAQMVANFDAGGAAINALAGVAGASVRVADLAVDA | |
| dimethylbenzimid | | DPPDDRIGAHKVRRGSGDITVQDALTAEETERALSAGAA | |
| azole | | IADEEVDAGADLLIAGDMGIGNTTAAAVLVAALTNVEPV | |
| phosphoribosyl- | | VAVGFGTGIDDAGWARKTAAVRDALFRARRVLPDPVAL | |
| transferase | | LRCAGGADLAALAGFCAQAAVRRTPLLLDGMAVTAAA | |
| | | LVAEHLAPGARLWWQAGHRSTEPGHALALTALDLEPIL | |
| | | DLRMRLGEGTGAALALPIVRAAVAALSSMATFAQAGVS | |
| | | DPSAHP (SEQ ID NO: 185) | |
| TLR4 Agonist 5 | SEQ ID NOS: | MRNVRKTLILAAVTGTLVTIPSATAHADAEQVGFDPNVP | TLR4 |
| Resuscitation- | 62-67 | AAGPAPDAPPPPAPDAPPAPAPDAPPAPDAPPPPAPDAPP | |
| promoting factor | | PAPDAPPAPGPDVPPPPAPKAYSVNWDAIAQCESGGNWG | |
| RpfE | | INTGNGYAGGLQFTSSTWHANGGSGSPAGASREEQIRVA | |
| | | ENVLHSQGIGAWPVCGRRG (SEQ ID NO: 186) | |
| TLR4 Agonist 6 | SEQ ID NOS: | MAKLSTDELLDAFKEMTLLELSDFVKKFEETFEVTAAAP | TLR4 |
| 50S ribosomal | 68-73 | VAVAAAGAAPAGAAVEAAEEQSEFDVILEAAGDKKIGVI | |
| protein L7/ | | KVVREIVSGLGLKEAKDLVDGAPKPLLEKVAKEAADEA | |
| L12RpIL (SA1) | | KAKLEAAGATVTVK (SEQ ID NO: 187) | |
| TLR4 Agonist 7 | SEQ ID NOS: | MAENPNIDDLPAPLLAALGAADLALATVNDLIANLRERA | TLR4 |
| Heparin-binding | 74-79 | EETRAETRTRVEERRARLTKFQEDLPEQFIELRDKFTTEE | |
| hemagglutinin | | LRKAAEGYLEAATNRYNELVERGEAALQRLRSQTAFED | |
| | | ASARAEGYVDQAVELTQEALGTVASQTRAVGERAAKLV | |
| | | GIELPGKAEAAGKKAQKAIAKAPAKKASAKKAPAKKAP | |
| | | AKKAAAKKVTQK (SEQ ID NO: 188) | |
| TLR4 Agonist 8 | SEQ ID NOS: | MNKIYRIIWNSALNAWVVVSELTRNHTKRASATVATAV | TLR4 |
| Chaperone protein | 80-85 | LATLLFATVQANATDDDDLYLEPVQRTAVVLSFRSDKEG | |
| DnaJ | | TGEKEGTEDSNWAVYFDEKRVLKAGAITLKAGDNLKIK | |
| | | QNTNENTNDSSFTYSLKKDLTDLTSVETEKLSFGANGNK | |
| | | VNITSDTKGLNFAKETAGTNGDPTVHLNGIGSTLTDTLLN | |
| | | TGATTNVTNDNVTDDEKKRAASVKDVLNAGWNIKGVK | |
| | | PGTTASDNVDFVRTYDTVEFLSADTKTTTVNVESKDNGK | |
| | | KTEVKIGAKTSVIKEKDGKLVTGKGKDENGSSTDEGEGL | |
| | | VTAKEVIDAVNKAGWRMKTTTANGQTGQADKFETVTS | |
| | | GTNVTFASGKGTTATVSKDDQGNITVKYDVNVGDALNV | |
| | | NQLQNSGWNLDSKAVAGSSGKVISGNVSPSKGKMDETV | |
| | | NINAGNNIEITRNGKNIDIATSMAPQFSSVSLGAGADAPT | |
| | | LSVDDEGALNVGSKDTNKPVRITNVAPGVKEGDVTNVA | |
| | | QLKGVAQNLNNRIDNVDGNARAGIAQAIATAGLVQAYL | |
| | | PGKSMMAIGGDTYRGEAGYAIGYSSISDGGNWIIKGTAS | |
| | | GNSRGHFGASASVGYQW (SEQ ID NO: 189) | |
| TLR4 Agonist 9 | SEQ ID NOS: | MNNTEFYDRLGVSKNASADEIKKAYRKLSKKYHPDINK | TLR4 |
| Streptococcus | 86-91 | EPGAEDKYKEVQEAYETLSDDQKRAAYDQYGAAGANG | |
| pneumoniae DnaJ | | GFGGAGGFGGFNGAGGFGGFEDIFSSFFGGGGSSRNPNA | |
| | | PRQGDDLQYRVNLTFEEAIFGTEKEVKYHREAGCRTCNG | |
| | | SGAKPGTSPVTCGRCHGAGVINVDTQTPLGMMRRQVTC | |
| | | DVCHGRGKEIKYPCTTCHGTGHEKQAHSVHVKIPAGVET | |
| | | GQQIRLAGQGEAGFNGGPYGDLYVVVSVEASDKFEREG | |
| | | TTIFYNLNLNFVQAALGDTVDIPTVHGDVELVIPEGTQTG | |
| | | KKFRLRSKGAPSLRGGAVGDQYVTVNVVTPTGLNDRQK | |
| | | VALKEFAAAGDLKVNPKKKGFFDHIKDAFDGE (SEQ ID | |
| | | NO: 190) | |
| TLR4 Agonist 10 | SEQ ID NOS: | MANKAVNDFILAMNYDKKKLLTHQGESIENRFIKEGNQL | TLR4 |
| Pneumolysin | 92-97 | PDEFVVIERKKRSLSTNTSDISVTATNDSRLYPGALLVVD | |
| | | ETLLENNPTLLAVDRAPMTYSIDLPGLASSDSFLQVEDPS | |
| | | NSSVRGAVNDLLAKWHQDYGQVNNVPARMQYEKITAH | |
| | | SMEQLKVKFGSDFEKTGNSLDIDFNSVHSGEKQIQIVNFK | |
| | | QIYYTVSVDAVKNPGDVFQDTVTVEDLKQRGISAERPLV | |
| | | YISSVAYGRQVYLKLETTSKSDEVEAAFEALIKGVKVAP | |
| | | QTEWKQILDNTEVKAVILGGDPSSGARVVTGKVDMVED | |
| | | LIQEGSRFTADHPGLPISYTTSFLRDNVVATFQNSTDYVE | |
| | | TKVTAYRNGDLLLDHSGAYVAQYYITWDELSYDHQGKE | |
| | | VLTPKAWDRNGQDLTAHFTTSIPLKGNVRNLSVKIRECT | |
| | | GLAWEWWRTVYEKTDLPLVRKRTISIWGTTLYPQVEDK | |
| | | VEND (SEQ ID NO: 191) | |
| TLR4 Agonist 11 | SEQ ID NOS: | MGKGDPKKPRGKMSSYAFFVQTCREEHKKKHPDASVNF | TLR4 |
| High Mobility | 98-103 | SEFSKKCSERWKTMSAKEKGKFEDMAKADKARYEREM | |
| Group Protein B1 | | KTYIPPKGETKKKFKDPNAPKRPPSAFFLFCSEYRPKIKGE | |
| | | HPGLSIGDVAKKLGEMWNNTAADDKQPYEKKAAKLKE | |
| | | KYEKDIAAYRAKGKPDAAKKGVVKAEKSKKKKEEEEDE | |
| | | EDEEDEEEEEDEEDEDEEEDDDDE (SEQ ID NO: 192) | |
| TLR2 Agonist 1 | SEQ ID NOS: | MNKTLIALAVSAAAVATGAYADGINQSGDKAGSTVYSA | TLR2 |
| Vibrio cholerae | 104-109 | KGTSLEVGGRAEARLSLKDGKAQDNSRVRLNFLGKAEIN | |
| OmpU | | DSLYGVGFYEGEFTTNDQGKNASNNSLDNRYTYAGIGG | |
| | | TYGEVTYGKNDGALGVITDFTDIMSYHGNTAAEKIAVA | |
| | | DRVDNMLAYKGQFGDLGVKASYRFADRNAVDAMGNV | |
| | | VTETNAAKYSDNGEDGYSLSAIYTFGDTGFNVGAGYAD | |
| | | QDDQNEYMLAASYRMENLYFAGLFTDGELAKDVDYTG | |
| | | YELAAGYKLGQAAFTATYNNAETAKETSADNFAIDATY | |
| | | YFKPNFRSYISYQFNLLDSDKVGKVASEDELAIGLRYDF | |
| | | (SEQ ID NO: 193) | |
| TLR2 Agonist 2 | SEQ ID NOS: | MTRYQDDFYDAINGEWQQTAEIPADKSQTGGFVDLDQEI | TLR2 |
| Streptococcus | 110-115 | EDLMLATTDKWLAGEEVPEDAILENFVKYHRLVRDFDK | |
| pneumoniae PepO | | READGITPVLPLLKEFQELETFADFTAKLAEFELAGKPNF | |
| | | LPFGVSPDFMDARINVLWASAPSTILPDTTYYAEEHPQRE | |
| | | ELLTLWKESSANLLKAYDFSDEEIEDLLEKRLELDRRVA | |
| | | AVVLSNEESSEYAKLYHPYSYEDFKKFAPALPLDDFFKA | |
| | | VIGQLPDKVIVDEERFWQAAEQFYSEEAWSLLKATLILSV | |
| | | VNLSTSYLTEDIRVLSGAYSRALSGVPEAKDKVKAAYHL | |
| | | AQEPFKQALGLWYAREKFSPEAKADVEKKVATMIDVYK | |
| | | ERLLKNDWLTPETCKQAIVKLNVIKPYIGYPEELPARYKD | |
| | | KVVNETASLFENALAFARVEIKHSWSKWNQPVDYKEWG | |
| | | MPAHMVNAYYNPQKNLIVFPAAILQAPFYDLHQSSSANY | |
| | | GGIGAVIAHEISHAFDTNGASFDENGSLKDWWTESDYAA | |
| | | FKEKTQKVIDQFDGQDSYGATINGKLTVSENVADLGGIA | |
| | | AALEAAKREADFSAEEFFYNFGRIWRMKGRPEFMKLLAS | |
| | | VDVHAPAKLRVNVQVPNFDDFFTTYDVKEGDGMWRSP | |
| | | EERVIIW (SEQ ID NO: 194) | |
| TLR2 Agonist 3 | SEQ ID NOS: | MKKTAIAIAVALAGFATVAQAAPKDNTWYTGAKLGWS | TLR2 |
| Shigella flexneri | 116-121 | QYHDTGFIPNNGPTHENQLGAGAFGGYQVNPYVGFEMG | |
| OmpA | | YDWLGRMPYKGDNINGAYKAQGVQLTAKLGYPITDDL | |
| | | DIYTRLGGMVWRADTKANVPGGASFKDHDTGVSPVFAG | |
| | | GVEYAITPEIATRLEYQWTNNIGDANTIGTRPDNGLLSLG | |
| | | VSYRFGQGEAAPVVAPAPAPEVQTKHFTLKSDVLFNFNK | |
| | | ATLKPEGQAALDQLYSQLSNLDPKDGSVVVLGYTDRIGS | |
| | | DAYNQGLSERRAQSVVDYLISKGIPADKISARGMGESNP | |
| | | VTGNTCDNVKQRAALIDCLAPDRRVEIEVKGIKDVVTQP | |
| | | QA (SEQ ID NO: 195) | |
| TLR2 Agonist 4 | SEQ ID NOS: | MKKTAIAITVALAGFATVAQAAPKDNTWYTGAKLGWS | TLR2 |
| Shigella | 122-127 | QYHDTGFIDNNGPTHENQLGAGAFGGYQVNPYVGFEMG | |
| dysenteriae Porin | | YDWLGRMPYKGSVENGAYKAQGVQLTAKLGYPITDDL | |
| | | DVYTRLGGMVWRADTKAHNNVTGESEKNHDTGVSPVF | |
| | | AGGVEWAITPEIATRLEYQWTNNIGDAHTIGTRPDNGLL | |
| | | SLGVSYRFGQGEAAPVVAPAPAPAPEVQTKHFTLKSDVL | |
| | | FNFNKATLKPEGQAALDQLYSQLSNLDPKDGSVVVLGY | |
| | | TDRIGSDAYNQGLSERRAQSVVDYLISKGIPADKISARGM | |
| | | GESNPVTGNTCDNVKQRAALIDCLAPDRRVEIEVKGIKD | |
| | | VVTQPQA (SEQ ID NO: 196) | |
| TLR2 Agonist 5 | SEQ ID NOS: | VKRTEDTSSDKWGVTQNIQFDFVKDKKYNKDALILKMQ | TLR2 |
| Staphylococcus | 128-133 | GFINSKTTYYNYKNTDHIKAMRWPFQYNIGLKTNDPNV | |
| aureus PVL | | DLINYLPKNKIDSVNVSQTLGYNIGGNFNSGPSTGGNGSF | |
| | | NYSKTISYNQQNYISEVEHQNSKSVQWGIKANSFITSLGK | |
| | | MSGHDPNLFVGYKPYSQNPRDYFVPDNELPPLVHSGFNP | |
| | | SFIATVSHEKGSGDTSEFEITYGRKMDVTHATRRTTHYG | |
| | | NSYLEGSRIHNAFVNRNYTVKYEVNWKTHEIKVKGHN | |
| | | (SEQ ID NO: 197) | |
| TLR2 Agonist 6 | SEQ ID NOS: | MKKSLIALTLAALPVAAMADVTLYGTIKAGVETSRSVEH | TLR2 |
| Neisseria | 134-139 | NGGQVVSVETGTGIVDLGSKIGFKGQEDLGNGLKAIWQ | |
| meningitidis PorB | | VEQKASIAGTDSGWGNRQSFIGLKGGFGKLRVGRLNSVL | |
| | | KDTGDINPWDSKSDYLGVNKIAEPEARLISVRYDSPEFAG | |
| | | LSGSVQYALNDNAGKYNSESYHAGFNYKNGGFFVQYG | |
| | | GAYKRHVRVDENVNIEKYQIHRLVSGYDNDALHASVAV | |
| | | QQQDAKLVEDNYSHNSQTEVAATLAYRFGNVTPRVSYA | |
| | | HGFKGSFDDADLSNDYDQVVVGAEYDFSKRTSALVSAG | |
| | | WLQEGKGENKFVSTAGGVGLRHKF (SEQ ID NO: 198) | |
| TLR2 Agonist 7 | SEQ ID NOS: | MTEQQWNFAGIEAAASAIQGNVTSIHSLLDEGKQSLTKL | TLR2 |
| Mycobacterium | 140-145 | AAAWGGSGSEAYQGVQQKWDATATELNNALQNLARTI | |
| tuberculosis | | SEAGQAMASTEGNVTGMFA (SEQ ID NO: 199) | |
| ESAT6 | | | |
| TLR2 Agonist 8 | SEQ ID NOS: | MNQHFDVLIIGAGLSGIGTACHVTAEFPDKTIALLERRER | TLR2 |
| Mycobacterium | 146-151 | LGGTWDLFRYPGVRSDSDMFTFGYKFRPWRDVKVLAD | |
| tuberculosis | | GASIRQYIADTATEFGVDEKIHYGLKVNTAEWSSRQCRW | |
| MymA (Rv3083) | | TVAGVHEATGETRTYTCDYLISCTGYYNYDAGYLPDFPG | |
| | | VHRFGGRCVHPQHWPEDLDYSGKKVVVIGSGATAVTLV | |
| | | PAMAGSNPGSAAHVTMLQRSPSYIFSLPAVDKISEVLGRF | |
| | | LPDRWVYEFGRRRNIAIQRKLYQACRRWPKLMRRLLLW | |
| | | EVRRRLGRSVDMSNFTPNYLPWDERLCAVPNGDLFKTL | |
| | | ASGAASVVTDQIETFTEKGILCKSGREIEADIIVTATGLNI | |
| | | QMLGGMRLIVDGAEYQLPEKMTYKGVLLENAPNLAWII | |
| | | GYTNASWTLKSDIAGAYLCRLLRHMADNGYTVATPRDA | |
| | | QDCALDVGMFDQLNSGYVKRGQDIMPRQGSKHPWRVL | |
| | | MHYEKDAKILLEDPIDDGVLHFAAAAQDHAAA (SEQ ID | |
| | | NO: 200) | |
| TLR2 Agonist 9 | SEQ ID NOS: | MKKLALVLGLLLVVGSVASAKEVMPAPTPAPEKVVEYV | TLR2 |
| Fusobacterium | 152-157 | EKPVIVYRDREVAPAWRPNGSVDVQYRWYGEVEKKNP | |
| nucleatum FomA | | KDDKDENWATGKVNAGRLQTLTKVNFTEKQTLEVRTR | |
| | | NHHTLNDTDANNKKSNGAADEYRLRHFYNFGKLGSSKV | |
| | | NATSRVEFKQKTNDGEKSLGASVLFDFADYIYSNNFFKV | |
| | | DKLGLRPGYKYVWKGHGNGEEGTPTVHNEYHLAFESDF | |
| | | TLPFNFALNLEYDLSYNRYREKFETTDGLKKAEWYGELT | |
| | | AVLSNYTPLYKAGAFELGFNAEGGYDTYNMHQYKRIGG | |
| | | EDGTSVDRRDYELYLEPTLQVSYKPTDFVKLYAAAGAD | |
| | | YRNRITGESEVKRWRWQPTASAGMKVTF (SEQ ID NO: | |
| | | 201) | |
| TLR2 Agonist 10 | SEQ ID NOS: | MKKLLKSALLFAATGSALSLQALPVGNPAEPSLLIDGTM | TLR2 |
| Chlamydia | 158-163 | WEGASGDPCDPCATWCDAISIRAGYYGDYVFDRVLKVD | |
| trachomatis | | VNKTFSGMAATPTQATGNASNTNQPEANGRPNIAYGRH | |
| MOMP | | MQDAEWFSNAAFLALNIWDRFDIFCTLGASNGYFKASSA | |
| | | AFNLVGLIGFSAASSISTDLPMQLPNVGITQGVVEFYTDT | |
| | | SFSWSVGARGALWECGCATLGAEFQYAQSNPKIEMLNV | |
| | | TSSPAQFVIHKPRGYKGASSNFPLPITAGTTEATDTKSATI | |
| | | KYHEWQVGLALSYRLNMLVPYIGVNWSRATFDADTIRI | |
| | | AQPKLKSEILNITTWNPSLIGSTTALPNNSGKDVLSDVLQI | |
| | | ASIQINKMKSRKACGVAVGATLIDADKWSITGEARLINE | |
| | | RAAHMNAQFRF (SEQ ID NO: 202) | |
| TLR2 Agonist 11 | SEQ ID NOS: | MNTNLYRLVFSHVRGMLVPVSEHCTVGNTFCGRTRGQA | TLR2 |
| Bordetella | 164-169 | RSGARATSLSVAPNALAWALMLACTGLPLVTHAQGLVP | |
| pertussis FHA | | QGQTQVLQGGNKVPVVNIADPNSGGVSHNKFQQFNVAN | |
| | | PGVVFNNGLTDGVSRIGGALTKNPNLTRQASAILAEVTD | |
| | | TSPSRLAGTLEVYGKGADLIIANPNGISVNGLSTLNASNL | |
| | | TLTTGRPSVNGGRIGLDVQQGTVTIERGGVNATGLGYFD | |
| | | VVARLVKLQGAVSSKQGKPLADIAVVAGANRYDHATRR | |
| | | ATPIAAGARGAAAGAYAIDGTAAGAMYGKHITLVSSDS | |
| | | GLGVRQLGSLSSPSAITVSSQGEIALGDATVQRGPLSLKG | |
| | | AGVVSAGKLASGGGAVNVAGGGAVKIASASSVGNLAV | |
| | | QGGGKVQATLLNAGGTLLVSGRQAVQLGAASSRQALSV | |
| | | NAGGALKADKLSATRRVDVDGKQAVALGSASSNALSVR | |
| | | AGGALKAGKLSATGRLDVDGKQAVTLGSVASDGALSVS | |
| | | AGGNLRANELVSSAQLEVRGQREVALDDASSARGMTVV | |
| | | AAGALAARNLQSKGAIGVQGGEAVSVANANSDAELRVR | |
| | | GRGQVDLHDLSAARGADISGEGRVNIGRARSDSDVKVS | |
| | | AHGALSIDSMTALGAIGVQAGGSVSAKDMRSRGAVTVS | |
| | | GGGAVNLGDVQSDGQVRATSAGAMTVRDVAAAADLAL | |
| | | QAGDALQAGFLKSAGAMTVNGRDAVRLDGAHAGGQLR | |
| | | VSSDGQAALGSLAAKGELTVSAARAATVAELKSLDNISV | |
| | | TGGERVSVQSVNSASRVAISAHGALDVGKVSAKSGIGLE | |
| | | GWGAVGADSLGSDGAISVSGRDAVRVDHARSLADISLG | |
| | | AEGGATLGAVEAAGSIDVRGGSTVAANSLHANRDVRVS | |
| | | GKDAVRVTAATSGGGLHVSSGRQLDLGAVQARGALAL | |
| | | DGGAGVALQSAKASGTLHVQGGEHLDLGTLAAVGAVD | |
| | | VNGTGDVRVAKLVSDAGADLQAGRSMTLGIVDTTGDLQ | |
| | | ARAQQKLELGSVKSDGGLQAAAGGALSLAAAEVAGALE | |
| | | LSGQGVTVDRASASRARIDSTGSVGIGALKAGAVEAASP | |
| | | RRARRALRQDFFTPGSVVVRAQGNVTVGRGDPHQGVLA | |
| | | QGDIIMDAKGGTLLLRNDALTENGTVTISADSAVLEHSTI | |
| | | ESKISQSVLAAKGDKGKPAVSVKVAKKLFLNGTLRAVN | |
| | | DNNETMSGRQIDVVDGRPQITDAVTGEARKDESVVSDA | |
| | | ALVADGGPIVVEAGELVSHAGGIGNGRNKENGASVTVR | |
| | | TTGNLVNKGYISAGKQGVLEVGGALTNEFLVGSDGTQRI | |
| | | EAQRIENRGTFQSQAPAGTAGALVVKAAEAIVHDGVMA | |
| | | TKGEMQIAGKGGGSPTVTAGAKATTSANKLSVDVASWD | |
| | | NAGSLDIKKGGAQVTVAGRYAEHGEVSIQGDYTVSADAI | |
| | | ALAAQVTQRGGAANLTSRHDTRFSNKIRLMGPLQVNAG | |
| | | GPVSNTGNLKVREGVTVTAASFDNETGAEVMAKSATLT | |
| | | TSGAARNAGKMQVKEAATIVAASVSNPGTFTAGKDITVT | |
| | | SRGGFDNEGKMESNKDIVIKTEQFSNGRVLDAKHDLTVT | |
| | | ASGQADNRGSLKAGHDFTVQAQRIDNSGTMAAGHDATL | |
| | | KAPHLRNTGQVVAGHDIHIINSAKLENTGRVDARNDIAL | |
| | | DVADFTNTGSLYAEHDATLTLAQGTQRDLVVDQDHILP | |
| | | VAEGTLRVKAKSLTTEIETGNPGSLIAEVQENIDNKQAIV | |
| | | VGKDLTLSSAHGNVANEANALLWAAGELTVKAQNITNK | |
| | | RAALIEAGGNARLTAAVALLNKLGRIRAGEDMHLDAPRI | |
| | | ENTAKLSGEVQRKGVQDVGGGEHGRWSGIGYVNYWLR | |
| | | AGNGKKAGTIAAPWYGGDLTAEQSLIEVGKDLYLNAGA | |
| | | RKDEHRHLLNEGVIQAGGHGHIGGDVDNRSVVRTVSAM | |
| | | EYFKTPLPVSLTALDNRAGLSPATWNFQSTYELLDYLLD | |
| | | QNRYEYIWGLYPTYTEWSVNTLKNLDLGYQAKPAPTAP | |
| | | PMPKAPELDLRGHTLESAEGRKIFGEYKKLQGEYEKAK | |
| | | MAVQAVEAYGEATRRVHDQLGQRYGKALGGMDAETK | |
| | | EVDGIIQEFAADLRTVYAKQADQATIDAETDKVAQRYKS | |
| | | QIDAVRLQAIQPGRVTLAKALSAALGADWRALGHSQLM | |
| | | QRWKDFKAGKRGAEIAFYPKEQTVLAAGAGLTLSNGAI | |
| | | HNGENAAQNRGRPEGLKIGAHSATSVSGSFDALRDVGLE | |
| | | KRLDIDDALAAVLVNPHIFTRIGAAQTSLADGAAGPALA | |
| | | RQARQAPETDGMVDARGLGSADALASLASLDAAQGLEV | |
| | | SGRRNAQVADAGLAGPSAVAAPAVGAADVGVEPVTGD | |
| | | QVDQPVVAVGLEQPVATVRVAPPAVALPRPLFETRIKFID | |
| | | QSKFYGSRYFFEQIGYKPDRAARVAGDNYFDTTLVREQV | |
| | | RRALGGYESRLPVRGVALVAKLMDSAGTVGKALGLKV | |
| | | GVAPTAQQLKQADRDFVWYVDTVIDGQKVLAPRLYLTE | |
| | | ATRQGITDQYAGGGALIASGGDVTVNTDGHDVSSVNGLI | |
| | | QGRSVKVDAGKGKVVVADSKGAGGGIEADDEVDVSGR | |
| | | DIGIEGGKLRGKDVRLKADTVKVATSMRYDDKGRLAAR | |
| | | GDGALDAQGGQLHIEAKRLETAGATLKGGKVKLDVDD | |
| | | VKLGGVYEAGSSYENKSSTPLGSLFAILSSTTETNQSAHA | |
| | | NHYGTRIEAGTLEGKMQNLEIEGGSVDAAHTDLSVARD | |
| | | ARFKAAADFAHAEHEKDVRQLSLGAKVGAGGYEAGFSL | |
| | | GSESGLEAHAGRGMTAGAEVKVGYRASHEQSSETEKSY | |
| | | RNANLNFGGGSVEAGNVLDIGGADINRNRYGGAAKGNA | |
| | | GTEEALRMRAKKVESTKYVSEQTSQSSGWSVEVASTAS | |
| | | ARSSLLTAATRLGDSVAQNVEDGREIRGELMAAQVAAE | |
| | | ATQLVTADTAAVALSAGISADFDSSHSRSTSQNTQYLGG | |
| | | NLSIEATEGDATLVGAKFGGGDQVSLKAAKSVNLMAAE | |
| | | STFESYSESHNFHASADANLGANAVQGAVGLGLTAGMG | |
| | | TSHQITNETGKTYAGTSVDAANVSIDAGKDLNLSGSRVR | |
| | | GKHVVLDVEGDINATSKQDERNYNSSGGGWDASAGVAI | |
| | | QNRTLVAPVGSAGFNFNTEHDNSRLTNDGAAGVVASDG | |
| | | LTGHVKGDANLTGATIADLSGKGNLKVDGAVNAQNLK | |
| | | DYRDKDGGSGGLNVGISSTTLAPTVGVAFGRVAGEDYQ | |
| | | AEQRATIDVGQTKDPARLQVGGGVKGTLNQDAAQATV | |
| | | VQRNKHWAGGGSEFSVAGKSLKKKNQVRPVETPTPDVV | |
| | | DGPPSRPTTPPASPQPIRATVEVSSPPPVSVATVEVVPRPK | |
| | | VETGSAASASAGGAQVVPVTPPKVEVAKVEVVPRPKVE | |
| | | TAQPLPPRPVVAEKVTTPAVQPQLAKVETVQPVKPETTK | |
| | | PLPKPLPVAKVTKAPPPVVETAQPLPPVKPQKATPGPVAE | |
| | | VGKATVTTVQVQSAPPKPAPVAKQPAPAPKPKPKPKPKA | |
| | | ERPKPGKTTPLSGRHVVQQQVQVLQRQASDINNTKSLPG | |
| | | GKLPKPVTVKLTDENGKPQTYTINRREDLMKLNGK VLST | |
| | | KTTLGLEQTFRLRVEDIGGKNYRVFYETNK (SEQ ID NO: | |
| | | 203) | |
| TLR2 Agonist 12 | SEQ ID NOS: | MKFGSKIRRLAVAAVAGAIALGASFAVAQAPTFFRIGTG | TLR2 |
| Brucella abortus | 170-175 | GTAGTYYPIGGLIANAISGAGEKGVPGLVATAVSSNGSV | |
| BCSP31 | | ANINAIKSGALESGFTQSDVAYWAYNGTGLYDGKGKVE | |
| | | DLRLLATLYPETIHIVARKDANIKSVADLKGKRVSLDEPG | |
| | | SGTIVDARIVLEAYGLTEDDIKAEHLKPGPAGERLKDGAL | |
| | | DAYFFVGGYPTGAISELAISNGISLVPISGPEADKILEKYSF | |
| | | FSKDVVPAGAYKDVAETPTLAVAAQWVTSAKQPDDLIY | |
| | | NITKVLWNEDTRKALDAGHAKGKLIKLDSATSSLGIPLH | |
| | | PGAERFYKEAGVLK (SEQ ID NO: 204) | |

In some embodiments, the nucleic acids provided herein (e.g., RNA adjuvants) comprise a sequence that is at least 85% identical to any one of SEQ ID NOS: 2 to 175. In some embodiments, the nucleic acids provided herein (e.g., RNA adjuvants) comprise a sequence that is at least 90% identical to any one of SEQ ID NOS: 2 to 175. In some embodiments, the nucleic acids provided herein (e.g., RNA adjuvants) comprise a sequence that is at least 95% identical to any one of SEQ ID NOS: 2 to 175. In some embodiments, the nucleic acids provided herein (e.g., RNA adjuvants) comprise a sequence selected from any one of SEQ ID NOS: 2 to 175. In some embodiments, the nucleic acids provided herein (e.g., RNA adjuvants) comprise SEQ ID NO: 2, a variant, or a functional fragment thereof. In some embodiments, the nucleic acids provided herein (e.g., RNA adjuvants) comprise SEQ ID NO: 62, a variant, or a functional fragment thereof. In some embodiments, the nucleic acids provided herein (e.g., RNA adjuvants) comprise SEQ ID NO: 98, a variant, or a functional fragment thereof.

In some embodiments, the nucleic acids provided herein (e.g., RNA adjuvants) encode for an amino acid sequence that is at least 75% identical to a sequence selected from any one of SEQ ID NOS: 176 to 204. In some embodiments, the nucleic acids provided herein (e.g., RNA adjuvants or DNA) encode for any one of SEQ ID NO: 176, SEQ ID NO: 181, SEQ ID NO: 200, SEQ ID NO: 201, SEQ ID NO: 202, a variant, or a functional fragment thereof. In some embodiments, a composition provided herein comprises a protein comprising an amino acid sequence that is at least 75%, at least 80%, at least 85%, at least 90%, at least 95%, at least 96%, at least 97%, at least 98%, at least 99%, or 10000 identical to any one of SEQ ID NOS: 176 to 204. In some embodiments, a composition provided herein comprises a protein comprising an amino acid sequence that is at least 85% identical to any one of SEQ ID NO: 176, SEQ ID NO: 181, SEQ ID NO: 200, SEQ ID NO: 201, SEQ ID NO: 202. In some embodiments, a composition provided herein comprises a nucleic acid encoding a comprising an amino acid sequence that is at least 85% identical to any one of SEQ ID NO: 176, SEQ ID NO: 181, SEQ ID NO: 200, SEQ ID NO: 201, SEQ ID NO: 202.

Further provided herein are compositions comprising at least one protein construct. In some embodiments, the composition comprises two or more protein constructs (e.g., dimers). In some embodiments, the composition comprises three or more protein constructs (e.g., trimers). In some embodiments, the composition further comprises a linker between each protein construct. In some embodiments, the composition comprises one or more protein construct selected from Table 2.

TABLE 2
Protein Constructs.

| | |
|---|---|
| ApoF-T20 | MRSLSVLALLLLLLLAPASAAPGRMKQIEDKIEEILSKIYHIENEIARIKKLIGERGGS |
| | GSGGARVGRSLPTEDCENEKEQAVH (SEQ ID NO: 205) |
| CCK-T20 | MRSLSVLALLLLLLLAPASAAPGRMKQIEDKIEEILSKIYHIENEIARIKKLIGERGGS |
| | GSGGADYMGWMDFGRRSAEEYEYPS(SEQ ID NO: 206) |
| COMP-T20 | MRSLSVLALLLLLLLAPASAAPGRMKQIEDKIEEILSKIYHIENEIARIKKLIGERGGS |
| | GSGGAHQDSRDNCPTVPNSAQQDSD (SEQ ID NO: 207) |
| ApoF-T50 | MRSLSVLALLLLLLLAPASAAPGRMKQIEDKIEEILSKIYHIENEIARIKKLIGERGGS |
| | GSGGALLAREQQSTGRVGRSLPTEDCENEKEQAVHNVVQLLPGVGTFYNLGTALY |
| | (SEQ ID NO: 208) |
| PLP-T50 | MRSLSVLALLLLLLLAPASAAPGRMKQIEDKIEEILSKIYHIENEIARIKKLIGERGGS |
| | GSGGASLGAHVAGEAGSKTPGLSRITGLDPVEASFESTPEEVRLDPSDADFVDVI |
| | (SEQ ID NO: 209) |
| COMP-T50 | MRSLSVLALLLLLLLAPASAAPGRMKQIEDKIEEILSKIYHIENEIARIKKLIGERGGS |
| | GSGGADSDQDKDGDGHQDSRDNCPTVPNSAQQDSDSDGQGDACDEDDDNDGVP |
| | DS (SEQ ID NO: 210) |
| MAT2-T50 | MRSLSVLALLLLLLLAPASAAPGRMKQIEDKIEEILSKIYHIENEIARIKKLIGERGGS |
| | GSGGALAEDGKRCVAVDYCASENHGCEHECVNADGSYLCQCHEGFALNPDKKTC |
| | T (SEQ ID NO: 211) |
| Subt-T50 | MRSLSVLALLLLLLLAPASAAPGRMKQIEDKIEEILSKIYHIENEIARIKKLIGERGGS |
| | GSGGALLLGPAGARAQEDEDGDYEEL(SEQ ID NO: 212) |
| PLP-D50 | MRSLSVLALLLLLLLAPASAAPGRMKQLEDKIEELLSKIYHLENEIARLKKLIGERG |
| | GSGSGGASKTPGLSRITGLDPVEASFESTPEEVRLDPSDADFVDVIHTDAAPLIP |
| | (SEQ ID NO: 213) |
| PLP-D50 | MRSLSVLALLLLLLLAPASAAPGRMKQLEDKIEELLSKIYHLENEIARLKKLIGERG |
| (truncated) | GSGSGGA (SEQ ID NO: 214) |
| IL27-D50 | MRSLSVLALLLLLLLAPASAAPGRMKQLEDKIEELLSKIYHLENEIARLKKLIGERG |
| | GSGSGGANLPEEEEEEEEEEEEERKGLLPGALGSALQGPAQVS (SEQ ID NO: 215) |
| ApoF-D50 | MRSLSVLALLLLLLLAPASAAPGRMKQLEDKIEELLSKIYHLENEIARLKKLIGERG |
| | GSGSGGAQVLIQHLRGLQKGRSTERNVSVEALASALQLLAREQQSTGRVGRSLPTE |
| | D (SEQ ID NO: 216) |
| ApoF-D50-del | MRSLSVLALLLLLLLAPASAAPGRMKQLEDKIEELLSKIYHLENEIARLKKLIGERG |
| | GSGSGGAQVLIQHLRGLQKGRSTERNVSRVGRSLPTED (SEQ ID NO: 217) |

In some embodiments, the nucleic acids provided herein (e.g., DNA, RNA, or a combination thereof) encode for an amino acid sequence comprising at least 75% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 80% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 85% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 90% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 95% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 96% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 97% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 98% sequence identity to a sequence selected from any one of SEQ ID NOS: 205 to 217. In some embodiments, the nucleic acids provided herein (e.g., DNA, RNA, or a combination thereof) encode for an amino acid sequence comprising at least 99% sequence identity to a sequence selected from any one of SEQ ID NOS: 205 to 217. In some embodiments, the nucleic acids provided herein (e.g., DNA, RNA, or a combination thereof) encode for an amino acid sequence selected from any one of SEQ ID NOS: 205 to 217. In some embodiments, the nucleic acids provided herein (e.g., DNA, RNA, or a combination thereof) encode for a leader peptide sequence. In some embodiments, the leader peptide sequence comprises an amino acid sequence of: MRSLSVLALLLLLLLAPASA (SEQ ID NO: 223). In some embodiments, the nucleic acids provided herein (e.g., DNA, RNA, or a combination thereof) further encode a zipper. For example, the zipper for the trimer peptides can comprise an amino acid sequence of: RMKQIEDKIEEILSKIYHIENEIARIKKLIGER (SEQ ID NO: 224) and a zipper for a dimer can comprise and amino acid sequence of: RMKQLEDKIEELLSKIYHLENEIARLKKLIGER (SEQ ID NO: 225).

Provided herein are synthetic proteins comprising at least one protein construct or nucleic acid encoding for at least one protein construct, wherein the at least one protein construct comprises an amino acid sequence that is at least 75%, at least 80%, at least 85%, at least 90%, at least 95%, at least 96%, at least 97%, at least 98%, at least 99%, or 100% identical to a sequence selected from SEQ ID NOS: 176 to 217. In some embodiments, the synthetic proteins provided herein comprise a sequence that is at least at least 75%, at least 80%, at least 85%, at least 90%, at least 95%, at least 96%, at least 97%, at least 98%, at least 99%, or 100% identical to SEQ ID NO: 206, SEQ ID NO: 210, SEQ ID NO: 212, SEQ ID NO: 213, SEQ ID NO: 215, SEQ ID NO: 217, or a variant thereof. Further provided herein are compositions comprising two or more, three or more, four or more, or five or more protein constructs. In some embodiments, the nucleic acid encoding the at least one protein construct is a DNA. In some embodiments, the nucleic acid encoding the at least on protein construct is an RNA. In some embodiments, the nucleic acid comprises a self-replicating RNA sequence or a DNA encoding the self-replicating RNA sequence.

### (2) Nucleic Acids Encoding an Antigen

Provided herein are compositions comprising one or more nucleic acids. In some embodiments, the one or more nucleic acids comprise deoxyribonucleic acid (DNA). In some embodiments, the one or more nucleic acids comprise ribonucleic acid (RNA). In some embodiments, the one or more nucleic acids comprise a mixture of DNA and RNA.

In some embodiments, a nucleic acid provided herein comprises an RNA adjuvant sequence (e.g., encoding a TLR agonist protein) and an antigen provided herein. In some embodiments, the nucleic acids provided herein comprise a polynucleotide comprise one or more modified nucleotides or nucleobases, and/or their analogs. A polynucleotide provided herein can comprise modified nucleotides, such as methylated nucleotides and their analogs. If present, modification to the nucleotide structure may be imparted before or after assembly of compositions provided herein. Modified nucleobases which can be incorporated into modified nucleosides and nucleotides and be present in the RNA molecules include: 1-methyladenosine, 2-methylthio-N6-hydroxynorvalyl carbamoyladenosine, 2-methyladenosine, 2-O-ribosylphosphate adenosine, N6-methyl-N6-threonylcarbamoyladenosine, N6-acetyladenosine, N6-glycinylcarbamoyladenosine, N6-isopentenyladenosine, N6-methyladenosine, N6-threonylcarbamoyladenosine, N6,N6-dimethyladenosine, N6-(cis-hydroxyisopentenyl)adenosine, N6-hydroxynorvalylcarbamoyladenosine, 1,2-O-dimethyladenosine, N6,2-O-dimethyladenosine, 2-O-methyladenosine, N6,N6, O-2-trimethyladenosine, 2-methylthio-N6-(cis-hydroxyisopentenyl) adenosine, 2-methylthio-N6-methyladenosine, 2-methylthio-N6-isopentenyladenosine, 2-methylthio-N6-threonyl carbamoyladenosine, 2-thiocytidine, 3-methylcytidine, N4-acetylcytidine, 5-formylcytidine, N4-methylcytidine, 5-methylcytidine, 5-hydroxymethylcytidine, lysidine, N4-acetyl-2-O-methylcytidine, 5-formyl-2-O-methylcytidine, 5,2-O-dimethylcytidine, 2-O-methylcytidine, N4,2-O-dimethylcytidine, N4,N4,2-O-trimethylcytidine, 1-methylguanosine, N2,7-dimethylguanosine, N2-methylguanosine, 2-O-ribosylphosphate guanosine, 7-methylguanosine, under modified hydroxywybutosine, 7-aminomethyl-7-deazaguanosine, 7-cyano-7-deazaguanosine, N2,N2-dimethylguanosine, 4-demethylwyosine, epoxyqueuosine, hydroxywybutosine, isowyosine, N2,7,2-O-trimethylguanosine, N2,2-O-dimethylguanosine, 1,2-O-dimethylguanosine, 2-O-methylguanosine, N2,N2,2-O-trimethylguanosine, N2,N2,7-trimethylguanosine, peroxywybutosine, galactosyl-queuosine, mannosyl-queuosine, queuosine, archaeosine, wybutosine, methylwyosine, wyosine, 2-thiouridine, 3-(3-amino-3-carboxypropyl)uridine, 3-methyluridine, 4-thiouridine, 5-methyl-2-thiouridine, 5-methylaminomethyluridine, 5-carboxymethyluridine, 5-carboxymethylaminomethyluridine, 5-hydroxyuridine, 5-methyluridine, 5-taurinomethyluridine, 5-carbamoylmethyluridine, 5-(carboxyhydroxymethyl)uridine methyl ester, dihydrouridine, 5-methyldihydrouridine, 5-methylaminomethyl-2-thiouridine, 5-(carboxyhydroxymethyl)uridine, 5-(isopentenylaminomethyl)uridine, 5-(isopentenylaminomethyl)-2-thiouridine, 3,2-O-dimethyluridine, 5-carboxymethylaminomethyl-2-O-methyluridine, 5-carbamoylmethyl-2-O-methyluridine, 5-methoxycarbonylmethyl-2-O-methyluridine, 5-(isopentenylaminomethyl)-2-O-methyluridine, 5,2-O-dimethyluridine, 2-O-methyluridine, 2-thio-2-O-methyluridine, uridine 5-oxyacetic acid, 5-methoxycarbonylmethyluridine, uridine 5-oxyacetic acid methyl ester, 5-methoxyuridine, 5-aminomethyl-2-thiouridine, 5-carboxymethylaminomethyl-2-thiouridine, 5-methylaminomethyl-2-selenouridine, 5-methoxycarbonylmethyl-2-thiouridine, 5-taurinomethyl-2-thiouridine, pseudouridine, 1-methyl-3-(3-amino-3-carboxypropyl)pseudouridine, 1-methylpseudouridine, 3-methylpseudouridine, 2-O-methylpseudouridine, inosine, 1-methylinosine, 1,2-O-dimethylinosine and 2-O-methylinosine. In some embodiments, a nucleic acid provided herein comprises a pseudouridine modification to ensure the stability of mRNA. Many of these modified nucleobases and their corresponding ribonucleosides are available from commercial suppliers. If desired, the nucleic acid can contain phosphoramidate, phosphorothioate, and/or methylphosphonate linkages. The RNA sequence or DNA sequence encoding an RNA adjuvant can be modified with respect to its codon usage, for example, to increase translation efficacy and half-life of the RNA. A poly A tail (e.g., of about 30 adenosine residues or more) may be attached to the 3′ end of the RNA to increase its half-life. Cap structure can provide stability and translational efficacy to the RNA molecule. In some embodiments, the RNA adjuvant provided herein or DNA encoding an RNA adjuvant is chemically modified. In some embodiments, the nucleic acid encoding an antigen is chemically modified. In some embodiments, the nucleic acid encoding an antigen comprises DNA. In some embodiments, the nucleic acid encoding an antigen comprises RNA. In some embodiments, the chemical modification to an RNA sequence provided herein comprises a poly-A tail, a chemically modified nucleobase provided herein, or a 5′ terminal cap.

In some embodiments, the RNA provided herein is an mRNA. In some embodiments, the mRNA is a non-replicating mRNA. In some embodiments, the mRNA is a self-amplifying mRNA. The non-replicating mRNA and the self-amplifying mRNA are capable of utilizing the host cell translational machinery for the production of the antigen target and launch of an adaptive immune response. Non-replicating mRNA encode for the protein antigen(s) of interest, while self-amplifying mRNA are also capable of encoding proteins, allowing for RNA replication.

Provided herein are compositions, wherein the compositions comprise an RNA encoding an antigen or a plurality of RNAs each encoding for a different antigen. The compositions provided herein can be multivalent for use as a vaccine to induce an immune response to multiple antigens (e.g., viral, bacterial, and/or tumor antigens). Further provided herein are compositions comprising an RNA adjuvant provided herein and an RNA encoding an antigen. In some embodiments, the RNA sequence encoding the antigen is operably linked to the RNA adjuvant. Further provided herein are compositions comprising an RNA adjuvant provided herein and a protein antigen or a fragment thereof. The protein antigen can comprise an inactivated bacterium or virus, a fragment of a bacterial toxin, or a fragment of an antigen expressed by a microorganism.

In some embodiments, the antigens provided herein are from a microorganism. In some embodiments, the antigen is a viral antigen, a bacterial antigen, a fungal antigen, or a parasitic antigen.

In some embodiments, the antigens provided herein are a bacterial antigen. Non-limiting examples of infectious bacteria include: E. coli, Pseudomonas aeruginosa, Helicobacter pylori, Borrelia burgdorferi, Legionella pneumophilia, Mycobacteria sps (such as. M. tuberculosis, M avium, M. intracellular e, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus epidermidis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringers, Clostridium tetani, Enterobacter aerogenes, Klebsiella pneumoniae, Brucella abortus, Pasteur ella multocida, Bacteroides sp., Fusobacterium nucleatum, Streptobacillus moniliformis, Treponema pallidium, Treponema pertenue, Leptospira, Nocardia brasiliensis, Borrelia hermsii, Borrelia burgdorferi, and Actinomyces israelii.

In some embodiments, the antigens provided herein are a viral antigen. In some embodiments, the antigen comprises a spike protein, a glycoprotein, a hemagglutinin protein, or a viral envelope protein. In some embodiments, the viral antigen is from an adenovirus, an astrovirus, a bocavirus, a Banna virus, a BK virus, a Coltivirus, a coronavirus, a coxsackievirus, a cytomegalovirus, a Dengue virus, an Ebola virus, an enterovirus, an Epstein-Barr virus, a Hanta virus, a hepatitis A virus, a hepatitis B virus, a hepatitis C virus, a herpes simplex virus, a human immunodeficiency virus (HIV), an influenza virus, a JC virus, a Lassa virus, a Marburg virus, a measles virus, a Middle East Respiratory Syndrome (MERS) coronavirus, a Norwalk virus, a norovirus, a parvovirus, a papillomavirus, a polio virus, a rabies virus, a rhinovirus, a rotavirus, a Rubella virus, a severe acute respiratory syndrome (SARS) virus, a smallpox virus, Varicella-zoster virus, a Yellow fever virus, or any combination thereof. In some embodiments, the SARS virus is a SARS-CoV-2 virus. Additional non-limiting examples of viruses include Retroviridae (for example, HIV); Picornaviridae (for example, polio viruses, hepatitis A virus; enteroviruses, human coxsackie viruses, rhinoviruses, echoviruses); Caliciviridae (such as strains that cause gastroenteritis); Togaviridae (for example, equine encephalitis viruses, rubella viruses); Flaviviridae (for example, dengue viruses, encephalitis viruses, yellow fever viruses, West Nile virus, Zika virus); Coronaviridae (for example, coronaviruses); Rhabdoviridae (for example, vesicular stomatitis viruses, rabies viruses); Filoviridae (for example, Ebola viruses); Paramyxoviridae (for example, parainfluenza viruses, mumps virus, measles virus, respiratory syncytial virus); Orthomyxoviridae (for example, influenza viruses); Bunyaviridae (for example, Hantaan viruses, bunyaviruses, phleboviruses and Nairo viruses); Arenaviridae (hemorrhagic fever viruses); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and HSV-2, varicella zoster virus, cytomegalovirus (CMV), herpes viruses); Poxviridae (variola viruses, vaccinia viruses, pox viruses); and Iridoviridae (such as African swine fever virus); cytomegalovirus (CMV) pneumonia, enteritis and retinitis; Epstein-Barr virus (EBV) lymphoproliferative disease; varicella zoster virus, (VZV); HSV-1 and -2 mucositis; HSV-6 encephalitis, BK-virus hemorrhagic cystitis; and hepatitis A, B or C.

Exemplary viral antigens that can be encoded by the RNA molecules provided herein include but are not limited to: spike (S) protein, nucleocapsid protein (NP), hemagglutinin (HA), neuraminidase (NA), Den1 viral envelope protein, a Den2 viral envelope protein, a Den3 viral envelope protein, Den4 viral envelope, lipopeptide, gene products of HIV gag, pol, and env genes, the Nef protein, reverse transcriptase, nucleoprotein, matrix protein (M1), membrane protein (M2), transcriptase components (PB1, PB2 and PA), parvovirus antigens (e.g., VP-1, VP-2, VP-3, NS-1, and NS-2), RSV-F, RSV-G, rubella viral antigens (e.g., proteins E1 and E2), glycoprotein B, HPV antigens (e.g., HPV-16, HPV-18, HPV-31, HPV-33 and HPV-35), Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA EBV-VCA, HTLV-antigen (e.g., TAX), hepatitis B core antigen, and hepatitis B envelope antigen, Herpes Simplex 1/2 (HSV1/2) HSV-2 glycoproteins B,C,D,E (gB2, gC2, gD2, gE2), Cytomegalovirus (CMV) glycoprotein B (gB), and pentameric complex (PC).

In some embodiments, the antigens provided herein are fungal antigens. Non-limiting examples of fungi include but are not limited to: Cryptococcus neoformans, Histoplasma capsulatum, Coccidioides immitis, Blastomyces dermatitidis, Pneumocystis carinii, Chlamydia trachomatis, and Candida albicans. In some embodiments, the compositions provided herein comprise an antigen from other infectious organisms. Additional examples of infectious organisms, include but are not limited to protozoan parasites such as Plasmodium falciparum, Schistosoma mansoni, Trypanosoma cruzi, Trichinella spiralis, Strongyloides ratti, and Toxoplasma gondii, among others.

Further provided herein are compositions for use in the treatment of cancer. In some embodiments, a composition comprises an RNA encoding an oncoviral antigen. In some embodiments, the oncoviral antigen is from a human papillomavirus (HPV) antigen, a Kaposi Sarcoma-Associated Herpesvirus (KSHV) antigen, a Merkel Cell Polyomavirus (MCV) antigen, a Human T-Cell Lymphotropic Virus Type 1 (HTLV-1) antigen, or an Epstein-Barr Virus (EBV) antigen.

In some embodiments, a composition provided herein comprises an RNA encoding a tumor antigen. In some embodiments, the tumor antigen is a surface protein, a cytosolic protein, or a transmembrane protein. Tumor antigens are proteins expressed by cancer cells. Some tumor antigens, called neoantigens are proteins generated in cancer cells in the presence of point mutations, frame-shift mutations, and gene rearrangements. The RNA molecules provided herein can encode for one tumor antigen or a plurality of tumor antigens. Non-limiting examples of a tumor antigen include mucin (e.g., MUC1), carcinoembryonic antigen (CEA), human epidermal growth factor receptor 2 (Her2/neu), telomerase (TERT), survivin, melanoma-associated antigen A1 (MAGE-A1), MAGE-A3, Wilms tumor 1 (WT1), preferentially expressed antigen of melanoma (PRAME), New York esophagus 1 (NY-ESO-1), tumor protein p53, tyrosinase, 5100 protein, Melanoma-associated antigen recognized by T cells 1 (MART-1), or any combination thereof.

Furthermore, the RNA molecules provided herein can encode for a protein that regulates the immune response to a cancer cell or a tumor. In some embodiments, a composition provided herein comprises an RNA encoding an immune regulatory protein. In some embodiments, a composition comprises one or more RNA sequences encoding for an immune regulatory protein. In some embodiments, the immune regulatory protein is a CD83, a 4-1BB ligand, a cytokine, or any combination thereof. Exemplary cytokines useful in the treatment of cancer include but are not limited to: IL-1α, IL-1β, IL-2, IL-3, IL-4, IL-5, IL-6, IL-7, IL-8, IL-9, IL-10, IL-13, IL-14, IL-15, IL-17, IL-18, IL-21, IL-23, IL-24 CCL3, CCL5, and CXCR4.

The compositions provided herein can further comprise a delivery vehicle. Non-limiting examples of delivery vehicles that can be used include: solvents, diluents, aqueous solutions, saline solutions, particles, emulsifying agents, surfactants, lipid carriers, lipidoids, engineered vesicles, cells, extracellular vesicles, yeast cells, antibodies, aptamers, vectors, viral vectors, adenoviral vectors, lentiviral vectors, micelles, liposomes, and transfection agents (e.g., polyvalent cationic lipid compositions, such as polyethylenimine (PEI), LIPOFECTIN®, LIPOFECTACE®, LIPOFECTAMINE™, CELLFECTlN®, DMRIE-C, DMRIE, DOTAP, DOSPA, and DOSPER, and dendrimer compositions, particularly G5-G 10 dendrimers, including dense star dendrimers, PAMAM dendrimers, grafted dendrimers, and dendrimers known as dendrigrafts and SUPERFECT®).

Provided herein are vectors, wherein the vectors comprise: one or more nucleic acids encoding: a TLR5 agonist provided herein, a TLR4 agonist provided herein, a TLR2 agonist provided herein, or a combination thereof. In some embodiments, the vector is a plasmid. In some embodiments, the vector is a virus-like particle. In some embodiments, the vector is a viral vector. In some embodiments, the viral vector comprises an adenovirus, an adeno-associated virus (AAV), a recombinant AAV (rAAV), a lentivirus, an alphavirus, a flavivirus, a rhabdovirus, a herpes simplex virus, a Kunjin virus, a measles virus, a Lassa virus or a virus-like particle thereof. In some embodiments, the AAV or rAAV comprises one or more of an AAV1, AAV2, AAV3, AAV4, AAV5, AAV6, AAV7, AAV8, AAV9, AAV10, AAV11, AAV12, AAV13, or an AAV10rh. In some embodiments, the viral vector is a self-replicating viral vector (e.g., an alphavirus) or a portion thereof.

In some embodiments, the one or more nucleic acids comprise DNA. In some embodiments the one or more nucleic acids comprise RNA. In some embodiments, the vector comprises a DNA sequence encoding for an RNA sequence that is at least 85% identical to any one of SEQ ID NOS: 2 to 37. In some embodiments, the vector comprises a sequence that is at least 85% identical to SEQ ID NO: 226. In some embodiments, the vector comprises a nucleic acid sequence that is at least 85% identical to any one of SEQ ID NOS: 2 to 37. In some embodiments, the vector comprises a DNA sequence encoding for an RNA sequence that is at least 85% identical to any one of SEQ ID NOS: 38 to 103. In some embodiments, the vector comprises a nucleic acid sequence that is at least 85% identical to any one of SEQ ID NOS: 38 to 103. In some embodiments, the vector comprises a DNA sequence encoding for an RNA sequence that is at least 85% identical to any one of SEQ ID NOS: 104 to 175. In some embodiments, the vector comprises a nucleic acid sequence that is at least 85% identical to any one of SEQ ID NOS: 104 to 175. In some embodiments, the vector comprises a nucleic acid sequence encoding a viral antigen, a bacterial antigen, a fungal antigen, a parasite antigen, or a tumor antigen. In some embodiments, the vector comprises a sequence encoding for a SARS-CoV-2 spike protein or any antigen provided herein.

Liposomes and/or nanoparticles also can be employed with administration of compositions herein. Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 angstroms containing an aqueous solution in the core.

Nanoparticle carriers may also be used as a delivery vehicle for the RNA molecules provided herein. In some embodiments, the nanoparticle is a gold nanoparticle, a platinum nanoparticle, an iron-oxide nanoparticle, a lipid nanoparticle, a selenium nanoparticle, a glycol chitosan nanoparticle (CNP), a cathepsin B sensitive nanoparticle, a hyaluronic acid nanoparticle, a paramagnetic nanoparticle, or a polymeric nanoparticle. Additional carriers and nanoparticles are discussed further below.

### (3) Carriers

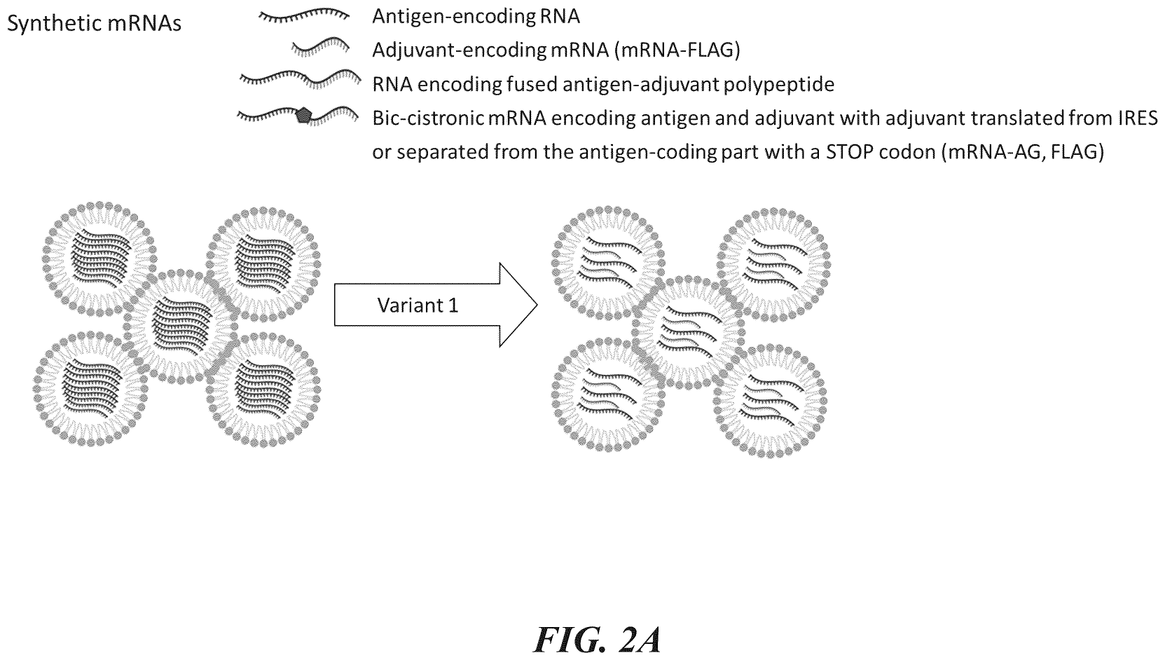

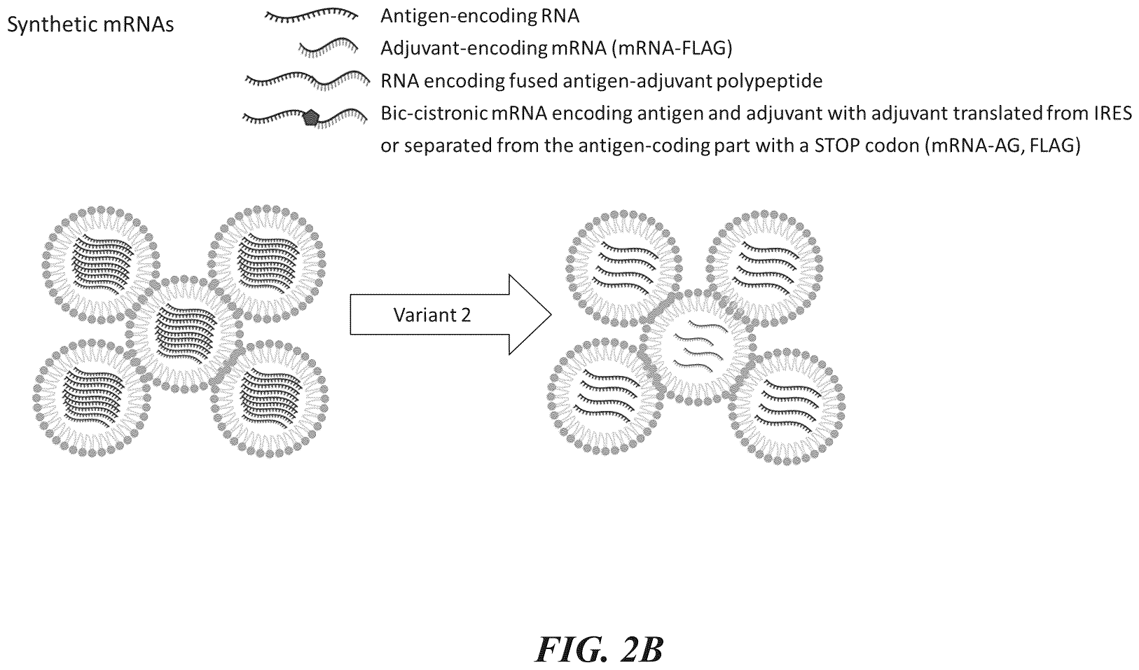

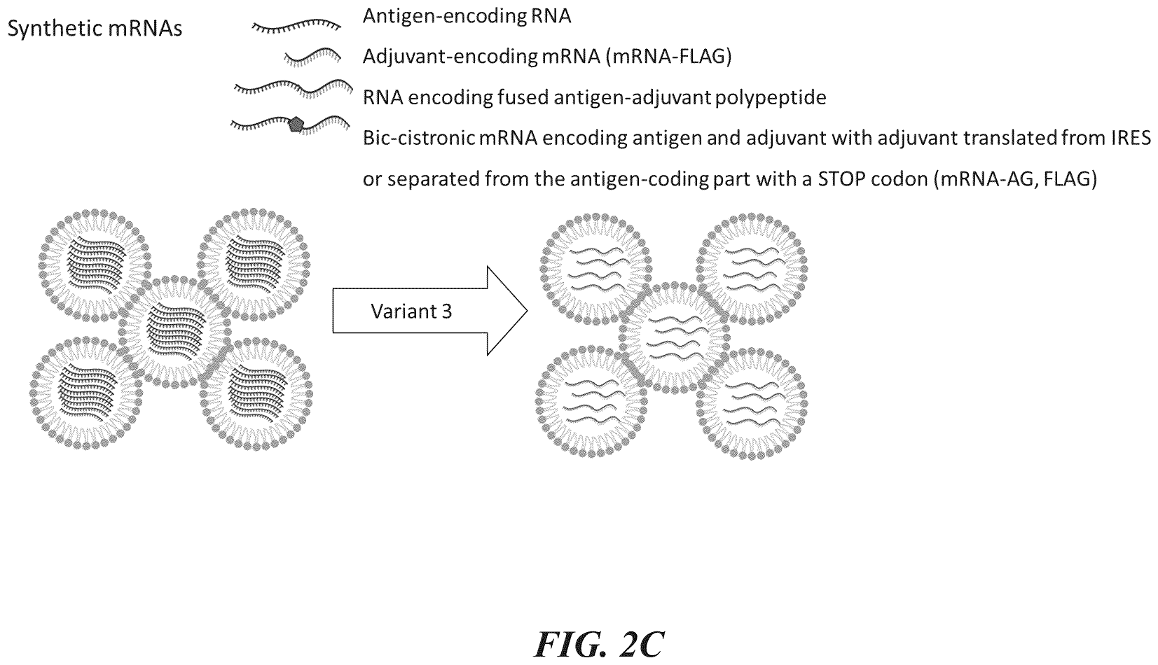

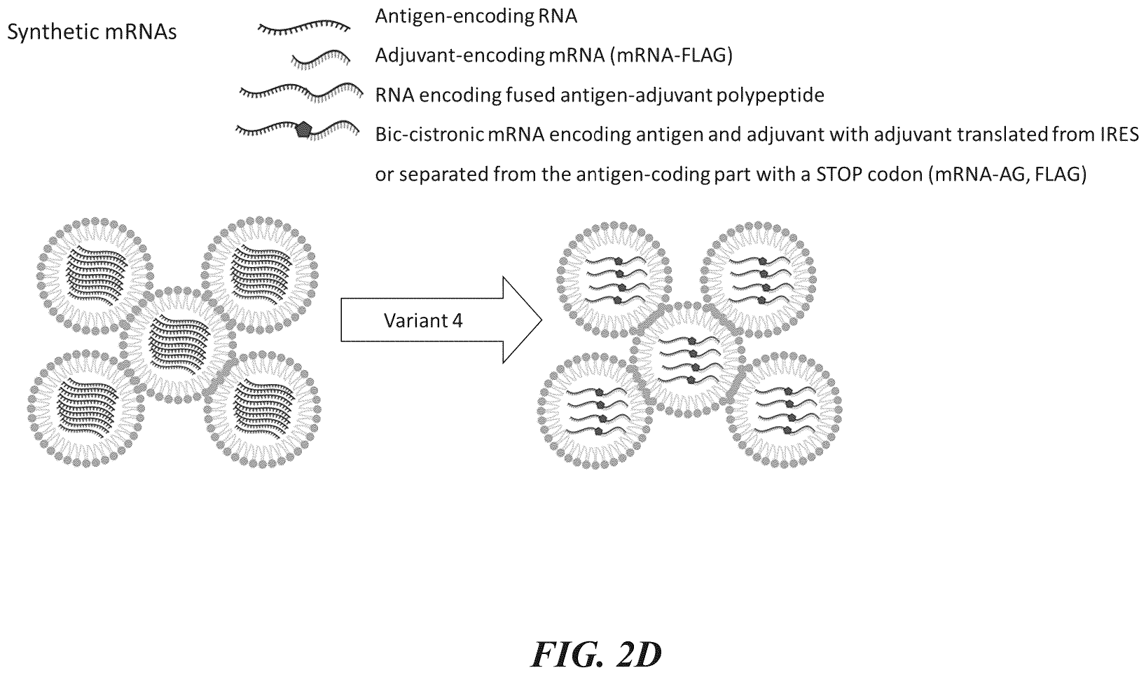

Provided herein are compositions comprising a carrier. A carrier facilitates delivery of a nucleic acid provided herein into a cell or tissue. In some embodiments, the carrier is a lipid carrier or a lipid nanoparticle. In some embodiments, a nucleic acid provided herein (e.g., DNA, RNA, or an RNA adjuvant provided herein) is in complex with a lipid carrier. In some embodiments, a nucleic acid provided herein (e.g., DNA, RNA, or an RNA adjuvant provided herein) is encapsulated within a lipid carrier. In some embodiments, a nucleic acid encoding an antigen provided herein is in complex with a lipid carrier. In some embodiments, a nucleic acid encoding an antigen provided herein is encapsulated within a lipid carrier. In some embodiments, a vector provided herein is in complex with a lipid carrier. In some embodiments, a vector provided herein is encapsulated within a lipid carrier. In some embodiments, a protein antigen provided herein is in complex with a lipid carrier. In some embodiments, a protein antigen provided herein is encapsulated within a lipid carrier. In some embodiments, the nucleic acid encoding for the antigen is encapsulated within the same lipid carrier as the RNA adjuvant, the TLR agonist, or a nucleic acid encoding the TLR agonist. In some embodiments, the RNA encoding for the antigen is encapsulated within a different lipid carrier as the RNA adjuvant. Exemplary configurations of carrier-RNA complexes are provided in FIGS. 2A-2D.

In some embodiments, the lipid carriers provided herein comprise a cationic lipid, a polyethylene glycol (PEG)-modified lipid, a sterol and a non-cationic lipid. In some embodiments, the lipid carrier is a cationic lipid nanoparticle, an ionizable lipid nanoparticle, a PEG lipid carrier, a polymeric lipid nanoparticle, a polymer-conjugated lipid carrier, a synthetic vesicle, a liposome, an exosome, an endosome, a lipoplex, a lipidoid, a derivative, variant, or any combination thereof.

In some embodiments, the lipid carrier comprises a cationic lipid. In some embodiments, the lipid carriers provided herein comprise an ionizable cationic lipid. In some embodiments, the cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some embodiments, the cationic lipid carrier has a molar ratio of about 20-60% cationic lipid:about 5-25% non-cationic lipid:about 25-55% sterol; and about 0.5-15% PEG-modified lipid. In some embodiments, the cationic lipid carrier comprises a molar ratio of about 50% cationic lipid, about 1.5% PEG-modified lipid, about 38.5% cholesterol and about 10% non-cationic lipid. In some embodiments, the cationic lipid carrier comprises a molar ratio of about 55% cationic lipid, about 2.5% PEG lipid, about 32.5% cholesterol and about 10% non-cationic lipid. In some embodiments, the cationic lipid is an ionizable cationic lipid and the non-cationic lipid is a neutral lipid, and the sterol is a cholesterol. In some embodiments, the cationic lipid nanoparticle has a molar ratio of about 50:38.5:10:1.5 of cationic lipid:cholesterol:PEG2000-DMG:DSPC. In some embodiments, the cationic lipid carrier has a mean diameter of about 50 nanometers (nm) up to about 200 nm. In some embodiments, the cationic lipid nanoparticle has a mean diameter of about 80 nm up to 100 nm. The compositions provided herein can comprise about 2 mg/mL of RNA or less and about 30 up to about 50 mg/mL lipids.

In some embodiments, the lipid carriers provided herein comprise cholesterol. In some embodiments, the lipid carriers provided herein comprise a PEGylated lipid. In some embodiments, the lipid carriers provided herein comprise distearoylphosphatidylcholine (DSPC). In some embodiments, the lipid carriers provided herein comprise a ionizable cationic lipid, cholesterol, a PEGylated lipid and distearoylphosphatidylcholine (DSPC).

Lipid nanoparticle (LNP) formulations can further comprise coating with a surfactant or polymer in order to improve the delivery of the nanoparticle. In some embodiments, the nanoparticle may be coated with a hydrophilic coating such as, but not limited to, PEG coatings and/or coatings that have a neutral surface charge. The hydrophilic coatings may help to deliver nanoparticles with larger payloads. In some embodiments, the lipid nanoparticles comprise hydrophilic polymer particles.

The nanoparticle formulations provided herein can further comprise a carbohydrate nanoparticle comprising a carbohydrate carrier and an RNA encoding an antigen and/or RNA adjuvant provided herein. As a non-limiting example, the carbohydrate carrier may include, but is not limited to, an anhydride-modified phytoglycogen or glycogen-type material, phytoglycogen octenyl succinate, phytoglycogen beta-dextrin, anhydride-modified phytoglycogen beta-dextrin.

The lipid nanoparticle can further comprise a polymeric material (i.e., a polymeric core) and/or a polymer-vitamin conjugate and/or a tri-block co-polymer. The polymeric material may include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates. The polymeric material may be biodegradable and/or biocompatible. The polymeric material may additionally be irradiated. As a non-limiting example, the polymeric material may be gamma irradiated. Non-limiting examples of specific polymers include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, poly(ortho)esters, poly(butyric acid), poly(valeric acid), poly(lactide-co-caprolactone), PEG-PLGA-PEG and trimethylene carbonate, polyvinylpyrrolidone.

The lipid nanoparticle can comprise surface altering agents such as, but not limited to, polynucleotides, anionic proteins (e.g., bovine serum albumin), surfactants (e.g., cationic surfactants such as for example dimethyldioctadecylammonium bromide), sugars or sugar derivatives (e.g., cyclodextrin), nucleic acids, polymers (e.g., heparin, polyethylene glycol and poloxamer), mucolytic agents (e.g., N-acetylcysteine, mug wort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β4 domase alfa, neltenexine, erdosteine) and various DNases including rhDNase. The surface altering agent may be embedded or enmeshed in the particle's surface or disposed (e.g., by coating, adsorption, covalent linkage, or other process) on the surface of the lipid nanoparticle.

In some embodiments, the carrier is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids.

In some embodiments, the compositions provided herein are formulated as a solid lipid nanoparticle. A solid lipid nanoparticle (SLN) may be spherical with an average diameter between 10 to 1000 nm. SLN possess a solid lipid core matrix that can solubilize lipophilic molecules and may be stabilized with surfactants and/or emulsifiers. The type of lipid carrier used in the compositions provided herein will depend on the target site, mode of administration, and RNA delivery to the target site that induce an immune response in a subject.

### (4) Yeast Cell Compositions

Provided herein are compositions comprising: (a) a yeast cell, wherein the yeast cell comprises a permeable cell wall; and (b) one or more nucleic acids (e.g., an RNA adjuvant), wherein the one or more nucleic acids encode for at least a functional fragment of a TLR4 agonist, a TLR2 agonist, a TLR5 agonist, or a combination thereof. In some embodiments, one or more nucleic acids (e.g., an RNA adjuvant) are encapsulated within the yeast cell. In some embodiments, the one or more nucleic acids (e.g., an RNA adjuvant) are in complex with the yeast cell.

In order to prepare a composition comprising a yeast cell as a delivery vehicle or carrier, the yeast cell can be contacted with a permeabilization agent prior to complexing or encapsulation of the nucleic acids (e.g., RNA adjuvants encoding a TLR agonist) provided herein. In some embodiments, the permeabilization agent is beta-glucanase. In some embodiments, the beta-glucanase is a b-1-3-glucanase. In some embodiments, the yeast cell is a Pichia pastoris cell, a Saccharomyces cerevisiae cell, or a Kluyveromyces lactis cell.

In some embodiments, a composition provided herein further comprises a nucleic acid (e.g., an RNA sequence) encoding for an antigen provided herein (e.g., a viral antigen, e.g, a SARS-CoV-2 spike protein or a fragment thereof). In some embodiments, the nucleic acids provided herein comprise a sequence that is at least 85% identical to any one of SEQ ID NOS: 2 to 175. In some embodiments, the nucleic acids provided herein comprise a sequence that is at least 90% identical to any one of SEQ ID NOS: 2 to 175. In some embodiments, the nucleic acids provided herein comprise a sequence that is at least 95% identical to any one of SEQ ID NOS: 2 to 175. In some embodiments, the nucleic acids provided herein comprise any one of SEQ ID NOS: 2 to 175. In some embodiments, the nucleic acids provided herein encode an amino acid sequence selected from any one of SEQ ID NOS: 170 to 210. In some embodiments, the compositions provided herein further comprise an enteric-coated microsphere. In some embodiments, the yeast cell is encapsulated within the enteric-coated microsphere.

In some embodiments, a composition provided herein is formulated for oral administration to a subject. In some embodiments, the composition comprising a yeast cell provided herein is admixed with a food composition. In some embodiments, where the food compositions are prepared or formulated as a functional food (e.g., a nutritional supplement). In other embodiments, such compositions may be prepared or formulated, for example, as a pharmaceutical, a dietary supplement and/or a medical food.

### (5) Pharmaceutical Vaccine Compositions, Dosing, and Administration

Provided herein are pharmaceutical compositions and a vaccine compositions comprising one or more nucleic acids provided herein or a vector provided herein; and a pharmaceutically acceptable diluent, carrier, or excipient. Further provided herein is a pharmaceutical composition or a vaccine composition comprising two or more of the following: (a) a nucleic acid encoding a TRL agonist (e.g., an RNA adjuvant); (b) a nucleic acid sequence (e.g., an RNA sequence) encoding for an antigen; and/or (c) a carrier; and (d) pharmaceutically acceptable diluent, carrier, or excipient. Further provided herein are pharmaceutical compositions comprising a vector comprising or encoding any one of the nucleic acids (e.g., RNA adjuvants) or proteins provided herein; and a pharmaceutically acceptable diluent, carrier, or excipient.

In some embodiments, compositions provided herein (e.g., RNA adjuvants) are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

The RNA adjuvants, nucleic acids, compositions, vaccine compositions, and pharmaceutical compositions for administering to a subject in need thereof may be formulated in unit dosage form for ease of administration and uniformity of dosage. A unit dosage form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, such as mice, rabbits, dogs, pigs, or non-human primates. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

Provided herein are nucleic acids, RNA adjuvants, compositions, vaccine compositions, and pharmaceutical compositions for administering to a subject in need thereof (e.g., as a vaccine or as a treatment for an infectious disease or a cancer). In some embodiments, administration of a composition provided herein is local administration. In some embodiments, administration of a composition provided herein is systemic administration. In some embodiments, a composition provided herein is formulated for administration/for use in administration via subcutaneous, intradermal, intramuscular, inhalation, intravenous, intraperitoneal, or oral route. In some cases, a treatment regime may be dosed according to a body weight of a subject. For example, body mass index can be used. BMI is calculated by: BMI=weight (kg)/[height (m)]².

By way of example only, exemplary dosages for the nucleic acids provided herein and RNA vaccines are provided below. In some embodiments, a nucleic acid encoding an RNA adjuvant or TLR agonist provided herein and/or a nucleic encoding an antigen provided herein is administered in an amount of at least about 1 nanograms (ng) or more, about 5 ng or more, about 10 ng or more, about 20 ng or more, about 30 ng or more, about 40 ng or more, about 50 ng or more, about 60 ng or more, about 70 ng or more, about 80 ng or more, about 90 ng or more, 100 ng or more, 110 ng or more, 120 ng or more, 130 ng or more, 140 ng or more, 150 ng or more, 160 ng or more, 170 ng or more, 180 ng or more, 190 ng or more, 200 ng or more, 210 ng or more, 220 ng or more, 230 ng or more, 240 ng or more, 250 ng or more, 260 ng or more, 270 ng or more, 280 ng or more, 290 ng or more, 300 ng or more, 350 ng or more, 400 ng or more, up to 500 ng. In some embodiments, a nucleic acid provided herein is administered in an amount of at least about 1 nanogram (ng) or more, 10 ng or more, 50 ng or more, 100 ng or more, 150 ng or more, 200 ng or more, 250 ng or more, 500 ng or more, or 1 (μg) microgram. In some embodiments, a nucleic acid provided herein is administered in an amount of at least about 1 microgram (μg) or more, about 5 μg or more, about 10 μg or more, about 20 μg or more, about 30 μg or more, about 40 μg or more, about 50 μg or more, about 60 μg or more, about 70 μg or more, about 80 μg or more, about 90 μg or more, up to 100 μg.

In some embodiments, compositions provided herein are administered intravenously or intramuscularly. In some embodiments, compositions provided herein are administered at a concentration of at least about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, about 1200 mg/kg, about 1300 mg/kg, about 1400 mg/kg, about 1500 mg/kg, about 2000 mg/kg, about 2200 mg/kg, about 2400 mg/kg, up to about 2500 mg/kg.

In some embodiments, compositions provided herein are administered orally. In some embodiments, compositions provided herein are administered orally at a concentration of at least about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 200 mg/kg, about 300 mg/kg, about 400 mg/kg, about 500 mg/kg, about 600 mg/kg, about 700 mg/kg, about 800 mg/kg, about 900 mg/kg, about 1000 mg/kg, about 1100 mg/kg, about 1200 mg/kg, about 1300 mg/kg, about 1400 mg/kg, about 1500 mg/kg, about 2000 mg/kg, about 2200 mg/kg, about 2400 mg/kg, up to about 2500 mg/kg.

In some embodiments, the dose of the composition provided herein can be administered once per day or divided into subdoses and administered in multiple doses, e.g., daily, twice daily, weekly, biweekly, monthly, bimonthly, yearly, etc. In some embodiments, the administering is repeated at least about every 1 year (8760 hours, 365 days), 2 years, 3 years, 4 years, 5 years or more. In some embodiments, the subject is administered an additional dose of the nucleic acid, pharmaceutical composition, or the vaccine composition. In some embodiments, the administering comprises administration of a first dose of the pharmaceutical composition and administering of a second dose of the pharmaceutical composition at least about 2 weeks after the first dose. In some embodiments, the composition is a booster vaccine composition that is a second, third, or fourth dose of the composition administered to the subject.

The RNA adjuvants and RNA vaccine compositions (e.g., RNA encoding an antigen) can be administered within the same composition, concurrently, or sequentially. The RNA encoding the TLR agonist provided herein can be on the same nucleic acid strand as an RNA sequence encoding the antigen or a different nucleic acid strand. When administered sequentially, the time between administrations is selected to benefit, among others, the therapeutic efficacy, and/or safety of the combination treatment. In certain embodiments, the RNA adjuvant compositions provided herein can be administered first followed by an RNA vaccine, or alternatively, the RNA vaccine is administered first followed by the RNA adjuvant compositions of the present disclosure (e.g., a composition comprising an RNA sequence listed in Table 1). By way of example and not limitation, the time between administrations is about 1 hour (hr), about 2 hours (hrs), about 4 hrs, about 6 hrs, about 12 hrs, about 16 hrs or about 20 hrs. In certain embodiments, the time between administrations is about 24 hours (1 day), about 2 days, about 3 days, about 4 days, about 5 days, about 6 days, or about 7 or more days. In some embodiments, the time between administrations is about 1 week (7 days), 2 weeks, 3 weeks, or 4 weeks or more. In some embodiments, the time between administrations is about 1 month (30 days), 2 months (60 days), or more. When administered concurrently, the RNA adjuvant composition can be administered separately, at the same time as the second RNA vaccine, by the same or different routes, or administered in a single pharmaceutical composition by the same route. In certain embodiments, the amount and frequency of administration of the RNA vaccine can use standard dosages and standard administration frequencies used for that particular RNA vaccine. In some embodiments, the dose of the RNA vaccine is reduced when administered with an RNA adjuvant composition provided herein. This is due to the enhanced effect of the RNA adjuvant-RNA vaccine combination on the innate immune response.

### (6) Therapeutic Applications

The compositions and adjuvants provided herein are formulated for use in the treatment of a disease or a condition. Also provided herein are methods of treating a disease or a disorder in a subject. In some embodiments, the disease or the disorder is cancer or an infection. The adjuvants provided herein can be administered to a subject alone or in combination with an additional agent (e.g., a chemotherapeutic agent, a vaccine, an anti-viral medication, or an immunotherapy) in the same composition or in separate compositions.

Provided herein are methods of treating a disease or disorder in a subject comprising: administering to the subject an agent in combination with an adjuvant provided herein. In some embodiments, the agent is administered prior to administration of the adjuvant. In some embodiments, the agent is administered concurrently with the adjuvant. In some embodiments, the agent is administered after administration of the adjuvant. In some embodiments, a composition provided herein comprises an agent and an adjuvant in unit dose form.

In some embodiments, following administration of an effective amount of an agent or a composition provided herein to a subject in the presence of an antigen, an initial innate immune response is induced in the subject. In some embodiments, the innate immune response comprises an increase in the level or activity of Nuclear factor kappa B (NF-κB) in an epithelial cell of the subject relative to the level or activity of NF-κB in the absence of the composition.

In some embodiments, the subject has, is diagnosed with, or is at risk of developing an infection. In some embodiments, the infection is a viral infection. In some embodiments, the viral infection is an upper respiratory viral infection. In some embodiments, the upper respiratory viral infection is COVID-19, SARS infection, MERS infection, influenza, parainfluenza, respiratory syncytial virus (RSV) infection, pneumonia, or a rhinoviral infection. In some embodiments, the infection is a bacterial infection, a fungal infection, a parasitic infection, or a yeast infection. In some embodiments, the subject has contracted an infectious disease by way of contact with another infected subject. In some embodiments, the subject has contracted the infectious disease from a different species carrying the microorganism. In some embodiments, the subject is a mammal. In some embodiments, the mammal is a human.

Provided herein are methods of stimulating an immune response in a subject, the methods comprising: administering to the subject a composition provided herein in an amount sufficient for stimulating the immune response.

Provided herein are methods of enhancing an immune response to an antigen encoded by an RNA in an RNA vaccine composition, the method comprising: (a) administering to the subject an effective amount of the composition provided herein or the vaccine composition provided herein; and (b) administering to the subject an RNA vaccine composition, wherein the RNA vaccine composition comprises an RNA encoding for an antigen, thereby enhancing the immune response to the antigen encoded by the RNA.

In some embodiments, the methods further comprise administering to the subject an RNA vaccine composition. In some embodiments, the methods further comprise administering to the subject a protein antigen vaccine composition. In some embodiments, the antigen is a spike protein, a glycoprotein, a viral envelope protein, or a cancer cell protein. In some embodiments, the spike protein is from a SARS-CoV-2 virus. In some embodiments, the composition induces an immune response to viral antigen, wherein the viral antigen is selected from an adenovirus, an astrovirus, a bocavirus, a Banna virus, a BK virus, a Coltivirus, a coronavirus, a coxsackievirus, a cytomegalovirus, a Dengue virus, an Ebola virus, an enterovirus, an Epstein-Barr virus, a Hanta virus, a hepatitis A virus, a hepatitis B virus, a hepatitis C virus, a herpes simplex virus, a human immunodeficiency virus (HIV), an influenza virus, a JC virus, a Lassa virus, a Marburg virus, a measles virus, a Middle East Respiratory Syndrome (MERS) coronavirus, a Norwalk virus, a norovirus, a parvovirus, a papillomavirus, a polio virus, a rabies virus, a rhinovirus, a rotavirus, a Rubella virus, a severe acute respiratory syndrome (SARS) virus, a smallpox virus, Varicella-zoster virus, a Yellow fever virus, or any combination thereof.

Further provided herein is a method for the treatment of cancer in a subject, wherein the method comprises administering a composition provided herein, thereby treating the cancer. In some embodiments, the subject has, is diagnosed with, or is at risk of developing cancer. In some embodiments, the administering is intratumoral administration, intrasplenic administration, intradermal administration, intranodal administration, intramuscular administration, oral administration, intranasal administration, or intravenous administration. In some embodiments, the subject has a solid tumor or a blood cancer. In some embodiments, the subject has, is suspected of having, or is diagnosed with prostate cancer, ovarian cancer, breast cancer, lung cancer, leukemia, brain cancer, bladder cancer, colorectal cancer, endometrial cancer, esophageal cancer, head and neck cancer, kidney cancer, liver cancer, lymphoma, melanoma, mesothelioma, multiple myeloma, pancreatic cancer, soft tissue sarcoma, skin cancer, stomach cancer, thyroid cancer, or uterine cancer. In some embodiments, the subject has a solid tumor. In some embodiments, the subject has a metastatic cancer. In some embodiments the composition for use in the treatment of cancer comprises an RNA or a DNA encoding for a tumor antigen provided herein. In some embodiments the composition for use in the treatment of cancer comprises an RNA encoding for an immune regulatory protein provided herein.

### EXEMPLARY EMBODIMENTS

Provided herein are compositions for inducing an immune response following administration to a subject, wherein the compositions comprise: (a) an RNA adjuvant encoding a protein, wherein the protein comprises at least a functional fragment of a toll-like receptor 5 (TLR5) agonist, wherein the functional fragment activates a TLR5 pathway in a cell upon contact with the cell; and (b) one or more delivery vehicle, wherein following administration of an effective amount of the composition to a subject in the presence of an antigen, an initial innate immune response is induced in the subject. Further provided herein are compositions, wherein the one or more delivery vehicle is formulated for intranasal administration, oral administration, intradermal administration, intranodal administration, intrasplenic administration, intratumoral administration, intravenous administration, or intramuscular administration. Further provided herein are compositions, wherein the functional fragment of the TLR5 agonist activates a TLR5 pathway in a cell upon contacting the cell. Further provided herein are compositions, wherein the cell is an epithelial cell. Further provided herein are compositions, wherein the functional fragment of the TLR5 agonist comprises a functional derivative of a bacterial motility protein. Further provided herein are compositions, wherein the bacterial motility protein is a bacterial flagellin protein, a derivative, or a functional fragment thereof. Further provided herein are compositions, wherein the protein is a truncated form of a bacterial flagellin protein, a secreted form of a bacterial flagellin protein, or a combination thereof. Further provided herein are compositions, wherein the functional fragment of a toll-like receptor 5 (TLR5) agonist comprises a D0 region and a D1 region of the bacterial flagellin protein. Further provided herein are compositions, wherein the functional fragment of a toll-like receptor 5 (TLR5) agonist comprises a D0 region, a D1 region, and a D2 region of the bacterial flagellin protein. Further provided herein are compositions, wherein the functional fragment of a toll-like receptor 5 (TLR5) agonist comprises a D0 region, a D1 region, a D2 region, and a D3 region of the bacterial flagellin protein. Further provided herein are compositions, wherein the synthetic protein further comprises a signal sequence. Further provided herein are compositions, wherein the signal sequence comprises an amino acid sequence of: MRSLSVLALLLLLLLAPASA (SEQ ID NO: 1). Further provided herein are compositions, wherein the RNA adjuvant comprises a sequence that is at least 85% identical to any one of SEQ ID NOS: 2 to 37. Further provided herein are compositions for inducing an immune response, wherein the RNA adjuvant comprises a sequence that is at least 90% identical to any one of SEQ ID NOS: 2 to 37. Further provided herein are compositions, wherein the RNA adjuvant comprises a sequence that is at least 95% identical to any one of SEQ ID NOS: 2 to 37. Further provided herein are compositions, wherein the RNA adjuvant comprises a sequence that is at least 96% identical to any one of SEQ ID NOS: 2 to 37. Further provided herein are compositions, wherein the RNA adjuvant comprises a sequence that is at least 97% identical to any one of SEQ ID NOS: 2 to 37. Further provided herein are compositions, wherein the RNA adjuvant comprises a sequence that is at least 98% identical to any one of SEQ ID NOS: 2 to 37. Further provided herein are compositions, wherein the RNA adjuvant comprises a sequence that is at least 99% identical to any one of SEQ ID NOS: 2 to 37. Further provided herein are compositions, wherein the RNA adjuvant comprises a sequence selected from any one of SEQ ID NOS: 2 to 37. Further provided herein are compositions, wherein the one or more delivery vehicles comprise a diluent, an aqueous solution, a particle, an emulsion, a suspension, a lipid carrier, a vesicle, a vector, or any combination thereof. Further provided herein are compositions, wherein the antigen is a viral antigen, a bacterial antigen, a fungal antigen, or a parasitic antigen. Further provided herein are compositions, wherein the antigen is a tumor antigen. Further provided herein are compositions, wherein the compositions further comprise an RNA encoding the antigen, wherein the RNA encoding the antigen is within the same delivery vehicle as the RNA adjuvant. Further provided herein are compositions, wherein the compositions further comprise an RNA encoding the antigen, wherein the RNA encoding the antigen is within a different delivery vehicle as the RNA adjuvant. Further provided herein are compositions, wherein the RNA adjuvant further comprises an RNA sequence encoding the antigen. Further provided herein are compositions, wherein the RNA sequence encoding the antigen is operably linked to the RNA adjuvant. Further provided herein are compositions, wherein the innate immune response comprises an increase in the level or activity of Nuclear factor kappa B (NF-κB) in an epithelial cell of the subject relative to the level or activity of NF-κB in the absence of the composition.

Provided herein are compositions for inducing an immune response following administration to a subject, wherein the compositions comprise: (a) a nucleic acid sequence encoding a protein, wherein the protein comprises at least a functional fragment of a toll-like receptor 5 (TLR5) agonist, wherein the functional fragment activates a TLR5 pathway in a cell upon contact with the cell; and (b) one or more delivery vehicle, wherein following administration of an effective amount of the composition to a subject in the presence of an antigen, an initial innate immune response is induced in the subject. Further provided herein are compositions, wherein the nucleic acid sequence comprises an RNA sequence or a DNA sequence. Further provided herein are compositions, wherein the protein is a synthetic protein.

Provided herein are compositions, wherein the compositions comprise: (a) a lipid carrier; and (b) an RNA sequence encoding at least a functional fragment of a bacterial motility protein, wherein the RNA adjuvant is a TLR5 agonist. Further provided herein are compositions, wherein the RNA adjuvant is encapsulated within the lipid carrier. Further provided herein are compositions, wherein the RNA adjuvant is in complex with the lipid carrier. Further provided herein are compositions, wherein the lipid carrier is a cationic lipid nanoparticle, an ionizable lipid nanoparticle, a polyethylene glycol (PEG) lipid carrier, a polymeric lipid nanoparticle, a polymer-conjugated lipid carrier, a synthetic vesicle, a liposome, an exosome, an endosome, a lipoplex, a lipidoid, a derivative, variant, or any combination thereof. Further provided herein are compositions, wherein the at least a functional fragment of the bacterial motility protein is a bacterial flagellin protein, a derivative, or a functional fragment thereof. Further provided herein are compositions, wherein the bacterial motility protein is a truncated form of a bacterial flagellin protein, a secreted form of a bacterial flagellin protein, or a combination thereof. Further provided herein are compositions, wherein the functional fragment of the bacterial motility protein comprises a D0 region and a D1 region of the bacterial flagellin protein. Further provided herein are compositions, wherein the functional fragment of the bacterial motility protein comprises a D0 region, a D1 region, and a D2 region of the bacterial flagellin protein. Further provided herein are compositions, wherein the functional fragment of the bacterial motility protein comprises a D0 region, a D1 region, a D2 region, and a D3 region of the bacterial flagellin protein. Further provided herein are compositions, wherein the RNA adjuvant comprises an mRNA. Further provided herein are compositions, wherein the composition further comprises one or more RNA molecules each encoding for an antigen. Further provided herein are compositions, wherein the compositions further comprise one or more antigens. Further provided herein are compositions, wherein the RNA encoding for an antigen is an mRNA. Further provided herein are compositions, wherein the mRNA is chemically modified. Further provided herein are compositions, wherein the chemical modification comprises a poly-A tail, a chemically modified nucleobase, or a 5′ terminal cap. Further provided herein are compositions, wherein the antigen is a viral antigen, a bacterial antigen, a fungal antigen, a parasitic antigen, or a tumor antigen. Further provided herein are compositions, wherein the viral antigen is from an adenovirus, an astrovirus, a bocavirus, a Banna virus, a BK virus, a Coltivirus, a coronavirus, a coxsackievirus, a cytomegalovirus, a Dengue virus, an Ebola virus, an enterovirus, an Epstein-Barr virus, a Hanta virus, a hepatitis A virus, a hepatitis B virus, a hepatitis C virus, a herpes simplex virus, a human immunodeficiency virus (HIV), an influenza virus, a JC virus, a Lassa virus, a Marburg virus, a measles virus, a Middle East Respiratory Syndrome (MERS) coronavirus, a Norwalk virus, a norovirus, a parvovirus, a papillomavirus, a polio virus, a rabies virus, a rhinovirus, a rotavirus, a Rubella virus, a severe acute respiratory syndrome (SARS) virus, a smallpox virus, Varicella-zoster virus, a Yellow fever virus, or any combination thereof. Further provided herein are compositions, wherein the SARS virus is a SARS-CoV-2 virus. Further provided herein are compositions, wherein the antigen comprises a spike protein, a glycoprotein, or a hemagglutinin protein. Further provided herein are compositions, wherein the RNA encoding for the antigen is encapsulated within the same lipid carrier as the RNA adjuvant. Further provided herein are compositions, wherein the RNA encoding for the antigen is encapsulated within a different lipid carrier as the RNA adjuvant. Further provided herein are compositions, wherein the RNA adjuvant comprises a sequence that is at least 85% identical to any one of SEQ ID NOS: 2 to 37. Further provided herein are compositions, wherein the RNA adjuvant comprises a sequence that is at least 90% identical to any one of SEQ ID NOS: 2 to 37. Further provided herein are compositions, wherein the RNA adjuvant comprises a sequence that is at least 95% identical to any one of SEQ ID NOS: 2 to 37. Further provided herein are compositions, wherein the RNA adjuvant comprises a sequence selected from any one of SEQ ID NOS: 2 to 37. Further provided herein are compositions, wherein the compositions are in the form of a suspension, an aqueous solution, or an emulsion.

Provided herein are compositions, wherein the compositions comprise: (a) a carrier; and (b) a nucleic acid encoding at least a functional fragment of a bacterial motility protein, wherein the functional fragment of the bacterial motility protein is a TLR5 agonist. Further provided herein are compositions, wherein the carrier is a lipid carrier. Further provided herein are compositions, wherein the nucleic acid comprises RNA, DNA, or a combination thereof.

Provided herein are compositions for inducing an immune response following administration to a subject, wherein the compositions comprise: (a) an RNA adjuvant encoding for a protein, wherein the protein comprises at least a functional fragment of a toll-like receptor 4 (TLR4) agonist; and (b) one or more delivery vehicle formulated for intranasal administration, oral administration, intradermal administration, intranodal administration, intrasplenic administration, intratumoral administration, intravenous administration, or intramuscular administration, wherein following administration of an effective amount of the composition to a subject in the presence of an antigen, an initial innate immune response is induced in the subject. Further provided herein are compositions for inducing an immune response, wherein the TLR4 agonist comprises at least a functional fragment of a high mobility group box 1 (HMGB1) protein, a nicotinate-nucleotide—dimethylbenzimidazole phosphoribosyltransferase, a transglycosylase, a 50S ribosomal protein, a heparin-binding hemagglutinin protein, a YadA family autotransporter adhesin, a dnaJ protein, a pneumolysin protein, an Omp19 protein, a 6,7-dimethyl-8-ribityllumazine synthase, or any combination thereof the TLR4 agonist is from a bacterium. Further provided herein are compositions for inducing an immune response, wherein the TLR4 agonist is from a human. Further provided herein are compositions for inducing an immune response, wherein the RNA comprises a sequence that is at least 85% identical to any one of SEQ ID NOS: 38 to 103. Further provided herein are compositions for inducing an immune response, wherein the RNA comprises a sequence that is at least 90% identical to any one of SEQ ID NOS: 38 to 103. Further provided herein are compositions for inducing an immune response, wherein the RNA comprises a sequence that is at least 95% identical to any one of SEQ ID NOS: 38 to 103. Further provided herein are compositions for inducing an immune response, wherein the RNA comprises a sequence selected from any one of SEQ ID NOS: 38 to 103. Further provided herein are compositions for inducing an immune response, wherein the delivery vehicle is a diluent, an aqueous solution, a particle, an emulsion, a suspension, a lipid carrier, a vesicle, or any combination thereof. Further provided herein are compositions for inducing an immune response, wherein the antigen is a viral antigen, a bacterial antigen, a fungal antigen, a parasitic antigen, or a tumor antigen. Further provided herein are compositions for inducing an immune response, wherein the innate immune response comprises an increase in the level or activity of Nuclear factor kappa B upon administration of the composition relative to the level or activity of NF-κB in the absence of the composition.

Provided herein are compositions, wherein the compositions comprise: (a) a lipid carrier; and (b) an RNA sequence encoding at least a functional fragment of a TLR4 agonist. Further provided herein are compositions, wherein the TLR4 agonist comprises at least a functional fragment of a high mobility group box 1 (HMGB1) protein, a nicotinate-nucleotide—dimethylbenzimidazole phosphoribosyltransferase, a transglycosylase, a 50S ribosomal protein, a heparin-binding hemagglutinin protein, a YadA family autotransporter adhesin, a dnaJ protein, a pneumolysin protein, an Omp19 protein, a 6,7-dimethyl-8-ribityllumazine synthase, or any combination thereof. Further provided herein are compositions, wherein the TLR4 agonist is from a bacterium. Further provided herein are compositions, wherein the TLR4 agonist is from a human. Further provided herein are compositions, wherein the RNA comprises a sequence that is at least 85% identical to any one of SEQ ID NOS: 38 to 103. Further provided herein are compositions, wherein the RNA comprises a sequence that is at least 90% identical to any one of SEQ ID NOS: 38 to 103. Further provided herein are compositions, wherein the RNA comprises a sequence that is at least 95% identical to any one of SEQ ID NOS: 38 to 103. Further provided herein are compositions, wherein the RNA comprises a sequence selected from any one of SEQ ID NOS: 38 to 103. Further provided herein are compositions, wherein the RNA adjuvant is encapsulated within the lipid carrier. Further provided herein are compositions, wherein the RNA adjuvant is in complex with the lipid carrier. Further provided herein are compositions, wherein the lipid carrier is a cationic lipid nanoparticle, an ionizable lipid nanoparticle, a polyethylene glycol (PEG) lipid carrier, a polymeric lipid nanoparticle, a polymer-conjugated lipid carrier, a synthetic vesicle, a liposome, an exosome, an endosome, a lipoplex, a lipidoid, a derivative, variant, or any combination thereof. Further provided herein are compositions, wherein the RNA adjuvant comprises an mRNA. Further provided herein are compositions, wherein the composition further comprises one or more RNA molecules each encoding for an antigen. Further provided herein are compositions, wherein the composition further comprises one or more antigens. Further provided herein are compositions, wherein the RNA encoding for an antigen is an mRNA. Further provided herein are compositions, wherein the mRNA is chemically modified. Further provided herein are compositions, wherein the chemical modification comprises a poly-A tail, a chemically modified nucleobase, or a 5′ terminal cap. Further provided herein are compositions, wherein the antigen is a viral antigen, a bacterial antigen, a fungal antigen, a parasitic antigen, or a tumor antigen. Further provided herein are compositions, wherein the viral antigen is from an adenovirus, an astrovirus, a bocavirus, a Banna virus, a BK virus, a Coltivirus, a coronavirus, a coxsackievirus, a cytomegalovirus, a Dengue virus, an Ebola virus, an enterovirus, an Epstein-Barr virus, a Hanta virus, a hepatitis A virus, a hepatitis B virus, a hepatitis C virus, a herpes simplex virus, a human immunodeficiency virus (HIV), an influenza virus, a JC virus, a Lassa virus, a Marburg virus, a measles virus, a Middle East Respiratory Syndrome (MERS) coronavirus, a Norwalk virus, a norovirus, a parvovirus, a papillomavirus, a polio virus, a rabies virus, a rhinovirus, a rotavirus, a Rubella virus, a severe acute respiratory syndrome (SARS) virus, a smallpox virus, Varicella-zoster virus, a Yellow fever virus, or any combination thereof. Further provided herein are compositions, wherein the SARS virus is a SARS-CoV-2 virus. Further provided herein are compositions, wherein the antigen comprises a spike protein, a glycoprotein, a hemagglutinin protein or a cancer cell protein. Further provided herein are compositions, wherein the RNA encoding for the antigen is encapsulated within the same lipid carrier as the RNA adjuvant. Further provided herein are compositions, wherein the RNA encoding for the antigen is encapsulated within a different lipid carrier as the RNA adjuvant. Further provided herein are compositions, wherein the compositions are in the form of a suspension, an aqueous solution, or an emulsion.

Provided herein are compositions, wherein the compositions comprise: (a) a carrier; and (b) a nucleic acid encoding at least a functional fragment of a TLR4 agonist. Further provided herein are compositions, wherein the carrier is a lipid carrier. Further provided herein are compositions, wherein the nucleic acid comprises RNA, DNA, or a combination thereof.

Provided herein are compositions for inducing an immune response following administration to a subject, wherein the compositions comprise: (a) an RNA adjuvant encoding a protein, wherein the protein comprises at least a functional fragment of a toll-like receptor 2 (TLR2) agonist; and (b) one or more delivery vehicle, wherein following administration of an effective amount of the composition to a subject in the presence of an antigen, an initial innate immune response is induced in the subject. Further provided herein are compositions, wherein the one or more delivery vehicle are formulated for intranasal administration, oral administration, intradermal administration, intranodal administration, intrasplenic administration, intratumoral administration, intravenous administration, or intramuscular administration. Further provided herein are compositions, wherein the TLR2 agonist comprises at least a functional fragment of an outer membrane protein (Omp), a Panton-Valentine bi-component leukocidin (PVL) protein, a porn protein, a TRAP transporter protein, a hemagglutinin protein, an oxidoreductase protein, or a type VII secretion system. Further provided herein are compositions, wherein the TLR2 agonist is from a bacterium. Further provided herein are compositions wherein the RNA adjuvant comprises a sequence that is at least 85% identical to any one of SEQ ID NOS: 104 to 175. Further provided herein are compositions, wherein the RNA adjuvant comprises a sequence that is at least 90% identical to any one of SEQ ID NOS: 104 to 175. Further provided herein are compositions, wherein the RNA adjuvant comprises a sequence that is at least 95% identical to any one of SEQ ID NOS: 104 to 175. Further provided herein are compositions, wherein the RNA comprises a sequence selected from any one of SEQ ID NOS: 104 to 175. Further provided herein are compositions, wherein the one or more delivery vehicles comprise a diluent, an aqueous solution, a particle, an emulsion, a suspension, a lipid carrier, a vesicle, or any combination thereof. Further provided herein are compositions, wherein the antigen is a viral antigen, a bacterial antigen, a fungal antigen, a parasitic antigen, or a tumor antigen. Further provided herein are compositions, wherein the innate immune response comprises an increase in the level or activity of Nuclear factor kappa B in a subject upon administration of the composition relative to the level or activity of NF-kB in the absence of administration of the composition.

Provided herein are compositions, wherein the compositions comprise: (a) a lipid carrier; and (b) an RNA sequence encoding at least a functional fragment of a TLR2 agonist. Further provided herein are compositions, wherein the TLR2 agonist comprises at least a functional fragment of an outer membrane protein (Omp), a Panton-Valentine bi-component leukocidin (PVL) protein, a porin protein, a TRAP transporter protein, a hemagglutinin protein, an oxidoreductase protein, or a type VII secretion system. Further provided herein are compositions, wherein the TLR2 agonist is from a bacterium. Further provided herein are compositions, wherein the RNA sequence comprises a sequence that is at least 85% identical to any one of SEQ ID NOS: 104 to 175. Further provided herein are compositions, wherein the RNA sequence comprises a sequence that is at least 90% identical to any one of SEQ ID NOS: 104 to 175. Further provided herein are compositions, wherein the RNA sequence comprises a sequence that is at least 95% identical to any one of SEQ ID NOS: 104 to 175. Further provided herein are compositions, wherein the RNA sequence comprises a sequence selected from any one of SEQ ID NOS: 104 to 175. Further provided herein are compositions, wherein the RNA sequence is encapsulated within the lipid carrier. Further provided herein are compositions, wherein the RNA sequence is in complex with the lipid carrier. Further provided herein are compositions, wherein the lipid carrier is a cationic lipid nanoparticle, an ionizable lipid nanoparticle, a polyethylene glycol (PEG) lipid carrier, a polymeric lipid nanoparticle, a polymer-conjugated lipid carrier, a synthetic vesicle, a liposome, an exosome, an endosome, a lipoplex, a lipidoid, a derivative, variant, or any combination thereof. Further provided herein are compositions, wherein the RNA sequence comprises an mRNA. Further provided herein are compositions, wherein the composition further comprises one or more nucleic acids each encoding for an antigen. Further provided herein are compositions, wherein the composition further comprises one or more antigens. Further provided herein are compositions, wherein the RNA encoding for an antigen is an mRNA. Further provided herein are compositions, wherein the mRNA is chemically modified. Further provided herein are compositions, wherein the chemical modification comprises a poly-A tail, a chemically modified nucleobase, or a 5′ terminal cap. Further provided herein are compositions, wherein the antigen is a viral antigen, a bacterial antigen, a fungal antigen, a parasitic antigen, or a tumor antigen. Further provided herein are compositions, wherein the viral antigen is from an adenovirus, an astrovirus, a bocavirus, a Banna virus, a BK virus, a Coltivirus, a coronavirus, a coxsackievirus, a cytomegalovirus, a Dengue virus, an Ebola virus, an enterovirus, an Epstein-Barr virus, a Hanta virus, a hepatitis A virus, a hepatitis B virus, a hepatitis C virus, a herpes simplex virus, a human immunodeficiency virus (HIV), an influenza virus, a JC virus, a Lassa virus, a Marburg virus, a measles virus, a Middle East Respiratory Syndrome (MERS) coronavirus, a Norwalk virus, a norovirus, a parvovirus, a papillomavirus, a polio virus, a rabies virus, a rhinovirus, a rotavirus, a Rubella virus, a severe acute respiratory syndrome (SARS) virus, a smallpox virus, Varicella-zoster virus, a Yellow fever virus, or any combination thereof. Further provided herein are compositions, wherein the SARS virus is a SARS-CoV-2 virus. Further provided herein are compositions, wherein the antigen comprises a spike protein, a glycoprotein, a hemagglutinin protein, or a cancer cell protein. Further provided herein are compositions, wherein the RNA sequence encoding for the antigen is encapsulated within the same lipid carrier as the RNA sequence. Further provided herein are compositions, wherein the RNA sequence encoding for the antigen is encapsulated within a different lipid carrier as the RNA sequence encoding for the TLR2 agonist. Further provided herein are compositions, wherein the compositions are in the form of a suspension, an aqueous solution, or an emulsion.

Provided herein are compositions, wherein the compositions comprise: (a) a carrier; and (b) a nucleic acid encoding for at least a functional fragment of a TLR2 agonist. Further provided herein are compositions, wherein the carrier is a lipid carrier. Further provided herein are compositions, wherein the nucleic acid comprises RNA, DNA, or a combination thereof.

Provided herein are compositions for inducing an immune response upon administration to a subject, the compositions comprising: (a) one or more carriers; (b) one or more nucleic acids, wherein the one or more nucleic acids encode for: (i) at least a functional fragment of a TLR4 agonist, a TLR2 agonist, a TLR5 agonist, or any combination thereof; (ii) a viral antigen; or (iii) a combination of (i) and (ii); and (c) a delivery vehicle, wherein following the administration of an effective amount of the composition, an innate immune response is induced in the subject in the presence of the viral antigen, thereby inducing an immune response to the viral antigen. Further provided herein are compositions, wherein the one or more nucleic acids comprise RNA, DNA, or a combination thereof. Further provided herein are compositions, wherein the compositions comprise an RNA sequence encoding the functional fragment of a TLR4 agonist, a TLR2 agonist, a TLR5 agonist, or any combination thereof. Further provided herein are compositions, wherein the compositions comprise an RNA encoding the viral antigen. Further provided herein are compositions, wherein the one or more carriers comprise a lipid carrier. Further provided herein are compositions, wherein the delivery vehicle is formulated for intranasal administration, oral administration, intradermal administration, intranodal administration, intrasplenic administration, intratumoral administration, intravenous administration, or intramuscular administration. Further provided herein are compositions, wherein the functional fragment of a TLR4 agonist, a TLR2 agonist, a TLR5 agonist, or any combination thereof activates a TLR4, a TLR2, or a TLR5 pathway in a cell upon contact with the cell. Further provided herein are compositions, wherein the cell is an epithelial cell. Further provided herein are compositions, wherein the viral antigen is a spike protein, a glycoprotein, or a viral envelope protein. Further provided herein are compositions, wherein the spike protein is from a SARS-CoV-2 virus. Further provided herein are compositions, wherein the RNA encoding the viral antigen is an mRNA. Further provided herein are compositions, wherein the RNA encoding the viral antigen is chemically modified. Further provided herein are compositions, wherein the chemical modification comprises a poly-A tail, a chemically modified nucleobase, or a 5′ terminal cap. Further provided herein are compositions, wherein the one or more carriers are in complex with the one or more RNA adjuvants. Further provided herein are compositions, wherein the one or more carriers are in complex with the one or more nucleic acids encoding a viral antigen. Further provided herein are compositions, wherein the one or more nucleic acids are encapsulated by the one or more lipid carriers. Further provided herein are compositions, wherein the one or more nucleic acids encoding a viral antigen are encapsulated within the one or more carriers. Further provided herein are compositions, wherein the one or more nucleic acids the one or more nucleic acids encoding the TLR5 agonist, the TLR4 agonist, or the TLR2 agonist is operably linked to the one or more nucleic acids encoding the viral antigen. Further provided herein are compositions, wherein the one or more nucleic acids comprise a sequence selected from SEQ ID NOS: 2 to 175, or a functional fragment thereof. Further provided herein are compositions, wherein upon administration of the composition to a subject, an innate immune response is induced in the subject that is greater than the innate immune response in a subject that is not administered an effective amount of the composition. Further provided herein are compositions, wherein upon administration of the composition to a subject, an innate immune response is induced in the subject that is greater than the innate immune response in a subject that is only administered a composition comprising a nucleic acid encoding the viral antigen alone. Further provided herein are compositions, wherein the innate immune response comprises an increase in the level or activity of Nuclear factor kappa B upon administration of the effective amount of the composition relative to the level or activity of NF-κB in the absence of the composition.

Provided herein are compositions, wherein the compositions comprise: (a) an RNA adjuvant comprising a nucleic acid sequence selected from any one of SEQ ID NOS: 2 to 175; and (b) a delivery vehicle. Further provided herein are compositions, wherein the delivery vehicle is selected from a diluent, an aqueous solution, a particle, an emulsion, a suspension, a lipid carrier, a vesicle, a yeast cell, or any combination thereof.

Provided herein are compositions, wherein the compositions comprise: (a) a DNA sequence encoding for an RNA adjuvant comprising a nucleic acid sequence selected from any one of SEQ ID NOS: 2 to 175; and (b) a delivery vehicle. Further provided herein are compositions, wherein the delivery vehicle is selected from a diluent, an aqueous solution, a particle, an emulsion, a suspension, a lipid carrier, a vesicle, a yeast cell, or any combination thereof.

Provided herein are compositions, wherein the compositions comprise: (a) a protein comprising an amino acid sequence that is at least 75%, at least 80%, at least 85%, at least 90%, at least 95%, at least 96%, at least 97%, at least 98%, at least 99%, or at least 100% identical to a sequence selected from any one of SEQ ID NOS: 176 to 217; and (b) a delivery vehicle. Further provided herein are compositions, wherein the delivery vehicle is selected from a diluent, an aqueous solution, a particle, an emulsion, a suspension, a lipid carrier, a vesicle, a yeast cell, or any combination thereof.

Provided herein are compositions, wherein the compositions comprise: (a) a DNA sequence encoding a synthetic protein comprising an amino acid sequence selected from any one of SEQ ID NOS: 176 to 217; and (b) a delivery vehicle. Further provided herein are compositions, wherein the delivery vehicle is selected from a diluent, an aqueous solution, a particle, an emulsion, a suspension, a lipid carrier, a vesicle, a yeast cell, or any combination thereof.

Provided herein are compositions, wherein the compositions comprise: (a) an RNA sequence encoding a synthetic protein comprising an amino acid sequence selected from any one of SEQ ID NOS: 176 to 217; and (b) a delivery vehicle. Further provided herein are compositions, wherein the delivery vehicle is selected from a diluent, an aqueous solution, a particle, an emulsion, a suspension, a lipid carrier, a vesicle, a yeast cell, or any combination thereof. Further provided herein are compositions, wherein the compositions comprise one or more nucleic acids, and wherein the one or more nucleic acids comprise RNA, DNA, or a combination thereof. Further provided herein are nucleic acids encoding an amino acid sequence that is at least 80% identical to SEQ ID NO: 206. Further provided herein are nucleic acids encoding an amino acid sequence that is at least 80% identical to SEQ ID NO: 210. Further provided herein are nucleic acids encoding an amino acid sequence that is at least 75% identical to SEQ ID NO: 212. Further provided herein are nucleic acids encoding an amino acid sequence that is at least 85% identical to SEQ ID NO: 213. Further provided herein are nucleic acids encoding an amino acid sequence that is at least 75% identical to SEQ ID NO: 215. Further provided herein are nucleic acids encoding an amino acid sequence that is at least 75% identical to SEQ ID NO: 217.

Provided herein are compositions, wherein the compositions comprise: a synthetic protein comprising a protein construct, wherein the protein construct comprises an amino acid sequence selected from SEQ ID NOS: 176 to 217. Further provided herein are compositions comprising two or more, three or more, four or more, or five or more protein constructs. Further provided herein are nucleic acids encoding for a protein construct that is at least 80% identical to SEQ ID NO: 206. Further provided herein are nucleic acids encoding for a protein construct that is at least 80% identical to SEQ ID NO: 210. Further provided herein are nucleic acids encoding for a protein construct that is at least 75% identical to SEQ ID NO: 212. Further provided herein are nucleic acids encoding for a protein construct that is at least 85% identical to SEQ ID NO: 213. Further provided herein are nucleic acids encoding for a protein construct that is at least 75% identical to SEQ ID NO: 215. Further provided herein are nucleic acids encoding for a protein construct that is at least 75% identical to SEQ ID NO: 217.

Provided herein are compositions, wherein the compositions comprise: (a) a yeast cell, wherein the yeast cell comprises a permeable cell wall; and (b) one or more RNA adjuvant, wherein the one or more RNA adjuvant for at least a functional fragment of a TLR4 agonist, a TLR2 agonist, a TLR5 agonist, or a combination thereof. Provided herein are compositions, wherein the compositions comprise: (a) a yeast cell, wherein the yeast cell comprises a permeable cell wall; and (b) one or more nucleic acids, wherein the nucleic acids encode for at least a functional fragment of a TLR4 agonist, a TLR2 agonist, a TLR5 agonist, or a combination thereof. Provided herein are compositions, wherein the compositions comprise: (a) a yeast cell, wherein the yeast cell comprises a permeable cell wall; and (b) one or more synthetic proteins, wherein the one or more synthetic proteins comprise at least a functional fragment of a TLR4 agonist, a TLR2 agonist, a TLR5 agonist, or a combination thereof. Further provided herein are compositions, wherein the compositions further comprise an RNA encoding for a viral antigen. Further provided herein are compositions, wherein the compositions further comprise a DNA encoding for a viral antigen. Further provided herein are compositions, wherein the compositions further comprise a chemically modified RNA encoding for a viral antigen. Further provided herein are compositions, wherein the one or more RNA adjuvant or nucleic acids comprise a sequence that is at least 85% identical to any one of SEQ ID NOS: 2 to 175. Further provided herein are compositions, wherein the one or more RNA adjuvant or nucleic acids comprise a sequence that is at least 90% identical to any one of SEQ ID NOS: 2 to 175. Further provided herein are compositions, wherein the one or more RNA adjuvant or nucleic acids comprise a sequence that is at least 95% identical to any one of SEQ ID NOS: 2 to 175. Further provided herein are compositions, wherein the one or more RNA adjuvant or nucleic acids comprise any one of SEQ ID NOS: 2 to 175. Further provided herein are compositions, wherein the one or more RNA adjuvant or nucleic acids encode for an amino acid sequence that is at least 75%, at least 80%, at least 85%, at least 90%, at least 95%, at least 96%, at least 97%, at least 98%, at least 99%, or 100% identical to a sequence selected from any one of SEQ ID NOS: 176 to 217. Further provided herein are nucleic acids encoding an amino acid sequence that is at least 80% identical to SEQ ID NO: 206. Further provided herein are nucleic acids encoding an amino acid sequence that is at least 80% identical to SEQ ID NO: 210. Further provided herein are nucleic acids encoding an amino acid sequence that is at least 75% identical to SEQ ID NO: 212. Further provided herein are nucleic acids encoding an amino acid sequence that is at least 85% identical to SEQ ID NO: 213. Further provided herein are nucleic acids encoding an amino acid sequence that is at least 75% identical to SEQ ID NO: 215. Further provided herein are nucleic acids encoding an amino acid sequence that is at least 75% identical to SEQ ID NO: 217. Further provided herein are compositions, wherein the one or more RNA adjuvant or nucleic acids are encapsulated within the yeast cell. Further provided herein are compositions, wherein the compositions further comprise an enteric-coated microsphere, wherein the yeast cell is encapsulated within the enteric-coated microsphere. Further provided herein are compositions, wherein the composition is formulated for oral administration to a subject.

Provided herein are compositions for inducing an immune response upon administration to a subject, wherein the compositions comprise: (a) one or more carriers; (b) one or more nucleic acids, wherein the one or more nucleic acids encode for: (i) at least a functional fragment of a TLR4 agonist, a TLR2 agonist, a TLR5 agonist, or any combination thereof; (ii) a tumor antigen; (iii) an immune regulatory protein; or (iv) any combination of (i)-(iii); and (c) a delivery vehicle, wherein following the administration of an effective amount of the composition, an innate immune response is induced in the subject in the presence of the tumor antigen, thereby inducing an immune response to the tumor antigen. Further provided herein are compositions, wherein the one or more carriers comprise a lipid carrier. Further provided herein are compositions, wherein the immune regulatory protein is CD83, 4-1BB ligand, or a cytokine. Further provided herein are compositions, wherein the tumor antigen is mucin 1 (MUC1), carcinoembryonic antigen (CEA), human epidermal growth factor receptor 2 (Her2/neu), telomerase (TERT), survivin, melanoma-associated antigen A1 (MAGE-A1), MAGE-A3, Wilms tumor 1 (WT1), preferentially expressed antigen of melanoma (PRAME), New York esophagus 1 (NY-ESO-1), tumor protein p53, tyrosinase, S100 protein, Melanoma-associated antigen recognized by T cells 1 (MART-1), or any combination thereof. Further provided herein are compositions, wherein the one or more nucleic acids comprise RNA, DNA, or a combination thereof. Further provided herein are compositions, wherein the one or more nucleic acids encoding the tumor antigen and/or immune regulatory protein is chemically modified. Further provided herein are compositions, wherein the chemical modification comprises a poly-A tail, a chemically modified nucleobase, or a 5′ terminal cap. Further provided herein are compositions, wherein the one or more lipid carriers are in complex with the one or more RNA adjuvants. Further provided herein are compositions, wherein the one or more lipid carriers are in complex with the at least one RNA encoding a tumor antigen and/or immune regulatory protein. Further provided herein are compositions, wherein the one or more RNA adjuvants are encapsulated by the one or more lipid carriers. Further provided herein are compositions, wherein the at least one RNA encoding a tumor antigen and/or immune regulatory protein is encapsulated by the one or more lipid carriers. Further provided herein are compositions, wherein the one or more RNA adjuvant is operably linked to the at least one RNA encoding a viral antigen, a derivative, or a functional fragment thereof. Further provided herein are compositions, wherein the one or more RNA adjuvant comprises a sequence selected from SEQ ID NOS: 2 to 175, or a functional fragment thereof. Further provided herein are compositions, wherein the innate immune response is induced in the subject is greater than the innate immune response in a subject that is not administered an effective amount of the composition.

Provided herein are vectors, wherein the vectors comprise: one or more nucleic acids encoding: a TLR5 agonist, a TLR4 agonist, a TLR2 agonist, or a combination thereof. Further provided herein are vectors, wherein the vector comprises a viral vector. Further provided herein are vectors, wherein the viral vector comprises an adenovirus, an adeno-associated virus (AAV), a recombinant AAV (rAAV), a lentivirus, an alphavirus, a flavivirus, a rhabdovirus, a Kunjin virus, a measles virus, a Lassa virus or a virus-like particle thereof. Further provided herein are vectors, wherein the TLR5 agonist comprises a functional derivative of a bacterial motility protein. Further provided herein are vectors, wherein the TLR5 agonist comprises a DNA sequence encoding for an RNA sequence that is at least 85% identical to any one of SEQ ID NOS: 2 to 37. Further provided herein are vectors, wherein the TLR5 agonist comprises a sequence that is at least 85% identical to any one of SEQ ID NOS: 2 to 37. Further provided herein are vectors, wherein the TLR4 agonist comprises at least a functional fragment of a high mobility group box 1 (HMGB1) protein, a nicotinate-nucleotide—dimethylbenzimidazole phosphoribosyltransferase, a transglycosylase, a 50S ribosomal protein, a heparin-binding hemagglutinin protein, a YadA family autotransporter adhesin, a dnaJ protein, a pneumolysin protein, an Omp19 protein, a 6,7-dimethyl-8-ribityllumazine synthase, or any combination thereof. Further provided herein are vectors, wherein the one or more nucleic acids comprises a DNA sequence encoding for an RNA sequence that is at least 85% identical to any one of SEQ ID NOS: 38 to 103. Further provided herein are vectors, wherein the one or more nucleic acids comprises a sequence that is at least 85% identical to any one of SEQ ID NOS: 38 to 103. Further provided herein are vectors, wherein the TLR2 agonist comprises at least a functional fragment of an outer membrane protein (Omp), a Panton-Valentine bi-component leukocidin (PVL) protein, a porin protein, a TRAP transporter protein, a hemagglutinin protein, an oxidoreductase protein, or a type VII secretion system. Further provided herein are vectors, wherein the one or more nucleic acids comprise a DNA sequence encoding for an RNA sequence that is at least 85% identical to any one of SEQ ID NOS: 104 to 175. Further provided herein are vectors, wherein the one or more nucleic acids comprise a sequence that is at least 85% identical to any one of SEQ ID NOS: 104 to 175. Further provided herein are vectors, wherein the vector further comprises a nucleic acid sequence encoding a viral antigen, a bacterial antigen, a fungal antigen, a parasite antigen, or a tumor antigen. In some embodiments, the nucleic acid sequence is an RNA sequence or a DNA sequence. Further provided herein are vectors, wherein the viral antigen comprises a SARS-CoV-2 spike protein.

Provided herein are compositions, wherein the compositions comprise a vector provided herein; and a lipid carrier.

Provided herein are vaccine compositions, wherein the vaccine compositions comprise a composition provided herein; and a pharmaceutically acceptable excipient.

Provided herein are vaccine compositions, wherein the vaccine compositions comprise: (a) a lipid carrier; (b) one or more RNA adjuvant, wherein the one or more RNA adjuvant comprises a sequence that is at least 85% identical to the sequence of any one of SEQ ID NOS: 2 to 175; and (c) one or more RNA encoding an antigen. Further provided herein are vaccine compositions, wherein the one or more RNA encoding an antigen each encode a viral antigen, a bacterial antigen, a fungal antigen, a parasitic antigen, a tumor antigen, or any combination thereof.

Provided herein are methods for stimulating an immune response in a subject, the methods comprising: administering to the subject an effective amount of a composition provided herein or the vaccine composition provided herein, thereby stimulating the immune response. Further provided herein are methods for stimulating an immune response in a subject, wherein the administering is systemic administration or local administration. Further provided herein are methods for stimulating an immune response in a subject, wherein the administering is intramuscular administration, intravenous administration, oral administration, intradermal administration, intratumoral administration, inhalation, subcutaneous administration, or intraperitoneal administration. Further provided herein are methods for stimulating an immune response in a subject, wherein the administering is daily, weekly, monthly, or yearly. Further provided herein are methods for stimulating an immune response in a subject, wherein the method further comprises administering to the subject an RNA vaccine composition. Further provided herein are methods for stimulating an immune response in a subject, wherein the subject has or is at risk of developing an infection. Further provided herein are methods for stimulating an immune response in a subject, wherein the infection is a viral infection. Further provided herein are methods for stimulating an immune response in a subject, wherein the viral infection is an upper respiratory viral infection. Further provided herein are methods for stimulating an immune response in a subject, wherein the upper respiratory viral infection is COVID-19, SARS infection, MERS infection, influenza, parainfluenza, respiratory syncytial virus (RSV) infection, pneumonia, or a rhinoviral infection. Further provided herein are methods for stimulating an immune response in a subject, wherein the infection is a bacterial infection, a fungal infection, a parasitic infection, or a yeast infection. Further provided herein are methods for stimulating an immune response in a subject, wherein the antigen is a spike protein, a glycoprotein, a viral envelope protein, or a tumor antigen. Further provided herein are methods for stimulating an immune response in a subject, wherein the spike protein is from a SARS-CoV-2 virus. Further provided herein are methods for stimulating an immune response in a subject, wherein the subject is a mammal. Further provided herein are methods for stimulating an immune response in a subject, wherein the subject is a human. Further provided herein are methods for stimulating an immune response in a subject, wherein the subject is administered an additional dose of the composition or the vaccine composition.

Provided herein are methods of enhancing an immune response to an antigen encoded by an RNA in an RNA vaccine composition, the method comprising: (a) administering to the subject an effective amount of a composition provided herein or the vaccine composition provided herein; and (b) administering to the subject an RNA vaccine composition, wherein the RNA vaccine composition comprises an RNA encoding for an antigen, thereby enhancing the immune response to the antigen encoded by the RNA. Further provided herein are methods of enhancing an immune response to an antigen encoded by an RNA in an RNA vaccine composition, wherein the administering is systemic administration or local administration. Further provided herein are methods of enhancing an immune response to an antigen encoded by an RNA in an RNA vaccine composition, wherein the administering is intramuscular administration, intravenous administration, oral administration, intradermal administration, intratumoral administration, inhalation, subcutaneous administration, or intraperitoneal administration. Further provided herein are methods of enhancing an immune response to an antigen encoded by an RNA in an RNA vaccine composition, wherein the administering is daily, weekly, monthly, or yearly. Further provided herein are methods of enhancing an immune response to an antigen encoded by an RNA in an RNA vaccine composition, wherein the subject has or is at risk of developing an infection. Further provided herein are methods of enhancing an immune response to an antigen encoded by an RNA in an RNA vaccine composition, wherein the infection is a viral infection. Further provided herein are methods of enhancing an immune response to an antigen encoded by an RNA in an RNA vaccine composition, wherein the viral infection is an upper respiratory viral infection. Further provided herein are methods of enhancing an immune response to an antigen encoded by an RNA in an RNA vaccine composition, wherein the upper respiratory viral infection is COVID-19, SARS infection, MERS infection, influenza, parainfluenza, respiratory syncytial virus (RSV) infection, pneumonia, or a rhinoviral infection. Further provided herein are methods of enhancing an immune response to an antigen encoded by an RNA in an RNA vaccine composition, wherein the infection is a bacterial infection, a fungal infection, a parasitic infection, or a yeast infection Further provided herein are methods of enhancing an immune response to an antigen encoded by an RNA in an RNA vaccine composition, wherein the antigen is a spike protein, a glycoprotein, a viral envelope protein, or a tumor antigen. Further provided herein are methods of enhancing an immune response to an antigen encoded by an RNA in an RNA vaccine composition, wherein the spike protein is from a SARS-CoV-2 virus. Further provided herein are methods of enhancing an immune response to an antigen encoded by an RNA in an RNA vaccine composition, wherein the subject is a mammal. Further provided herein are methods of enhancing an immune response to an antigen encoded by an RNA in an RNA vaccine composition, wherein the subject is a human. Further provided herein are methods of enhancing an immune response to an antigen encoded by an RNA in an RNA vaccine composition, wherein the subject is administered an additional dose of the composition or the vaccine composition. Further provided herein are methods of enhancing an immune response to an antigen encoded by an RNA in an RNA vaccine composition, wherein the subject has, is diagnosed with, or is at risk of developing cancer. Further provided herein are methods of enhancing an immune response to an antigen encoded by an RNA in an RNA vaccine composition, wherein the subject has is a solid tumor or a blood cancer. Further provided herein are methods of enhancing an immune response to an antigen encoded by an RNA in an RNA vaccine composition, wherein the cancer is lung cancer, breast cancer, brain cancer, pancreatic cancer, prostate cancer, skin cancer, bladder cancer, lung cancer, liver cancer, ovarian cancer, renal cancer, endometrial cancer, colorectal cancer, gastric cancer, skin cancer, head and neck cancer, or thyroid cancer.

Provided herein are methods for the treatment of an infection in a subject, the methods comprising: administering to a subject an effective amount of a composition provided herein or the vaccine composition provided herein, thereby treating an infection in the subject. Further provided herein are methods for the treatment of an infection in a subject, wherein the administering is systemic administration or local administration. Further provided herein are methods for the treatment of an infection in a subject, wherein the administering is intramuscular administration, intravenous administration, oral administration, intradermal administration, intratumoral administration, inhalation, subcutaneous administration, or intraperitoneal administration. Further provided herein are methods for the treatment of an infection in a subject, wherein the administering is daily, weekly, monthly, or yearly. In some embodiments, the administering is at least about every 6 hours, 12 hours, 24 hours (1 day), 48 hours (2 days), 72 hours (3 days), 96 hours (4 days), 120 hours (5 days), 144 hours (6 days), 168 hours (7 days), 240 hours (10 days), 336 hours (14 days), 504 hours (21 days), 672 hours (28 days), 720 hours (30 days, one month), 840 hours (35 days), 1344 hours (56 days), 8760 hours (365 days, 1 year), or more. Further provided herein are methods for the treatment of an infection in a subject, wherein the methods further comprise administering to the subject an RNA vaccine composition. wherein the infection is a viral infection. Further provided herein are methods for the treatment of an infection in a subject, wherein the viral infection is an upper respiratory viral infection. Further provided herein are methods for the treatment of an infection in a subject, wherein the infection is an oncoviral infection. Further provided herein are methods for the treatment of an infection in a subject, wherein the upper respiratory viral infection is COVID-19, SARS infection, MERS infection, influenza, parainfluenza, respiratory syncytial virus (RSV) infection, pneumonia, or a rhinoviral infection. Further provided herein are methods for the treatment of an infection in a subject, wherein the infection is a bacterial infection, a fungal infection, a parasitic infection, or a yeast infection Further provided herein are methods for the treatment of an infection in a subject, wherein the antigen is a spike protein, a glycoprotein, a viral envelope protein, or a tumor antigen. Further provided herein are methods for the treatment of an infection in a subject, wherein the spike protein is from a SARS-CoV-2 virus. Further provided herein are methods for the treatment of an infection in a subject, wherein the oncoviral infection is from a human papillomavirus (HPV), a Kaposi Sarcoma-Associated Herpesvirus (KSHV), a Merkel Cell Polyomavirus (MCV), a Human T-Cell Lymphotropic Virus Type 1 (HTLV-1), or an Epstein-Barr Virus (EBV). Further provided herein are methods for the treatment of an infection in a subject, wherein the subject is a mammal. Further provided herein are methods for the treatment of an infection in a subject, wherein the subject is a human. Further provided herein are methods for the treatment of an infection in a subject, wherein the subject is administered an additional dose of the composition or the vaccine composition.

Provided herein are methods for the treatment of a cancer in a subject, the methods comprising: administering to a subject an effective amount of a composition provided herein or the vaccine composition provided herein, thereby treating a cancer in the subject. Further provided herein are methods for the treatment of cancer, wherein the administering is systemic administration or local administration. Further provided herein are methods for the treatment of cancer, wherein the administering is intramuscular administration, intravenous administration, oral administration, intradermal administration, intratumoral administration, inhalation, subcutaneous administration, or intraperitoneal administration. Further provided herein are methods for the treatment of cancer, wherein the administering is daily, weekly, monthly, or yearly. In some embodiments, the administering is at least about every 6 hours, 12 hours, 24 hours (1 day), 48 hours (2 days), 72 hours (3 days), 96 hours (4 days), 120 hours (5 days), 144 hours (6 days), 168 hours (7 days), 240 hours (10 days), 336 hours (14 days), 504 hours (21 days), 672 hours (28 days), 720 hours (30 days, one month), 840 hours (35 days), 1344 hours (56 days), or 8760 hours (365 days, 1 year). Further provided herein are methods for the treatment of cancer, wherein the method further comprises administering to the subject an RNA vaccine composition. Further provided herein are methods for the treatment of cancer, wherein the subject has, is diagnosed with, or is at risk of developing cancer. Further provided herein are methods for the treatment of cancer, wherein the subject has is a solid tumor or a blood cancer. Further provided herein are methods for the treatment of cancer, wherein the cancer is lung cancer, breast cancer, brain cancer, pancreatic cancer, prostate cancer, skin cancer, bladder cancer, lung cancer, liver cancer, ovarian cancer, renal cancer, endometrial cancer, colorectal cancer, gastric cancer, skin cancer, head and neck cancer, or thyroid cancer. Further provided herein are methods for the treatment of cancer, wherein the composition comprises an mRNA encoding a tumor antigen. Further provided herein are methods for the treatment of cancer, wherein the tumor antigen is mucin 1 (MUC1), carcinoembryonic antigen (CEA), human epidermal growth factor receptor 2 (Her2/neu), telomerase (TERT), survivin, melanoma-associated antigen A1 (MAGE-A1), MAGE-A3, Wilms tumor 1 (WTT), preferentially expressed antigen of melanoma (PRAME), New York esophagus 1 (NY-ESO-1), tumor protein p53, tyrosinase, S100 protein, Melanoma-associated antigen recognized by T cells 1 (MART-1), or any combination thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention.

### EXAMPLES

### Example 1: Mechanisms of RNA Adjuvant Induction of the Innate and Adaptive Immune Response

The functional mechanisms of RNA adjuvants are described below.

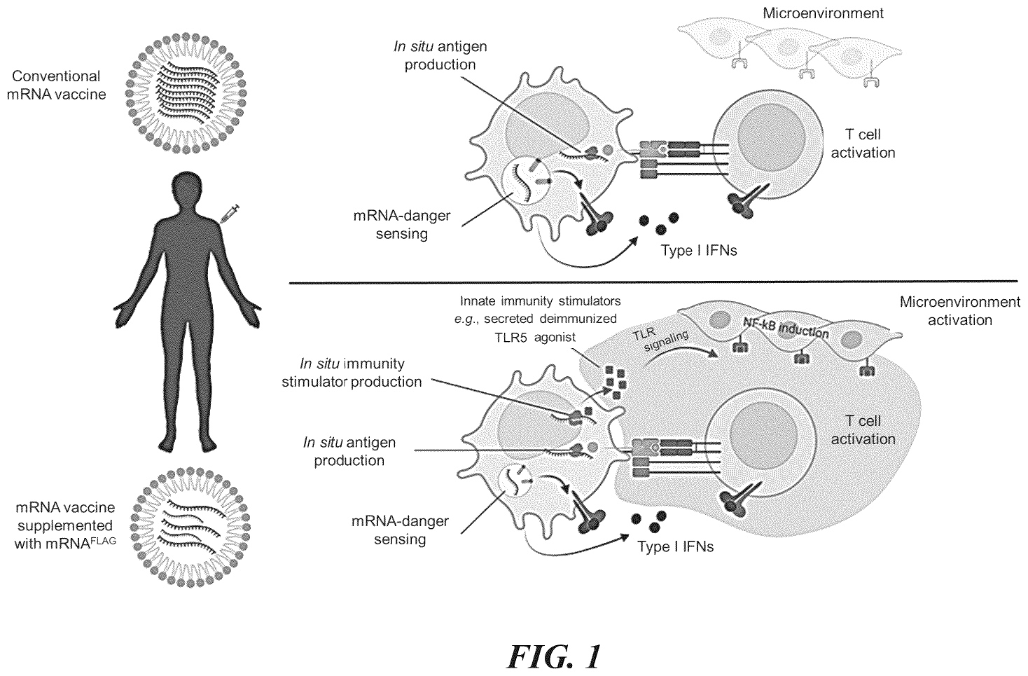

FIG. 1 shows a schematic of the mRNA adjuvant approach to mRNA vaccine improvement. FIG. 1 (top section) provides the mechanism of immunostimluation by conventional mRNA vaccines, where the lipid nanoparticle or carrier for the RNA coding for the antigen is internalized by the target cell and recognized by cellular signaling mechanisms such as TLR7/8 and RIG-1, which leads to downstream interferon type 1 response (IFN). For conventional mRNA vaccines, the mRNA is transported into a host cell via a lipid nanoparticle. The host cell translational machinery translates the mRNA to the antigen protein which is then presented by cell in association with major histocompatibility complex (MHC) molecules. MHC class II molecules present the antigen to CD4+ helper T cells. MHC class I molecules present the antigen to CD8+ cytotoxic T cells. Following interaction of the presented antigen and MHC class II molecules with the CD4+ helper T cell receptor, the CD4+ lymphocyte is activated, IL-2 is released, and IL-2 receptors are expressed on the CD4+ lymphocyte surface. The IL-2 produced by the activated cell stimulates its own receptors, as well as those of mononuclear phagocytes, increasing their microbicidal activity. IL-2 also stimulates B cells to synthesize antibody. Whereas B cells may recognize a protein antigen in its native state, T cells only recognize the peptides, that result from antigen processing, in the context of major histocompatibility complex molecules. Traditional vaccines can activate the production of interferons (IFN), which can block efficient translation and reduce antigen production in the host cell. The IFN signaling causes a Th-1 polarized response.

FIG. 1 (bottom section) provides the mechanism of an mRNA vaccine powered by the mRNA^{Flag} adjuvant, which leads to the induction of NF-κB signaling via TLR5, TLR4 or TLR2 stimulation and release of immunostimulatory cytokines and chemokines are expected to complement and act in a synergistic manner with the mRNA-mediated IFN response to mobilize the immune response to the site of mRNA vaccine-encoded antigen production. Leading to the improvement of quality of immune response due to a balanced Th1-/Th2-polarized immune response enabled by the TLR5-agonistic adjuvant.

### Example 2: RNA Adjuvant Generation, Purification, and Characterization

The generation of RNA adjuvants and RNA-encoded antigens are described. The RNAs described are synthesized by in vitro transcription of a DNA fragment encoding for all elements. Cap structures can be covalently attached to the 5′ UTR either co- or post-transcriptionally via different capping enzymes.

The final RNA product is purified using affinity chromatography on oligo-dT in order to remove the impurities generated during transcription (such as double-stranded RNA), which could potently activate the innate immune response.

Additional modifications to the RNA can be made. Untranslated regions (UTRs) of the RNA are optimized using machine learning techniques trained on ribosomal loading profiles of a reporter gene library, in which the 5′ UTRs contain completely random sequences. This is further tested and validated on libraries of human UTRs and variants associated with diseases in humans. A reporter gene can also be used and linked at the 3′ end to random cDNAs obtained by reverse transcription of fragmented mRNA isolated from human dendritic cells for 3′ UTR modifications. Adding a second 3′ UTR from a different gene can further enhance mRNA stability and protein expression. In addition, the RNA can be optimized with a poly-A tail by testing the levels of a reporter protein expressed from mRNAs differing in poly-A tail lengths. Modified nucleobases can be used to further stabilize the RNA adjuvants and RNA encoding antigens described herein.

Characterization of the RNA can be accomplished using a procedure selected from polynucleotide mapping, reverse transcriptase sequencing, charge distribution analysis, and detection of RNA impurities, wherein characterizing comprises determining the RNA transcript sequence, determining the purity of the RNA transcript, or determining the charge heterogeneity of the RNA Transcript.

### Example 3: RNA Synthesis and Purification

The mRNA compositions provided herein can be synthesized using a T7 phage. Briefly, the mRNA is codon-optimized, synthesized, and cloned into an mRNA production plasmid. After ligation into expression vectors, mRNAs are produced using T7 RNA polymerase on linearized plasmids. The purification of mRNA is performed via a bead-based dialysis or spin column chromatography.

### Example 4: RNA Complexing with Lipid Carriers

RNA adjuvants and mRNAs encoding for a SAR-CoV-2 spike protein are complexed with a lipid carrier. The lipid carrier can be composed of ionizable cationic lipids, cholesterol, phospholipids (such as distearoylphosphatidylcholine), and polyethylene glycol (PEG)-lipid. The spike protein can include a signal sequence targeting the antigen to the endoplasmic reticulum of a cell, S1, and S2 domains, as well as the receptor binding domain (RBD) followed by the short transmembrane and cytoplasmic domains. The RNA can further encode for a furin cleavage site that stabilizes the pre-fusion conformation of the spike protein.

The RNA adjuvant sequences are provided in Table 2 and in the sequences section of this document. The RNAs can be admixed with the desired lipid carrier to form a vaccine composition. The resulting configurations of the RNA adjuvants with RNAs encoding spike are provided in FIGS. 2A-2D.

Configuration 1: The RNA adjuvants are encapsulated within the same lipid carrier as the antigen-encoding RNAs (FIG. 2A).

Configuration 2: The RNA adjuvants are encapsulated within different lipid carriers than the antigen-encoding RNAs (FIG. 2B).

Configuration 3: The RNA adjuvant and the antigen-encoding RNA are on the same nucleic acid strand and encapsulated within the lipid carrier (FIG. 2C).

Configuration 4: The RNA is bi-cistronic. In this configuration, the RNA sequences encoding the adjuvant are translated from an internal ribosomal entry site (IRES) or the RNA adjuvant is separated from the antigen-coding region of the RNA with a STOP codon, a configuration enabling reduced relative translation of the downstream open reading frame (FIG. 2D).

### Example 5: TLR5 Agonist Adjuvant Compositions and NF-κB Signaling In Vitro

An mRNA encoding a TLR5 agonist adjuvant (TLR5 Agonist 1) was generated (SEQ ID NO: 2, SEQ ID NO: 176) by in vitro transcription (IVT). The mRNA was produced by transcribing a linearized plasmid DNA template (e.g., SEQ ID NO: 226). E. coli containing the plasmid are expanded in a bioreactor via fermentation, and then harvested to produce purified plasmid. The mRNA was produced by T7 RNA polymerase from the purified plasmid as described above in Example 2 and Example 3.

HEK293 cells were infected with a lentivirus comprising an NF-κB-driven luciferase reporter gene (referred to hereinafter as HEK293-NFkB-Luc cells) to generate a stable cell line. Upon, activation of a toll-like receptor (TLR) by an agonist, the luciferase activity increases in the HEK293-NFkB-Luc cells as measured by a luminescence detector. Furthermore, upon TLR activation by an mRNA adjuvant, the amount of secreted protein encoded by the mRNA adjuvant also increases as measured by an enzyme-linked immunosorbent assay (ELISA).

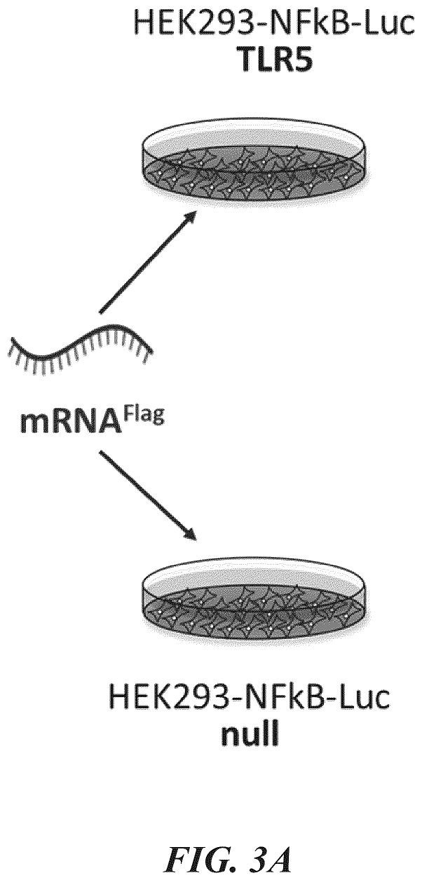

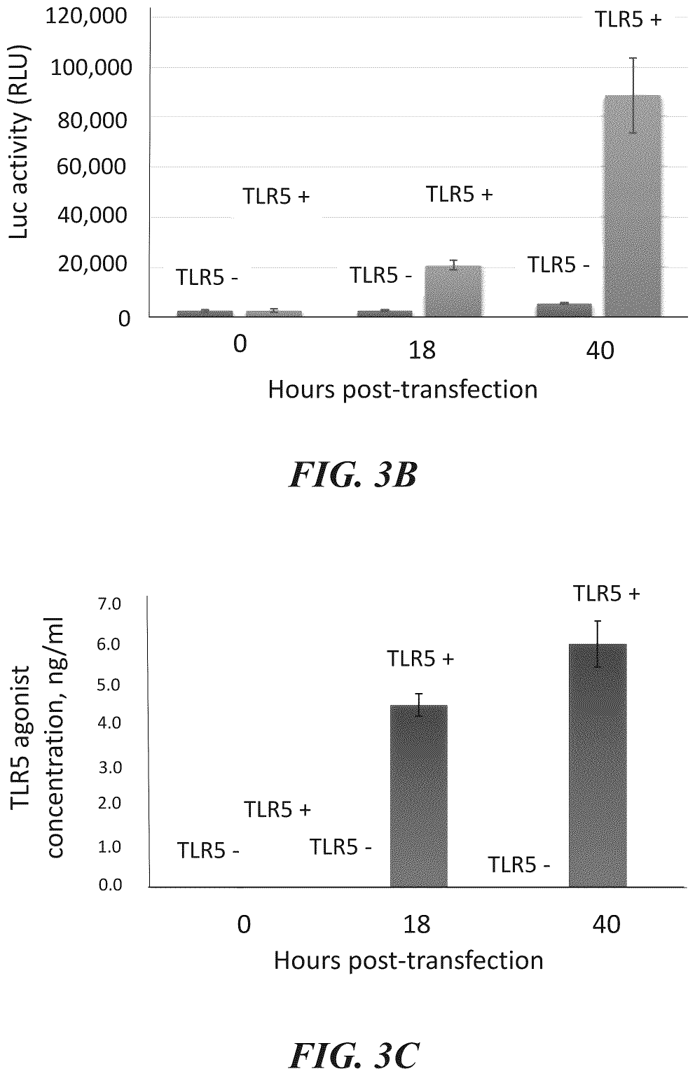

To determine the effect of mRNA adjuvants on TLR5-induced NF-κB signaling in vitro, HEK293-NFkB-Luc cells were transfected with TLR5 Agonist 1 using polyethylenimine (PEI) and compared with control HEK293-NFkB-Luc cells (null) (FIG. 3A). Luciferase activity readouts were performed and measured at 0, 18, and 40 hours post-transfection with the mRNA. NF-κB was activated by TLR5 Agonist 1 relative to control cells (FIG. 3B). Similarly, protein secretion of the TLR5 agonist was increased for cells that were transfected with TLR5 Agonist 1 relative to control cells at 18 and 40 hours post-transfection (FIG. 3C).

Next, TLR5 Agonist 1 mRNA was complexed with a lipid carrier to determine whether lipid nanoparticle packaging improved mRNA delivery. Lipid nanoparticles comprising a cationic lipid surface that includes PEG and DSPC and cholesterol were generated and purified.

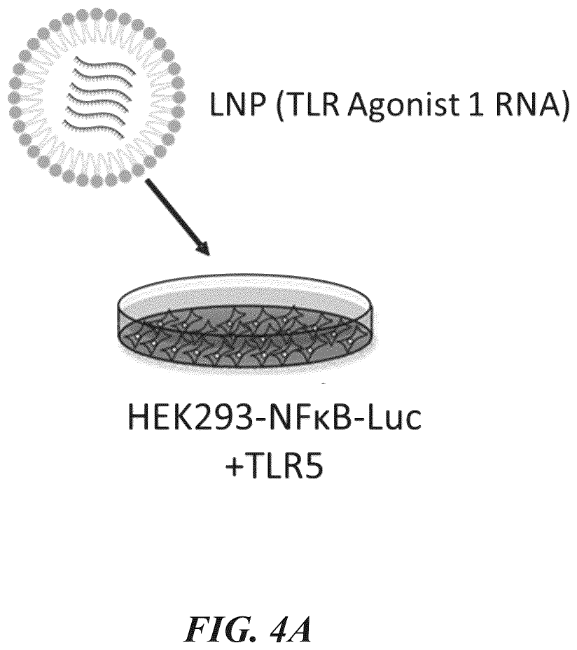

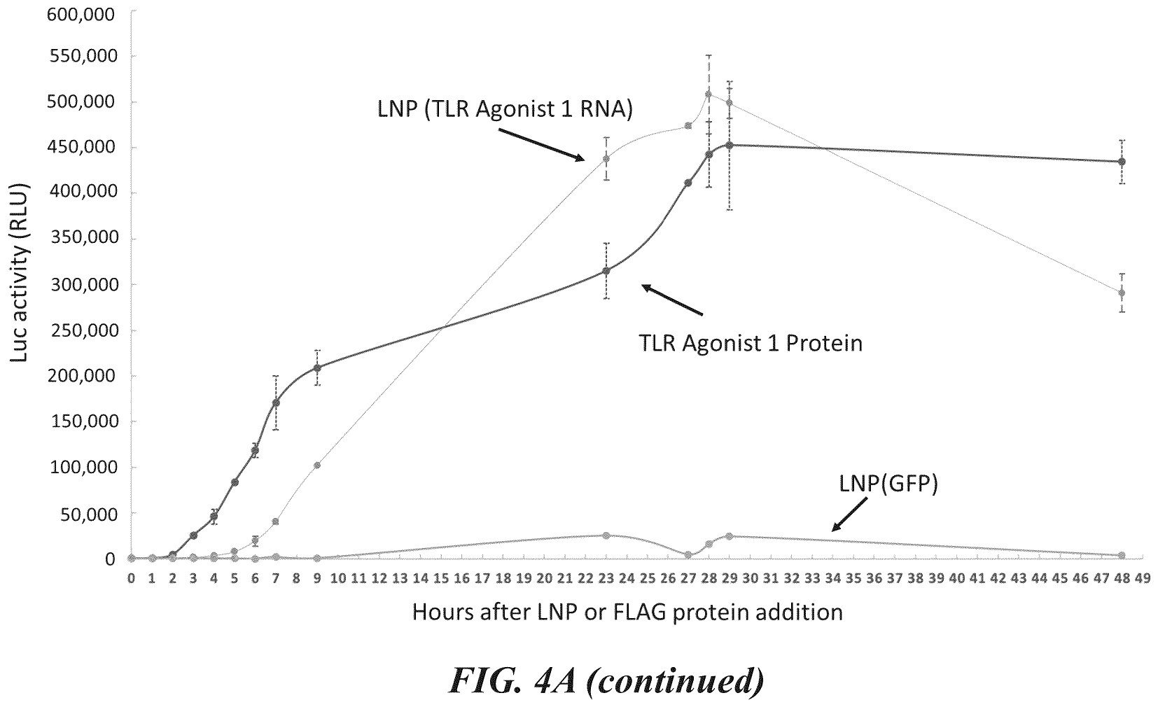

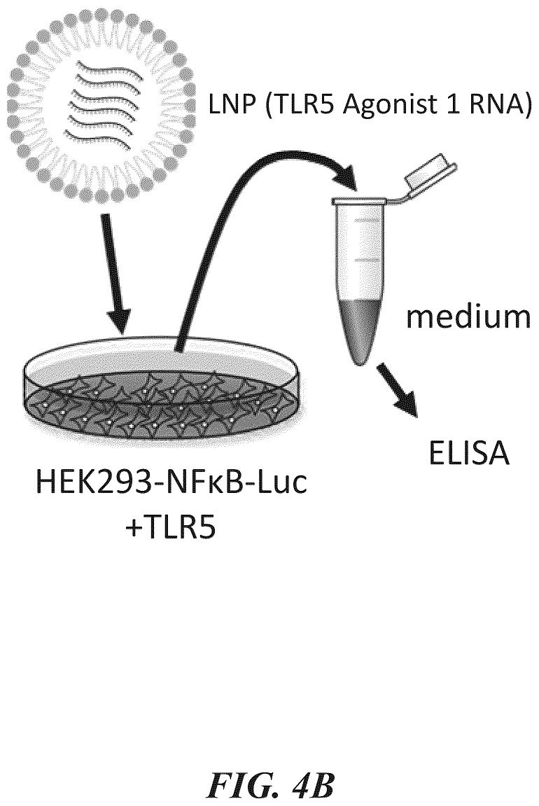

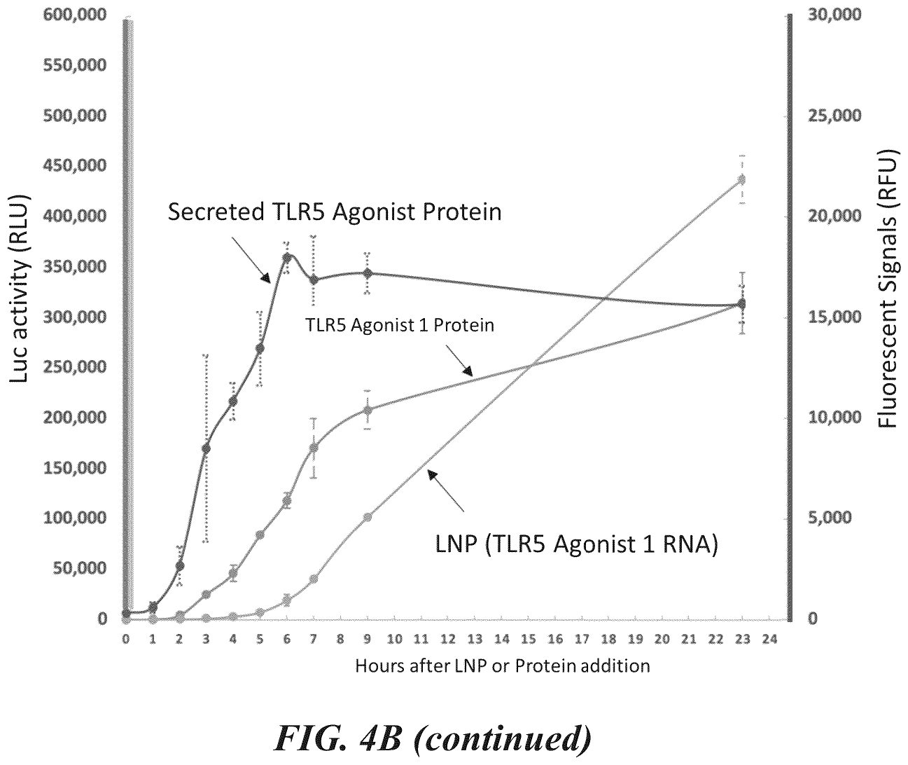

The purified LNPs complexed with TLR5 Agonist 1 were delivered to HEK293-NFkB-Luc cells. Time dependence of luciferase reporter activity was measured using HEK293-NFkB-Luc cells before (0 hours) and after 1 hour incubation with TLR5 Agonist 1 protein (500 ng/ml), LNP encapsulated RNA TLR5 Agonist 1 (LNP (TLR5 Agonist 1 RNA), 45 ng/ml), or LNP controls (LNP(GFP)). Luciferase activity was measured every hour up to 49 hours post-transfection for each condition (FIG. 4A). Medium was collected from each of the treated cell lines for ELISA analysis of TLR5 protein secretion. The LNP without the mRNA adjuvant (LNP (GFP)) did not activate the NF-κB-responsive luciferase reporter. In contrast, cell lines treated with either the LNP encapsulated mRNA TLR5 agonist (LNP (TLR5 Agonist 1 RNA) or the TLR5 Agonist 1 protein exhibited increased luciferase activity relative to controls. This result indicated that the effect observed was specific to the TLR5 agonist adjuvant. These results also showed that LNP delivery of RNA encoding TLR5 agonists can elicit robust secretion of TLR5 agonist protein relative to delivery of the protein alone or LNP controls for enhanced TLR5/NFκB cell signaling (FIG. 4B).

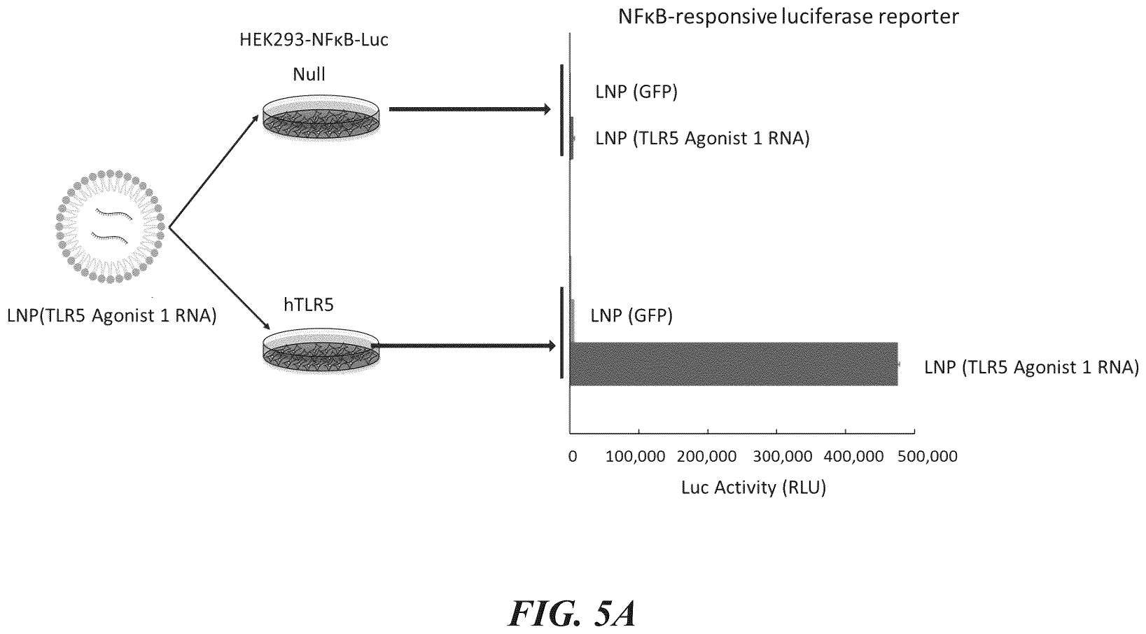

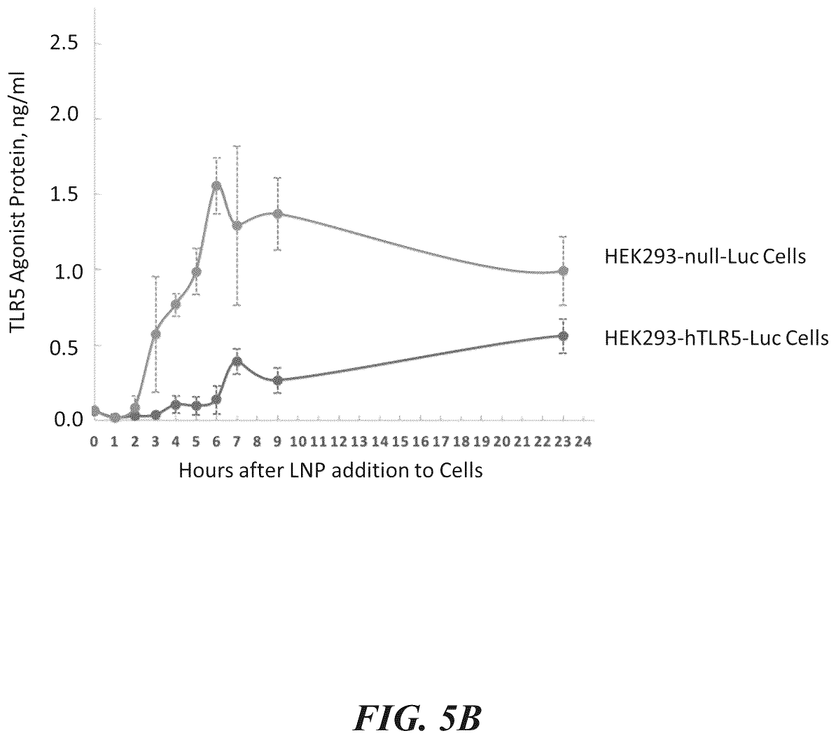

LNP-encapsulated TLR5 Agonist 1 RNA was administered to HEK293-NFkB-Luc cells expressing human TLR5 or HEK293-NFkB-Luc cells that did not express human TLR5 (FIG. 5A-5B). The LNP (TLR5 Agonist 1 RNA) increased both TLR5 luciferase activity (FIG. 5A) and TLR5 Agonist 1 protein secretion in human TLR5-expressing cells relative to cells that did not express human TLR5 (FIG. 5B).

### Example 6: In Vivo Biodistribution of LNP-mRNA Adjuvants

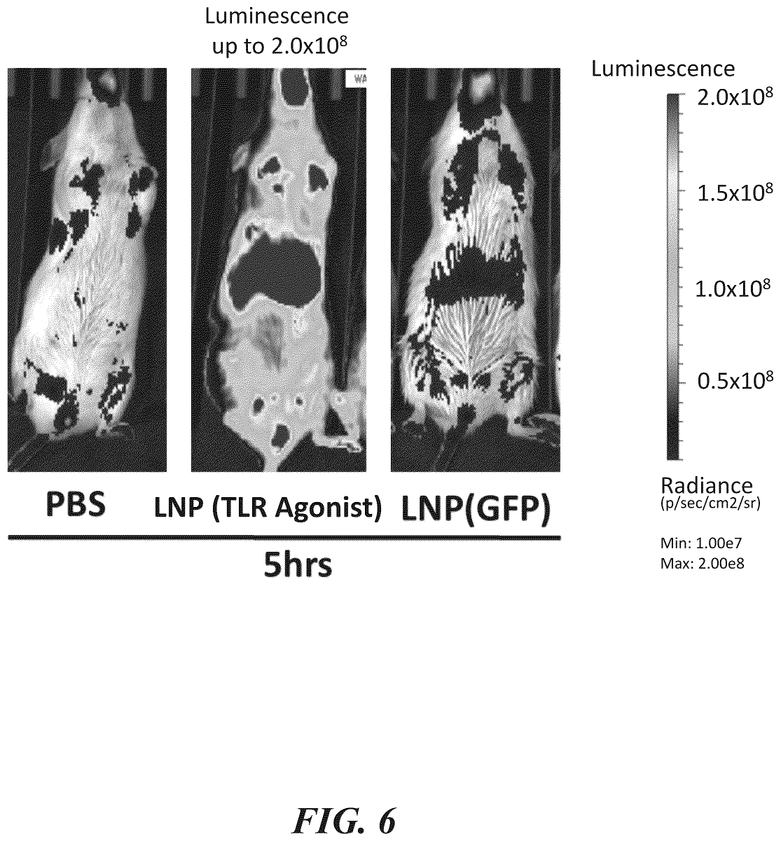

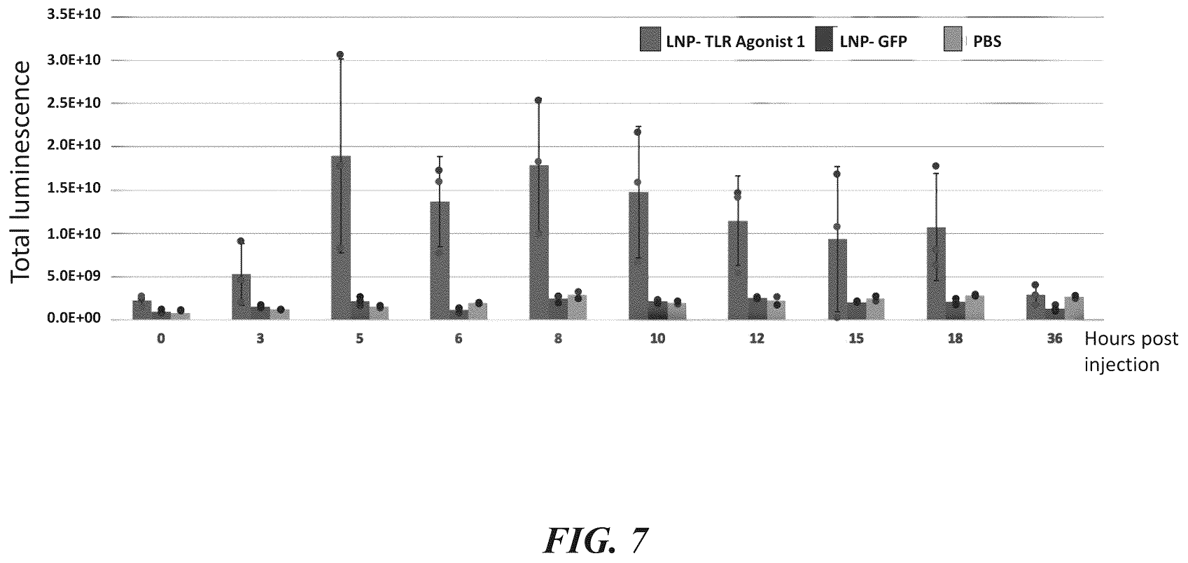

A mouse model was used to test in vivo biodistribution of RNA delivered via lipid nanoparticles. 38-week-old BALB/c-Tg(Rela-luc)31Xen females were injected intramuscularly (IM) with 20 μl of LNP-TLR5 Agonist 1(100 ng/μl of RNA), LNP-GFP, or PBS (FIG. 6). Animals were imaged under anesthesia using Perkin Elmer IVIS Spectrum In Vivo Imager for 5 minutes following injection with D-Luciferin Firefly, potassium salt (Gold Bio). Total luminescence was measured for LNP-TLR5 Agonist 1-treated animals; LNP-GFP treated animals; and PBS-treated animals for 0, 3, 5, 6, 8, 10, 12, 15, 18, and 36 hours post-injection. Animals treated with LNP-TLR5 agonist mRNA exhibited high levels of D-luciferin firefly with a broad distribution of the TLR5 protein throughout the animal (FIG. 7). The production and secretion of the TLR5 agonist and TLR5-dependent NF-κB activation was confirmed in vitro and in vivo. No adverse events were detected in the treated animals.

It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
(a) an RNA adjuvant encoding a synthetic protein, wherein the synthetic protein comprises at least a functional fragment of a toll-like receptor 5 (TLR5) agonist; and
(b) one or more delivery vehicle formulated for intranasal administration, oral administration, intradermal administration, intranodal administration, intrasplenic administration, intratumoral administration, intravenous administration, or intramuscular administration,
wherein following administration of an effective amount of the composition to a subject in the presence of an antigen, an initial innate immune response is induced in the subject.

2. The composition of claim 1, wherein the at least a functional fragment of the TLR5 agonist comprises a functional derivative of a bacterial motility protein.

3. The composition of claim 2, wherein the bacterial motility protein is a bacterial flagellin protein, a derivative, or a functional fragment thereof.

4. The composition of claim 1, wherein the synthetic protein is a truncated form of a bacterial flagellin protein, a secreted form of a bacterial flagellin protein, or a combination thereof.

5. The composition of claim 1, wherein the functional fragment of a toll-like receptor 5 (TLR5) agonist comprises a D0 region and a D1 region of the bacterial flagellin protein.

6. The composition of claim 1, wherein the functional fragment of a toll-like receptor 5 (TLR5) agonist comprises a D0 region, a D1 region, and a D2 region of the bacterial flagellin protein.

7. The composition of claim 1, wherein the functional fragment of a toll-like receptor 5 (TLR5) agonist comprises a D0 region, a D1 region, a D2 region, and a D3 region of the bacterial flagellin protein.

8. (canceled)

9. (canceled)

10. The composition of claim 1, wherein the RNA comprises a sequence that is at least 85% identical to any one of SEQ ID NOS: 2 to 37.

11. (canceled)

12. (canceled)

13. (canceled)

14. The composition of claim 1, wherein the one or more delivery vehicle is a diluent, an aqueous solution, a particle, an emulsion, a suspension, a lipid carrier, a vesicle, a vector, or any combination thereof.

15. The composition of claim 1, further comprising:
(i) one or more antigens; or
(ii) one or more RNA sequences each encoding for an antigen.

16. The composition of claim 15, wherein the one or more antigens comprise a viral antigen, a bacterial antigen, a fungal antigen, a parasitic antigen, or a tumor antigen.

17. The composition of claim 1, further comprising an RNA encoding the antigen, wherein the RNA encoding the antigen is within the same delivery vehicle as the RNA adjuvant.

18. The composition of claim 1, further comprising an RNA encoding the antigen, wherein the RNA encoding the antigen is within a different delivery vehicle as the RNA adjuvant.

19. The composition of claim 1, wherein the RNA adjuvant further comprises an RNA sequence encoding the antigen, and wherein the RNA sequence encoding the antigen is operably linked to the RNA adjuvant.

20.-21. (canceled)

22. The composition of claim 1, wherein the composition further comprises
a lipid carrier.

23. The composition of claim 22, wherein the RNA adjuvant is encapsulated within the lipid carrier.

24. The composition of claim 22, wherein the RNA adjuvant is in complex with the lipid carrier.

25.-167. (canceled)

168. A pharmaceutical composition comprising the composition of claim 1; and a pharmaceutically acceptable excipient.

169.-171. (canceled)

172. A method of enhancing an immune response to an antigen encoded by an RNA in an RNA vaccine composition, the method comprising:
(a) administering to the subject an effective amount of the composition of claim 1; and
(b) administering to the subject an RNA vaccine composition, wherein the RNA vaccine composition comprises an RNA encoding for an antigen, thereby enhancing the immune response to the antigen encoded by the RNA.

173.-195. (canceled)
